(19) United States
(12) Patent Application Publication
(10) Pub. No.: US 2003/0104473 A1
(43) Pub. Date: Jun. 5, 2003

(54) Common ligand mimics: benzimidazoles

(76) Inventors: Qing Dong, San Diego, CA (US); Hengyuan Lang, San Diego, CA (US); Lan Xie, Beijing (CN); Lin Yu, San Diego, CA (US)

Correspondence Address: CAMPBELL & FLORES LLP, 7th Floor, 4370 La Jolla Village Drive, San Diego, CA, 92122, US

(21) Appl. No.: 10/097,181

(22) Filed: Mar. 12, 2002

(30) Foreign Application Priority Data
Apr. 24, 2001 (JP) 2001-126437

(51) Int. Cl. C 07D 4 5/04; G01N 33/53

(52) U.S. Cl. 435/7.1; 548/113; 548/304.7

(57) ABSTRACT

The present invention provides common ligand mimics that act as common ligands for a receptor family. The present invention also provides bi-ligands containing these common ligand mimics. Bi-ligands of the invention provide enhanced affinity and/or selectivity of ligand binding to a receptor or receptor family through the synergistic action of the common ligand mimic and specificity ligand that compose the bi-ligand. The present invention also provides combinatorial libraries containing the common ligand mimics and bi-ligands of the invention. Further, the present invention provides methods for manufacturing the common ligand mimics and bi-ligands of the invention and methods for assaying the combinatorial libraries of the invention.

### BACKGROUND OF THE INVENTION

### FIELD OF THE INVENTION

The present invention relates generally to receptor/ligand interactions and to combinatorial libraries of ligand compounds. The present invention also relates to the manufacture of benzimidazoles and combinatorial libraries containing such compounds.

### BACKGROUND INFORMATION

Two general approaches have traditionally been used for drug discovery: screening for lead compounds and structure-based drug design. Both of these approaches are laborious and time-consuming and often produce compounds that lack the desired affinity or specificity.

Screening for lead compounds involves generating a pool of candidate compounds, often using combinatorial chemistry approaches in which compounds are synthesized by combining chemical groups to generate a large number of diverse candidate compounds that bind to the target or that inhibit binding to the target. The candidate compounds are screened with a drug target of interest to identify lead compounds that bind to the target or inhibit binding to the target. However, the screening process to identify a lead compound can be laborious and time consuming.

Structure-based drug design is an alternative approach to identifying drug candidates. Structure-based drug design uses three-dimensional structural data of the drug target as a template to model compounds that bind to the drug target and alter its activity. The compounds identified as potential drug candidates using structural modeling are used as lead compounds for the development of drug candidates that exhibit a desired activity toward the drug target.

Identifying compounds using structure-based drug design can be advantageous when compared to the screening approach in that modifications to the compound can often be predicted by modeling studies. However, obtaining structures of relevant drug targets and of drug targets complexed with test compounds is extremely time-consuming and laborious, often taking years to accomplish. The long time period required to obtain structural information useful for developing drug candidates is particularly limiting with regard to the growing number of newly discovered genes, which are potential drug targets, identified in genomics studies.

Despite the time-consuming and laborious nature of these approaches to drug discovery, both screening for lead compounds and structure-based drug design have led to the identification of a number of useful drugs, such as receptor agonists and antagonists. However, many of the drugs identified by these approaches have unwanted toxicity or side effects. Therefore, there is a need in the art for drugs that have high specificity and reduced toxicity. For example, in addition to binding to the drug target in a pathogenic organism or cancer cell, in some cases the drug also binds to an analogous protein in the patient being treated with the drug, which can result in toxic or unwanted side effects. Therefore, drugs that have high affinity and specificity for a target are particularly useful because administration of a more specific drug at lower dosages will minimize toxicity and side effects.

In addition to drug toxicity and side effects, a number of drugs that were previously highly effective for treating certain diseases have become less effective during prolonged clinical use due to the development of resistance. Drug resistance has become increasingly problematic, particularly with regard to administration of antibiotics. A number of pathogenic organisms have become resistant to several drugs due to prolonged clinical use and, in some cases, have become almost totally resistant to currently available drugs. Furthermore, certain types of cancer develop resistance to cancer therapeutic agents. Therefore, drugs that are refractile to the development of resistance would be particularly desirable for treatment of a variety of diseases.

One approach to developing such drugs is to find compounds that bind to a target protein such as a receptor or enzyme. When such a target protein has two adjacent binding sites, it is especially useful to find “bi-ligand” drugs that can bind at both sites simultaneously. However, the rapid identification of bi-ligand drugs having the optimum combination of affinity and specificity has been difficult. Bi-ligand drug candidates have been identified using rational drug design, but previous methods are time-consuming and require a precise knowledge of structural features of the receptor. Recent advances in nuclear magnetic spectroscopy (NMR) have allowed the determination of the three-dimensional interactions between a ligand and a receptor in a few instances. However, these efforts have been limited by the size of the receptor and can take years to map and analyze the complete structure of the complexes of receptor and ligand.

Thus, there exists a need for compounds that bind to multiple members of a receptor family. There is also a need for receptor bi-ligands containing such compounds coupled to ligands having a high specificity for the receptor.

There is a further need in the art for methods of preparing such compounds and bi-ligands. There is also a need in the art for methods of preparing combinatorial libraries of the bi-ligands and methods of screening these libraries to find bi-ligands that interact with a drug target with improved affinity and/or specificity. The present invention satisfies these needs and provides related advantages as well.

### SUMMARY OF THE INVENTION

The present invention provides compounds that function as mimics to a natural common ligand for a receptor family. These compounds interact with a conserved binding site on multiple receptors within the receptor family.

In one aspect, the present invention provides compounds that are common ligand mimics for NAD. NAD is a natural common ligand for many oxidoreductases. Thus, compounds of the invention that are common ligand mimics for NAD interact selectively with conserved sites on oxidoreductases.

In one embodiment, the present invention provides benzimidazole compounds of Formula I,

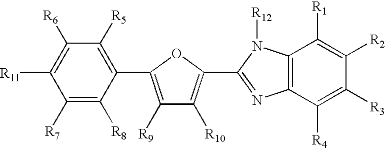

wherein R₁ to R₁₁ each independently are H, alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S (O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X. R₁₂, R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring.

In a second aspect, the present invention provides methods for preparing compounds of Formula I. These methods generally comprise two steps. First, 5-trimethylstannanyl-furan-2-carbaldehyde is reacted with a benzene derivative in the presence of tetrakis(triphenyl-phosphine) palladium to form a furanyl intermediate. Suitable benzene derivatives include halobenzenes. Any halobenzene can be used in the reaction. For example, iodobenzenes or bromobenzenes, such as 4-bromobenzoate can be employed. Then, the furanyl intermediate is reacted with a benzodiamine, such as 1,2-phenylenediamine in the presence of benzoquinone. Where the compound produced is a methyl ester, it can then be reacted with lithium hydroxide to form the corresponding benzoic acid.

In a third aspect, the present invention provides bi-ligands containing a common ligand mimic and a specificity ligand which interact with distinct sites on a receptor. In one embodiment, the present invention provides bi-ligands that are the reaction products of compounds of Formula I with specificity ligands. In yet another aspect, the invention provides methods for preparing bi-ligands that are reaction products of the common ligand mimics of general Formula I and a pyridine dicarboxylate specificity ligand.

The present invention further provides combinatorial libraries containing one or more common ligand variants of the compounds of the invention. In one embodiment, the combinatorial libraries of the invention contain one or more common ligand variants of the compounds of Formula I.

The present invention also provides combinatorial libraries comprised of one or more bi-ligands that are reaction products of common ligand mimics and specificity ligands. In one embodiment, such combinatorial libraries contain one or more bi-ligands that are the reaction product of compounds of Formula I and specificity ligands.

The present invention also provides methods for producing and screening combinatorial libraries of bi-ligands for binding to a receptor and families of such receptors.

### BRIEF DESCRIPTION OF THE DRAWINGS

FIG. 1 shows Scheme 1 for the synthesis of benzimidazole compounds of Formula I where R₁ to R₁₁ each independently are H, alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R,₁₅, PO₄H₂, H₂PO₃, H₂PO₂, HPO₄R₁₅, PO₂R₁₄R₁₅, CN, or X. R₁₂, R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring. The reaction steps are as follows: (a) 5-trimethylstannanyl-furan-2-carbaldehyde is formed from 4-methlypiperidine, (b) the 5-trimethylstannanyl-furan-2-carbaldehyde is then reacted with 4-bromobenzoate in the presence of tetrakis(triphenylphosphine)palladium to form a furanyl intermediate, (3) the intermediate is reacted with a phenylenediamine, and (4) the methyl ester of step (3) optionally is reacted with lithium hydroxide to form the corresponding benzoic acid.

FIG. 2 shows Scheme 2 for the synthesis of bi-ligands containing benzimidazole common ligand mimics and pyridine dicarbolxylate specificity ligands. The reaction steps are as follows: (a) a pyridine dicarboxylate specificity ligand is reacted with a benzimidazole common ligand mimic in the presence of HOBt.H₂O in dichloroethane, followed by reaction with potassium hydroxide.

FIG. 3 shows a reaction scheme for preparation of a benzimidazole common ligand mimic of the invention having a carboxylic acid substituent.

FIG. 4 shows a reaction scheme for modification of substituents attached to the common ligand mimics of the invention.

FIGS. 5a-c show various reaction schemes by which combinatorial libraries of the present invention can be made. FIG. 5a shows the reaction scheme for reaction of common ligand mimics of the present invention having a carboxylic acid group with an amine in the presence of hydroxybenzotriazole (HOBt). FIG. 5b shows the reaction of common ligand mimics of the invention having an amine terminal amide substituent with a carboxylic acid in the presence of HOBt. FIG. 5c shows the reaction scheme for reaction of common ligand mimics of the invention having an amine terminal amide substituent with an isocyanate or thioisocyanate.

FIG. 6 shows the reaction scheme for the reaction 4-[5-(5-nitro-1H-benzoimidazol-2-yl)-furan-2-yl]-benzoic acid with an amine in the presence of hydroxybenzotriazole (HOBt). This is a specific example of the reaction depicted in FIG. 5a.

FIG. 7 shows the results of a oxidoreductase enzymatic panel study of selected benzimidazole compounds of the invention.

FIG. 8 shows DHPR assay results for selected benzimidazole common ligand mimics of the invention.

FIG. 9 shows the results of a dehydrogenase assay of selected bi-ligands of the invention.

FIGS. 10a-10b shows the names and corresponding structures for exemplified benzimidazole common ligand mimics of the invention.

FIG. 11 shows examples of bi-ligands of the invention.

### DETAILED DESCRIPTION OF THE INVENTION

The present invention is directed to bi-ligands and the development of combinatorial libraries associated with these bi-ligands. The invention advantageously can be used to develop bi-ligands that bind to two distinct sites on a receptor, a common site and a specificity site. Tailoring of the two portions of the bi-ligand provides optimal binding characteristics. These optimal binding characteristics provide increased diversity within a library, while simultaneously focusing the library on a particular receptor family or a particular member of a receptor family. The two portions of the bi-ligand, a common ligand mimic and a specificity ligand act synergistically to provide higher affinity and/or specificity than either ligand alone.

The technology of the present invention can be applied across receptor families or can be used to screen for specific members of a family. For example, the present invention can be used to screen libraries for common ligand mimics that bind to any oxidoreductase. Alternatively, the present invention can be used to screen for a particular oxidoreductase that will bind a particular specificity ligand.

The present invention provides common ligand mimics that bind selectively to a conserved site on a receptor. The compounds advantageously can be used to develop combinatorial libraries of bi-ligands more efficiently than conventional methods. The present invention takes advantage of NMR spectroscopy to identify the interactions between the common ligand mimic and the receptor, which allows for improved tailoring of the ligand to the receptor.

The present invention also provides bi-ligands containing these common ligand mimics. The bi-ligands of the invention contain a common ligand mimic coupled to a specificity ligand. These bi-ligands provide the ability to tailor the affinity and/or specificity of the ligands to the binding sites on the receptor.

The present invention further provides combinatorial libraries containing bi-ligands of the invention as well as formation of such libraries from the common ligand mimics of the invention. These libraries provide an enhanced number of bi-ligands that will bind multiple members of a receptor family than is provided with standard combinatorial techniques due to specific positioning of the specificity ligand on the common ligand mimic. Optimal positioning of the specificity ligand can be determined through NMR studies of the receptor and the common ligand mimic to be employed.

The present invention also provides methods for the preparation of benzimidazole common ligand mimics useful in the present invention and methods for the preparation of bi-ligands containing these common ligand mimics. In general, such methods involve formation of a furanyl intermediate followed by reaction of the intermediate with a phenylenediamine. The present invention also provides methods for modification of the common ligand mimics to form additional common lignad mimics having different bi-ligand directing/binding substituents. The common ligand mimics can be used to create bi-ligands having improved affinity, improved specificity, or both. These and other aspects of the invention are described below.

The present invention provides common ligand mimics. As used herein, the term “ligand” refers to a molecule that can selectively bind to a receptor. The term “selectively” means that the binding interaction is detectable over non-specific interactions as measured by a quantifiable assay. A ligand can be essentially any type of molecule such as an amino acid, peptide, polypeptide, nucleic acid, carbohydrate, lipid, or small organic compound. The term ligand refers both to a molecule capable of binding to a receptor and to a portion of such a molecule, if that portion of a molecule is capable of binding to a receptor. For example, a bi-ligand, which contains a common ligand and specificity ligand, is considered a ligand, as would the common ligand and specificity ligand portions since they can bind to a conserved site and specificity site, respectively. As used herein, the term “ligand” excludes a single atom, for example, a metal atom. Derivatives, analogues, and mimetic compounds also are included within the definition of this term. These derivatives, analogues and mimetic compounds include those containing metals or other inorganic molecules, so long as the metal or inorganic molecule is covalently attached to the ligand in such a manner that the dissociation constant of the metal from the ligand is less than 10⁻¹⁴ M. A ligand can be multi-partite, comprising multiple ligands capable of binding to different sites on one or more receptors, such as a bi-ligand. The ligand components of a multi-partite ligand can be joined together directly, for example, through functional groups on the individual ligand components or can be joined together indirectly, for example, through an expansion linker.

As used herein, the term “common ligand” refers to a ligand that binds to a conserved site on receptors in a receptor family. A “natural common ligand” refers to a ligand that is found in nature and binds to a common site on receptors in a receptor family. As used herein, a “common ligand mimic (CLM)” refers to a common ligand that has structural and/or functional similarities to a natural common ligand but is not naturally occurring. Thus, a common ligand mimic can be a modified natural common ligand, for example, an analogue or derivative of a natural common ligand. A common ligand mimic also can be a synthetic compound or a portion of a synthetic compound that is structurally similar to a natural common ligand.

As used herein, a “common ligand variant” refers to a derivative of a common ligand. A common ligand variant has structural and/or functional similarities to a parent common ligand. A common ligand variant differs from another variant, including the parent common ligand, by at least one atom. For example, as with NAD and NADH, the reduced and oxidized forms differ by an atom and are therefore considered to be variants of each other. A common ligand variant includes reactive forms of a common ligand mimic, such as an anion or cation of the common ligand mimic. As used herein, the term “reactive form” refers to a form of a compound that can react with another compound to form a chemical bond, such as an ionic or covalent bond. For example, where the common ligand mimic is an acid of the form ROOH or an ester of the form ROOR′, the common ligand variant can be ROO⁻.

As used herein, the term “conserved site” on a receptor refers to a site that has structural and/or functional characteristics common to members of a receptor family. A conserved site contains amino acid residues sufficient for activity and/or function of the receptor that are accessible to binding of a natural common ligand. For example, the amino acid residues sufficient for activity and/or function of a receptor that is an enzyme can be amino acid residues in a substrate binding site of the enzyme. Also, the conserved site in an enzyme that binds a cofactor or coenzyme can be amino acid residues that bind the cofactor or coenzyme.

As used herein, the term “receptor” refers to a polypeptide that is capable of selectively binding a ligand. The function or activity of a receptor can be enzymatic activity or ligand binding. Receptors can include, for example, enzymes such as kinases, dehydrogenases, oxidoreductases, GTPases, carboxyl transferases, acyl transferases, decarboxylases, transaminases, racemases, methyl transferases, formyl transferases, and a-ketodecarboxylases.

Furthermore, the receptor can be a functional fragment or modified form of the entire polypeptide so long as the receptor exhibits selective binding to a ligand. A functional fragment of a receptor is a fragment exhibiting binding to a common ligand and a specificity ligand. As used herein, the term “enzyme” refers to a molecule that carries out a catalytic reaction by converting a substrate to a product.

Enzymes can be classified based on Enzyme Commission (EC) nomenclature recommended by the Nomenclature Committee of the International Union of Biochemistry and Molecular Biology (IUBMB)(see, for example, www.expasy.ch/sprot/enzyme.html)(which is incorporated herein by reference). For example, oxidoreductases are classified as oxidoreductases acting on the CH—OH group of donors with NAD⁺ or NADP⁺ as an acceptor (EC 1.1.1); oxidoreductases acting on the aldehyde or oxo group of donors with NAD⁺ or NADP⁺ as an acceptor (EC 1.2.1); oxidoreductases acting on the CH—CH group of donors with NAD⁺ or NADP⁺ as an acceptor (EC 1.3.1); oxidoreductases acting on the CH—NH₂ group of donors with NAD⁺ or NADP⁺ as an acceptor (EC 1.4.1); oxidoreductases acting on the CH—NH group of donors with NAD⁺ or NADP⁺ as an acceptor (EC 1.5.1); oxidoreductases acting on NADH or NADPH (EC 1.6); and oxidoreductases acting on NADH or NADPH with NAD⁺ or NADP⁺ as an acceptor (EC 1.6.1).

Additional oxidoreductases include oxidoreductases acting on a sulfur group of donors with NAD⁺ or NADP⁺ as an acceptor (EC 1.8.1); oxidoreductases acting on diphenols and related substances as donors with NAD⁺ or NADP⁺ as an acceptor (EC 1.10.1); oxidoreductases acting on hydrogen as donor with NAD⁺ or NADP⁺ as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NAD⁺ or NADP⁺ as an acceptor (EC 1.16.1); oxidoreductases acting on —CH₂ groups with NAD⁺ or NADP⁺ as an acceptor (EC 1.17.1); and oxidoreductases acting on reduced ferredoxin as donor, with NAD⁺ or NADP⁺ as an acceptor (EC 1.18.1).

Enzymes can also bind coenzymes or cofactors such as nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, and tetrahydrofolate or other cofactors or substrates such as ATP, GTP and S-adenosyl methionine (SAM). In addition, enzymes that bind newly identified cofactors or enzymes can also be receptors.

As used herein, the term “receptor family” refers to a group of two or more receptors that share a common, recognizable amino acid motif. A motif in a related family of receptors occurs because certain amino acid residues, or residues having similar chemical characteristics, are required for the structure, function and/or activity of the receptor and are, therefore, conserved between members of the receptor family. Methods of identifying related members of a receptor family are well known to those skilled in the art and include sequence alignment algorithms and identification of conserved patterns or motifs in a group of polypeptides, which are described in more detail below. Members of a receptor family also can be identified by determination of binding to a common ligand.

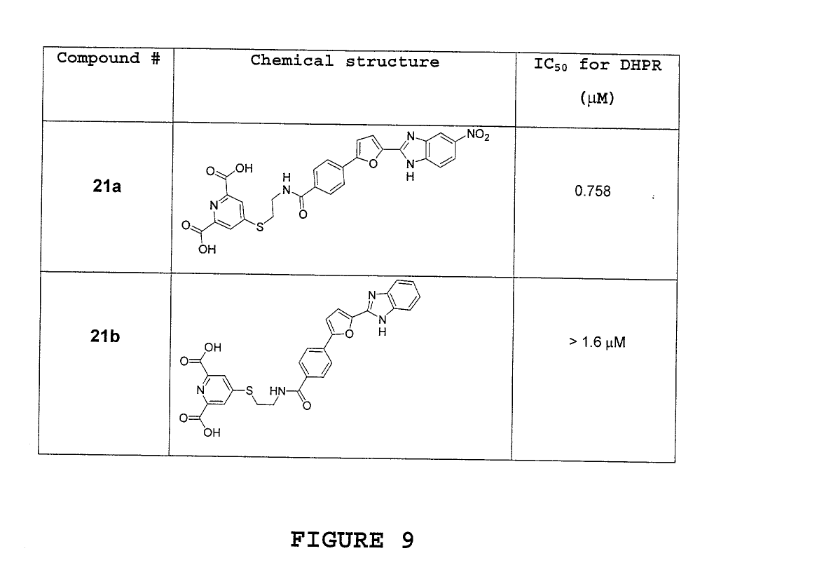

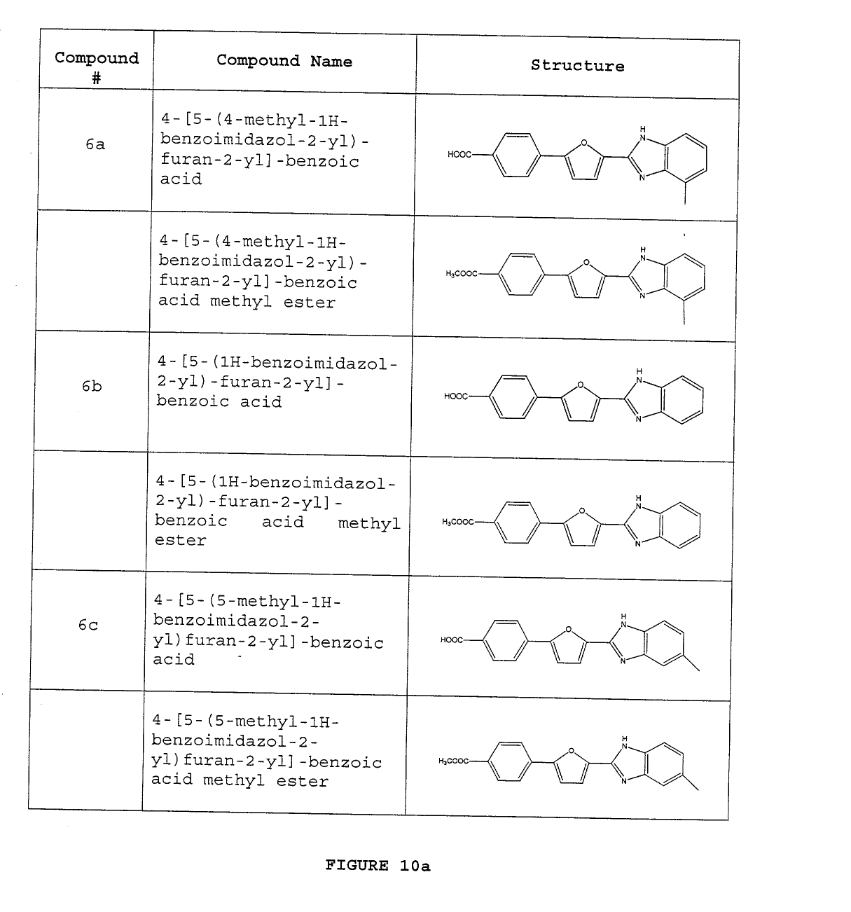

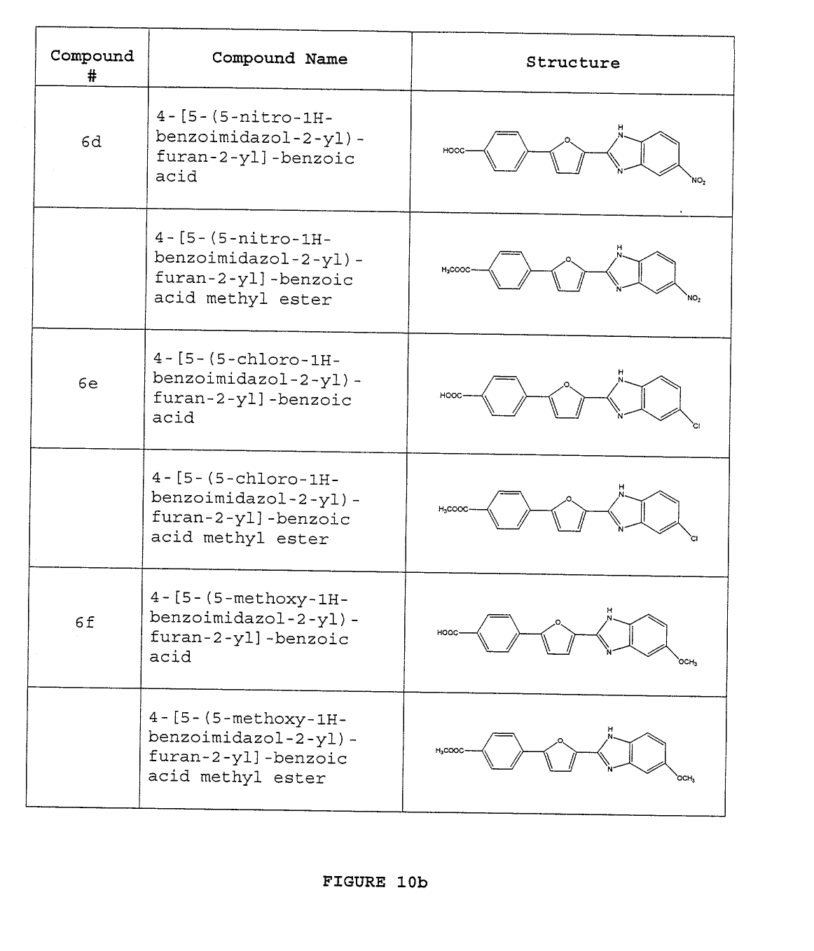

In another aspect, the present invention provides bi-ligands that contain a common ligand mimic as described above and a specificity ligand. As used herein, the term “bi-ligand” refers to a ligand comprising two ligands that bind to independent sites on a receptor. One of the ligands of a bi-ligand is a specificity ligand capable of binding to a site that is specific for a given member of a receptor family when joined to a common ligand. The second ligand of a bi-ligand is a common ligand mimic that binds to a conserved site in a receptor family. The common ligand mimic and specificity ligand are bonded together. Bonding of the two ligands can be direct or indirect, such as through a linking molecule or group. A depiction of exemplary bi-ligands is shown in FIG. 9.

As used herein the term “specificity” refers to the ability of a ligand to differentially bind to one receptor over another receptor in the same receptor family. The differential binding of a particular ligand to a receptor is measurably higher than the binding of the ligand to at least one other receptor in the same receptor family. A ligand having specificity for a receptor refers to a ligand exhibiting specific binding that is at least two-fold higher for one receptor over another receptor in the same receptor family.

As used herein, the term “specificity ligand” refers to a ligand that binds to a specificity site on a receptor. A specificity ligand can bind to a specificity site as an isolated molecule or can bind to a specificity site when attached to a common ligand, as in a bi-ligand. When a specificity ligand is part of a bi-ligand, the specificity ligand can bind to a specificity site that is proximal to a conserved site on a receptor.

As used herein, the term “specificity site” refers to a site on a receptor that provides the binding site for a ligand exhibiting specificity for a receptor. A specificity site on a receptor imparts molecular properties that distinguish the receptor from other receptors in the same receptor family. For example, if the receptor is an enzyme, the specificity site can be a substrate binding site that distinguishes two members of a receptor family which exhibit substrate specificity. A substrate specificity site can be exploited as a potential binding site for the identification of a ligand that has specificity for one receptor over another member of the same receptor family. A specificity site is distinct form the common ligand binding site in that the natural common ligand does not bind to the specificity site.

As used herein, the term “linker” refers to a chemical group that can be attached to either the common ligand or the specificity ligand of a bi-ligand. The provides the functional groups through which the common ligand mimic and the specificity ligand are idirectly bound to one another. The linker can be a simple functional group, such as COOH, NH₂, OH, or the like. Alternatively, the linker can be a complex chemical group containing one or more unsaturation, one or more substituent, and/or one or more heterocyclic atom. Nonlimiting examples of complex linkers are depicted in Tables 5 to 11.

The present invention provides common ligand mimics that are common mimics of NAD and combinatorial libraries containing these common ligand mimics. For example, in one embodiment, compounds of the invention are ligands for conserved sites on oxidoreductases.

Examples of such receptors include, but are not limited to, HMG CoA reductase (HMGCoAR), inosine-5′-monophosphate dehydrogenase (IMPDH), 1-deoxy-D-xylulose-5-phosphate reductase (DOXPR), dihydrodipicolinate reductase (DHPR), dihydrofolate reductase (DHFR), 3-isopropylmalate (IPMDH), glyceraldehyde-3-phosphate dehydrogenase (GAPDH), aldose reductase (AR), alcohol dehydrogenase (ADH), and lactate dehydrogenase (LDH) and enoyl ACP reductase.

The present invention also provides compounds and combinatorial libraries of compounds of the formula:

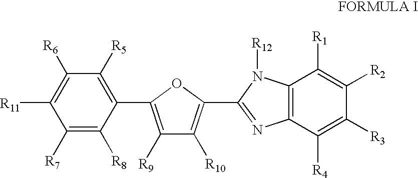

wherein R₁to R₁₁ each independently are H, alkyl, alkenyl, alkynyl, aryl, heterocycle, COOH, COOAlkyl, CONR₁₃R₁₄, C(O)R₁₅, OH, OAlkyl, OAc, SH, SR,₁₅, SO₃H, S(O)R,₁₅, SO₂NR₁₃R₁₄, S(O)₂R₁₅, NH₂, NHR₁₅, NR₁₃R14, NHCOR₁₅, N₃, NO₂, PH₃, PH₂R₁₅, PO₄H₂, H₂PO₃, H₂PO₂, HPO₄R₁₅, PO₂R₁₄R ₁₅, CN, or X. R₁₂, R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring.

As used herein, “alkyl” means a carbon chain having from one to twenty carbon atoms. The alkyl group of the present invention can be straight chain or branched. It can be unsubstituted or can be substituted. When substituted, the alkyl group can have up to ten substituent groups, such as COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R ₁₅, CN, or X where R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring.

Additionally, the alkyl group present in the compounds of the invention, whether substituted or unsubstituted, can have one or more of its carbon atoms replaced by a heterocyclic atom, such as an oxygen, nitrogen, or sulfur atom. For example, alkyl as used herein includes groups such as (OCH₂CH₂)ₙ or (OCH₂CH₂ CH₂)ₙ, where n has a value such that there are twenty or less carbon atoms in the alkyl group. Similar compounds having alkyl groups containing a nitrogen or sulfur atom are also encompassed by the present invention.

As used herein “alkenyl” means an unsaturated alkyl groups as defined above, where the unsaturation is in the form of a double bond. The alkenyl groups of the present invention can have one or more unsaturations. Nonlimiting examples of such groups include CH═CH₂, CH₂CH₂CH═CHCH₂CH₃, and CH₂CH═CHCH₃. As used herein “alkynyl” means an unsaturated alkyl group as defined above, where the unsaturation is in the form of a triple bond. Alkynyl groups of the present invention can include one or more unsaturations. Nonlimiting examples of such groups include C≡CH, CH₂CH₂C≡CCH₂CH₃, and CH₂C≡CCH₃.

The compounds of the present invention can include compounds in which R₁ to R₁₁ each independently are complex substituents containing one or more unsaturation, one or more substituent, and/or one or more heterocyclic atom. These complex substituents are also referred to herein as “linkers” or “expansion linkers.” Nonlimiting examples of complex substituents that can be used in the present invention are presented in Tables 5 to 11.

As used herein, “aromatic group” refers to a group that has a planar ring with 4n+2 pi-electrons, where in is a positive integer. The term “aryl” as used herein denotes a nonheterocyclic aromatic compound or group, for example, a benzene ring or naphthalene ring.

As used herein, “heterocyclic group” or “heterocycle” refers to an aromatic compound or group containing one or more heterocyclic atom. Nonlimiting examples of heterocyclic atoms that can be present in the heterocyclic groups of the invention include nitrogen, oxygen and sulfur. In general, heterocycles of the present invention will have from five to seven atoms and can be substituted or unsubstituted. When substituted, substituents include, for example, those groups provided for R₁ to R₁₁. Nonlimiting examples of heterocyclic groups of the invention include pyroles, pyrazoles, imidazoles, pyridines, pyrimidines, pyridzaines, pyrazines, triazines, furans, oxazoles, thiazoles, thiophenes, diazoles, triazoles, tetrazoles, oxadiazoles, thiodiazoles, and fused heterocyclic rings, for example, indoles, benzofurans, benzothiophenes, benzoimidazoles, benzodiazoles, benzotriazoles, and quinolines.

As used herein, the variable “X” indicates a halogen atom. Halogens suitable for use in the present invention include chlorine, fluorine, iodine, and bromine, with bromine being particularly useful. As used herein, “Ac” denotes an acyl group. Suitable acyl groups can have, for example, an alkyl, alkenyl, alkynyl, aromatic, or heterocyclic group as defined above attached to the carbonyl group.

The benzimidazole ring system in Formula I can be substituted with one or multiple substituents. Variation in the substitution on the benzimidazole provides compounds that allow for addition of a specificity ligand to directed sites on the benzimidazole to increase the binding of the specificity ligand. Direction of the specificity ligand improves the ease and efficiency of manufacture of combinatorial libraries containing bi-ligands having the common ligand mimic bound to a specificity ligand.

In one embodiment, only one of R₁ to R₄ on the benzyl ring of the benzimidazole is a substituent other than hydrogen. In such instances, R₁ to R₄ each independently can be alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X where R₁₃, R₁₄, and R₁₅ are as defined in Formula I. For example, R₁ to R₄ each independently can be a methyl, methoxy, halogen, thiol, or nitro group. When compounds of the invention contain an active hydroxy group, they also can be present in the form of an ether or ester, for example, an alkyl ether or alkyl ester. Thus, the invention encompasses compounds in which R₁ to R₄ can be an OAlkyl group or a COOAlkyl group. Non-limiting examples of OAlkyl groups include OMe (OCH₃), OEt (OCH₂CH₃), OPr (OCH₂CH₂CH₃), and the like. Non-limiting examples of COOAlkyl groups include COOMe, COOEt, COOPr, COOBu, COO-tBu, and the like.

In another embodiment, two or more of R₁ to R₄ are substituents other than hydrogen. In such instances, the substituent groups can be the same or different. For example, the phenyl ring of the benzimidazole can be substituted with two alkyl groups. Alternatively, the benzimidazole can be substituted with an OH group and an alkyl group. Any combination of the above listed substituents for R₁ to R₄, including complex substituents such as those in Tables 5 to 11, is contemplated by the present invention. Similarly, where the compounds of the invention contain three or more substituents any combination of R₁ to R₄ is encompassed by the invention.

Similarly, the unfused phenyl ring of the benzimidazole compounds of the present invention can be substituted with one or more substituents. In one embodiment of the invention, only one of R₅ to R₈ and R₁₁ is a substituent other than hydrogen. In such instances, R₅ to R₈ and R₁₁ can be alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X where R₁₃, R₁₄, and R₁₅ are as defined in Formula I. When R₅ to R₈ or R₁₁ contains an active hydroxy group, it also can be present in the form of an ether or ester, for example, an alkyl ether or alkyl ester. Thus, the invention encompasses compounds in which R₅ to R₈ and R₁₁ can be and OAlkyl group or a COOAlkyl group. In one specific embodiment, the invention provides compounds in which R₁₁, is COOH or COOAlkyl.

In another embodiment, two or more of R₅ to R₈ and R₁₁, are substituents other than hydrogen. In such instances, the substituent groups can be the same or different. For example, the phenyl ring can be substituted with two OAlkyl groups, such as two OMe groups or one OMe group and one OPr group. Alternatively, the phenyl ring of the compounds can be substituted with an OH group and either a COOH or COOAlkyl group. Any combination of the above listed substituents for R₅ to R₈ and R₁₁, inlcuding complex substituents such as those in Tables 5 to 11, is contemplated by the present invention. Similarly, where the compounds of the invention contain three or more substituents any combination of R₅ to R₈ and R₁₁ is encompassed by the invention.

In a like manner, the furan ring in Formula I can be substituted with one or two substituents. In one embodiment of the invention, only one of R₉ or R₁₀ is a substituent other than hydrogen. In such instances, R₉ or R₁₀ can be alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R14, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X where R₁₃, R₁₄, and R₁₅ are as defined in Formula I. When R₉ or R₁₀ contains an active hydroxy group, it also can be present in the form of an ether or ester, for example, an alkyl ether or alkyl ester. Thus, the invention encompasses compounds in which R₉ and R₁₀ can be OAlkyl group or a COOAlkyl group.

In another embodiment, both of R₉ and R₁₀ are substituents other than hydrogen. In such instances, the substituent groups can be the same or different. Any combination of the above listed substituents for R₉ or R₁₀, including complex substituents such as those in Tables 5 to 11, is contemplated by the present invention.

In one aspect, the invention provides compounds in which R₁ to R₁₁ are not all hydrogen. In other words, the invention includes compounds in which at least one of R₁ to R₁₁ is a substituent other than hydrogen.

When compounds of the invention contain a linker, the linker can be present, for example, at any position on the phenyl ring of the compounds, i.e., any of R₅ to R₈ and R₁₁ can be a complex linker. The invention will now be discussed further in terms of exemplified linkers, or complex substituents, that can be attached to the common ligand mimics of the invention. The variables R₅ to R₈ and R₁₁ are not depicted in these compounds for simplification. However, it is to be understood that the following compounds include any combination of R₅ to R₈ and R₁₁ in those positions which do not contain the linker.

In one embodiment, the invention provides compounds and combinatorial libraries of compounds having formula Ia

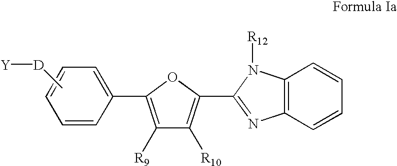

wherein D is alkylene, alkenylene, alkynylene, aryl or heterocycle. Y is OH, NHR₁₅, SH, COOH, SO₂OH, X, CN, COR₁₅, N₃, CONH₂, CONHR₁₅, C≡CH, or CH≡CH₂. R₅ to R₁₅ are as defined above for Formula I.

As used herein, the terms “alkylene,” “alkenylene,” and “alkynylene” refer to alkyl, alkenyl, and alkynyl groups as defined above in which one additional atom has been removed such that the group is divalent. Nonlimiting examples of such groups include —CH₂CH₂CH₂—, —CH₂CH═CHCH₂—, and —CH₂C≡CCH₂—.

In a second embodiment, the invention provides compounds and combinatorial libraries of compounds having formula Ib

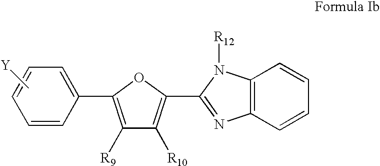

wherein Y is OH, NHR₁₅, SH, COOH, SO₂OH, X, CN, COR₁₅, N₃, CONH₂, CONHR₁₅, C≡CH, or CH═CH₂. R₅ to R₁₅ are as defined above for Formula I.

In the following formulas, the variable E can be present or absent. When present, E is defined as provided. When E is absent, the atom immediately distal to E is attached directly to the phenyl ring.

In a third embodiment, the invention provides compounds and combinatorial libraries of compounds having formula Ic

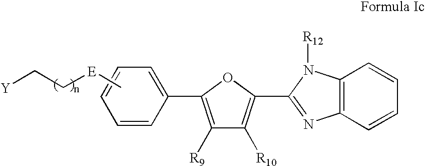

wherein E is O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH. Y is -OH, NHR₁₅, SH, COOH, SO₂OH, X, CN, COR₁₅, N₃, CONH₂, CONHR₁₅, C≡CH, or CH═CH₂; and n is an integer between 0 and 5, inclusive. R₅ to R₁₅ are as defined above for Formula I.

In another embodiment, the invention provides compounds and combinatorial libraries of compounds having formula Id

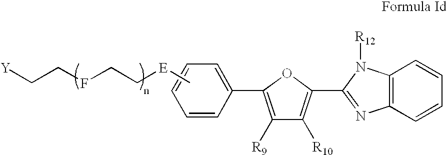

wherein E and F each independently are O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH. Y is OH, NHR₁₅, SH, COOH, SO₂OH, X, CN, COR₁₅, N₃, CONH₂, CONHR₁₅, C≡CH, or CH═CH₂; and n is an integer between 0 and 5, inclusive. R₅ to R₁₅ are as defined above for Formula I.

In another embodiment, the invention provides compounds and combinatorial libraries of compounds having formula Ie

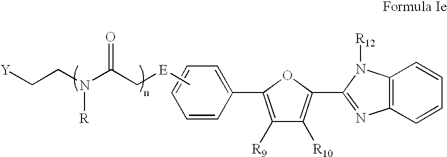

wherein E is O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH. Y is OH, NHR₁₅, SH, COOH, SO₂OH, X, CN, COR₁₅, N₃, CONH₂, CONHR₁₅, C≡CH, or CH═CH₂; and n is an integer between 0 and 5, inclusive. R is alkyl, alkenyl, alkynyl, aryl or heterocycle; and R₅ to R₁₅ are as defined above for Formula I.

In another embodiment, the invention provides compounds and combinatorial libraries of compounds having formula If

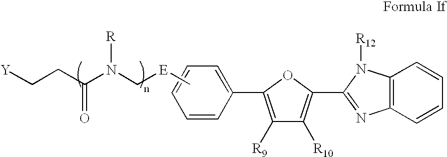

wherein E is O, S, NR₁₅, CR₁₄R₁₅,CONR₁₅, SO₂NR₁₅, NR₁₄CONR₁₅, NR₁₄CNHNR₁₅, NR₁₅COO, C≡C, or CH═CH. Y is OH, NHR₁₅, SH, COOH, SO₂OH, X, CN, COR₁₅, N₃, CONH₂, CONHR₁₅, C≡CH, or CH═CH₂; and n is an integer between 0 and 5, inclusive. R is alkyl, alkenyl, alkynyl, aryl or heterocycle; and R₅ to R₁₅ are as defined above for Formula I.

In another embodiment, the invention provides compounds and combinatorial libraries of compounds having formula Ig

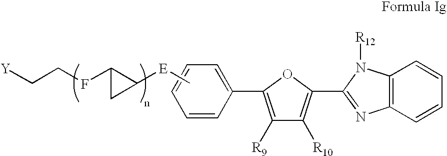

wherein E and F each independently are O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH. Y is OH, NHR₁₅, SH, COOH, SO₂OH, X, CN, COR₁₅, N₃, CONH₂, CONHR₁₅, C≡CH, or C═CH₂; and n is an integer between 0 and 5, inclusive. R₅ to R₁₅ are as defined above for Formula I.

In yet another embodiment, the invention provides compounds and combinatorial libraries of compound having formula Ih

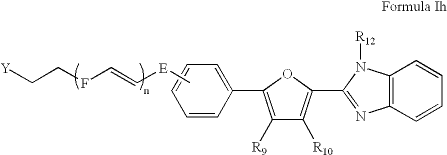

wherein E is O, S, NR₁₅, CR₁₄R₁₅, CONR₁₅,SO₂NR₁₅,NR₁₄CONR₁₅, NR₁₄CNHNR₁₅, NR₁₅COO, C≡C, or CH═CH. Each F independently is CR₁₄R₁₅, CONR₁₅, C≡C, or CH═CH. Y is OH, NHR₁₅, SH, COOH, SO₂OH, X, CN, COR₁₅, N₃, CONH₂, CONHR₁₅, C≡CH, or C═CH₂; and n is an integer between 0 and 5, inclusive. R₅ to R₁₅ are as defined above for Formula I.

In a further embodiment, the invention provides compounds and combinatorial libraries of compounds having formula Ii

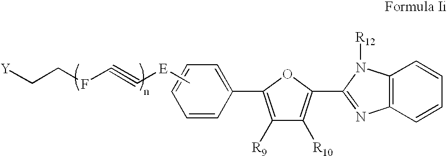

wherein E is O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH. Each F independently is CR₁₄R₁₅, CONR₁₅,C≡C, or CH═CH. Y i s OH, NHR₁₅, SH, COOH, SO₂OH, X, CN, COR₁₅,N₃, CONH₂, CONHR₁₅, C≡CH, or C═CH₂; and n is an integer between 0 and 5, inclusive. R₅ to R₁₅ are as defined above for Formula I.

In another embodiment, the invention provides compounds and combinatorial libraries of compounds having formula Ij

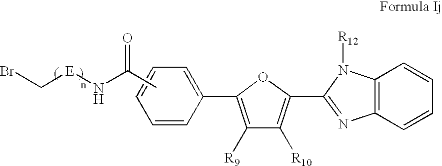

wherein E is O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH, and n is an integer between 0 and 5, inclusive. R₅ to R₁₅ are as defined above for Formula I.

In yet another embodiment, the invention provides compounds and combinatorial libraries of compounds having formula Ik

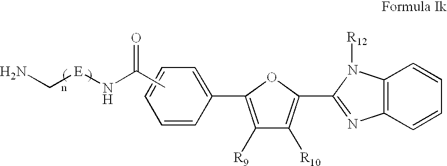

wherein E is O, S, NR₁₅, CR₁₄R₁₅, CONR₁₅, SO₂NR₁₅, NR₁₄CONR₁₅, NR₁₄CNHNR₁₅, NR₁₅COO, C≡C, or CH═CH, and n is an integer between 0 and 5, inclusive. R₅ to R₁₅ are as defined above for Formula I.

In a further embodiment, the invention provides compounds and combinatorial libraries of compounds having formula Il

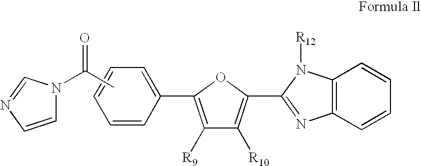

wherein R₅ to R₁₅ are as defined above for Formula I.

Nonlimiting examples of common ligand mimics of the invention include 4-[5-(4-methyl-1H-benzoimidazol-2-yl)-furan-2-yl]-benzoic acid; 4-[5-(4-methyl-1H-benzoimidazol-2-yl)-furan-2-yl]-benzoic acid methyl ester; 4-[5-(1H-benzoimidazol-2-yl)-furan-2-yl]-benzoic acid; 4-[5-(1H-benzoimidazol-2-yl)-furan-2-yl]-benzoic acid methyl ester; 4-[5-(5-methyl-1H-benzoimidazol-2-yl)furan-2-yl]-benzoic acid; 4-[5-(5-methyl-1H-benzoimidazol-2-yl)furan-2-yl]-benzoic acid methyl ester; 4-[5-(5-nitro-1H-benzoimidazol-2-yl)-furan-2-yl]-benzoic acid; 4-[5-(5-nitro-1-benzoimidazol-2-yl)-furan-2-yl]-benzoic acid methyl ester; 4-[5-(5-chloro-1H-benzoimidazol-2-yl)-furan-2-yl]-benzoic acid; 4-[5-(5-chloro-1H-benzoimidazol-2-yl)-furan-2-yl]-benzoic acid methyl ester; 4-[5-(5-methoxy-1H-benzoimidazol-2-yl)-furan-2-yl]-benzoic acid; and 4-[5-(5-methoxy-1H-benzoimidazol-2-yl)-furan-2-yl]-benzoic acid methyl ester.

One or more of the compounds of the invention, even within a given library, can be present as a salt. The term “salt” encompasses those salts that form within the carboxylate anions and amine nitrogens and includes salts formed with the organic and inorganic anions and cations discussed below. Furthermore, the term includes salts that form by standard acid-based reactions with basic groups (such as amino groups) and organic or inorganic acids. Such acids include, hydrochloric, hydrofluoric, trifluoroacetic, sulfuric, phosphoric, acetic, succinic, citric, lactic, maleic, fumaric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the sodium, potassium, barium, aluminum, and calcium); ammonium and organic cations, such as mono-, di-, and tri-alkyl amines. Examples of suitable alkyl amines include, but are not limited to, trimethylamine, cyclohexylamine, dibenzylamine, bis(2-hydroxyethyl)amine, and the like. See for example “Pharmaceutical Salts,” Berge et al., J. Pharm. Sci., 66:1-19 (1977), which is incorporated herein by reference. Other cations encompassed by the above term include the protonated form of procaine, quinine, and N-methylglucosamine, and the protonated forms of basic amino acids such as glycine, ornithine, histidine, phenylglycine, lysine, and arginine. Furthermore, any zwitterionic form of the instant compounds formed by a carboxylic acid and an amino group is referred to by this term. For example, a cation for a carboxylate anion will exist when a position is substituted by a (quarternary ammonium)methyl group.

The compounds of the invention can also exist as solvates and hydrates. Thus, these compounds may crystallize with, for example, waters of hydration, or one, a number of, or any fraction thereof, of molecules of the mother liquor solvent. The solvates and hydrates of such compounds are included within the scope of this invention.

One or more compounds of the invention, even when in a library, can be in the biologically active ester form. Such as the non-toxic, metabolically-labile, ester-form. Such esters induce increased blood levels and prolong efficacy of the corresponding nonesterified forms of the compounds. Ester groups which can be used include the lower alkoxymethyl groups, for example, methoxymethyl, ethoxymethyl, isopropoxymethyl and the like; the —(C₁-C₁₂)alkoxyethyl groups, for example, methoxyethyl, ethoxyethyl, propoxyethyl, isopropoxyethyl and the like; the —(C₁-C₁₀)alkylthiomethyl groups, for example, methylthiomethyl, ethylthiomethyl, iso-propylmethyl and the like; and the acyloxymethyl groups, for example, pivaloyloxymethyl, pivaloyloxyethyl, acetoxymethyl, and acetoxyethyl. Salts, solvates, hydrates, biologically active esters of the compounds of the invention are common ligand variants of the compounds as defined above.

In another aspect, the present invention provides bi-ligands that contain a common ligand mimic as described above and a specificity ligand. In the bi-ligands of the invention, the common ligand mimic and the specificity ligand can be attached directly or indirectly. The common ligand mimic and specificity ligand are attached via a covalent bond formed from the reaction of one or more functional groups on the common ligand mimic with one or more functional groups on the specificity ligand. Direct attachment of the individual ligands in the bi-ligand can occur through reaction of simple functional groups on the ligands. Indirect attachment of the individual ligands in the bi-ligand can occur through a linker molecule. Such linkers include those provided in Tables 5 to 11. These linkers bind to each of the common ligand mimic and the specificity ligand through functional groups on the linker and the individual ligands. Some of the common ligand mimics of the present invention having substituents that include linker molecules, e.g. the common ligand mimics of Tables 5 to 11. Tailoring of the specific type and length of the linker attaching the common ligand mimic and specificity ligand allows tailoring of the bi-ligand to optimize binding of the common ligand mimic to a conservative site on the receptor and binding of the specificity ligand to a specificity site on the receptor.

The present invention provides specificity ligands that are specific for NAD receptors and combinatorial libraries containing these specificity ligands. For example, in one embodiment, compounds of the invention are ligands for specificity sites on oxidoreductases like those described above.

In another embodiment of the present invention, the protected specificity ligand is a compound having formula

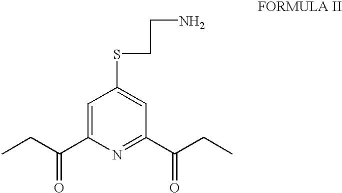

Specificity ligands, such as that of Formula II can also exist as salts, or in other reactive forms and can be reacted with the common ligand mimics of the invention to provide bi-ligands of the invention.

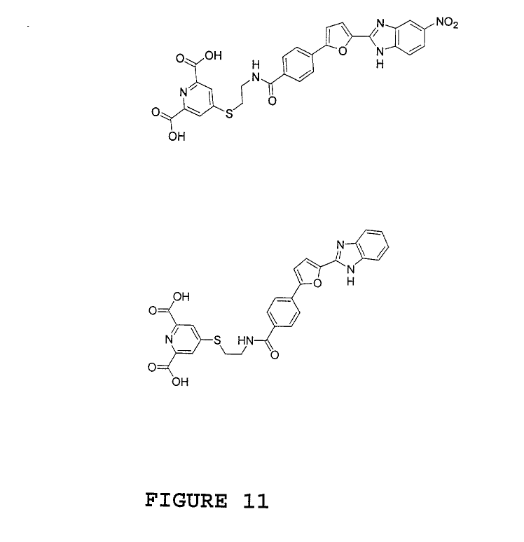

Bi-ligands of the invention can be bi-ligands for any receptor. In one embodiment, the bi-ligand is a bi-ligand that binds an oxidoreductase. In another embodiment, bi-ligands of the present invention comprise a benzimidazole compound, as a common ligand mimic, and a specificity ligand. For example, bi-ligands of the invention can contain a common ligand mimic of Formula I coupled to a specificity ligand. The specificity ligand can be any specificity ligand, for example a ligand that binds to a specificity site on an oxidoreductase. In such an embodiment, the specificity ligand can be a pyridine dicarboxylate. Examples of particular bi-ligands that fall within the invention are provided in FIG. 11.

The compounds of the present invention can be produced by any feasible method. For example, the compounds of the present invention can be produced by the following methods. Generally, these methods include the formation of an intermediate compound, followed by reaction of the intermediate with a phenylenediamine to form a benzimidazole methyl ester. The methyl ester then optionally can be treated with lithium hydroxide to form the corresponding benzoic acid. Tailoring of the methods of the invention to produce a particular compound within the scope of the invention is within the level of skill of the ordinary artisan.

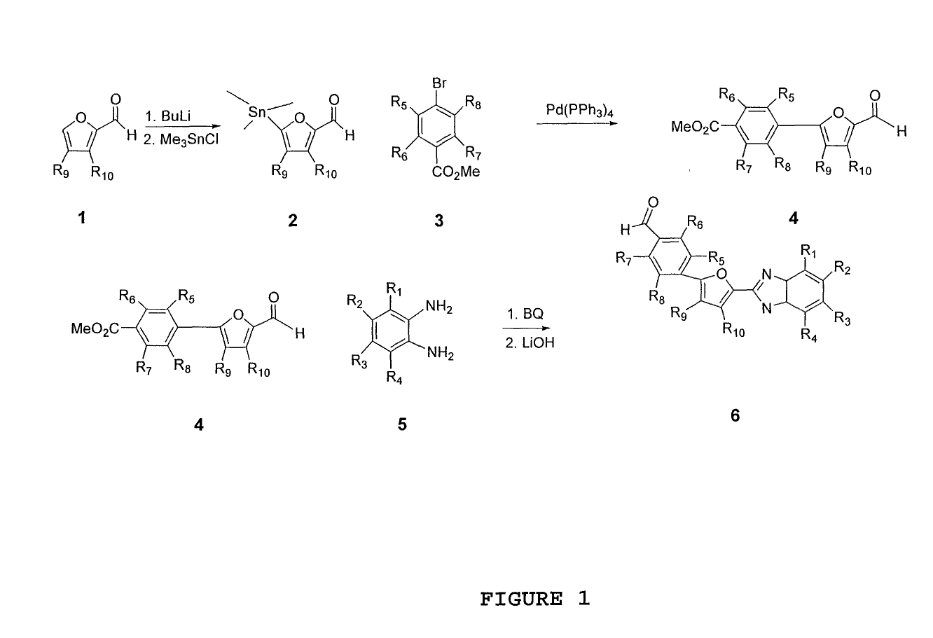

In one aspect, as shown in FIG. 1, the present invention provides a method for the manufacture of benzimidazole compounds. The process involves formation of an intermediate. The intermediate then is reacted with a phenylenediamine to form a benzimidazole-benzoic acid methyl ester. The methyl ester optionally can be converted to the corresponding benzoic acid.

The intermediate compound of the present invention can be formed, for example, in the following manner. A mixture of 5-trimethylstannanyl-furan-2-carbaldehyde, tetrakis(triphenylphosphine)palladium, and a 4-halobenzoate, such as methyl 4-bromobenzoate, is formed. The reaction can be performed in a solvent under an inert atmosphere. For example, the reaction can be performed in dimethylformamide (DMF) in nitrogen (N₂). The reaction mixture is heated at a temperature of about 50 to 100° C. for a period of about 1 to about 24 hours. For instance, the reaction can be heated at a temperature of 60° C. for about 20 hours. The intermediate product can then be dried, for example by evaporation under reduced pressure. The residue can then be purified, for example, by chromatography with a mixture of EtOAc/hexane (1:3). Formation of intermediates of the invention is further described in Example 2.

The 5-trimethylstannanyl-furan-2-carbaldehyde used in the above method can be prepared by any known method. In one embodiment of the present invention, this compound also can be prepared according to the following method.

A solution of 4-methylpiperidine in a solvent, such as THF, is formed at temperature of about −60 to about −100° C. under an inert atmosphere. For instance, the solution can be formed at a temperature of about −78° C. under a nitrogen atmosphere. Butyl lithium (BuLi) in hexane is then added to the solution, followed by the addition of 2-furaldehyde.

While maintaining the reaction temperature, another portion of BuLi is added to the reaction mixture. The mixture is then allowed to warm to a temperature of about −10 to 40° C. and stirred for a period of about 1 to 24 hours. For example, the reaction mixture can be warmed to a temperature of about −20° C. and stirred for a period of about 5 hours.

The reaction mixture is then cooled again to a temperature of about −60 to 100° C., for example −78° C., and added to a solution of Me₃SnCl in the same solvent. The reaction mixture is then allowed to warm gradually to room temperature and stirred overnight.

The reaction is then quenched, for example, by adding cold brine or cold water followed by extraction with ethyl acetate or dichloromethane. The extracted organic phase then can be dried and concentrated using conventional methods. If desired, the product can be purified by chromatography or by any other suitable means. This process for the manufacture of 5-trimethylstannanyl-furan-2-carbaldehyde is further described in Example 1.

Intermediate compounds formed by the methods of the invention described above can subsequently be used in the following methods of the invention to produce benzimidazole derivatives of the invention.

Such compounds can be formed, for example, by the following method. The intermediate compound is mixed with a phenylenediamine in a solvent, such as ethanol. The mixture is heated at reflux for a period of about 1 to 24 hours, for instance, for a period of about 4 hours.

The solvent is then removed, and the residue dissolved in dichloromethane. The residue can then be washed with brine (2×10 ml), concentrated, and purified, for example, by flash chromatography with a 1:1 mixture of EtOAc/hexane. The formation of benzimidazole compounds of the invention is further described in Examples 2 to 7.

The methyl ester compounds prepared by the reaction can be converted to the corresponding benzoic acid. In such instances, the present invention provides a method by which this conversion can occur. The methyl ester is suspended in a solvent, such as methanol or a methanol/THF mixture. A solution of LiOH in water is then added to the solution. The reaction mixture is stirred at room temperature for a period of time of about 1 to 24 hours. For example, the reaction can be stirred at room temperature for a period of about 20 hours.

The solution is then acidified to a pH of about 1 and quickly extracted. The solution can be acidified, for example, with a solution of citric acid or 2N HCl. Extraction of the product can be accomplished with ethyl acetate or dichloromethane. The extracted organic layers can then be dried, for example, over MgSO₄. If desired, the resulting benzoic acid can be filtered and concentrated in vacuo.

The methods of the present invention now will be described in terms of specific embodiments for the preparation of a compound of formula I

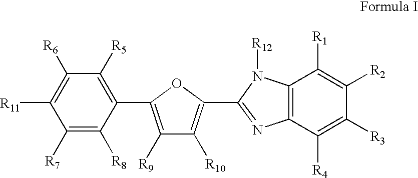

wherein R₁ to R₁₁ each independently are H, alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S (O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅,PO₂R₁₄R₁₅, CN, or X. R₁₂, R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring. These embodiments exemplify the invention and do not limit the scope of the invention.

In one embodiment, the method involves reacting 5-trimethylstannanyl-furan-2-carbaldehyde and a halobenzoate, such as 4-bromobenzoate, in the presence of tetrakis(triphenylphosphine)palladium to form a furanyl intermediate. This reaction optionally is performed in a solvent and/or optionally in an inert atmosphere, such as nitrogen.

The 5-trimethylstannanyl-furan-2-carbaldehyde used in the reaction can be produced in any manner. Optionally, the 5-trimethylstannanyl-furan-2-carbaldehyde is produced by the following method. The compounds 2-furaldehyde are reacted in a solvent, such as tetrahydrofuran, under an inert atmosphere, like nitrogen, in the presence of butyl lithium at a temperature of about −60 to −100° C., for example −78° C. The reaction mixture is stirred while it is allowed to warm to a temperature of about −10 to −40° C., for example −20° C. The reaction mixture again is cooled to a temperature of about −60 to −100° C., for example −78° C., followed by the addition of a solution of Me₃SnCl. The mixture is then warmed and quenched by addition of cold brine. The organic phase containing the 5-trimethylstannanyl-furan-2-carbaldehyde was extracted with ethyl acetate. The 5-trimethylstannanyl-furan-2-carbaldehyde is optionally dried and/or purified by chromatography prior to use.

The furanyl intermediate formed in the first step of the process is then reacted with a phenyldiamine, such as 2,3-diaminotoluene and benzoquinone to form a benzimidazole benzoic acid methyl ester. The methyl ester is optionally reacted with lithium hydroxide to free the acid group and form the corresponding benzoic acid.

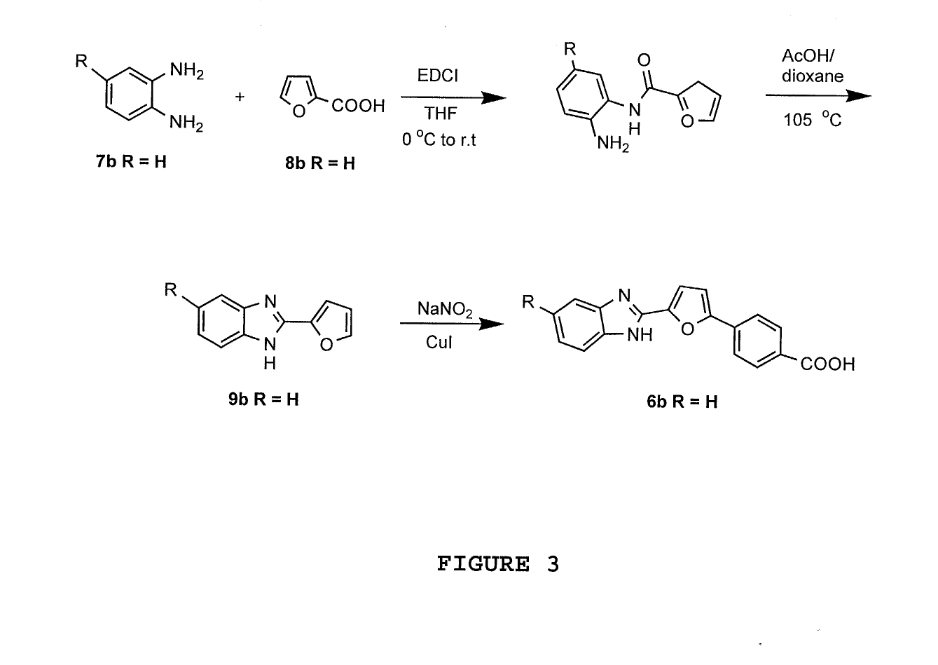

Common ligand mimics of the present invention can be prepared by alternative methods. For example, common ligand mimics of the present invention having any of R₅ to R₈ or R₁₁ as a carboxylic acid group can be prepared by the following alternative method for which the reaction scheme is provided in FIG. 3. A solution of 1,2-phenylenediamine and 2-furoic acid is prepared in a solvent, such as THF, DMF, or DCM at a temperature of about −20 to 0° C. EDCI (1-(3-dimethylaminopropyl)-3-ethylcarbodiimide hydrochloride is then added to the solution, which is allowed to warm to room temperature. The reaction is continued for a period of about 2 to about 20 hours, and then the solvent is evaporated. The resultant residue is dissolved in ethyl acetate, washed with water, and dried over MgSO₄.

A solution of amide in dioxane and TFA is heated at a temperature of about 100 to 120° C. for a period of about 20 hours. The solvent is then evaporated, and a small amount of ethyl acetate is added to the product, resulting in a yellow solid. The solid then can be filtered to provide the desired product.

A suspension of 4-aminobenzoic acid is formed in a mixture of water and concentrated HCl. Sodium nitrite is gradually added to the suspension at a temperature of 0° C. A solution of amide in acetone is then added to the suspension, followed by addition of a mixture of CuI and CuCl₂ over a period of 10 minutes at a temperature of 0° C. The reaction is stirred for a period of about 1 hour at room temperature. The precipitate then can be collected by filtration, washed with water and acetone, and dried to yield a pure compound. This common ligand mimic can then be used to prepare common ligand mimics of the invention containing more complex substituent linkers as described below.

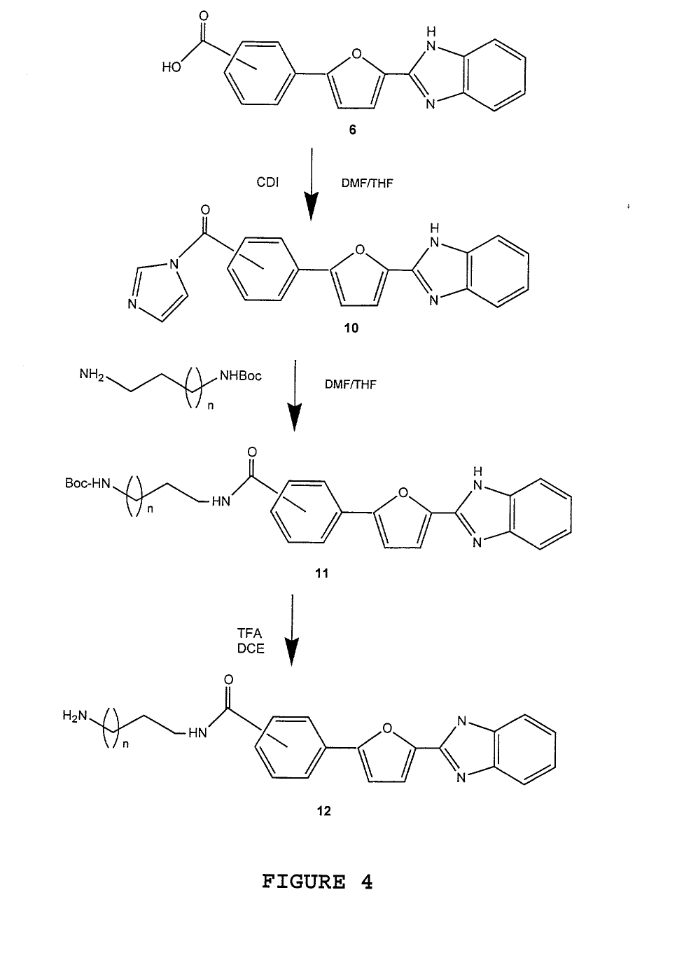

As shown in FIG. 4, a common ligand mimic of the present invention containing a carboxylic acid group is dissolved in a solvent, such as dimethylformamide or tetrahydrofuran. The compound is then reacted with 1,1′-carbonyldiimidazole in tetrahydrofuran at a temperature of about 40 to 80° C. The reaction mixture is agitated for a period of time, for example 20 minutes to 3 hours.

The mixture is then covered and refrigerated for a period of time at a temperature of about −10 to 4° C. For example the reaction mixture can be refrigerated overnight at a temperature of about −10° C. The precipitate can then be collected by filtration and washed with THF to form an intermediate compound.

The intermediate compound is then placed in a mixture of DMF and THF. Boc-protected diamines (t-butyl carbamate protected diamines) are added to the mixture, and the mixture is heated to a temperature of about 40 to 80° C. for a period of about 1 to 3 hours, followed by evaporation of the solvent, for example, under reduced pressure. For example, the mixture can be heated at a temperature of about 65° C. for a period of about 1 hour.

Next, a solution of 50% trifluoacetic acid in dichloroethane (100 ml) is added to the precipitate and reacted for a period of about 20 to 60 minutes, followed by evaporation of the remaining solvent. For example, the mixture can be reacted for a period of about 10 minutes, followed by evaporation of extra solvent. The precipitate can then be dissolved in a solvent, such as DMF, by heating. The solution is cooled to room temperature, and a Na₂CO₃ solution added. When a precipitate forms, it is filtered. If necessary, additional solvent and water can be added. The final product can then be washed with a mixture of water and alcohol, such as water and MeOH, and then dried. This method is described further in Example 11.

Bi-ligands of the present invention can be produced by any feasible method. For example, the compounds of the present invention can be produced by the following methods. These methods are exemplified using a common ligand mimic of Formula I and a pyridine dicarboxylate specificity ligand. However, one having ordinary skill in the art will appreciate that variations in such methods can be employed to produce bi-ligands having other common ligand mimics or other specificity ligands.

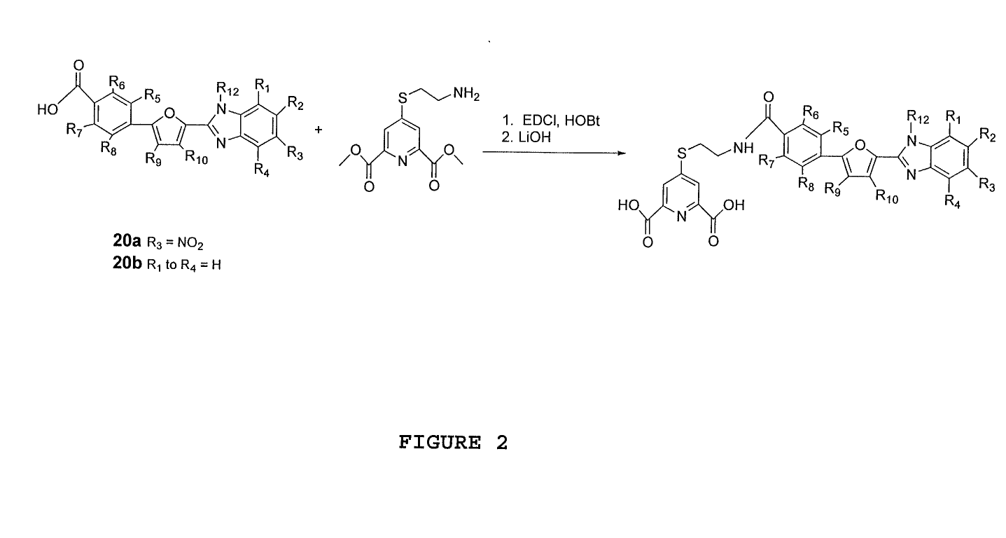

As shown in FIG. 2, a common ligand mimic of the invention, such as a benzimidazole compound of Formula I can be reacted with a pyridine dicarboxylate compound in a solvent in the presence of butanol. Suitable solvents include dimethylformamide, HOBt.H₂O. Suitable solvents include dimethylformamide, tetrahydrofuran, and dichloromethane. For example, the reaction of dicarbolxylic acid and pyridine can be performed in dimethylformamide with the addition of HOBt.H₂O. Triethylamine and 1-dimethylaminopropyl-3-ethyl-carbodiimide (EDCI) are then added to the mixture. The reaction is then stirred at room temperature for a period of about 2 to 50 hours. For example, the reaction can be stirred at room temperature for a period of about 16 hours or about 31 hours.

A precipitate is formed by adding aqueous 2N HCl to the reaction mixture. The reaction precipitate is collected and washed with aqueous HCl, such as a 0.5N HCl solution. Then, the recovered solid can be suspended in a mixture of alcohol, such as methanol, water, and LiOH. This suspension is stirred at room temperature for a period of about 1 to 24 hours. For instance, the suspension can be stirred at room temperature for a period of about 4 hours. The solution is then acidified, for example with aqueous 2N HCl. The resulting precipitated product can then be filtered and dried. Formation of the bi-ligands of the invention is further described in Examples 8 and 9.

As used herein, a “combinatorial library” is an intentionally created collection of differing molecules that can be prepared by the means provided below or otherwise and screened for biological activity in a variety of formats (e.g., libraries of soluble molecules, libraries of compounds attached to resin beads, silica chips or other solid supports). A “combinatorial library,” as defined above, involves successive rounds of chemical syntheses based on a common starting structure. The combinatorial libraries can be screened in any variety of assays, such as those detailed below as well as others useful for assessing their biological activity. The combinatorial libraries will generally have at least one active compound and are generally prepared such that the compounds are in equimolar quantities.

Compounds described in previous work that are not taught as part of a collection of compounds or not taught as intended for use as part of such a collection are not part of a “combinatorial library” of the invention. In addition, compounds that are in an unintentional or undesired mixture are not part of a “combinatorial library” of the invention.

The present invention provides combinatorial libraries containing two or more compounds. The present invention also provides combinatorial libraries containing three, four, or five or more compounds. The present invention further provides combinatorial libraries that can contain ten or more compounds, for example, fifty or more compounds. If desired, the combinatorial libraries of the invention can contain 100,000 or more, or even 1,000,000 or more, compounds.

In one embodiment, the present invention provides combinatorial libraries containing common ligand variants of compounds of Formula I. These common ligand variants are active forms of the compounds of Formula I that are capable of binding to a specificity ligand to form a bi-ligand. For example, where one of R₁ to R₄ is a OH or OAlkyl group, the common ligand variant can be a compound containing the group O-. Common ligand variants of the invention include common ligand mimics in which the substituents on the compounds are complex ligands such as those attached to the compounds listed in Tables 5 to 11.

In another embodiment, the present invention provides combinatorial libraries containing bi-ligands of the invention. The bi-ligands are the reaction product of a common ligand mimic and a specificity ligand that interact with distinct sites on a single receptor. For example, the common ligand mimic can be one or more common ligand mimic for NAD which binds to a conserved site on a dehydrogenase, like ADH. In such a bi-ligand, the specificity ligand is one or more ligands that bind a specificity site on ADH.

Such combinatorial libraries can contain bi-ligands having a single common ligand mimic bonded to multiple specificity ligands. Alternatively, the combinatorial libraries can contain bi-ligands having a single specificity ligand bonded to multiple common ligand mimics. In another aspect, the combinatorial libraries can contain multiple common ligand mimics and multiple specificity ligands for one or more receptors.

The use of a common ligand mimic of the invention to produce the combinatorial library allows generation of combinatorial libraries having improved affinity and/or specificity. Selection and tailoring of the substituents on the common ligand mimic also allows for production of combinatorial libraries in a more efficient manner than heretofore possible.

Bi-ligand libraries of the invention can be prepared prepared in a variety of different ways. For example, two methods employing a resin, such as HOBt resin, carbodiimide resin, or DIEA (diisopropyldiisoamine) resin can be used to form bi-ligand libraries. In one such method, bi-ligand libraries can be prepared via direct coupling of amines to common ligand mimics of the invention having a carboxylic acid group.

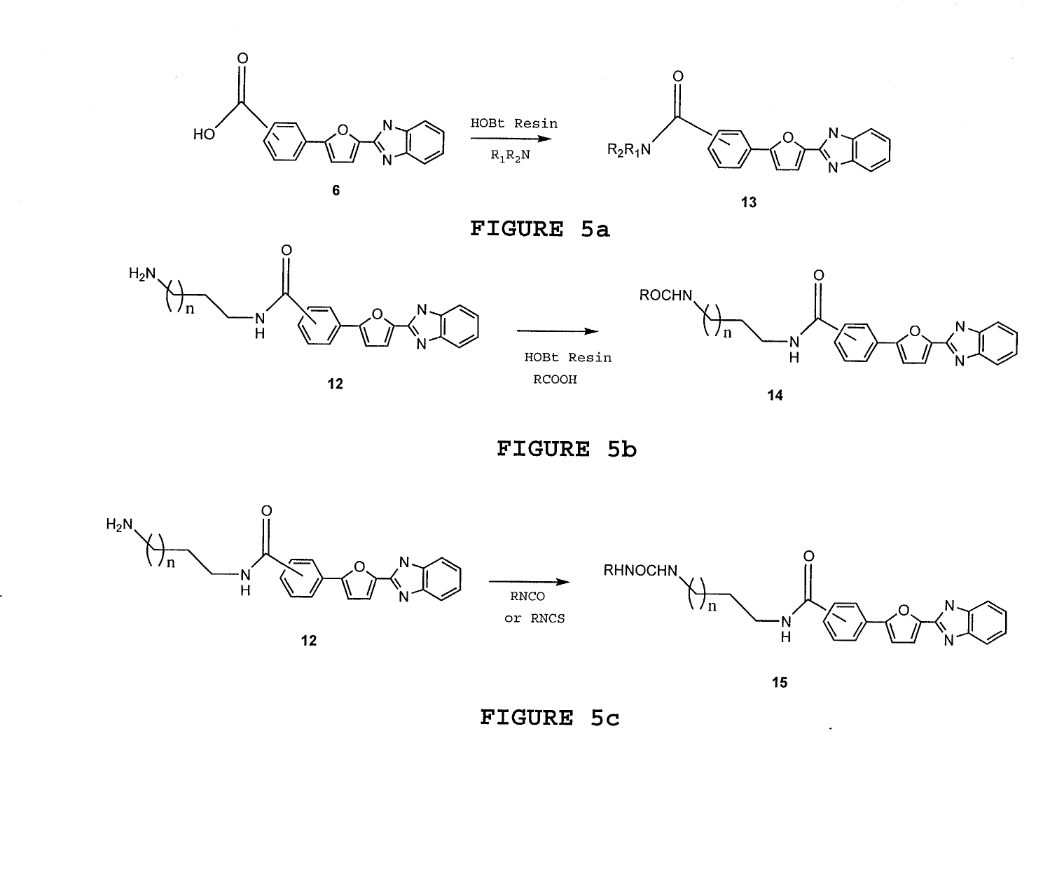

As shown in FIG. 5a, bi-ligand libraries can be prepared in the following manner. HOBt resin is swelled in a dry solvent, such as a mixture of dry THF and dry DMF, and added to a solution of a common ligand mimic of the invention that is dissolved in a solvent, such as a mixture of DMF and DIC. The solution is shaken at room temperature overnight and then washed with 3x dry DMF and 3x dry THF. The resin is added to a solution of an amine in a solvent, for example dry DMF. The mixture is shaken again at room temperature overnight. The resin then can be filtered and washed with solvent, and the filtrate can be collected and vacuum dried to provide bi-ligands of the invention. Nonlimiting examples of amines useful for the preparation of bi-ligand libraries include those in Table 1.

TABLE 1

| | | |
|---|---|---|
| cyclopropylamine | nipecotamide | 3-chloro-p-anisidine |
| isopropylamine | N-butylamine | 5-amino-1-napthol |
| N,N-diethyl-N′- | 2-(2-aminoethyl)-1- | 2-amino-5,6-dimethyl- |
| methylethylenediamine | methylpyrrolidine | benzimidazole |
| N-(3-aminopropyl)-N- | 2-(aminomethyl)-1- | N,N-diethyl-p- |
| methylaniline | ethylpyrrolidine | phenylenediamine |
| hydroxylamine | N-(2-aminoethyl)- | 1-(2-pyridyl) |
| hydrochloride | piperidine | piperazine |
| 4-amino-1,2,4- | 4-(2-aminoethyl) | 3,5- |
| triazole | morpholine | dimethoxybenzylamine |
| N-methylallylamine | propylamine | pyrrolidine |
| 3-pyrroline | 3-aminobenzamide | 1-phenylpiperazine |
| diethylamine | ethyl 3-aminobutyrate | 4-butoxyaniline |
| isobutylamine | 5-aminoindan | cyclopentylamine |
| 1-(3-aminopropyl) | trans-2- | 2,4-dimethoxy |
| pyrrolidine | phenylcyclopropylamine | benzylamine |
| N-methylpropylamine | 3-phenyl-1-propylamine | 4-pentylaniline |
| sec-butylamine | beta-methylphenethylamine | ethyl 4-aminobutyrate |
| 2-methoxyethylamine | N-methylphenethylamine | 1-cyclohexylpiperazine |
| cyclobutylamine | p-isopropylaniline | 4-piperidinopiperidine |
| 2,3- | 2-amino-5-trifluoromethyl- | 2-amino-5- |
| dimethoxybenzylamine | 1,3,4-thiadiazole | chlorobenzoxazole |
| ethyl 4-amino-1- | N,N-dimethyl-1,4- | 2-(aminomethyl) |
| piperidinecarboxylate | phenylenediamine | benzimidazole |
| morpholine | N-(4- | 2-aminobiphenyl |
| | pyridylmethyl) ethylamine | |
| 1-ethylpropylamine | 4-aminobenzamide | 3-aminobiphenyl |
| neopentylamine | 3,4-(methylenedioxy)- | N-undecylamine |
| | aniline | |
| N-ethylisopropylamine | 4-hydroxybenzamide | piperidine |
| N-methylbutylamine | 6-aminonicotinamide | 4-cyclohexylaniline |
| 2-amino-1- | 4-fluorophenethylamine | 2-(trifluoromethyl) |
| methyloxypropane | hydrochloride | benzylamine |
| 3-methoxypropylamine | 3-amino-4-methylbenzyl | 2, 4-dimethyl-6- |
| | alcohol | aminophenol |
| thiazolidine | 3-methoxybenzylamine | 2,4-dichlorobenzylamine |
| 3-amino-1,2,4-triazine | 4-ethoxyaniline | 3,4-dichlorobenzylamine |
| furfurylamine | 4-methoxy-2-methylaniline | 4-aminoquinaldine |
| diallylamine | 4-methoxybenzylamine | 4-(methylthio)aniiine |
| 2-methylpiperidine | m-phenetidine | 1-benzylpiperazine |
| 3-methylpiperidine | 5-amino-2-methoxyphenol | 4-piperidino aniline |
| 4-methylpiperidine | tyramine | 4-(trifluoromethoxy)- |
| | | aniline |
| cyclohexylamine | 2-fluorophenethylamine | 4-hexylaniline |
| hexamethyleneimine | 3-fluorophenethylamine | 4-amino-2,6- |
| | | dichlorophenol |
| 1-aminopiperidine | 3-(methylthio) aniline | 4-morpholinoaniline |
| 2-amino-4-methoxy-6- | (3S)-(+)-1-benzyl-3- | N-(2-aminoethyl)-N- |
| methylpyrimidine | aminopyrrolidine | ethyl-m-toluidine |
| tetrahydrofurfurylamine | 1-methylpiperazine | 4-chlorobenzylamine |
| 1,3-dimethylbutylamine | dipropylamine | 1-(2-furoyl)piperazine |
| 3-chlorobenzylamine | 2-chlorobenzylamine | 1-(2-fluorophenyl) |
| | | piperazine |
| 4-aminomorpholine | 3,3,5- | 1-(4-fluorophenyl) |
| | trimethylcyclohexylamine | piperazine |
| N-(3′-aminopropyl)-2- | 4-aminophenylacetic acid | 2-(3,4-dimethoxyphenyl) |
| pyrrolidinone | ethyl ester | ethylamine |
| 3-dimethyl amino | N-acetylethylenediamine | 2-amino-fluorene |
| propylamine | | |
| N-isopropylethylene | 2,4-difluorobenzyiamine | 3,4,5-trimethoxyaniline |
| diamine | | |
| o-toluidine | N-phenyl-p-phenylenediamine | 4-aminodiphenylmethane |
| 1-aminonaphthalene | 2,6-difiuorobenzylamine | aminodiphenylmethane |
| 5-amino-1-pentanol | 3,4-difluorobenzylamine | 2,5-difluorobenzylamine |
| 3-ethoxypropylamine | 2-(aminomethyl)-1,3- | 3-phenoxyaniline |
| | dioxolane | |
| 3-(methylthio) | 2-aminonaphthalene | 4-phenoxyaniline |
| propylamine | | |
| benzylamine | p-phenetidine hydrochloride | 1-(3- |
| | | chlorophenyl) piperazine |
| m-toluidine | 8-aminoquinoline | 4-amino-1- |
| | | benzylpiperidine |
| 3-fluoroaniline | N-(3-aminopropyl) | 4-aminohippuric acid |
| | morpholine | |
| p-toluidine | 7-amino-4-methylcoumarin | 2-amino-9-fluorenone |
| 1-amino-5,6,7,8- | 4-piperidone monohydrate | 2-methyl-1-(3- |
| tetrahydronaphthalene | hydrochloride | methylphenyl) piperazine |
| 2-(aminomethyl)pyridine | 2-amino-1- | 3,4,5- |
| | methylbenzimidazole | trimethoxybenzylamine |
| 3-(aminomethyl)pyridine | 4-phenylbutylamine | 2,2-diphenylethylamine |
| 4-(aminomethyl)pyridine | 4-amino-N-methylphthalimide | 3-benzyloxyaniline |
| 1,2,3,4-tetrahydro-1- | 4-(2-aminoethyl)benzene | 4-amino-4′- |
| naphthylamine | sulfonamide | methyldiphenylether |
| 2-amino-4- | N-propylcyclopropane | 1-methyl-3- |
| methylbenzothiazole | methylamine | phenylpropylamine |
| 2-thiophenemethylamine | 4-tert-butylaniline | exo-2-aminonorbornane |
| 2-methylcyclohexylamine | 4′-aminoacetanilide | 1,4-benzodioxan-5-amine |
| 3,5-dimethylpiperidine | N-(4-aminobenzoyl)-beta- | piperonylamine |
| | aianine | |
| 4-methylcyclohexylamine | methyl 3-amino-benzoate | 5-phenoxy-o-anisidine |
| N-isopropyl-N-phenyl-p- | 2-methoxy-N-phenyl-1,4- | 4-amino-4′- |
| phenylenediamine | phenylenediamine | chlorodiphenylether |
| cyclohexanemethylamine | 2-ethoxybenzylamine | 1-piperonylpiperazine |
| heptamethyleneimine | 2-methoxyphenethylamine | 4-amino-4′- |
| | | methoxystilbene |
| 1-(4- | 4-isopropoxyaniline | cycloheptylamine |
| nitrophenyl)piperazine | | |
| 1-piperazinecarbox | 4-methoxyphenethylamine | (−)-cis-myrtanylamine |
| aldehyde | | |
| 2-amino-4- | 3,5-dimethoxyaniline | 4-(4-nitrophenoxy)- |
| methylthiazole | | aniline |
| 1,3,3-trimethyl-6- | alpha-(cyanoimino)-3,4- | 4-amino-4′- |
| azabicyclo [3,2,1]octane | dichlorophenethylamine | nitrodiphenylsulfide |
| 1-methylhomopiperazine | 1-ethylpiperazine | 2-amino-7-bromofluorene |
| N-(2-aminoethyl) | 4-tert-butylcyclohexylamine | 2-(3-chlorophenyl) |
| pyrrolidine | | ethylamine |
| 2-amino-5-phenyl-1,3,4- | 2-amino-4,5,6,7- | (1R,2S)-(+)-cis-1-amino- |
| thiadiazole suliate | tetrahydrobenzo (b) thiophene- | 2-indanol |
| | 3-carbonitrile | |
| 1-amino-4- | 2-(4-chlorophenyl) | n-undecylamine |
| methylpiperazine | ethylamine | |
| 2-heptylamine | 1-(3-aminopropyl)-2- | 2,6-dimethylmorpholine |
| | pipecoline | |
| N,N,N′-trimethyl-1,3- | 4-amino-2,2,6,6- | d(+)-alpha- |
| propanediamine | tetramethylpiperidine | methylbenzylamine |
| N-methylhexylamine | ethyl nipecotate | dl-1-amino-2-propanol |
| 1-(3-aminopropyl)-4- | N,N-dimethyl-N′- | dl-alpha- |
| methyl-piperazine | ethylethylenediamine | methylbenzylamine |
| 3-aminobenzyl alcohol | N,N-diethylethylenediamine | o-anisidine |
| (R)-(+)-2-amino-3- | 2-(furfurylthio) ethylamine | 3-amino-4-methylbenzyl |
| phenylpropanol | | alcohol |
| 2-(2-aminoethyl)-1,3- | 2,3-dimethyl | 3-amino5,5-dimethyl-2- |
| dioxolane | cyclohexylamine | cyclohexen-1-one |
| 6-amino-1-hexanol | N-methyl-b-alaninenitrile | 3-aminophenol |
| 3-isopropoxy | 1-methyl-4- | (R)-(+)-1- |
| propylamine | (methylamino)piperidine | phenylpropylamine |
| 2-methylbenzylamine | 1-amino-2-butanol | 2 -piperidineethanol |
| (R)-1-(4-methylphenyl) | 2-amino-2-methyl-1-propanol | 2,3-dimethyl-4- |
| ethylamine | | aminophenol |
| 3-methylbenzylamine | 4-amino-1-butanol | 1-aminoindan |
| 4-methylbenzylamine | 3-(ethylamino) propionitrile | phenethylamine |
| N-methylbenzylamine | 4-hydroxypiperidine | 3,4-dimethylaniline |
| (+/−)-2-amino-1-butanol | N-(2-hydroxyethyl) | 1-naphthalene |
| | piperazine | methylamine |
| 2-(2-aminoethyl) | S(+)-1-cyclohexyl | 2-aminophenethyl alcohol |
| pyridine | ethylamine | |
| 6-amino-m-cresol | 4-aminophenol | decylamine |
| m-anisidine | 2-ethylpiperidine | 4-aminophenethyl alcohol |
| p-anisidine | N-methylcyclohexylamine | diethanolamine |
| methyl 4-aminobenzoate | 3-piperidinemethanol | 2-(methylthio)aniline |
| 5-amino-o-cresol | 2,4-dimethylaniline | 4-amino-2-chlorophenol |
| 4-fluorobenzylamine | 2,5-dimethylaniline | dibenzylamine |
| 1-(3-aminopropyl)- | 6-amino-3′,4′(methylene- | 2-(aminomethyl)-5- |
| imidazole | dioxy) acetophenone | methylpyrazine |
| 2-(1-cyclohexenyl) | 3-amino-4-hydroxybenzoic | (R)-(+)-1-(4- |
| ethylamine | acid | methoxyphenyl) ethylamine |
| 2,(2-thienyl)ethylamine | (1R, 2S)-1-amino-2-indanol | 4-ethynylaniline |
| 1-(3,4-dichlorophenyl) | N-(4-amino-2- | 1(−)-2amino-3-phenyl-1- |
| piperazine | chlorophenyl) morpholine | propanol |
| 1-acetylpiperazine | N-benzyl-2-phenylethylamine | 5-tert-butyl-o-anisidine |
| isonipecotamide | 5-phenyl-o-anisidine | 4-amino salicylic acid |
| 2-amino-m-cresol | cyclooctylamine | 2,4-dimethoxyaniline |
| 2-methoxy-6- | 3-hydroxytyramine | 4-amino-3-hydroxybenzoic |
| methylaniline | hydrobromide | acid |
| 2-aminonorbornane | 2-[2-(aminomethyl) | 1-amino-2- |
| hydrochloride | phenylthio]benzyl alcohol | methylnaphthalene |
| 5-aminoindazole | 2-amino-1,3-propanediol | 3-amino-5-phenylpyrazole |
| 5-aminobenzotriazole | 3-amino-1,2-propanediol | veratrylamine |
| methyl 4-aminobutyrate | 3-bromobenzylamine | 3-amino-1-phenyl-2- |
| hydrochloride | hydrochloride | pyrazolin-5-one |
| 2-chloro-4,6- | 1-(2-methoxyphenyl) | 5-amino-1-methyl-3- |
| dimethylaniline | piperazine hydrochloride | (thien-2-yl) pyrazole |
| (1S,2S)-(+)-2-amino-1- | 4-benzyloxyaniline | 3,5-bis(trifluoro- |
| phenyl-1,3-propanediol | hydrochloride | methyl) -benzylamine |
| 2-bromobenzylamine | (S)-(+)-2-amino-3- | 3-aminopyrrolidine |
| hydrochloride | cyclohexyl-1-propanol HCl | dihydrochloride |

| | |
|---|---|
| N-(4-methoxyphenyl)-p-phenylenediamine hydrochloride | 2-piperidinemethanol |

In another of such methods, bi-ligand libraries can be prepared by reacting carboxylic acids to common ligand mimics of the present invention having an amine or amide containing substituent.

As shown in FIG. 5b, bi-ligand libraries of the invention can also be prepared in the following manner. HOBt resin is swelled a dry solvent, such as dry THF, and added to a solution of a carboxylic acid in a solvent, such as a mixture of dry DMF and DIC. The solution is shaken at room temperature overnight and then washed with 3X dry DMF and 1X dry THF. The resin is added to a solution of a common ligand mimic of the invention in a solvent, for example dry DMF. The solution is again shaken at room temperature overnight. The resin then can be filtered and washed with solvent, followed by collection and vacuum drying of the filtrate to provide bi-ligands of the invention. Nonlimiting examples of carboxylic acids useful for the preparation of bi-ligand libraries include those in Table 2.

TABLE 2

| | | |
|---|---|---|
| acetic acid | 5-Bromonicotinic acid | 4-Chlorobenzoic acid |
| 4-Chloro-3-nitrobenzoic | 4-(3-Hydroxyphenoxy) benzoic | 4-Biphenylcarboxylic |
| acid | Acid | acid |
| N-Acetylglycine | 3,5-Dihydroxybenzoic acid | 2-Bromobenzoic acid |
| Propionic acid | 2,4-Dihydroxybenzoic acid | 3-Bromobenzoic acid |
| Crotonic acid | 2,3-Dihydroxybenzoic acid | 4-Bromobenzoic acid |
| 4-pentenoic acid | 2-Chloro-5-nitrobenzoic | 4-Phenoxybenzoic acid |
| | acid | |
| methacrylic acid | 6-Mercaptonicotinic acid | 4-Mercaptobenzoic acid |
| Pyruvic acid | Cyclohexanepropionic acid | acrylic acid |
| 3-Hydroxy-2-methyl-4- | 1-(4-Chlorophenyl)-1- | 4-Hydroxy-3-(morpholino- |
| quinolinecarboxylic | cyclopropanecarboxylic acid | mehtyl)benzoic acid |
| acid | | |
| n-butyric acid | 3-Chlorobenzoic acid | isobutyric acid |
| methoxyacetic acid | 2-Chlorobenzoic acid | 3-Indolebutyric acid |
| mercaptoacetic acid | 5-Nitro-2-furoic acid | 2,6-Difluorobenzoic acid |
| 2,3-Difluorobenzoic | 6-Chloronicotinic acid | Ethoxyacetic acid |
| acid | | |
| trans-2,3- | 1,4-Dihydroxy-2-napthoic | 3,7-Dihydroxy-2-napthoic |
| dimethylacrylic acid | acid | acid |
| Cyclobutanecarboxylic | 2-methylcyclopropane | 2-Chloro-4-nitrobenzoic |
| acid | carboxylic acid | acid |
| cyclopropanecarboxylic | 4-(4-Hydroxyphenoxy)benzoic | 9H-Fluorene-9-carboxylic |
| acid | Acid | acid |
| 2-ketobutyric acid | 3,5-Difluorobenzoic acid | Pentafluorobenzoic acid |
| Isovaleric acid | 2,4-Difluorobenzoic acid | Indole-5-carboxylic acid |
| Trimethylacetic acid | 3,4,5-Trimethoxybenzoic | 3-Nitrobenzoic acid |
| 99% | acid | |
| 3-methoxypropionic acid | Indole-2-carboxylic acid | 3-Phenoxybenzoic acid |
| 3-Hydroxybutyric acid | 2-benzofurancarboxylic acid | 4-Phenylbutyric acid |
| 4,8-Dihydroxyquinoline- | 2,3,4-Trimethoxybenzoic | 3-(3,4-Dimethoxyphenyl) |
| 2-carboxylic acid | acid | propionic acid |
| (Methylthio)acetic acid | indazole-3-carboxylic acid | 3-chloropropionic acid |
| Pyrrole-2-carboxylic | Benzotriazole-5-carboxylic | 3-bromo-4-methylbenzoic |
| acid | acid | acid |
| 4-Aminobenzoic acid | Indoline-2-carboxylic acid | 3-Bromophenylacetic acid |
| 5-Acetylsalicylic acid | Pentafluoropropionic acid | 4-bromophenylacetic acid |
| 2-Furoic acid | 4-acetylbenzoic acid | 2-Iodobenzoic acid |
| Cyclopentanecarboxylic | 5-Norbornene-2,3- | 9-Plourenone-2- |
| acid | dicarboxylic acid | carboxylic acid |
| | monomethyl ester | |
| trans-3-Hexenoic acid | 3-(5-Nitro-2-furyl)acrylic | xanthene-9-carboxylic |
| 97% | Acid | acid |
| Piperonylic acid | 4-Carboxyphenylboronic acid | 3-Benzoylbenzoic acid |
| 2-tetrahydrofuroic acid | 4-Dimethylaminobenzoic acid | 4-benzoylbenzoic acid |
| 2-Phenoxybenzoic acid | 3-Dimethylaminobenzoic acid | 2-Butynoic acid |
| Tetrahydro-3-furoic | 3-Methoxyphenylacetic acid | 2-Hydroxyisobutyric acid |
| acid | | |
| hexanoic acid | 4-Ethoxybenzoic acid | 2,4-Hexadienoic acid |
| 2-Ethylbutyric acid | 4-methoxyphenylacetic acid | (Ethylthio)acetic acid |
| DL-3-Methylvaleric | (alpha, alpha, alpha-tetra- | 1-Cyclohexene-1- |
| acid, 97% | fluoro-p-tolyl)acetic acid | carboxylic acid |
| Tert-Butylacetic acid, | 1,4-Benzodioxan-2- | 2-Phenoxymethylbenzoic |
| 98% | carboxylic acid | Acid |
| 1-Acetylpiperidine-4- | (R)-(−)-5-oxo-2- | 2-hydroxy-2- |
| carboxylic acid | tetrahydro-furancarboxylic | methylbutyric acid |
| | acid | |
| Vanillic acid | 2,6-Dichloronicotinic acid | 3-Allyloxypropionic acid |
| Benzoic acid | 5-Methoxysalicylic acid | 5-Methylhexanoic acid |
| Picolinic acid, 99% | (4-Pyridylthio)acetic acid | 2-Aminonicotinic acid |
| Nicotinic acid | 2-(Methylthio) nicotinic | 6-Methylpicolinic acid |
| | acid | |
| 2-Pyrazinecarboxylic | 1-Methyl-1- | 2-Ethyl-2-hydroxybutyric |
| acid | cyclohexanecarboxylic acid | acid |
| 1-methyl-2- | 2-Hydroxy-6-methylpyridine- | 3-Cyclohexenecarboxylic |
| pyrrolecarboxylic acid | 3-carboxylic acid | acid |
| 1- | (R)-(+)-3-Methylsuccinic | 2-Hydroxyphenylacetic |
| Isoquinolinecarboxylic | acid-1-monomethyl ester | acid |
| acid | | |
| 4-butylbenzoic acid | Quinoline-4-carboxylic acid | 2,6-Dimethylbenzoic acid |
| 2-Thiophenecarboxylic | 1H-Indole-3-acetic acid | Thiophene-3-carboxylic |
| acid | | acid |
| 5-Fluoroindole-2- | 5-Hydroxy-2- | 2-(n-Propylthio) |
| carboxylic acid | indolecarboxylic acid | nicotinic acid |
| (S)-(−)-2-Pyrrolidone- | (R)-(−)-4-Methylglutaric | DL-2-Hydroxy-4- |
| 5-carboxylic acid | acid 1-monomethyl ester | (methylthio)butyric acid |
| Itaconic acid monoethyl | 5-methylisoxazole-4- | 2-Amino-6-fluorobenzoic |
| ester | carboxylic acid | acid |
| m-Toluic acid | 4-Acetamidobenzoic acid | 2-Mercaptonicotinic acid |
| p-Toluic acid | 4-Aminosalicylic acid | 6-Methylnicotinic acid |
| 2-Methylnicotinic acid | 3-Acetamidobenzoic acid | 2,5-Difluorobenzoic acid |
| 3-aminobenzoic acid | Succinamic acid | o-Toluic acid |
| 2-Chloroisonicotinic | 2-(4-Fluorobenzoyl)benzoic | 2-Fluorophenylacetic |
| acid | acid | acid |
| 3-Hydroxybenzoic acid | 3,4-Dimethoxybenzoic acid | 2-Acetylbenzoic acid |
| 4-Hydroxybenzoic acid | 3,5-Dimethoxybenzoic acid | 4-chlorosalicylic acid |
| 2,5-Dimethoxybenzoic | 3-(3,4-Dihydroxyphenyl) | 1-Phenyl-1-cyclopropane |
| acid | propionic acid | carboxylic acid |
| 5-Norbornene-2- | 5-Methyl-2- | 2,5-Dimethylphenylacetic |
| carboxylic acid | pyrazinecarboxylic acid | acid |
| (2-n- | 3-Hydroxy-4-nitrobenzoic | 2,4,6-Trimethylbenzoic |
| Butoxyethoxy) acetic | acid | acid |
| Acid | | |
| 5-Bromofuroic acid | 5-Nitrosalicylic acid | 2-Ethoxybenzoic acid |
| 6-Hydroxynicotinic acid | 4-Chloro-o-anisic acid | Salicylic acid |
| 2-Methoxyphenylacetic | 3-Chloro-4- | 3-Methyl-2- |
| acid | hydroxyphenylacetic acid | thiophenecarboxylic acid |
| 2,4- | trans-4-n-propylcyclohexane | 2-Amino-5-chlorobenzoic |
| Difluorophenylacetic | carboxylic acid | acid |
| acid | | |
| 2-Chloro-6-methyl-3- | 2-Hydroxyquinoline-4- | O-Chlorophenylacetic |
| pyridinecarboxylic acid | carboxylic acid | acid |
| 4-Fluorobenzoic acid | 3-indolepropionic acid | 4-Octyloxybenzoic acid |
| 3-Flurobenzoic acid | 2-Amino-4-chlorobenzoic | 5-Bromofuroic acid |
| | acid | |
| alpha, alpha,alpha- | Alpha, Alpha, Alpha- | Alpha, Alpha, Alpha- |
| trifluoro-p-toluic acid | Trifluoro-o-toluic acid | Trifluoro-m-toluic acid |
| 2-Thiopheneacetic acid | 2,5-Dimethyl-3-furoic acid | (+/−)-Citronellic acid |
| 3-Thiopheneacetic acid | Chromone-2-carboxylic acid | 2-Fluorobenzoic acid |
| 5-Bromo-2,4- | 2-[(4S)-2,2-Dimethyl-5-oxo- | 2,5-Difluorophenylacetic |
| dihydroxybenzoic acid | 1,3-dioxolane-4-yl]acetic | acid |
| monohydrate | acid | |
| (R)-(+)-2- | 3-Hydroxy-2- | 2,4,5-Trifluorobenzoic |
| Benzyloxypropionic acid | quinoxalinecarboxylic acid | acid |
| 4-cyanobenzoic acid | Coumarin-3-carboxylic acid | 2-Chloronicotinic acid |
| 3-Cyanobanzoic acid | 2,4-Dichlorobenzoic acid | 2-Chloro-6-fluorobenzoic |
| | | acid |
| phthalide-3-acetic acid | 2,5-Dichlorobenzoic acid | 3-indoleglyoxylic acid |
| 2,5-Dimethylphenoxy | 5-Methoxyindole-2- | 2,3,4-Trifluorobenzoic |
| acetic acid | carboxylic acid | acid |
| 2,5-Dimethylbenzoic | 2,6-Dichlorobenzoic acid | 4-Isobutylbenzoic acid |
| acid | | |
| 3,4-Dimethylbenzoic | 3,4-Dichlorobenzoic acid | 1-Naphthoic acid |
| acid | | |
| p-Tolylacetic acid | 2,3-Dichlorobenzoic acid | m-Tolylacetic acid |
| 4-acetylphenoxyacetic | 2,4-Dimethylphenoxyacetic | 2,4-Dimethoxybenzoic |
| acid | acid | acid |
| 2,4-Dimethylbenzoic | (−)-2-oxo-4- | 1-Adamantanecarboxylic |
| acid | thiazolidinecarboxylic acid | acid |
| 3,5-Dimethylbenzoic | 2,3-Dimethylphenoxyacetic | 2-Amino-5-nitrobenzoic |
| acid | acid | acid |
| 2-Bromoacrylic acid | 3-Methylhippuric acid | 3,5-Dichlorobenzoic acid |
| 3-(3-pyridyl) propionic | 4-(4-methoxyphenyl)butyric | 2, 3-Dimethoxybenzoic |
| acid | acid | acid |
| 1-Hydroxy-2-naphthoic | 2-(4-Hydroxyphenoxy) | 2-(allylthio)nicotinic |
| acid | propionic acid | acid |
| 3-methylsalicylic acid | N,N-dimethylsuccinamic acid | 2- (Ethylthio)nicotinic |
| | | acid |
| P-Anisic acid | 2-Mehtylhippuric acid | 6-bromohexanoic acid |
| o-Anisic acid | 5-Chloroindole-2-carboxylic | Itaconic acid mono-n- |
| | acid | butyl ester |
| 4-Nitrophenoxyacetic | trans-4-n-Butylcyclohexane | 2-(4-Chlorophenyl)-2- |
| acid | carboxylic acid | methylpropionic acid |
| 5-methylsalicylic acid | Rhodanine-N-acetic acid | 2-Chloromandelic acid |
| 6-Hydroxy-1-napthoic | 2-Chloro-4,5- | 2-Biphenylcarboxylic |
| acid | difluorobenzoic acid | acid |
| 3,5-dimethoxy-4- | 2,3,4,5-Tetrafluorobenzoic | 4-Bromo-2-fluorocinnamic |
| methylbenzoic acid acid | acid | |
| 1-Adamantaneacetic acid | 2-Chloro-4- | 1-Naphthaleneacetic acid |
| | fluorophenylacetic acid | |
| Cyclopentylacetic acid | (2,5-Dimethoxyphenyl)acetic | 2-Chloro-4- |
| | acid | fluorocinnamic acid |
| 1-Phenylcyclopentane | 2-(4-Chlorophenoxy)-2- | Cyclohexanecarboxylic |
| carboxylic acid | methylpropionic acid | acid |
| 1-(p-Tolyl)-1- | (2S)-4-(1,3- | 2,6-Dichloro-5- |
| cyclopentanecarboxylic | Dioxoisoindolin-2-yl)-2- | fluoropyridine-3- |
| acid | hydroxy butanoic acid | carboxylic acid |
| 2,6- | (4-Chlorophenylthio)acetic | 3-Hydroxy-7-methoxy-2- |
| Dichlorophenylacetic | acid | naphthoic acid |
| acid | | |
| (−)-Camphanic acid | 2,3-Diphenylpropionic acid | DL-2-Methylbutyric acid |
| 2-Amino-5-bromobenzoic | Beta-(4-Methylbenzyl) | Rhodanine-3-propionic |
| acid | mercaptopropionic acid | acid |
| 2,5-Dimethoxy cinnamic | 2,5-Dichlorophenyithio | trans-2-Methyl-2- |
| acid | glycolic acid | pentenoic acid |
| trans-2-Pentenoic acid | (−)-Camphanic acid | 2-Methyl-3-furoic acid |
| Valeric acid | mono-Ethyl malonate | trans-2-hexenoic acid |
| 3-(2- | 2-Chloro-6- | 4-Benzyloxyphenylacetic |
| benzothiazolylthio) | fluorophenylacetic acid | acid |
| propionic acid | | |
| 2,4,Dichlorophenylacetic | 5-Bromo-2-fluorocinnamic | 4-(4-tert- |
| acid | acid | butylphenyl)benzoic acid |
| (+/−)-2-(6-Methoxy-2- | 2-(carboxymethylthio)-4,6- | 1-Piperidinepropionic |
| naphthyl)propionic acid | dimethylpyridine | acid |
| | monohydrate | |
| 3-Cyclopentylpropionic | (2- | Alpha-Methylcinnamic |
| acid | Benzothiazolylthio) acetic | acid |
| | acid | |
| 2-Ethoxynaphthoic acid | DL-Lactic acid | 2-Methyihexanoic acid |
| trans-3-Furanacrylic | 1-(4-Methoxyphenyl)-1- | 3-Hydroxy-2-pyridine- |
| acid | cyclopentanecarboxylic acid | carboxylic acid |
| 2,3-Dichlorophenoxy | 2,4-Dichlorophenoxy acetic | 3-Mercaptoisobutyric |
| acetic acid | acid | Acid |
| 5-Fluoro-2- | (3,4-Dimethoxyphenyl) acetic | 2-Thiopheneglyoxylic |
| methylbenzoic acid | acid | acid |
| (2-Napthoxy)-acetic | o-Tolylacetic acid | 2-Hydroxyoctanoic acid |
| acid | | |
| Urocanic acid | Hydrocinnamic acid | N-Acetyl-1-proline |
| Dl-Mandelic acid | DL-2-Phenylpropionic acid | N-Methyl-maleamic acid |
| Coumalic acid | 4-(Methylamino)benzoic acid | 3,4-Difluorobenzoic acid |
| 4-Methyl-1-cyclohexane | Tetrahydro-2,2-dimethyl-5- | DL-2-phenoxypropionic |
| carboxylic acid | oxo-3-furancarboxylic acid | acid |
| m-Anisic acid | 3-Hydroxyphenylacetic acid | Indole-3-carboxylic acid |
| Cyclohexylacetic acid | Phenoxyacetic acid | 3-Fluorocinnamic acid |
| Cycloheptanecarboxylic | 3-Amino-1H-1,2,4-triazole- | 3-Fluoro-4-methylbenzoic |
| acid | 5-carboxylic acid | acid |
| 2-Octynoic acid | trans-Styrylacetic acid | 2-Methylcinnamic acid |
| 2-Propylpentanoic acid | 3-Fluorophenylacetic acid | 4-Acetylbutyric acid |
| 2-Methylheptanoic acid | Furylacrylic acid | Phenylpyruvic acid |
| Octanoic acid | Thiosalicylic acid | mono-Ethyl succinate |
| 3-(2-Thienyl) acrylic | Alpha-Methylhydrocinnamic | Alpha-Fluorocinnamic |
| acid | acid | acid |
| mono-Methyl glutarate | 3-(2-Thienyl)propanoic acid | 3-Phenoxypropionic acid |
| trans-3- (3- | trans-3-(3-Thienyl)acrylic | 3,4-(Methylenedioxy) |
| Pyridyl)acrylic acid | acid | phenylacetic acid |
| 3-Noradamantane | 4-Acetyl-3,5-dimethyl-2- | 3-(2-Hydroxyphenyl) |
| carboxylic acid | pyrrolecarboxylic acid | propionic acid |
| 2-Nitrobenzoic acid | DL-Atrolactic acid | 4-Methylsalicylic acid |
| 4- | 2-Methyl-1H-benzimidazole | 3-Fluoro-4- |
| (Dimethylamino)butyric | 5-carboxylic acid | methoxybenzoic acid |
| acid hydrochloride | | |
| 3-Chloro-4- | 4-(Dimethylamino) | 3,4-Difluorocinnamic |
| hydroxybenzoic acid | phenylacetic acid | acid |
| DL-3-Phenyllactic acid | 3-Benzoylpropionic acid | Homovanillic acid |
| 2-Methyl-terephthalic | 3-(Diethylamino) propionic | 3-(4-Methylbenzoyl) |
| acid | acid hydrochloride | propionic acid |
| 4-(2-Thienyl) butyric | 3,4-Dihydro-2,2-dimethyl-4- | Cyclohexanepentanoic |
| acid | oxo-2H-pyran-E-carboxylic | acid |
| | acid | |
| Cyclohexanebutyric acid | mono-Methyl phthalate | Undecanoic acid |
| 3-Chlorophenylacetic | 3,5-Difluorophenylacetic | 6-Hydroxy-2-naphthoic |
| acid | acid | acid |
| 3-Benzoylacrylic acid | 4-Amino-2-chlorobenzoic | 3-Indoleacrylic acid |
| | acid | |
| 3-Amino-4-chlorobenzoic | 4-(4-Methylphenyl)butyric | 3-Hydroxy-2-naphthoic |
| acid | acid | acid |
| 3,4- | 3-(4- | |
| Difluorophenylacetic | Methoxyphenyl)propionic | 2-Hydroxy-1-naphthoic |
| acid | acid | acid |
| 2,5-Dimethylphenoxy | trans-3-(4- | 5-Methyl-2-nitrobenzoic |
| acetic acid | Methylbenzoyl)acrylic acid | acid |
| 3-Quinolinecarboxylic | 3-(2- | 3,5-Dimethyl-p-anisic |
| acid | Methoxyphenyl)propionic | acid |
| | acid | |
| Decanoic acid | 2-Naphthoic acid | 4-Benzoylbutyric acid |
| 5-Chlorosalicylic acid | Quinaldic acid | N-Methylhippuric acid |
| 3-(3-Methoxyphenyl) | 5-Nitrothiophene-2- | 4-(Diethylamino) benzoic |
| propionic acid | carboxylic acid | acid |
| 2-Methyl-6-nitrobenzoic | Alpha, Alpha, Alpha-2- | N,N-Dimethyl-1- |
| acid | Tetrafluoro-p-toloic acid | phenylalanine |
| Ibuprofen | 2-Nitrophenylacetic acid | 4-Benzyloxybutyric acid |
| 3-Pyridylacetic acid | 2-Methyl-5-nitrobenzoic | Diethylphosphonoacetic |
| | acid | acid |
| 2-Oxo-6-pentyl-2H- | mono-Methyl cis-5- | 2-Methyl-3-nitrobenzoic |
| pyran-3-carboxylic acid | norbornene-endo-2,3- | acid |
| | dicarboxylate | |
| DL-2-(3-Chlorophenoxy) | 3,5-Dichloro-4- | trans-2-Chloro- |
| propionic acid | hydroxybenzoic acid | fluorocinnamic acid |
| 5-Bromo-2-thiophene | DL-4-Hydroxy-3- | 2-Phenylmercapto |
| carboxylic acid | methoxymandelic acid | methylbenzoic acid |
| 3,4-Diethoxybenzoic | Alpha-Phenyl-o-toluic acid | Diphenylacetic acid |
| acid | | |
| 5-Bromosalicylic Acid | Adipic acid monoethyl ester | Syringic acid |
| 3,5-Dichloroanthranilic | trans-2,4-Dimethoxycinnamic | 4-(4-Hydroxyphenyl) |
| acid | acid | benzoic Acid |
| Alpha-Phenylcinnamic | trans-2,3-dimethoxycinnamic | 3-(Phenylsulfonyl) |
| acid | acid | propionic acid |
| 3,3-Diphenylpropionic | (s)-(−)-2-[(Phenylamino) | 3-(Trifluoromethyl) |
| acid | carbonyloxy]propionic acid | cinnamic acid |
| Cyclohexylphenylacetic | 4-(3-Methyl-5-oxo-2- | 3,4-Dimethoxycinnamic |
| acid | pyrazoline-1-yl)benzoic | acid |
| | acid | |
| 4-(Trifluoromethyl) | Pentafluorophenoxyacetic | Trans-2,4- |
| mandelic acid | acid | Dichlorocinnamic acid |
| 2-Nitrophenylpyruvic | Aipha-Phenylcyclopentane | 3,4-Dichlorophenylacetic |
| acid | acetic acid | acid |
| 4-(Hexyloxy)benzoic | 4-Butoxyphenylacetic acid | 4-Bromocinnamic acid |
| acid | | |
| 7-Hydroxycoumarin-4- | 3-(3,4,5-Trimethoxyphenyl) | 2-Chloro-5- |
| acetic acid | propionic acid | (methylthio)benzoic acid |
| 1,3-dioxo-2- | 3,4,5-Trimethoxy | 3-Bromo-4-fluorocinnamic |
| isoindolineacetic acid | phenylacetic acid | acid |
| Anthracene-9-carboxylic | p-Bromophenoxyacetic acid | N-Carbobenzyloxy-L- |
| acid | proline | |
| (Phenylthio)acetic acid | 4-Butoxyphenylacetic acid | 3-Phenylbutyric acid |
| Acridine-9-carboxylic | 4-Benzyloxybenzoic acid | 3,4,5-Triethoxybenzoic |
| acid hydrate | | acid |

| 7-Chloro-4-hydroxy-3- | 1,4-dihydro-1-ehtyl-7-methyl-4-oxo-1,8-naphthyridine- | |
|---|---|---|
| quinolinecarboxylic | 3-carboxylic acid | |
| acid | | |
| gamma-Oxo-(1,1′- | 2-Ethoxycarbonylamino-3- | 3,5-Di-tert-butyl-4- |
| biphenyl)-4-butanoic | phenyl-propionic acid | hydroxybenzoic acid |
| aicd | | |
| 2-Cyclopentene-1-acetic | 3,4,5-Trimethoxycinnamic | 3-(BOC-amino)benzoic |
| acid | acid | acid |
| 4-Methoxysalicylic acid | 4-Fluorocinnamic acid | 4,5-Dibromo2-furoic acid |
| 2-Hydroxynicotinic acid | 4-Bromo-3,5- | 5-Phenylvaleric acid |
| | dihydroxybenzoic acid | |
| 4-Pentynoic acid | 4-Ethoxybenzoic acid | 4-Acetoxybenzoic acid |
| 3,3-Dimethylacrylic | Dicyclohexylacetic acid | 3-Acetoxybenzoic acid |
| acid | | |
| 4-Methoxy-2- | cis-2-(2-Thiophenecarbonyl)- | 4-Methyl-3-nitrobenzoic |
| methylbenzoic acid | 1-cyclohexanecarboxylic | acid |
| | acid | |
| 4-Methylvaleric acid | (2-Methylphenoxy)acetic | 4-Isopropoxybenzoic acid |
| | acid | |
| 3,3,3- | (4-Methylphenoxy) acetic | 4-Nitrophenylacetic acid |
| Trifluoropropionic acid | acid | |
| 2-Methyl-1-cyclohexane | 2,2,3,3-Tetramethyl | 3-Methyl-1-cyclohexane |
| carboxylic acid | cyclopropanecarboxylic acid | carboxylic acid |
| 4-Amino-3-nitrobenzoic | 5-Methyl-2- | 4-Methoxyphenoxyacetic |
| acid | thiophenecarboxylic acid | acid |
| 3-Methoxysalicylic acid | 4-Fluorophenylacetic acid | 2-Phenoxybutyric acid |
| 3,5-Dimethoxy-4- | (R)-(−)-2,2-Dimethyl-5- | 4-Hydroxymandelic acid |
| hydroxycinnamic acid | oxo-1,3-dioxolane-4-acetic | monohydrate |
| | acid | |
| (2-Methoxyphenoxyl) | 2,2-Dichloro-1-methylcyclo- | 4-Hydroxyphenylacetic |
| acetic acid | propanecarboxylic acid | acid |
| 2-Ethylbenzoic acid | 4-Fluorophenoxyacetic acid | 4-tert-Butylbenzoic acid |
| 5-Fluoro-2- | (R)-(+)-2-(4-Hydroxy | 2,6-Dimethoxynicotinic |
| methoxybenzoic acid | phenoxy)-propionic acid | acid |
| 2-Carboxyethyl | 4-Hydroxy-3-nitrobenzoic | 3,4-Difluorohydro |
| phosphonic acid | acid | cinnamic acid |
| 4-Hydroxy-3-methoxy | 3-Chloro-2-methylbenzoic | 2-Chloro-4-fluorobenzoic |
| benzoic acid | acid | acid |
| 4-Fluoro-3- | 2-Chloro-6-methylnicotinic | 4-Chlorophenoxyacetic |
| methylbenzoic acid | acid | acid |
| 3-Fluoro-2- | 2,2-Bis(hydroxymethyl) | 5-Chloro-2- |
| methylbenzoic acid | butyric acid | methoxybenzoic acid |
| 5-Amino-4-methyl- | (2,2-Dimethyl-5-[2,5- | (Alpha, Alpha, Alpha- |
| cyclohexa-1,5-diene- | dimethylphenoxy]-pentanoic | Trifluoro-m-tolyl)acetic |
| 1,4-dicarboxylic acid | acid) | acid |
| 4-Methoxycyclohexane | 1-Methylindole-3-carboxylic | (R)-(−)-3- |
| carboxylic acid | acid | Chloromandelic acid |
| 4-Propylbenzoic acid | 4-Chlorophenylacetic acid | 4-Bromornandelic acid |
| 2 -Methoxy-4- | 4-Oxo-4H-1-benzopyran-2- | 2-Mercapto-4-methyl-5- |
| (methylthio)-benzoic | carboxylic acid | thiazoleacetic acid |
| acid | | |
| 2-(Trifluoromethyl) | 4-Methoxy-3-nitrobenzoic | 3,4-Dichlorocinnarnic |
| cinnamic acid | acid | acid |
| 3-Methylcyclohexane | 4-Methoxy-2- | 5-Methoxy-2-methyl-3- |
| carboxylic acid | quinolinecarboxylic acid | indoleacetic acid |
| 2-(4-Nitrophenyl) | 4-(4-Methoxyphenyl)butyric | 4-Carboxybenzene |
| propionic acid | acid | sulfonamide |
| 2-Hydroxy-5-(1H-pyrrol- | 3-Chloro-4- | 5-Chloro-2-nitrobenzoic |
| 1-yl)-benzoic acid | hydroxyphenylacetic acid | acid |
| 2-Methyl-3-indoleacetic | 2-Fluoro- | 4-Amino-5-chloro-2- |
| acid | 3 (trifluoromethyl)-benzoic | methoxybenzoic acid |
| | acid | |
| 4-Chloro-2- | 2-(2-Nitrophenoxy)acetic | 3-Acetoxy-2- |
| fluorocinnamic acid | acid | methylbenzoic acid |
| 2,4,6-Trichlorobenzoic | 3,4-Dichlorophenoxyacetic | 2-Bibenzylcarboxylic |
| acid | acid | acid |
| 2-Chloro-5- | (S)-(+)-6-Methoxy-alpha- | 4-(3,4-Dimethoxyphenyl)- |
| (trifluoromethyl) benzoic | methyl-2-naphthalenacetic | butyric acid |
| acid | acid | |
| 4-Ethylbiphenyl-4′- | 2-Bromo-5-methoxybenzoic | 5-Bromo-2-chlorobenzoic |
| carboxylic acid | acid | acid |
| 3,5-Dinitro-p-toluic | 1-Methyl-2- | 1-Methyl-3-indoleacetic |
| acid | nitroterephthalate | acid |
| 4-Pentylbenzoic acid | 4-n-Heptyloxybenzoic acid | 4-Biphenylacetic acid |

Alternatively, bi-ligand libraries of the invention can be built through the direct reaction of isocyanates or thioisocyanates using a combination of solid phase chemistry and solution phase chemistry.

As shown in FIG. 5c, bi-ligand libraries of the invention can further be prepared in the following manner. A solution of an isocyanate or thioisocyanate and a common ligand mimic of the invention is formed in a solvent, such as DMSO. The isocyanate and common ligand mimic are allowed to react overnight, followed by the addition of aminomethylated polystyrene Resin (NovaBiochem, Cat. No. 01-64-0383). This mixture is then shaken at room temperature for a period of time, for example about 4 hours. The resin then can be filtered and dried under reduced pressure to yield the desired product. Nonlimiting examples of isocyanates and thioisocyanates are provided in Table 3.

TABLE 3

| | |
|---|---|
| allyl isocyanate | 3-chloro-4-methylphenyl isocyanate |
| N-propyl isocyanate | 1-naphthyl isocyanate |
| pentyl isocyanate | 3-chloro-4-fluorophenyl isocyanate |
| phenyl isocyanate | 2,6-diethylphenyl isocyanate |
| m-tolyl isocyanate | 1-adamantyl isocyanate |
| p-tolyl isocyanate | 2-methyl-4-nitrophenyl isocyanate |
| o-tolyl isocyanate | 2-methyl-5-nitrophenyl isocyanate |
| benzyl isocyanate | 2-methyl-3-nitrophenyl isocyanate |
| 4-fluorophenyl isocyanate | 4-methyl-2-nitrophenyl isocyanate |
| heptyl isocyanate | 4-methyl-3-nitrophenyl isocyanate |
| 3-cyanophenyl isocyanate | 2,4-dimethoxyphenyl isocyanate |
| 2,6-dimethylphenyl isocyanate | 2,5-dimethoxyphenyl isocyanate |
| 2-ethylphenyl isocyanate | 2-fluoro-5-nitrophenyl isocyanate |
| 2,5-dimethylphenyl isocyanate | 4-fluoro-3-nitrophenyl isocyanate |
| 2,4-dimethylphenyl isocyanate | 5-chloro-2-methoxyphenyl isocyanate |
| 3,4-dimethylphenyl isocyanate | ethyl-6-isocyanatohexanoate |
| 4-ethylphenyl isocyanate | 4-(trifluoromethyl) phenyl isocyanate |
| 3-ethylphenyl isocyanate | 3-(trifluorornethyl) phenyl isocyanate |
| 2,3-dimethylphenyl isocyanate | 2-(trifluoromethyl) phenyl isocyanate |
| 2-methoxyphenyl isocyanate | 3,4-dichlorophenyl isocyanate |
| 3-methoxyphenyl isocyanate | 2,4-dichlorophenyl isocyanate |
| 4-methoxyphenyl isocyanate | 3,5-dichlorophenyl isocyanate |
| 5-chloro-3-methylphenyl | 2,3-dichlorophenyl isocyanate |
| isocyanate | |
| 2-chlorophenyl isocyanate | trichloroacetyl isocyanate |
| 3-chlorophenyl isocyanate | ethyl-4-isocyanatobenzoate |
| 2,4 -difluorophenyl isocyanate | Isopropyl isocyanate |
| 3,4-difluorophenyl isocyanate | Butyl isocyanate |
| 2,6-difluorophenyl isocyanate | cyclopentyl isocyanate |
| butyl isocyanatoacetate | cyclohexyl isocyanate |
| trans-2-phenylcyclopropyl | o-tolyl isocyanate |
| isocyanate | |
| Trichloromethyl isocyanate | 3-fluorophenyl isocyanate |
| 3-acetylphenyl isocyanate | 2-fluorophenyl isocyanate |
| 4-acetylphenyl isocyanate | ethyl 3-isocyanatopropionate |
| 2-isopropylphenyl isocyanate | 4-methylbenzyl isocyanate |
| 2-ethyl-6-methylphenyl isocyanate | phenethyl isocyanate |
| 2,4,6-trimethylphenyl isocyanate | 3-fluorobenzyl isocyanate |
| 4-ethoxyphenyl isocyanate | 4-fluorobenzyl isocyanate |
| 2-methoxy-5-utethylphenyl | 3-fluoro-4-rnethylphenyl isocyanate |
| isocyanate | |
| 2-ethoxyphenyl isocyanate | 2,4-difluorophenyl isocyanate |
| 4-methoxy-2-methylphenyl | 3,4-difluorophenyl isocyanate |
| isocyanate | |
| 4-methoxybenzyl isocyanate | 2,6-difluorophenyl isocyanate |
| 2-nitrophenyl isocyanate | 3,5-difluorophenyl isocyanate |
| 4-nitrophenyl isocyanate | octyl isocyanate |
| 3-nitrophenyl isocyanate | 1,1,3,3-tetramethylbutyl isocyanate |
| 4-(methylthio)phenyl isocyanate | trans-2-phenylcyclopropyl isocyanate |
| 2-(methylthio)phenyl isocyanate | trichloromethyl isocyanate |
| 5-chloro-2-methylphenyl | 4-isopropylphenyl isocyanate |
| isocyanate | |
| 4-chloro-2-methylphenyl | propyl isothiocyanate |
| isocyanate | |
| 2-isopropyl-6-methylphenyl | 3,4-(methylenedioxy) phenyl |
| isocyanate | isocyanate |
| 2-chloro-6-methylphenyl | 2-chloro-5-methylphenyl isocyanate |
| isocyanate | |
| 3-chloro-2-methylphenyl | 2-chlorobenzyl isocyanate |
| isocyanate | |
| isobutyl isothiocyanate | 3-chloro-4-fluorophenyl isocyanate |
| tert-butyl isothiocyanate | 2,6-diethylphenyl isocyanate |
| N-butyl isothiocyanate | 4-N-butylphenyl isocyanate |
| 2-methoxyethyl isothiocyanate | methyl-4-isocyanato-benzoate |
| N-amyl isothiocyanate | 3-carbomethoxyphenyl isocyanate |
| 3-methoxypropyl isothiocyanate | methyl-2-isocyanatobenzoate |
| phenyl isothiocyanate | 1-adamantyl isocyanate |
| cyclohexyl isothiocyanate | 2-methyl-4-nitrophenyl isocyanate |
| 2-tetrahydrofurfuryl isothiocyanate | 2-methyl-5-nitrophenyl isocyanate |
| o-tolyl isothiocyanate | 2-methyl-3-nitrophenyl isocyanate |
| benzyl isothiocyanate | 4-methyl-2 -nitrophenyl isocyanate |
| m-tolyl isothiocyanate | 4-methyl-3-nitrophenyl isocyanate |
| 4-fluorophenyl isothiocyanate | diethoxyphosphinyl isocyanate |
| 2-fluorophenyl isothiocyanate | 2,4-dimethoxyphenyl isocyanate |
| 3-fluorophenyl isothiocyanate | 2,5-dimethoxyphenyl isocyanate |
| heptyl isothiocyanate | 3,4-dimethoxyphenyl isocyanate |
| ethyl 3-isothiocyanatopropionate | 2-fluoro-5-nitrophenyl isocyanate |
| ethyl 2-isothiocyanatopropionate | 4-fluoro-3-nitrophenyl isocyanate |
| 4-cyanophenyl isothiocyanate | benzenesulphonyl isocyanate |
| 2-ethylphenyl isothiocyanate | 5-chloro-2-methoxyphenyl isocyanate |
| 2,6-dimethylphenyl isothiocyanate | 3-chloro-4-methoxyphenyl isocyanate |
| 2-phenylethyl isothiocyanate | ethyl-6-isocyanatohexanoate |
| 2,4-dimethylphenyl isothiocyanate | 4-(trifluoromethyl)phenyl isocyanate |
| 4-methylbenzyl isothiocyanate | 3-(trifluoromethyl) phenyl isocyanate |
| 2-phenylethyl isothiocyanate | 2-(trifluoromethyl)phenyl isocyanate |
| 3-methoxyphenyl isothiocyanate | 2-(trifluoromethyl)phenyl isocyanate |
| 2-methoxyphenyl isothiocyanate | 3,4-dichlorophenyl isocyanate |
| 4-methoxyphenyl isothiocyanate | 2,6-dichlorophenyl isocyanate |
| 4-chlorophenyl isothiocyanate | 2,4-dichlorophenyl isocyanate |
| 2-chlorophenyl isothiocyanate | 2,5-dichlorophenyl isocyanate |
| 3-chlorophenyl isothiocyanate | 3,5-dichlorophenyl isocyanate |
| 2,4-difluorophenyl isothiocyanate | 2,3-dichlorophenyl isocyanate |
| 2-morpholinoethyl isothiocyanate | trichloroacetyl isocyanate |
| 3-acetylphenyl isothiocyanate | 2-ethyl-6-isopropylphenyl isocyanate |
| 4-isopropylphenyl isothiocyanate | ethyl-3-isocyanatohenzoate |
| 2-isopropylphenyl isothiocyanate | ethyl-4-isocyanatohenzoate |
| 4-(dimethylamino)phenyl | 2-isopropyl-6-methylphenyl |
| isothiocyanate | isocyanate |
| 4-ethoxyphenyl isothiocyanate | ethyl-2-isocyanatohenzoate |
| 4-methoxybenzyl isothiocyanate | 4-butoxyphenyl isocyanate |
| 3-nitrophenyl isothiocyanate | 2-methoxy-5-nitrophenyl isocyanate |
| 4-nitrophenyl isothiocyanate | 2-biphenylylisocyanate |
| 2-(methylthio)phenyl | 4-biphenyl isocyanate |
| isothiocyanate | |
| 3-(methylthio)phenyl | p-toluenesulphonyl isocyanate |
| isothiocyanate | |
| 4-(methylthio)phenyl | o-toluenesulphonyl isocyanate |
| isothiocyanate | |
| 1-naphthyl isothiocyanate | undecyl isocyanate |
| 2-chlorobenzyl isothiocyanate | 2-bromophenyl isocyanate |
| 4-chlorobenzyl isothiocyanate | 3-bromophenyl isocyanate |
| 3-chloro-4-methylphenyl | 4,5-dimethyl-2-nitrophenyl |
| isothiocyanate | isocyanate |
| 4-chloro-2-methylphenyl | 5-chloro-2-methylphenyl |
| isothiocyanate | isothiocyanate |
| 4-bromophenyl isocyanate | 2-chloro-4-nitrophenyl isocyanate |
| 3-morpholinopropyl isothiocyanate | 2-chloro-5-nitrophenyl isocyanate |
| 4-N-butylphenyl isothiocyanate | 4-chloro-2-nitrophenyl isocyanate |
| allyl isothiocyanate | ethyl isothiocyanate |
| 2-methoxycarbonylphenyl | 2-chloro-6-methylphenyl |
| isothiocyanate | isothiocyanate |
| 1-adamantyl isothiocyanate | isopropyl isothiocyanate |
| 4-methyl-2-nitrophenyl | 4-chloro-3-nitrophenyl |
| isothiocyanate | isothiocyanate |
| 3,4-dimethoxyphenyl | 3-bromophenyl isothiocyanate |
| isothiocyanate | |
| 2,5-dimethoxyphenyl | 2-bromophenyl isothiocyanate |
| isothiocyanate | |
| 2,4-dimethoxyphenyl | 2,6-diisopropylphenyl isothiocyanate |
| isothiocyanate | |
| 5-chloro-2-methoxyphenyl | 2-(3,4-dimethoxyphenyl) ethyl |
| isothiocyanate | isothiocyanate |
| 2-(trifluoromethyl) phenyl | 4-bromo-2-methylphenyl |
| isothiocyanate | isothiocyanate |
| 4-(trifluoromethyl)phenyl | 2-bromo-4-methylphenyl |
| isothiocyanate | isothiocyanate |
| 2,6-dichlorophenyl isothiocyanate | cyclododecyl isothiocyanate |
| 2,3-dichlorophenyl isothiocyanate | 4-phenylazophenyl isothiocyanatellil |
| 3,5-dichlorophenyl isothiocyanate | 4-diethylaminophenyl isothiocyanate |

4-methoxy-2-nitrophenyl isothiocyanate

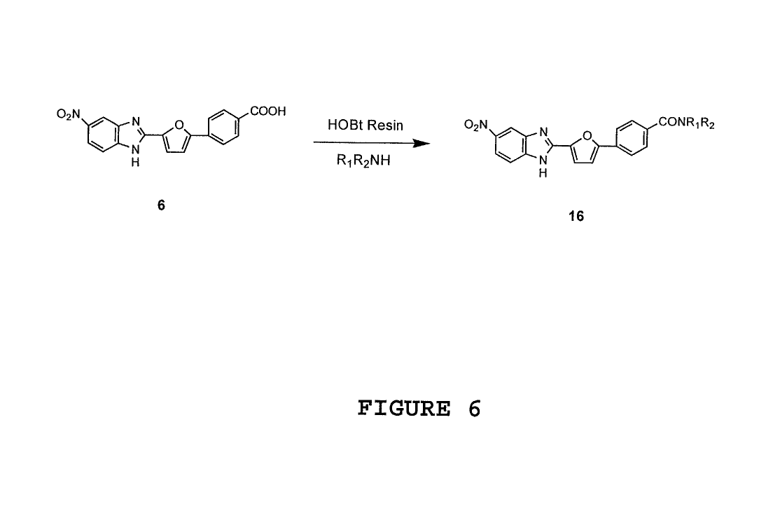

In accordance with the description provided above, combinatorial libraries have been prepared by reacting common ligand mimics of the invention containing a carboxylic acid substituent with amines. These combinatorial libraries are prepared by the reaction scheme depicted in FIG. 6, for example, as follows. HOBt resin is swelled in a solvent, such as a mixture of dry THF and dry DMF. The rein is then added to the common ligand mimic of the invention, which is dissolved in dry DMF containing DIC (N,N′-diisopropylcarbodiimide). The mixture is then shaken overnight at room temperature. The product is then washed three times with dry DMF and three times with dry THF. The resin mixture is then added to the amine, which was dissolved in a solvent, such as dry DMF. The mixture is again shaken overnight at room temperature. The resin then can be filtered and washed wish additional solvent. The filtrate then can be collected and vacuum dried. Combinatorial libraries of the invention have been prepared using the amines in Table 4. The preparation of combinatorial libraries employing this method is further described in Example 12.

TABLE 4

| | |
|---|---|
| Cyclopropylamine | 3-pyrroline |
| Isopropylamine | hydroxylamine hydrochloride |
| Propylamine | cyclobutylamine |
| Pyrrolidine | N-methylallyiamine |
| Diethylamine | morpholine |
| Isobutylamine | 1-ethylpropylamine |
| N-butylamine | neopentylamine |
| N-methylpropylamine | N-ethylisopropylamine |
| sec-butylamine | N-methylbutylamine |
| 2-methoxyethylamine | 2-amino-1-methyloxypropane |
| 4-amino-1,2,4-triazole | 3-methoxypropylamine |
| Cyclopentylamine | thiazolidine |
| Piperidine | 3-amino-1,2,4-triazine |
| Dipropylamine | furfurylamine |
| 4-aminomorpholine | diallylamine |
| N-acetylethylenediamine | 2-methylpiperidine |
| 3-dimethylaminopropylamine | 3-methylpiperidine |
| N-isopropylethylenediamine | 4-methylpiperidine |
| 4-amino-N-methylphthalimide | cyclohexylamine |
| 2-(aminomethyl)-1,3-dioxolane | hexamethyleneimine |
| 5-amino-1-pentanol | 1-aminopiperidine |
| 3-ethoxypropylamine | 1-methylpiperazine |
| 3-(methylthio) propylamine | tetrahydrofurfurylamine |
| Benzylamine | 1,3-dimethylbutylamine |
| m-toluidine | 1-(4-nitrophenyl)piperazine |
| o-toluidine | exo-2-aminonorbornane |
| p-toluidine | 2-thiophenemethylamine |
| 2-(aminomethyl)pyridine | 2-methylcyclohexylamine |
| 3-(aminomethyl)pyridine | 3,5-dimethylpiperidine |
| 4-(aminomethyl)pyridine | 4-methylcyclohexylamine |
| 3-fluoroaniline | cycloheptylamine |
| Cyclohexanemethylamine | N-propylcyclopropanemethyiamine |
| Heptamethyleneimine | 1-piperazinecarboxaldehyde |
| 2-amlno-4-methylthiazole | 2,6-dimethylmorpholine |
| 1-ethylpiperazine | 1-amino-4-methylpiperazine |
| 1-methylhomopiperazine | 2-heptylamine |
| N-(2-aminoethyl)pyrrolidine | N-methylhexylamine |
| N,N-diethylethylenediamine | N,N,N′-trimethyl-1,3-propanediamine |
| N,N-dimethyl-N-ethylethylenediamine | 2-methylbenzylamine |
| 4-ethynylaniline | 3,4-dimethylaniline |
| 2-(2-aminoethyl)-1,3-dioxolane | 3-methylbenzylamine |
| 6-amino-1-hexanol | 4-methylbenzylamine |
| 3-isopropoxypropylamine | N-methylbenzylainine |
| 2-(2-amanoethyl)pyridine | phenethylamine |
| 6-amino-m-cresol | 5-amino-o-cresol |
| m-anisidine | |
| p-anisidine | |
| 2-(aminomethyl)-5-methylpyrazine | 2-(1-cyclohexenyl)ethylamine |
| 2,(2-thienyl)ethylamine | 1-(3-aminopropyl)pyrrolidine |
| Cyclooctylamine | 2-(2-aminoethyl)-1-methylpyrrolidine |
| 1-acetylpiperazine | 2-(aminomethyl)-1-ethylpyrrolidine |
| Isonipecotamide | N-(2-aminoethyl)-piperidine |
| Nipecotamide | 4-(2-aminoethyl)morpholine |
| N,N-diethyl-N′-methylethylenediamine | 2-(aminomethyl)benzimidazole |
| ethyl 3-aminobutyrate | 3-aminobenzamide |
| 5-aminoindan | 4-aminobenzamide |
| trans-2-phenylcyclopropylamine | N-(4-pyridylmethyl)ethylamine |
| 3-phenyl-1-propylamine | N,N-dimethyl-1,4-phenylenediamine |
| beta-methylphenethylamine | 3,4-(methylenedioxy)-aniline |
| N-methylphenethylamine | 4 -hydroxybenzamide |
| p-isopropylaniline | 6-aminonicotinamide |
| 3-methoxybenzylamine | 2,4-dimethyl-6-aminophenol |
| 4-ethoxyaniline | 3-amino-4-methylbenzyl alcohol |
| 4-methoxy-2-methylaniline | 4-methoxybenzylamine |
| m-phenetidine | 2-chlorobenzylamine |
| 5-amino-2-methoxyphenol | 3-chlorobenzylamine |
| Tyramine | 4-chlorobenzylamine |
| 2-fluorophenethylamine | N-(3′-aminopropyl)-2-pyrrolidinone |
| 3-fluorophenethylamine | 2,4-difluorobenzylamine |
| 3-(methylthio) aniline | 2,5-difluorobenzylamine |
| 4-(methylthio) aniline | 2, 6-difluorobenzylamine |
| 2-amino-4-methoxy-6-methylpyrimidine | 3,4-difluorobenzylamine |
| 3,3,5-trimethylcyclohexylamine | 1,3,3-trimethyl-6- |
| | azabicyclo[3,2,1]octane |
| p-phenetidine hydrochloride | 2-aminonaphthalene |
| 8-aminoquinoline | N-(3-aminopropyl) morpholine |
| 2-ethoxybenzylamine | 7-amino-4-methylcoumarin |
| 2-methoxyphenethylamine | 4-piperidone monohydrate hydrochloride |
| 4-isopropoxyaniline | 2-amino-1-methylbenzimidazole |
| 4-methoxyphenethylamine | 1,2,3,4-tetrahydro-1-naphthylamine |
| 3,5-dimethoxyaniline | 1-amino-5,6,7,8-tetrahydronaphthalene |
| (−)-cis-myrtanylamine | 1-methyl-3-phenylpropylamine |
| 1-aminonaphthalene | 4-amino-2,2,6,6-tetramethylpiperidine |
| 4-tert-butylcyclohexylamine | 4-tert-butylaniline |
| 2-(3-chlorophenyl)ethylamine | 4-aminoacetanilide |
| 2-(4-chlorophenyl)ethylamine | 1,4-benzodioxan-5-amine |
| 1-(3-aminopropyl)-2-pipecoline | methyl 3-amino-benzoate |
| 4-phenylbutylamine | 1-(3-aminopropyl)-4-methyl-piperazine |
| ethyl nipecotate | 1-phenylpiperazine |
| 1-naphthalenemethylamine | 1-(2-pyridyl) piperazine |
| 2-(furfurylthio)ethylamine | 4-pentylaniline |
| Piperonylamine | N-(3-aminopropyl)-N-methylaniline |
| Decylamine | N,N-diethyl-p-phenylenediamine |
| 3-chloro-p-anisidine | 4-butoxyaniline |
| 5-amino-1-napthol | 2,3-dimethoxybenzylamine |
| 2-amino-5,6-dimethyl-benzimidazole | 2,4-dimethoxybenzylamine |
| 1-cyclohexylpiperazine | 3,5-dimethoxybenzylamine |
| 4-piperidinopiperidine | ethyl 4-aminobutyrate |
| 2-amino-5-chlorobenzoxazole | 2,4-dichlorobenzylamine |
| 2-amino-5-trifluoromethyl-1,3,4- | ethyl 4-amino-1-piperidinecarboxylate |
| thiadiazole | |
| 2-aminobiphenyl | 4-aminoquinaldine |
| 3-aminobiphenyl | (3S)-(+)-1-benzyl-3-aminopyrrolidine |
| N-undecylamine | 1-benzylpiperazine |
| 3,4-dichlorobenzylamine | 4-piperidino aniline |
| 2-(trifluoromethyl)benzylamine | 4-(trifluoromethoxy)-aniline |
| 4-cyclohexylaniline | 4-hexylaniline |
| 4-fluorophenethylamine hydrochloride | 4-amino-2,6-dichlorophenol |
| 1-(2-fluorophenyl)piperazine | 4-morpholinoaniline |
| 1-(4-fluorophenyl)piperazine | N-(2-aminoethyl)-N-ethyl-m-toluidine |
| 2-(3,4-dimethoxyphenyl)ethylamine | 4-aminophenylacetic acid ethyl ester |
| 2-amino-fluorene | 1-(2-furoyl)piperazine |
| 3,4,5-trimethoxyaniline | aminodiphenylmethane |
| 4-aminodiphenylmethane | N-phenyl-p-phenylenediamine |
| 3-phenoxyaniline | 2-amino-4-methylbenzothiazole |
| 4-phenoxyaniline | N-(4-aminobenzoyl)-beta-alanine |
| 2-methyl-1-(3- | 2-methoxy-N-phenyl-1,4- |
| methylphenyl)piperazine | phenylenediamine |
| 4-amino-1-benzylpiperidine | 5-phenoxy-o-anisidine |
| 4-aminohippuric acid | 4-amino-4′-chlorodiphenylether |
| 2-amino-9-fluorenone | 1-piperonylpiperazine |
| 1-(3-chlorophenyl)piperazine | 4-amino-4′-methoxystilbene |
| (R)-(+)-1-(4-methoxyphenyl) | N-isopropyl-N-phenyl-p- |
| ethylamine | phenylenediamine |
| 2-amino-4,5,6,7-tetrahydrobenzo | alpha-(cyanoimino)-3,4- |
| (b) thiophene-3-carbonitrile | dichlorophenethylamine |
| 3-benzyloxyaniline | 4-(4-nitrophenoxy)-aniline |
| 4-amino-4′-methyldiphenylether | 4-amino-4′-nitrodiphenylsulfide |
| 4-(2-aminoethyl)bensene sulfonamide | 2-amino-7-bromofluorene |
| n-undecylamine | (1R,2S)-(+)-cis-1-amino-2-indanol |
| 3,4,5-trimethoxybenzylamine | N-methyl-b-alaninenitrile |
| dl-1-amino-2-propanol | 2,2-diphenylethylamine |
| (+/−)-2-amino-1-butanol | 4-amino-1-butanol |
| 1-amino-2-butanol | 3-(ethylamino)propionitrile |
| 2-amino-2-methyl-1-propanol | 4-hydroxypiperidine |
| 2-ethylpiperidine | diethanolamine |
| N-methyl cyclohexylamine | 3-aminophenol |
| 3-piperidinemethanol | 4-aminophenol |
| 2,4-dimethylaniline | 2,3-dimethylcyclohexylamine |
| 2,5-dimethylaniline | S(+)-1-cyclohexylethylamine |
| d(+)-alpha-methylbenzylamine | 1-methyl-4-(methylamino)piperidine |
| dl-alpha-methylbenzylamine | 2-piperidineethanol |
| 3-aminobenzyl alcohol | N-(2-hydroxyethyl)piperazine |
| o-anisidine | 1-aminoindan |
| 2,3-dimethyl-4-aminophenol | (R)-(+)-1-phenylpropylamine |
| 2-aminophenethyl alcohol | (R)-1-(4-methylphenyl)ethylamine |
| 3-amino-4-methylbenzyl alcohol | 4-aminophenethyl alcohol |
| 3-amino5,5-dimethyl-2- | 2-amino-5-phenyl-1,3,4-thiadiazole |
| cyclohexen-1-one | sulfate |
| 2-(methylthio)aniline | (R)-(+)-2-amino-3-phenylpropanol |
| 4-amino-2-chlorophenol | 1(−)-2-amino-3-phenyl-1-propanol |
| (1R, 2S)-1-amino-2-indanol | 3-amino-4-hydroxybenzoic acid |
| methyl 4-aminobenzoate | 4-amino salicylic acid |
| 3-amino-5-phenylpyrazole | 4-amino-3-hydroxybenzoic acid |
| Veratrylamine | 2,4-dimethoxyanhline |
| 3-amino-1-phenyl-2-pyrazolin-5-one | 1-amino-2-methylnaphthalene |
| 6′-amino-3,4-(methylenedioxy) | (1S,2S)-(+)-2-amino-1-phenyl-1,3- |
| acetophenone | propanediol |
| 5-tert-butyl-o-anisidine | N-benzyl-2-phenylethylamine |
| Dibenzylamine | N-(4-amino-2-chlorophenyl)morpholine |
| 1-(3,4-dichlorophenyl) piperazine | 2-chloro-4,6-dimethylaniline |
| 2-[2-(aminomethyl)phenylthio]benzyl | (S)-(+)-2-amino-3-cyclohexyl-1- |
| alcohol | propanol HCl |
| 2-amino-1,3-propanedlol | 2-bromobenzylamine hydrochloride |
| 3-amino-1,2-propanediol | 3-bromobenzylamine hydrochloride |
| 5-amino-1-methyl-3-(thien-2- | 1-(2-methoxyphenyl)piperazine |
| yl)pyrazole | hydrochloride |
| 2-amino-m-cresoi | 4-benzyloxyanhline hydrochloride |
| 5-aminoindazole | 3-hydroxytyramine hydrobromide |
| 5-aminobenzotriazole | 5-phenyl-o-anisidlne |
| 2-methoxy-6-methylaniline | 2-piperidinemethanol |
| 2-aminonorbornane hydrochloride | 3,5-bis(trifluoromethyl)-benzylamine |
| methyl 4-aminobutyrate hydrochloride | 3-aminopyrrolidine dihydrochloride |

N-(4-methoxyphenyl)-p-phenylenediamine hydrochloride

The present invention is based on the development of bi-ligands that bind to two independent sites on a receptor. The combination of two ligands into a single molecule allows both ligands to simultaneously bind to the receptor and thus can provide synergistically higher affinity than either ligand alone (Dempsey and Snell, Biochemistry 2:1414-1419 (1963); and Radzicka and Wolfenden, Methods Enzymol. 249:284-303 (1995), each of which is incorporated herein by reference). The generation of libraries of bi-ligands focused for binding to a receptor family or a particular receptor in a receptor family has been described previously (see WO 99/60404, which is incorporated herein by reference). The common ligand mimics of the present invention allow for increased diversity of bi-ligand libraries while simultaneously preserving the ability to focus a library for binding to a receptor family.

As described previously (see WO 99/60404), when developing bi-ligands having binding activity for a receptor family, it is generally desirable to use a common ligand having relatively modest binding activity, for example, mM to A binding activity. This binding activity is increased when combined with a specificity ligand.

The common ligand mimic can be modified through the addition of substituents, which can also be called expansion linkers. Substitution of the common ligand mimic allows for tailoring of the bi-ligand by directing the attachment location of the specificity ligand on the common ligand mimic. Tailoring of the bi-ligand in this manner provides optimal binding of the common ligand mimic to the conserved site on the receptor and of the specificity ligand to the specificity site on the same receptor. Through such tailoring, libraries having improved diversity and improved receptor binding can be produced. The bi-ligands contained in such libraries also exhibit improved affinity and/or specificity.

A number of formats for generating combinatorial libraries are well known in the art, for example soluble libraries, compounds attached to resin beads, silica chips or other solid supports. As an example, the “split resin approach” may be used, as described in U.S. Pat. No. 5,010,175 to Rutter and in Gallop et al., J. Med. Chem., 37:1233-1251 (1994), incorporated by reference herein.

Methods for generating libraries of bi-ligands having diversity at the specificity ligand position have been described previously (see WO 99/60404, WO 00/75364, and U.S. Pat. No. 6,333,149, which issued Dec. 25, 2001). A library of bi-ligands is generated so that the binding affinity of the common ligand mimic and the specificity ligand can synergistically contribute to the binding interactions of the bi-ligand with a receptor having the respective conserved site and specificity site. Thus, the bi-ligands are generated with the specificity ligand and common ligand mimic oriented so that they can simultaneously bind to the specificity site and conserved site, respectively, of a receptor.

The present invention also provides methods of screening combinatorial libraries of bi-ligands comprising one or more common ligand mimic bound to a variety of specificity ligands and identification of bi-ligands having binding activity for the receptor. Thus, the present invention provides methods for generating a library of bi-ligands suitable for screening a particular member of a receptor family as well as other members of a receptor family.

Development of combinatorial libraries of bi-ligands of the invention begins with selection of a receptor family. Methods for determining that two receptors are in the same family, and thus constitute a receptor family, are well known in the art. For example, one method for determining if two receptors are related is BLAST, Basic Local Alignment Search Tool, available on the National Center for Biotechnology Information web page (www.ncbi.nlm.gov/BLAST/)(which is incorporated herein by reference) and modified BLAST protocols. A second resource for identifying members of a receptor family is PROSITE, available at ExPASy (www.expasy.ch/sprot/prosite.html)(which is incorporated herein by reference). A third resource for identifying members of a receptor family is Structural Classification of Proteins (SCOP) available at SCOP (scop.mrc lmb.cam.ac.uk/scop/) (which is incorporated herein by reference).

Once a receptor family has been identified, the next step in development of bi-ligands involves determining whether there is a natural common ligand that binds at least two members of the receptor family, and preferably to several or most members of the receptor family. In some cases, a natural common ligand for the identified receptor family is already known. For example, it is known that dehydrogenases bind to dinucleotides such as NAD or NADP. Therefore, NAD or NADP are natural common ligands to a number of dehydrogenase family members. Similarly, all kinases bind ATP, and, thus, ATP is a natural common ligand to kinases.

After a receptor family has been selected, at least two receptors in the receptor family are selected as receptors for identifying useful common ligand mimics. Selection criteria depend upon the specific use of the bi-ligands to be produced. Once common ligand mimics are identified, these compounds are screened for binding affinity to the receptor family.

Those common ligand mimics having the most desirable binding activity then can be modified by adding substituents that are useful for the attachment and orientation of a specificity ligand. For example, in the present invention, benizimidazole was determined to be a common ligand mimic for NAD. These compounds can be modified, for example, by the addition of substituents to the benzimidazole ring. For example, the benzimidazole can be substituted with an alkyl group, a nitro group, or a halogen. These groups provide attachment points for the specificity ligand. Substituents added to the benzimidazole ring can also act as blocking groups to prevent attachment of a specificity ligand at a particular site or can act to orient the specificity ligand in a particular manner to improve binding of the bi-ligand to the receptor.

Methods of screening for common ligand mimics and bi-ligands containing the common ligand mimics are well known in the art. For example, a receptor can be incubated in the presence of a known ligand and one or more potential common ligand mimics. In some cases, the natural common ligand has an intrinsic property that is useful for detecting whether the natural common ligand is bound. For example, the natural common ligand for dehydrogenases, NAD, has intrinsic fluorescence. Therefore, increased fluorescence in the presence of potential common ligand mimics due to displacement of NAD can be used to detect competition for binding of NAD to a target NAD binding receptor (Li and Lin, Eur. J. Biochem. 235:180-186 (1996); and Ambroziak and Pietruszko, Biochemistry 28:5367-5373 (1989), each of which is incorporated herein by reference).

In other cases, when the natural common ligand does not have an intrinsic property useful for detecting ligand binding, the known ligand can be labeled with a detectable moiety. For example, the natural common ligand for kinases, ATP, can be radiolabeled with ³²p, and the displacement of radioactive ATP from an ATP binding receptor in the presence of potential common ligand mimics can be used to detect additional common ligand mimics. Any detectable moiety, for example a radioactive or fluorescent label, can be added to the known ligand so long as the labeled known ligand can bind to a receptor having a conserved site. Similarly, a radioactive or fluorescent moiety can be added to NAD or a derivative thereof to facilitate screening of the NAD common ligand mimics and/or bi-ligands of the invention.

The pool of potential common ligand mimics screened for competitive binding with a natural common ligand can be a broad range of compounds of various structures. However, the pool of potential ligands can also be focused on compounds that are more likely to bind to a conserved site in a receptor family. For example, a pool of candidate common ligand mimics can be chosen based on structural similarities to the natural common ligand.

Thiazolidinedione and rhodanine were identified by the present inventors as common ligand mimics of NAD. Structural and functional studies of these compounds led to the identification of additional compounds having similar activity as common ligand mimics of NAD. One such compound is benzimidazole. Further structural and functional studies led to the development of benzimidazole derivatives as common ligand mimics of NAD. Methods for identifying molecules having similar structure are well known in the art and are commercially available (Doucet and Weber, in Computer-Aided Molecular Design: Theory and Applications, Academic Press, San Diego Calif. (1996), which is incorporated herein by reference; software is available from Molecular Simulations, Inc., San Diego Calif.). Furthermore, if structural information is available for the conserved site in the receptor, particularly with a known ligand bound, compounds that fit the conserved site can be identified through computational methods (Blundell, Nature 384 Supp:23-26 (1996), which is incorporated herein by reference). These methods also can be used to screen for specificity ligands and bi-ligands of the invention.

Once a library of bi-ligands is generated, the library can be screened for binding activity to a receptor in a corresponding receptor family. Methods of screening for binding activity that are well known in the art can be used to test for binding activity.

The common ligand mimics and bi-ligands of the present invention can be screened, for example, by the following methods. Screening can be performed through kinetic assays that evaluate the ability of the common ligand mimic or bi-ligand to react with the receptor. For example, where the receptor is and reductase or dehydrogenase for which NAD is a natural common ligand, compounds of the invention can be assayed for their ability to oxidize NADH or NADPH or for their ability to reduce NAD+. Such assays are described more fully in Examples 10 through 12.

### EXAMPLES

Starting materials were obtained from commercial suppliers and used without further purification. ¹H NMR spectra were acquired on a Bruker Avance 300 spectrometer at 300 MHz for ¹H NMR and 75 MHz for ¹³C NMR. Chemical shifts are recorded in parts per million (δ) relative to TMS (δ=0.0 ppm) for ¹H or to the residual signal of deuterated solvents (chloroform, δ=7.25 ppm for ¹H; δ=77.0 ppm for ¹³C). Coupling constant J is reported in Hz. Chromatography was performed on silica gel with ethyl acetate/hexane as elutant unless otherwise noted. Mass spectra were recorded on LCQ from Finnigan.

### Example 1

### Preparation of 5-trimethylstannanyl-furan-2-carbaldehyde (compound 2)

This example describes the synthesis of 5-trimethylstannanyl-furan-2-carbaldehyde, which is used as a reagent in the formation of benzimidazole compounds using the method described in FIG. 1.

A solution of butyl lithium (BuLi; 105 mmol, 2.5 M in hexanes) was added to a solution of 4-methylpiperidine (10.00 g, 100 mmol) in 50 ml of tetrahydrofuran (THF) under N₂ at −78° C., followed by the addition of 2-furaldehyde (8.73 g, 91 mmol). The solution was kept at −78° C. for 15 minutes, and then another portion of BuLi (105 mmol, 2.5 M solution in hexane) was added. The reaction mixture was allowed to warm to −20° C. and was stirred for 5 hours.

The solution was cooled to −78° C. and then added to a solution of Me₃SnCl (100 mmol, 1M solution in THF). The mixture was allowed to warm gradually to room temperature and then stirred overnight. The reaction was quenched by adding 150 ml of cold brine and extracted with EtOAc (3×100 ml). The combined organic phase was dried and concentrated.

Chromatography (EtOAc/Hexane 20:1) afforded 20.7 g (88.5%) of 5-trimethylstannanyl-furan-2-carbaldehyde. The product was analyzed by NMR as follows:

¹H NMR (300 MHz, CDCl₃) δ0.41 (s, 9H) , 6.74 (d, J=3.7, 1H), 7.25 (d, J=3.6, 1H), 9.67 (s, 1H); MS m/z 261 (M+1).

### Example 2

### Preparation of 4-[5-(4-methyl-1H-benzimidazol-2-yl)-furan-2-yl]-benzoic acid (compound 6a)

This example describes the synthesis of benzimidazole compounds following the reaction scheme shown in FIG. 1. Compound numbers correspond to those in the figure.

Step a: Formation of 4-(5-formyl-furan-2-yl)-benzoic acid methyl ester (compound 4)

A mixture of methyl 4-bromobenzoate (compound 3, 2.15 g, 10 mmol), 5-trimethylstannanyl-furan-2-carbaldehyde (compound 2, 2.5 g, 10 mmol), and tetrakis(triphenyl-phosphine)palladium (0.577 g, 1 mmol) was prepared in 20 ml of DMF. The mixture was heated under N₂ to 60° C. for 20 hours.

The solution was evaporated to dryness under reduce pressure, and the residue was purified by chromatography using a 1:3 mixture of EtOAc/hexane to give 2.185 g (95%) of methyl 4-(5-formyl-furan-2-yl)benzoic acid methyl ester (compound 4).

¹H NMR (300 MHz, CDCl₃) δ3.98 (s, 3H) , 6.97 (d, J=3.8, 1H), 7.36 (d, J=3.8, 1H), 7.91 (d, J=6.8, 2H), 8.14 (d, J=6.8, 2H), 9.72 (s, 1H); MS m/z 231 (M+1).

Step b: Formation of 4-[5-(4-methyl-1H-benzimidazol-2-yl)-furan-2-yl]-benzoic acid (compound 6a)

A solution of 4-(5-formyl-furan-2-yl)-benzoic acid methyl ester (compound 4, 58 mg, 0.25 mmol), 2,3-diaminotoluene (compound 5, 31 mg, 0.25 mmol) and benzoquinone (27 mg, 0.25 mmol) was prepared in 10 ml of ethanol. The solution was heated at reflux for 4 hours. The solvent was removed, and the residue was dissolved in 50 ml of dichloromethane (CH₂Cl₂). The, the residue was washed with brine (2×10 ml). Concentration and flash chromatography purification of the residue using EtOAC/Hexane (1:1) gave 4-[5-(4-methyl-1H-benzoimidazol-2-yl)-furan-2-yl]-benzoic acid methyl ester (40 mg, 48.2%) as a crude product.

The 4-[5-(4-methyl-1-benzoimidazol-2-yl)-furan-2-yl]-benzoic acid methyl ester (40 mg) was dissolved in a mixture of ethanol (5 ml) and 10% KOH (5 ml). The reaction mixture was heated at reflux for 2 hours and then poured into 1N HCl (30 ml). The product was extracted with EtOAc (3×10 ml). The combined organic phase was dried and concentrated. The residue was purified by HPLC to give 20 mg (52.2%) of 4-[5-(4-methyl-1-benzoimidazol-2-yl)-furan-2-yl]-benzoic acid (compounds 6a).

¹H NMR (300 MHz, CD₃OD) δ2.59 (s, 3H) , 7.19 (d, J=3.8, 1H), 7.21 (d, J=3.8, 1H), 7.45 (m, 2H), 8.06 (m, 4H); MS m/z 319 (M+1).

### Example 3

### Preparation of 4-[5-(1H-benzoimidazol-2-yl)-furan-2-yl] benzoic acid (compound 6b)

The compound 4-[5-(1-benzoimidazol-2-yl)-furan-2-yl] benzoic acid (compound 6b) was prepared following the procedure in Example 2, which is shown in the reaction scheme in FIG. 1. The compound was obtained in 91% yield, and NMR analysis gave the following:

¹H NMR (300 MHz, CD₃OD) δ7.28 (dd, J=6.2, 3.0, 2H), 7.4 (m, 2H), 7.65 (dd, J=6.0, 3.1, 2H), 8.06 (s, 1H); MS m/z 305 (M+1).

### Example 4

### Preparation of 4-[5-(5-methyl-1-benzoimidazol-2-yl)furan-2-yl]-benzoic acid (compound 6c)

The compound 4-[5-(5-methyl-1-benzoimidazol-2-yl)furan-2-yl]-benzoic acid (compound 6c) was prepared following the procedure in Example 2, which is shown in the reaction scheme in FIG. 1. The compound was obtained in 6.4% yield, and NMR analysis gave the following:

¹H NMR (300 MHz, CD₃OD) δ7.36 (m, 2H), 7.71 (m, 3H), 8.12 (m, 4H); MS m/z 319 (M+1).

### Example 5

### Preparation of 4-[5-(5-nitro-1H-benzoimidazol-2-yl)-furan-2-yl]-benzoic acid (compound 6d)

The compound 4-[5-(5-nitro-1-benzoimidazol-2-yl)-furan-2-yl]-benzoic acid (compound 6d) was prepared following the procedure in Example 2, which is shown in the reaction scheme in FIG. 1. The compound was obtained in 68% yield, and NMR analysis gave the following:

¹H NMR (300 MHz, CD₃OD) δ7.24 (d, J=3.8, 1H) , 7.46 ( d, J=3.7, 1H) , 7.76 ( d, J=8.9, 1H), 8.02 (d, J=8.5, 2H) , 8.14 (d, J=8.5, 2H), 8.29 (dd, J=5.8, 2.1, 1H) 8.52 (d, J=2.1, 1H); MS m/z 350 (M+1).

### Example 6

### Preparation of 4-[5-(5-chloro-1-benzoimidazol-2-yl)-furan-2-yl]-benzoic acid (compound 6e)

The compound 4-[5-(5-chloro-1-benzoimidazol-2-yl)-furan-2-yl]-benzoic acid (compound 6e) was prepared following the procedure in Example 2, which is shown in the reaction scheme in FIG. 1. The compound was obtained in 33% yield, and NMR analysis gave the following:

¹H NMR (300 MHz, CD₃OD) δ7.30 (d, J 3.7, 1H), 7.38 (dd, J=8.6, 1.8, 1H), 7.44 (d, J=3.7, 1H), 7.68 (m, 2H), 8.09 (dd, J=8.17, 2H), 8.17 (dd, J=8.17, 2 H); MS m/z 339 (M+1).

### Example 7

### Preparation of 4-[5-(5-methoxy-1-benzoimidazol-2-yl)-furan-2-yl]-benzoic acid (compound 6f)

The compound 4-[5-(5-methoxy-1H-benzoimidazol-2-yl)-furan-2-yl]-benzoic acid (compound 6f) was prepared following the procedure in Example 2, which is shown in the reaction scheme in FIG. 1. The compound was obtained in 54% yield, and NMR analysis gave the following:

¹H NMR (300 MHz, CD₃OD) δ3.86 (s, 3H) , 7.10 (d, J=1.7, 1H), 7.17 (dd, J=3.8, 1.8, 1H), 7.18 (d, J=3.2, 1H), 7.24 (d, J=1.7, 1H), 7.51 (d, J=8.8, 1H), 7.98 (dd, J=8.3, 2H), 8.11 (d, J=8.3, 2H); MS m/z 335 (M+1).

### Example 8

### Preparation of Common Ligand Mimics of the Invention Containing a Carboxylic Acid Linker

This example describes the synthesis of common ligand mimics of the present invention following the reaction scheme shown in FIG. 3. Compound numbers correspond to those in the figure.

A solution of 1,2-phenylenediamine (compound 7, 10 mmol) and 2-furoic acid (compound 8, 10 mmol) was prepared in THF (15 ml) at a temperature of 0° C. EDCI (1-(3-dimethylaminopropyl)-3-ethylcarbodiimide hydrochloride, 12 mmol) was added to the solution, which was allowed to warm to room temperature. The reaction was continued for a period of 3 hours. Then, the solvent was evaporated, and the residue was dissolved in ethyl acetate (150 ml). The product was washed twice with water (2×150 ml) and dried over MgSO₄. After evaporation of the solvent, the product (compound 9, 1.2 g) was obtained (65% yield).

A solution of amide (compound 7b, 1 g) in dioxane and TFA was heated at a temperature of about 100 to 120° C. for a period of about 20 hours. The solvent was then evaporated, and a small amount of ethyl acetate is added to the product, which was filtered to provide 0.72 g of the desired product.

A suspension of 4-aminobenzoic acid (2.14 g, 15.6 mmol) was formed in a mixture of 15 ml water and 8 ml concentrated HCl. Sodium nitrite (1.09 g, 15.6 mmol) was gradually added to the suspension at a temperature of 0° C. A solution of the amide (compound 7b, 2.87 g, 15.6 mmol) in 20 ml acetone was then added to the suspension, followed by addition of a mixture of CuI (0.30 g, 1.6 mmol) and CuCl₂ (0.27 g, 1.6 mmol) over a period of 10 minutes at a temperature of 0° C. The reaction was stirred at room temperature for a period of 1 hour. The precipitate was then collected by filtration, washed with water and acetone, and dried to yield a pure compound 6b (1.89 g, 40.0%). NMR analysis provided the following.

¹H NMR (DMSO-d₆)δ8.07 (dd, J=8 Hz, 4H), 7.80 (d, J=3.0 Hz, 3H), 7.53 (m, 3H), 3.61 (s, br, 1H). MS (M+1⁺)305.

### Example 9

### Preparation of 4-(2-{4-[5-(5-nitro-1H-benzoimidazol-2-yl)-furan-2-yl]-benzoylamino)-ethylsulfanyl)-pyridine-2,6-dicarboxylic acid (compound 21a)

This example describes the synthesis of bi-ligands of the invention following the reaction scheme shown in FIG. 2. Compound numbers correspond to those in the figure.

The compound 4-amino-pyridine-2,6-dicarboxylic acid dimethyl ester (compound 10, free base, 32 mg, 0.118 mmol), 4-[5-(5-nitro-1H-benzoimidazol-2-yl)-furan-2-yl]-benzoic acid (compound 6d, 41 mg, 0.117 mmol) and HOBt.H₂O (11 mg, 0.137 mmol) were dissolved in DMF (1ml). Triethylamine (20 μl, 0.144 mmol) and 1-dimethylaminopropyl-3-ethyl-carbodiimide (EDCI) (27 mg, 0.141 mmol) were added to the mixture that then was stirred at room temperature for 31 hours. The intermediate product was precipitated by the addition of aqueous 2N HCl. The precipitate (53 mg) was isolated by filtration and washed with aqueous 0.5N HCl. The resulting precipitate (48 mg) was mixed with water (0.5 ml), MeOH (0.5 ml), and LiOH (15 mg, 0.63 mmol), and the suspension was stirred at room temperature for 4 hours. The desired product; 4-(2-{4-[5-(5-nitro-1H-benzoimidazol-2-yl)-furan-2-yl]-benzoylamino)-ethylsulfanyl)-pyridine-2,6-dicarboxylic acid; was precipitated with aqueous 2N HCl, filtered, and dried to a brown powder (43 mg, 93%).

¹H NMR (300 MHz, DMSO-d₆) δ3.44 (m, 2H) , 3.63 (m, 2H) 7.42 (d, J=3.4, 1H) , 7.53 (d, J=3.3, 1H) , 7.80 (d, J=8.9, 1H), 7.97 (d, J=8.2, 2H), 8.05 (d, J=8.1, 2H) 8.09 (s, 2H), 8.17 (dd, J=8.8, 1.7, 1H), 849 (s, 1H), 8.89 (br.s., 1H) , 8.30 (brs, 1H) ; MS m/z 574 (M+1)

### Example 10

### Preparation of 4-(2-{4-[5-(1H-benzoimidazol-2-yl)-furan-2-yl]-benzoylamino}-ethylsulfanyl)-pyridine-2,6-dicarbolxylic acid (compound 21b)

This example describes the synthesis of bi-ligands of the invention following the reaction scheme shown in FIG. 2. Compound numbers correspond to those in the figure.

The compound 4-amino-pyridine-2,6-dicarboxylic acid dimethyl ester (compound 10, HCl salt, 50 mg, 0.163 mmol), 4-[5-(1H-benzoimidazol-2-yl)-furan-2-yl] benzoic acid (compound 6b, 49.8 mg, 0.164 mmol) and HOBt.H₂O (30 mg, 0.196 mmol) were dissolved in DMF (1 ml). Triethylamine (0.68 μl, 0.489 mmol) and EDCI (38 mg, 0.198 mmol) were added to the mixture, followed by stirring at room temperature for 16 hours.

Upon acidification with aqueous 2N HCl, a brown precipitate (76 mg) was formed and isolated by filtration. The brown product (69 mg) was mixed with water (0.5 ml), MeOH (0.5 ml), and LiOH (21 mg, 0.88 mmol). The resulting mixture was stirred at room temperature for 1.5 hours. Aqueous 2N HCl was added to the mixture and filtered to give 52 mg of crude product (66% yield, about 90% pure). Purification by preparative HPLC provided 2.7 mg of pure 4-(2-{4-[5-(1H-benzoimidazol-2-yl)-furan-2-yl]-benzoylamino}-ethylsulfanyl)-pyridine-2,6-dicarbolxylic acid (compound 21b)

¹H NMR (300 MHz, DMSO-d₆) δ3.61 (m, 2H) and one signal overlapped by water, 7.27 (m, 2H), 7.63 (m,2H), 7.36 (s,2H), 7.95 (d, J=8.2, 2H), 8.01 (d, J=8.3, 2H), 8.09 (s, 2H), 8.86 (br. t., 1H); MS m/z 441 (M+H-2CO₂)

### Example 11

### Preparation of Common Ligand Mimics having Amide Linkers

This example describes the synthesis of common ligand mimics of the invention containing a linker group following the reaction scheme shown in FIG. 4. Compound numbers correspond to the numbers in the figure.

In a 500 ml round-bottom flask, compound 6 is dissolved in dry DMF by heating. The solution is cooled to a temperature of 40 to 50° C. THF (ca 150 ml) and 1,1′-carbonyldiimidazole (4.5 g) are added to the solution. After shaking for 20 minutes, the flask is capped and refrigerated overnight at −10° C. The precipitate is collected by filtration and washed with THF to provide intermediate compound 10.

A mixture of dry DMF (30 ml) and dry THF (80 ml) is prepared in a 250 ml flask. Intermediate compound 10 is added to the mixture. Boc protected diamines (1.2 eq) are added to the mixture which then is heated at a temperature of 65° C. for a period of 1 hour. By this time, the undissolved solid has dissolved, and a clear solution is obtained. The solvent then is evaporated under reduced pressure to provide compound 11.

A solution of 50% trifluoacetic acid in dichloroethane (100 ml) is added compound 11 and reacted for 10 minutes. Extra solvent is evaporated, resulting in a yellow solid. The yellow solid is then dissolved in 40 to 50 ml of DMF by heating. The solution is cooled to room temperature, and a Na₂CO₃ solution (150-200 ml, 5%) is added. When a yellow precipitate forms, it is filtered. Otherwise, more DMF solvent is evaporated, and more water is added. The yellow solid, compound 12, is washed with a mixture of water and MeOH and then dried to provide 5 to 5.5 g of product compound 12.

Examples of compounds, which can be produced by the methods described in Example 12, include those in Tables 5 to 11.

TABLE 5

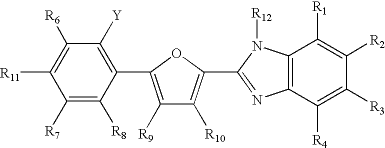

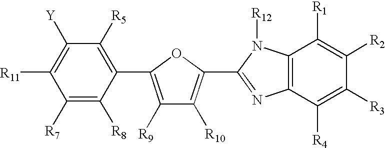

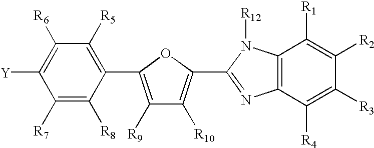

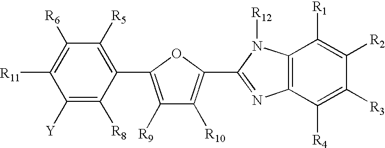

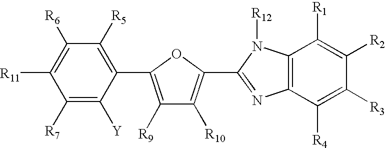

| | Y | | Y | | Y | | Y | | Y |
|---|---|---|---|---|---|---|---|---|---|
| 1 | OH | 2 | OH | 3 | OH | 4 | OH | 5 | OH |
| 1 | SH | 2 | SH | 3 | SH | 4 | SH | 5 | SH |
| 1 | COOH | 2 | COOH | 3 | COOH | 4 | COOH | 5 | COOH |
| 1 | SO₂H | 2 | SO₂H | 3 | SO₂H | 4 | SO₂H | 5 | SO₂H |
| 1 | Cl | 2 | Cl | 3 | Cl | 4 | Cl | 5 | Cl |
| 1 | Br | 2 | Br | 3 | Br | 4 | Br | 5 | Br |
| 1 | I | 2 | I | 3 | I | 4 | I | 5 | I |
| 1 | F | 2 | F | 3 | F | 4 | F | 5 | F |
| 1 | CN | 2 | CN | 3 | CN | 4 | CN | 5 | CN |
| 1 | N₃ | 2 | N₃ | 3 | N₃ | 4 | N₃ | 5 | N₃ |
| 1 | CONH₂ | 2 | CONH₂ | 3 | CONH₂ | 4 | CONH₂ | 5 | CONH₂ |
| 1 | CH═CH₂ | 2 | CH═CH₂ | 3 | CH═CH₂ | 4 | CH═CH₂ | 5 | CH═CH₂ |
| 1 | C≡CH | 2 | C≡CH | 3 | C≡CH | 4 | C≡CH | 5 | C≡CH |
| 1 | NH₂ | 2 | NH₂ | 3 | NH₂ | 4 | NH₂ | 5 | NH₂ |
| 1 | NHR | 2 | NHR | 3 | NHR | 4 | NHR | 5 | NHR |
| 1 | COH | 2 | COH | 3 | COH | 4 | COH | 5 | COH |
| 1 | COR | 2 | COR | 3 | COR | 4 | COR | 5 | COR |

R = alkyl, alkenyl, alkynyl, aryl, or heterocycle

TABLE 6

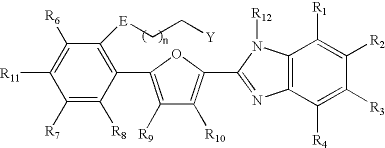

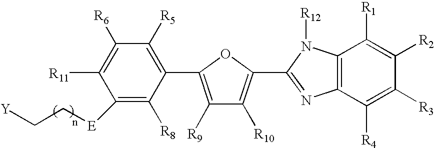

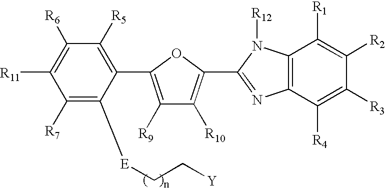

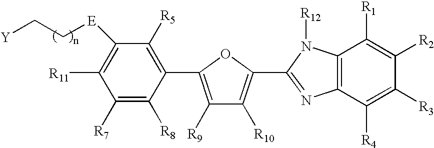

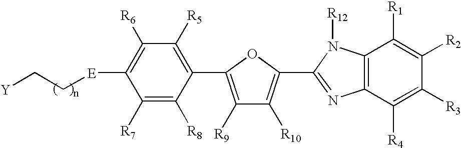

| n | E | Y | n | E | Y | N | E | Y | n | E | Y |
|---|---|---|---|---|---|---|---|---|---|---|---|
| 0 | O | OH | 0 | S | OH | 0 | NH | OH | 0 | NR | OH |
| 0 | O | SH | 0 | S | SH | 0 | NH | SH | 0 | NR | SH |
| 0 | O | COOH | 0 | S | COOH | 0 | NH | COOH | 0 | NR | COOH |
| 0 | O | SO₂H | 0 | S | SO₂H | 0 | NH | SO₂H | 0 | NR | SO₂H |
| 0 | O | Cl | 0 | S | Cl | 0 | NH | Cl | 0 | NR | Cl |
| 0 | O | Br | 0 | S | Br | 0 | NH | Br | 0 | NR | Br |
| 0 | O | I | 0 | S | I | 0 | NH | I | 0 | NR | I |
| 0 | O | F | 0 | S | F | 0 | NH | F | 0 | NR | F |
| 0 | O | CN | 0 | S | CN | 0 | NH | CN | 0 | NR | CN |
| 0 | O | N₃ | 0 | S | N₃ | 0 | NH | N₃ | 0 | NR | N₃ |
| 0 | O | CONH₂ | 0 | S | CONH₂ | 0 | NH | CONH₂ | 0 | NR | CONH₂ |
| 0 | O | CH═CH₂ | 0 | S | CH═CH₂ | 0 | NH | CH═CH₂ | 0 | NR | CH═CH₂ |
| 0 | O | C≡CH | 0 | S | C≡CH | 0 | NH | C≡CH | 0 | NR | C≡CH |
| 0 | O | NH₂ | 0 | S | NH₂ | 0 | NH | NH₂ | 0 | NR | NH₂ |
| 0 | O | NHR | 0 | S | NHR | 0 | NH | NHR | 0 | NR | NHR |
| 0 | O | COH | 0 | S | COH | 0 | NH | COH | 0 | NR | COH |
| 0 | O | COR | 0 | S | COR | 0 | NH | COR | 0 | NR | COR |
| 0 | CH₂ | OH | 0 | COR₁R₂ | OH | 0 | CONH | OH | 0 | CONR | OH |
| 0 | CH₂ | SH | 0 | COR₁R₂ | SH | 0 | CONH | SH | 0 | CONR | SH |
| 0 | CH₂ | COOH | 0 | COR₁R₂ | COOH | 0 | CONH | COOH | 0 | CONR | COOH |
| 0 | CH₂ | SO₂H | 0 | COR₁R₂ | SO₂H | 0 | CONH | SO₂H | 0 | CONR | SO₂H |
| 0 | CH₂ | Cl | 0 | COR₁R₂ | Cl | 0 | CONH | Cl | 0 | CONR | Cl |
| 0 | CH₂ | Br | 0 | COR₁R₂ | Br | 0 | CONH | Br | 0 | CONR | Br |
| 0 | CH₂ | I | 0 | COR₁R₂ | I | 0 | CONH | I | 0 | CONR | I |
| 0 | CH₂ | F | 0 | COR₁R₂ | F | 0 | CONH | F | 0 | CONR | F |
| 0 | CH₂ | CN | 0 | COR₁R₂ | CN | 0 | CONH | CN | 0 | CONR | CN |
| 0 | CH₂ | N₃ | 0 | COR₁R₂ | N₃ | 0 | CONH | N₃ | 0 | CONR | N₃ |
| 0 | CH₂ | CONH₂ | 0 | COR₁R₂ | CONH₂ | 0 | CONH | CONH₂ | 0 | CONR | CONH₂ |
| 0 | CH₂ | CH═CH₂ | 0 | COR₁R₂ | CH═CH₂ | 0 | CONH | CH═CH₂ | 0 | CONR | CH═CH₂ |
| 0 | CH₂ | C≡CH | 0 | COR₁R₂ | C≡CH | 0 | CONH | C≡CH | 0 | CONR | C≡CH |
| 0 | CH₂ | NH₂ | 0 | COR₁R₂ | NH₂ | 0 | CONH | NH₂ | 0 | CONR | NH₂ |
| 0 | CH₂ | NHR | 0 | COR₁R₂ | NHR | 0 | CONH | NHR | 0 | CONR | NHR |
| 0 | CH₂ | COH | 0 | COR₁R₂ | COH | 0 | CONH | COH | 0 | CONR | COH |
| 0 | CH₂ | COR | 0 | COR₁R₂ | COR | 0 | CONH | COR | 0 | CONR | COR |
| 0 | SO₂NH | OH | 0 | SO₂NR | OH | 0 | NHCONH | OH | 0 | NRCONR | OH |
| 0 | SO₂NH | SH | 0 | SO₂NR | SH | 0 | NHCONH | SH | 0 | NRCONR | SH |
| 0 | SO₂NH | COOH | 0 | SO₂NR | COOH | 0 | NHCONH | COOH | 0 | NRCONR | COOH |
| 0 | SO₂NH | SO₂H | 0 | SO₂NR | SO₂H | 0 | NHCONH | SO₂H | 0 | NRCONR | SO₂H |
| 0 | SO₂NH | Cl | 0 | SO₂NR | Cl | 0 | NHCONH | Cl | 0 | NRCONR | Cl |
| 0 | SO₂NH | Br | 0 | SO₂NR | Br | 0 | NHCONH | Br | 0 | NRCONR | Br |
| 0 | SO₂NH | I | 0 | SO₂NR | I | 0 | NHCONH | I | 0 | NRCONR | I |
| 0 | SO₂NH | F | 0 | SO₂NR | F | 0 | NHCONH | F | 0 | NRCONR | F |
| 0 | SO₂NH | CN | 0 | SO₂NR | CN | 0 | NHCONH | CN | 0 | NRCONR | CN |
| 0 | SO₂NH | N₃ | 0 | SO₂NR | N₃ | 0 | NHCONH | N₃ | 0 | NRCONR | N₃ |
| 0 | SO₂NH | CONH₂ | 0 | SO₂NR | CONH₂ | 0 | NHCONH | CONH₂ | 0 | NRCONR | CONH₂ |
| 0 | SO₂NH | CH═CH₂ | 0 | SO₂NR | CH═CH₂ | 0 | NHCONH | CH═CH₂ | 0 | NRCONR | CH═CH₂ |
| 0 | SO₂NH | C≡CH | 0 | SO₂NR | C≡CH | 0 | NHCONH | C≡CH | 0 | NRCONR | C≡CH |
| 0 | SO₂NH | NH₂ | 0 | SO₂NR | NH₂ | 0 | NHCONH | NH₂ | 0 | NRCONR | NH₂ |
| 0 | SO₂NH | NHR | 0 | SO₂NR | NHR | 0 | NHCONH | NHR | 0 | NRCONR | NHR |
| 0 | SO₂NH | COH | 0 | SO₂NR | COH | 0 | NHCONH | COH | 0 | NRCONR | COH |
| 0 | SO₂NH | COR | 0 | SO₂NR | COR | 0 | NHCONH | COR | 0 | NRCONR | COR |
| 0 | NHCNHNH | OH | 0 | NRCNHNR | OH | 0 | NHCOO | OH | 0 | NRCOO | OH |
| 0 | NHCNHNH | SH | 0 | NRCNHNR | SH | 0 | NHCOO | SH | 0 | NRCOO | SH |
| 0 | NHCNHNH | COOH | 0 | NRCNHNR | COOH | 0 | NHCOO | COOH | 0 | NRCOO | COOH |
| 0 | NHCNHNH | SO₂H | 0 | NRCNHNR | SO₂H | 0 | NHCOO | SO₂H | 0 | NRCOO | SO₂H |
| 0 | NHCNHNH | Cl | 0 | NRCNHNR | Cl | 0 | NHCOO | Cl | 0 | NRCOO | Cl |
| 0 | NHCNHNH | Br | 0 | NRCNHNR | Br | 0 | NHCOO | Br | 0 | NRCOO | Br |
| 0 | NHCNHNH | I | 0 | NRCNHNR | I | 0 | NHCOO | I | 0 | NRCOO | I |
| 0 | NHCNHNH | F | 0 | NRCNHNR | F | 0 | NHCOO | F | 0 | NRCOO | F |
| 0 | NHCNHNH | CN | 0 | NRCNHNR | CN | 0 | NHCOO | CN | 0 | NRCOO | CN |
| 0 | NHCNHNH | N₃ | 0 | NRCNHNR | N₃ | 0 | NHCOO | N₃ | 0 | NRCOO | N₃ |
| 0 | NHCNHNH | CONH₂ | 0 | NRCNHNR | CONH₂ | 0 | NHCOO | CONH₂ | 0 | NRCOO | CONH₂ |
| 0 | NHCNHNH | CH═CH₂ | 0 | NRCNHNR | CH═CH₂ | 0 | NHCOO | CH═CH₂ | 0 | NRCOO | CH═CH₂ |
| 0 | NHCNHNH | CC≡H | 0 | NRCNHNR | C≡CH | 0 | NHCOO | C≡CH | 0 | NRCOO | C≡CH |
| 0 | NHCNHNH | NH₂ | 0 | NRCNHNR | NH₂ | 0 | NHCOO | NH₂ | 0 | NHCOO | NH₂ |
| 0 | NHCNHNH | NHR | 0 | NRCNHNR | NHR | 0 | NHCOO | NHR | 0 | NRCOO | NHR |
| 0 | NHCNHNH | COH | 0 | NRCNHNR | COH | 0 | NHCOO | COH | 0 | NRCOO | COH |
| 0 | NHCNHNH | COR | 0 | NRCNHNR | COR | 0 | NHCOO | COR | 0 | NRCOO | COR |
| 0 | C≡C | OH | 0 | CH₂═CH₂ | OH | 1 | O | OH | 1 | S | OH |
| 0 | C≡C | SH | 0 | CH₂═CH₂ | SH | 1 | O | SH | 1 | S | SH |
| 0 | C≡C | COOH | 0 | CH₂═CH₂ | COOH | 1 | O | COOH | 1 | S | COOH |
| 0 | C≡C | SO₂H | 0 | CH₂═CH₂ | SO₂H | 1 | O | SO₂H | 1 | S | SO₂H |
| 0 | C≡C | Cl | 0 | CH₂═CH₂ | Cl | 1 | O | Cl | 1 | S | Cl |
| 0 | C≡C | Br | 0 | CH₂═CH₂ | Br | 1 | O | Br | 1 | S | Br |
| 0 | C≡C | I | 0 | CH₂═CH₂ | I | 1 | O | I | 1 | S | I |
| 0 | C≡C | F | 0 | CH₂═CH₂ | F | 1 | O | F | 1 | S | F |
| 0 | C≡C | CN | 0 | CH₂═CH₂ | CN | 1 | O | CN | 1 | S | CN |
| 0 | C≡C | N₃ | 0 | CH₂═CH₂ | N₃ | 1 | O | N₃ | 1 | S | N₃ |
| 0 | C≡C | CONH₂ | 0 | CH₂═CH₂ | CONH₂ | 1 | O | CONH₂ | 1 | S | CONH₂ |
| 0 | C≡C | CH═CH₂ | 0 | CH₂═CH₂ | CH═CH₂ | 1 | O | CH═CH₂ | 1 | S | CH═CH₂ |
| 0 | C≡C | C≡CH | 0 | CH₂═CH₂ | C≡CH | 1 | O | C≡CH | 1 | S | C≡CH |
| 0 | C≡C | NH₂ | 0 | CH₂═CH₂ | NH₂ | 1 | O | NH₂ | 1 | S | NH₂ |
| 0 | C≡C | NHR | 0 | CH₂═CH₂ | NHR | 1 | O | NHR | 1 | S | NHR |
| 0 | C≡C | COH | 0 | CH₂═CH₂ | COH | 1 | O | COH | 1 | S | COH |
| 0 | C≡C | COR | 0 | CH₂═CH₂ | COR | 1 | O | COR | 1 | S | COR |
| 1 | NH | OH | 1 | NR | OH | 1 | CH₂ | OH | 1 | COR₁R₂ | OH |
| 1 | NH | SH | 1 | NR | SH | 1 | CH₂ | SH | 1 | COR₁R₂ | SH |
| 1 | NH | COOH | 1 | NR | COOH | 1 | CH₂ | COOH | 1 | COR₁R₂ | COOH |
| 1 | NH | SO₂H | 1 | NR | SO₂H | 1 | CH₂ | SO₂H | 1 | COR₁R₂ | SO₂H |
| 1 | NH | Cl | 1 | nR | Cl | 1 | CH₂ | Cl | 1 | COR₁R₂ | Cl |
| 1 | NH | Br | 1 | NR | Br | 1 | CH₂ | Br | 1 | COR₁R₂ | Br |
| 1 | NH | I | 1 | NR | I | 1 | CH₂ | I | 1 | COR₁R₂ | I |
| 1 | NH | F | 1 | NR | F | 1 | CH₂ | F | 1 | COR₁R₂ | F |
| 1 | NH | CN | 1 | NR | CN | 1 | CH₂ | CN | 1 | COR₁R₂ | CN |
| 1 | NH | N₃ | 1 | NR | N₃ | 1 | CH₂ | N₃ | 1 | COR₁R₂ | N₃ |
| 1 | NH | CONH₂ | 1 | NR | CONH₂ | 1 | CH₂ | CONH₂ | 1 | COR₁R₂ | CONH₂ |
| 1 | NH | CH═CH₂ | 1 | NR | CH═CH₂ | 1 | CH₂ | CH═CH₂ | 1 | COR₁R₂ | CH═CH₂ |
| 1 | NH | C≡CH | 1 | NR | C≡CH | 1 | CH₂ | C≡CH | 1 | COR₁R₂ | C≡CH |
| 1 | NH | NH₂ | 1 | NR | NH₂ | 1 | CH₂ | NH₂ | 1 | COR₁R₂ | NH₂ |
| 1 | NH | NHR | 1 | NR | NHR | 1 | CH₂ | NHR | 1 | COR₁R₂ | NHR |
| 1 | NH | COH | 1 | NR | COH | 1 | CH₂ | COH | 1 | COR₁R₂ | COH |
| 1 | NH | COR | 1 | NR | COR | 1 | CH₂ | COR | 1 | COR₁R₂ | COR |
| 1 | CONH | OH | 1 | CONR | OH | 1 | SO₂NH | OH | 1 | SO₂NR | OH |
| 1 | CONH | SH | 1 | CONR | SH | 1 | SO₂NH | SH | 1 | SO₂NR | SH |
| 1 | CONH | COOH | 1 | CONR | COOH | 1 | SO₂NH | COOH | 1 | SO₂NR | COOH |
| 1 | CONH | SO₂H | 1 | CONR | SO₂H | 1 | SO₂NH | SO₂H | 1 | SO₂NR | SO₂H |
| 1 | CONH | Cl | 1 | CONR | Cl | 1 | SO₂NH | Cl | 1 | SO₂NR | Cl |
| 1 | CONH | Br | 1 | CONR | Br | 1 | SO₂NH | Br | 1 | SO₂NR | Br |
| 1 | CONH | I | 1 | CONR | I | 1 | SO₂NH | I | 1 | SO₂NR | I |
| 1 | CONH | F | 1 | CONR | F | 1 | SO₂NH | F | 1 | SO₂NR | F |
| 1 | CONH | CN | 1 | CONR | CN | 1 | SO₂NH | CN | 1 | SO₂NR | CN |
| 1 | CONH | N₃ | 1 | CONR | N₃ | 1 | SO₂NH | N₃ | 1 | SO₂NR | N₃ |
| 1 | CONH | CONH₂ | 1 | CONR | CONH₂ | 1 | SO₂NH | CONH₂ | 1 | SO₂NR | CONH₂ |
| 1 | CONH | CH═CH₂ | 1 | CONR | CH═CH₂ | 1 | SO₂NH | CH═CH₂ | 1 | SO₂NR | CH═CH₂ |
| 1 | CONH | C≡CH | 1 | CONR | C≡CH | 1 | SO₂NH | C≡CH | 1 | SO₂NR | C≡CH |
| 1 | CONH | NH₂ | 1 | CONR | NH₂ | 1 | SO₂NH | NH₂ | 1 | SO₂NR | NH₃ |
| 1 | CONH | NHR | 1 | CONR | NHR | 1 | SO₂NH | NHR | 1 | SO₂NR | NHR |
| 1 | CONH | COH | 1 | CONR | COH | 1 | SO₂NH | COH | 1 | SO₂NR | SOH |
| 1 | CONH | COR | 1 | CONR | COR | 1 | SO₂NH | COR | 1 | SO₂NR | COR |
| 1 | NHCONH | OH | 1 | NRCONR | OH | 1 | NHCNHNH | OH | 1 | NRCNHNR | OH |
| 1 | NHCONH | SH | 1 | NRCONR | SH | 1 | NHCNHNH | SH | 1 | NRCNHNR | SH |
| 1 | NHCONH | COOH | 1 | NRCONR | COOH | 1 | NHCNHNH | COOH | 1 | NRCNHNR | COOH |
| 1 | NHCONH | SO₂H | 1 | NRCONR | SO₂H | 1 | NHCNHNH | SO₂H | 1 | NRCNHNR | SO₂H |
| 1 | NHCONH | Cl | 1 | NRCONR | Cl | 1 | NHCNHNH | Cl | 1 | NRCNHNR | Cl |
| 1 | NHCONH | Br | 1 | NRCONR | Br | 1 | NHCNHNH | Br | 1 | NRCNHNR | Br |
| 1 | NHCONH | I | 1 | NRCONR | I | 1 | NHCNHNH | I | 1 | NRCNHNR | I |
| 1 | NHCONH | F | 1 | NRCONR | F | 1 | NHCNHNH | F | 1 | NRCNHNR | F |
| 1 | NHCONH | CN | 1 | NRCONR | CN | 1 | NHCNHNH | CN | 1 | NRCNHNR | CN |
| 1 | NHCONH | N₃ | 1 | NRCONR | N₃ | 1 | NHCNHNH | N₃ | 1 | NRCNHNR | N₃ |
| 1 | NHCONH | CONH₂ | 1 | NRCONR | CONH₂ | 1 | NHCNHNH | CONH₂ | 1 | NRCNHNR | CONH₂ |
| 1 | NHCONH | CH═CH₂ | 1 | NRCONR | CH═CH₂ | 1 | NHCNHNH | CH═CH₂ | 1 | NRCNHNR | CH═CH₂ |
| 1 | NHCONH | C≡CH | 1 | NRCONR | C≡CH | 1 | NHCNHNH | C≡CH | 1 | NRCNHNR | C≡CH |
| 1 | NHCONH | NH₂ | 1 | NRCONR | NH₂ | 1 | NHCNHNH | NH₂ | 1 | NRCNHNR | NH₂ |
| 1 | NHCONH | NHR | 1 | NRCONR | NHR | 1 | NHCNHNH | NHR | 1 | NRCNHNR | NHR |
| 1 | NHCONH | COH | 1 | NRCONR | COH | 1 | NHCNHNH | COH | 1 | NRCNHNR | COH |
| 1 | NHCONH | COR | 1 | NRCONR | COR | 1 | NHCNHNH | COR | 1 | NRCNHNR | COR |
| 1 | NHCOO | OH | 1 | NRCOO | OH | 1 | C≡C | OH | 1 | CH═CH₂ | OH |
| 1 | NHCOO | SH | 1 | NRCOO | SH | 1 | C≡C | SH | 1 | CH═CH₂ | SH |
| 1 | NHCOO | COOH | 1 | NRCOO | COOH | 1 | C≡C | COOH | 1 | CH═CH₂ | COOH |
| 1 | NHCOO | SO₂H | 1 | NRCOO | SO₂H | 1 | C≡ | SO₂H | 1 | CH═CH₂ | SO₂H |
| 1 | NHCOO | Cl | 1 | NRCOO | Cl | 1 | C≡C | Cl | 1 | CH═CH₂ | Cl |
| 1 | NHCOO | Br | 1 | NRCOO | Br | 1 | C≡C | Br | 1 | CH═CH₂ | Br |
| 1 | NHCOO | I | 1 | NRCOO | I | 1 | C≡C | I | 1 | CH═CH₂ | I |
| 1 | NHCOO | F | 1 | NRCOO | F | 1 | C≡C | F | 1 | CH═CH₂ | F |
| 1 | NHCOO | CN | 1 | NRCOO | CN | 1 | C≡C | CN | 1 | CH═CH₂ | CN |
| 1 | NHCOO | N₃ | 1 | NRCOO | N₃ | 1 | C≡C | N₃ | 1 | CH═CH₂ | N₃ |
| 1 | NHCOO | CONH₂ | 1 | NRCOO | CONH₂ | 1 | C≡C | CONH₂ | 1 | CH═CH₂ | CONH₂ |
| 1 | NHCOO | CH═CH₂ | 1 | NRCOO | CH═CH₂ | 1 | C≡C | CH═CH₂ | 1 | CH═CH₂ | CH═CH₂ |
| 1 | NHCOO | C≡CH | 1 | NRCOO | C≡CH | 1 | C≡C | CCH | 1 | CH═CH₂ | CCH |
| 1 | NHCOO | NH₂ | 1 | NRCOO | NH₂ | 1 | C≡C | NH₂ | 1 | CH═CH₂ | NH₂ |
| 1 | NHCOO | NHR | 1 | NRCOO | NHR | 1 | C≡C | NHR | 1 | CH═CH₂ | NHR |
| 1 | NHCOO | COH | 1 | NRCOO | COH | 1 | C≡C | COH | 1 | CH═CH₂ | COH |
| 1 | NHCOO | COR | 1 | NRCOO | COR | 1 | C≡C | COR | 1 | CH═CH₂ | COR |
| 2 | O | OH | 2 | S | OH | 2 | NH | OH | 2 | NR | OH |
| 2 | O | SH | 2 | S | SH | 2 | NH | SH | 2 | NR | SH |
| 2 | O | COOH | 2 | S | COOH | 2 | NH | COOH | 2 | NR | COOH |
| 2 | O | SO₂H | 2 | S | SO₂H | 2 | NH | SO₂H | 2 | NR | SO₂H |
| 2 | O | Cl | 2 | S | Cl | 2 | NH | Cl | 2 | NR | Cl |
| 2 | O | Br | 2 | S | Br | 2 | NH | Br | 2 | NR | Br |
| 2 | O | I | 2 | S | I | 2 | NH | I | 2 | NR | I |
| 2 | O | F | 2 | S | F | 2 | NH | F | 2 | NR | F |
| 2 | O | CN | 2 | S | CN | 2 | NH | CN | 2 | NR | CN |
| 2 | O | N₃ | 2 | S | N₃ | 2 | NH | N₃ | 2 | NR | N₃ |
| 2 | O | CONH₂ | 2 | S | CONH₂ | 2 | NH | CONH₂ | 2 | NR | CONH₂ |
| 2 | O | CH═CH₂ | 2 | S | CH═CH₂ | 2 | NH | CH═CH₂ | 2 | NR | CH═CH₂ |
| 2 | O | C≡CH | 2 | S | C≡CH | 2 | NH | C≡CH | 2 | NR | C≡CH |
| 2 | O | NH₂ | 2 | S | NH₂ | 2 | NH | NH₂ | 2 | NR | NH₂ |
| 2 | O | NHR | 2 | S | NHR | 2 | NH | NHR | 2 | NR | NHR |
| 2 | O | COH | 2 | S | COH | 2 | NH | COH | 2 | NR | COH |
| 2 | O | COR | 2 | S | COR | 2 | NH | COR | 2 | NR | COR |
| 2 | CH₂ | OH | 2 | COR₁R₂ | OH | 2 | CONH | OH | 2 | CONR | OH |
| 2 | CH₂ | SH | 2 | COR₁R₂ | SH | 2 | CONH | SH | 2 | CONR | SH |
| 2 | CH₂ | COOH | 2 | COR₁R₂ | COOH | 2 | CONH | COOH | 2 | CONR | COOH |
| 2 | CH₂ | SO₂H | 2 | COR₁R₂ | SO₂H | 2 | CONH | SO₂H | 2 | CONR | SO₂H |
| 2 | CH₂ | Cl | 2 | COR₁R₂ | Cl | 2 | CONH | Cl | 2 | CONR | Cl |
| 2 | CH₂ | Br | 2 | COR₁R₂ | Br | 2 | CONH | Br | 2 | CONR | Br |
| 2 | CH₂ | I | 2 | COR₁R₂ | I | 2 | CONH | I | 2 | CONR | I |
| 2 | CH₂ | F | 2 | COR₁R₂ | F | 2 | CONH | F | 2 | CONR | F |
| 2 | CH₂ | CN | 2 | COR₁R₂ | CN | 2 | CONH | CN | 2 | CONR | CN |
| 2 | CH₂ | N₃ | 2 | COR₁R₂ | N₃ | 2 | CONH | N₃ | 2 | CONR | N₃ |
| 2 | CH₂ | CONH₂ | 2 | COR₁R₂ | CONH₂ | 2 | CONH | CONH₂ | 2 | CONR | CONH₂ |
| 2 | CH₂ | CH═CH₂ | 2 | COR₁R₂ | CH═CH₂ | 2 | CONH | CH═CH₂ | 2 | CONR | CH═CH₂ |
| 2 | CH₂ | C≡CH | 2 | COR₁R₂ | C≡CH | 2 | CONH | C≡CH | 2 | CONR | C≡CH |
| 2 | CH₂ | NH₂ | 2 | COR₁R₂ | NH₂ | 2 | CONH | NH₂ | 2 | CONR | NH₂ |
| 2 | CH₂ | NHR | 2 | COR₁R₂ | NHR | 2 | CONH | NHR | 2 | CONR | NHR |
| 2 | CH₂ | COH | 2 | COR₁R₂ | COH | 2 | CONH | COH | 2 | CONR | COH |
| 2 | CH₂ | COR | 2 | COR₁R₂ | COR | 2 | CONH | COR | 2 | CONR | COR |
| 2 | SO₂NH | OH | 2 | SO₂NR | OH | 2 | NHCONH | OH | 2 | NRCONR | OH |
| 2 | SO₂NH | SH | 2 | SO₂NR | SH | 2 | NHCONH | SH | 2 | NRCONR | SH |
| 2 | SO₂NH | COOH | 2 | SO₂NR | COOH | 2 | NHCONH | COOH | 2 | NRCONR | COOH |
| 2 | SO₂NH | SO₂H | 2 | SO₂NR | SO₂H | 2 | NHCONH | SO₂H | 2 | NRCONR | SO₂H |
| 2 | SO₂NH | Cl | 2 | SO₂NR | Cl | 2 | NHCONH | Cl | 2 | NRCONR | Cl |
| 2 | SO₂NH | Br | 2 | SO₂NR | Br | 2 | NHCONH | Br | 2 | NRCONR | Br |
| 2 | SO₂NH | I | 2 | SO₂NR | I | 2 | NHCONH | I | 2 | NRCONR | I |
| 2 | SO₂NH | F | 2 | SO₂NR | F | 2 | NHCONH | F | 2 | NRCONR | F |
| 2 | SO₂NH | CN | 2 | SO₂NR | CN | 2 | NHCONH | CN | 2 | NRCONR | CN |
| 2 | SO₂NH | N₃ | 2 | SO₂NR | N₃ | 2 | NHCONH | N₃ | 2 | NRCONR | N₃ |
| 2 | SO₂NH | CONH₂ | 2 | SO₂NR | CONH₂ | 2 | NHCONH | CONH₂ | 2 | NRCONR | CONH₂ |
| 2 | SO₂NH | CH═CH₂ | 2 | SO₂NR | CH═CH₂ | 2 | NHCONH | CH═CH₂ | 2 | NRCONR | CH═CH₂ |
| 2 | SO₂NH | C≡CH | 2 | SO₂NR | CCH | 2 | NHCONH | CCH | 2 | NRCONR | CCH |
| 2 | SO₂NH | NH₂ | 2 | SO₂NR | NH₂ | 2 | NHCONH | NH₂ | 2 | NRCONR | NH₂ |
| 2 | SO₂NH | NHR | 2 | SO₂NR | NHR | 2 | NHCONH | NHR | 2 | NRCONR | NHR |
| 2 | SO₂NH | COH | 2 | SO₂NR | COH | 2 | NHCONH | COH | 2 | NRCONR | COH |
| 2 | SO₂NH | COR | 2 | SO₂NR | COR | 2 | NHCONH | COR | 2 | NRCONR | COR |
| 2 | NHCNHNH | OH | 2 | NRCNHNR | OH | 2 | NHCOO | OH | 2 | NRCOO | OH |
| 2 | NHCNHNH | SH | 2 | NRCNHNR | SH | 2 | NHCOO | SH | 2 | NRCOO | SH |
| 2 | NHCNHNH | COOH | 2 | NRCNHNR | COOH | 2 | NHCOO | COOH | 2 | NRCOO | COOH |
| 2 | NHCNHNH | SO₂H | 2 | NRCNHNR | SO₂H | 2 | NHCOO | SO₂H | 2 | NRCOO | SO₂H |
| 2 | NHCNHNH | Cl | 2 | NRCNHNR | Cl | 2 | NHCOO | Cl | 2 | NRCOO | Cl |
| 2 | NHCNHNH | Br | 2 | NRCNHNR | Br | 2 | NHCOO | Br | 2 | NRCOO | Br |
| 2 | NHCNHNH | I | 2 | NRCNHNR | I | 2 | NHCOO | I | 2 | NRCOO | I |
| 2 | NHCNHNH | F | 2 | NRCNHNR | F | 2 | NHCOO | F | 2 | NRCOO | F |
| 2 | NHCNHNH | CN | 2 | NRCNHNR | CN | 2 | NHCOO | CN | 2 | NRCOO | CN |
| 2 | NHCNHNH | N₃ | 2 | NRCNHNR | N₃ | 2 | NHCOO | N₃ | 2 | NRCOO | N₃ |
| 2 | NHCNHNH | CONH₂ | 2 | NRCNHNR | CONH₂ | 2 | NHCOO | CONH₂ | 2 | NRCOO | CONH₂ |
| 2 | NHCNHNH | CH═CH₂ | 2 | NRCNHNR | CH═CH₂ | 2 | NHCOO | CH═CH₂ | 2 | NRCOO | CH═CH₂ |
| 2 | NHCNHNH | CCH | 2 | NRCNHNR | C≡CH | 2 | NHCOO | C≡CH | 2 | NRCOO | C≡CH |
| 2 | NHCNHNH | NH₂ | 2 | NRCNHNR | NH₂ | 2 | NHCOO | NH₂ | 2 | NRCOO | NH₂ |
| 2 | NHCNHNH | NHR | 2 | NRCNHNR | NHR | 2 | NHCOO | NHR | 2 | NRCOO | NHR |
| 2 | NHCNHNH | COH | 2 | NRCNHNR | COH | 2 | NHCOO | COH | 2 | NRCOO | COH |
| 2 | NHCNHNH | COR | 2 | NRCNHNR | COR | 2 | NHCOO | COR | 2 | NRCOO | COR |
| 2 | C≡C | OH | 2 | CH₂═CH₂ | OH | 3 | O | OH | 3 | S | OH |
| 2 | C≡C | SH | 2 | CH₂═CH₂ | SH | 3 | O | SH | 3 | S | SH |
| 2 | C≡C | COOH | 2 | CH₂═CH₂ | COOH | 3 | O | COOH | 3 | S | COOH |
| 2 | C≡C | SO₂H | 2 | CH₂═CH₂ | SO₂H | 3 | O | SO₂H | 3 | S | SO₂H |
| 2 | C≡C | Cl | 2 | CH₂═CH₂ | Cl | 3 | O | Cl | 3 | S | Cl |
| 2 | C≡C | Br | 2 | CH₂═CH₂ | Br | 3 | O | Br | 3 | S | Br |
| 2 | C≡C | I | 2 | CH₂═CH₂ | I | 3 | O | I | 3 | S | I |
| 2 | C≡C | F | 2 | CH₂═CH₂ | F | 3 | O | F | 3 | S | F |
| 2 | C≡C | CN | 2 | CH₂═CH₂ | CN | 3 | O | CN | 3 | S | CN |
| 2 | C≡C | N₃ | 2 | CH₂═CH₂ | N₃ | 3 | O | N₃ | 3 | S | N₃ |
| 2 | C≡C | CONH₂ | 2 | CH₂═CH₂ | CONH₂ | 3 | O | CONH₂ | 3 | S | CONH₂ |
| 2 | C≡C | CH≡CH₂ | 2 | CH₂═CH₂ | CH═CH₂ | 3 | O | CH═CH₂ | 3 | S | CH═CH₂ |
| 2 | C≡C | C≡CH | 2 | CH₂═CH₂ | C≡CH | 3 | O | C≡CH | 3 | S | C≡CH |
| 2 | C≡C | NH₂ | 2 | CH₂═CH₂ | NH₂ | 3 | O | NH₂ | 3 | S | NH₂ |
| 2 | C≡C | NHR | 2 | CH₂═CH₂ | NHR | 3 | O | NHR | 3 | S | NHR |
| 2 | C≡C | COH | 2 | CH₂═CH₂ | COH | 3 | O | COH | 3 | S | COH |
| 2 | C≡C | COR | 2 | CH₂═CH₂ | COR | 3 | O | COR | 3 | S | COR |
| 3 | NH | OH | 3 | NR | OH | 3 | CH₂ | OH | 3 | COR₁R₂ | OH |
| 3 | NH | SH | 3 | NR | SH | 3 | CH₂ | SH | 3 | COR₁R₂ | SH |
| 3 | NH | COOH | 3 | NR | COOH | 3 | CH₂ | COOH | 3 | COR₁R₂ | COOH |
| 3 | NH | SO₂H | 3 | NR | SO₂H | 3 | CH₂ | SO₂H | 3 | COR₁R₂ | SO₂H |
| 3 | NH | Cl | 3 | NR | Cl | 3 | CH₂ | Cl | 3 | COR₁R₂ | Cl |
| 3 | NH | Br | 3 | NR | Br | 3 | CH₂ | Br | 3 | COR₁R₂ | Br |
| 3 | NH | I | 3 | NR | I | 3 | CH₂ | I | 3 | COR₁R₂ | I |
| 3 | NH | F | 3 | NR | F | 3 | CH₂ | F | 3 | COR₁R₂ | F |
| 3 | NH | CN | 3 | NR | CN | 3 | CH₂ | CN | 3 | COR₁R₂ | CN |
| 3 | NH | N₃ | 3 | NR | N₃ | 3 | CH₂ | N₃ | 3 | COR₁R₂ | N₃ |
| 3 | NH | CONH₂ | 3 | NR | CONH₂ | 3 | CH₂ | CONH₂ | 3 | COR₁R₂ | CONH₂ |
| 3 | NH | CH═CH₂ | 3 | NR | CH═CH₂ | 3 | CH₂ | CH═CH₂ | 3 | COR₁R₂ | CH═CH₂ |
| 3 | NH | C≡CH | 3 | NR | C≡CH | 3 | CH₂ | C≡CH | 3 | COR₁R₂ | C≡CH |
| 3 | NH | NH₂ | 3 | NR | NH₂ | 3 | CH₂ | NH₂ | 3 | COR₁R₂ | NH₂ |
| 3 | NH | NHR | 3 | NR | NHR | 3 | CH₂ | NHR | 3 | COR₁R₂ | NHR |
| 3 | NH | COH | 3 | NR | COH | 3 | CH₂ | COH | 3 | COR₁R₂ | COH |
| 3 | NH | COR | 3 | NR | COR | 3 | CH₂ | COR | 3 | COR₁R₂ | COR |
| 3 | CONH | OH | 3 | CONR | OH | 3 | SO₂NH | OH | 3 | SO₂NR | OH |
| 3 | CONH | SH | 3 | CONR | SH | 3 | SO₂NH | SH | 3 | SO₂NR | SH |
| 3 | CONH | COOH | 3 | CONR | COOH | 3 | SO₂NH | COOH | 3 | SO₂NR | COOH |
| 3 | CONH | SO₂H | 3 | CONR | SO₂H | 3 | SO₂NH | SO₂H | 3 | SO₂NR | SO₂H |
| 3 | CONH | Cl | 3 | CONR | Cl | 3 | SO₂NH | Cl | 3 | SO₂NR | Cl |
| 3 | CONH | Br | 3 | CONR | Br | 3 | SO₂NH | Br | 3 | SO₂NR | Br |
| 3 | CONH | I | 3 | CONR | I | 3 | SO₂NH | I | 3 | SO₂NR | I |
| 3 | CONH | F | 3 | CONR | F | 3 | SO₂NH | F | 3 | SO₂NR | F |
| 3 | CONH | CN | 3 | CONR | CN | 3 | SO₂NH | CN | 3 | SO₂NR | CN |
| 3 | CONH | N₃ | 3 | CONR | N₃ | 3 | SO₂NH | N₃ | 3 | SO₂NR | N₃ |
| 3 | CONH | CONH₂ | 3 | CONR | CONH₂ | 3 | SO₂NH | CONH₂ | 3 | SO₂NR | CONH₂ |
| 3 | CONH | CH═CH₂ | 3 | CONR | CH═CH₂ | 3 | SO₂NH | CH═CH₂ | 3 | SO₂NR | CH═CH₂ |
| 3 | CONH | C≡CH | 3 | CONR | C≡CH | 3 | SO₂NH | C≡CH | 3 | SO₂NR | C≡CH |
| 3 | CONH | NH₂ | 3 | CONR | NH₂ | 3 | SO₂NH | NH₂ | 3 | SO₂NR | NH₂ |
| 3 | CONH | NHR | 3 | CONR | NHR | 3 | SO₂NH | NHR | 3 | SO₂NR | NHR |
| 3 | CONH | COH | 3 | CONR | COH | 3 | SO₂NH | COH | 3 | SO₂NR | COH |
| 3 | CONH | COR | 3 | CONR | COR | 3 | SO₂NH | COR | 3 | SO₂NR | COR |
| 3 | NHCONH | OH | 3 | NRCONR | OH | 3 | NHCNHNH | OH | 3 | NRCNHNR | OH |
| 3 | NHCONH | SH | 3 | NRCONR | SH | 3 | NHCNHNH | SH | 3 | NRCNHNR | SH |
| 3 | NHCONH | COOH | 3 | NRCONR | COOH | 3 | NHCNHNH | COOH | 3 | NRCNHNR | COOH |
| 3 | NHCONH | SO₂H | 3 | NRCONR | SO₂H | 3 | NHCNHNH | SO₂H | 3 | NRCNHNR | CO₂H |
| 3 | NHCONH | Cl | 3 | NRCONR | Cl | 3 | NHCNHNH | Cl | 3 | NRCNHNR | Cl |
| 3 | NHCONH | Br | 3 | NRCONR | Br | 3 | NHCNHNH | Br | 3 | NRCNHNR | Br |
| 3 | NHCONH | I | 3 | NRCONR | I | 3 | NHCNHNH | I | 3 | NRCNHNR | I |
| 3 | NHCONH | F | 3 | NRCONR | F | 3 | NHCNHNH | F | 3 | NRCNHNR | F |
| 3 | NHCONH | CN | 3 | NRCONR | CN | 3 | NHCNHNH | CN | 3 | NRCNHNR | CN |
| 3 | NHCONH | N₃ | 3 | NRCONR | N₃ | 3 | NHCNHNH | N₃ | 3 | NRCNHNR | N₃ |
| 3 | NHCONH | CONH₂ | 3 | NRCONR | CONH₂ | 3 | NHCNHNH | CONH₂ | 3 | NRCNHNR | CONH₂ |
| 3 | NHCONH | CH═CH₂ | 3 | NRCONR | CH═CH₂ | 3 | NHCNHNH | CH═CH₂ | 3 | NRCNHNR | CH═CH₂ |
| 3 | NHCONH | C≡CH | 3 | NRCONR | C≡CH | 3 | NHCNHNH | C≡CH | 3 | NRCNHNR | C≡CH |
| 3 | NHCONH | NH₂ | 3 | NRCONR | NH₂ | 3 | NHCNHNH | NH₂ | 3 | NRCNHNR | NH₂ |
| 3 | NHCONH | NHR | 3 | NRCONR | NHR | 3 | NHCNHNH | NHR | 3 | NRCNHNR | NHR |
| 3 | NHCONH | COH | 3 | NRCONR | COH | 3 | NHCNHNH | COH | 3 | NRCNHNR | COH |
| 3 | NHCONH | COR | 3 | NRCONR | COR | 3 | NHCNHNH | COR | 3 | NRCNHNR | COR |
| 3 | NHCOO | OH | 3 | NRCOO | OH | 3 | C≡C | OH | 3 | CH₂═CH₂ | OH |
| 3 | NHCOO | SH | 3 | NRCOO | SH | 3 | C≡C | SH | 3 | CH₂═CH₂ | SH |
| 3 | NHCOO | COOH | 3 | NRCOO | COOH | 3 | C≡C | COOH | 3 | CH₂═CH₂ | COOH |
| 3 | NHCOO | SO₂H | 3 | NRCOO | SO₂H | 3 | C≡C | SO₂H | 3 | CH₂═CH₂ | SO₂H |
| 3 | NHCOO | Cl | 3 | NRCOO | Cl | 3 | C≡C | Cl | 3 | CH₂═CH₂ | Cl |
| 3 | NHCOO | Br | 3 | NRCOO | Br | 3 | C≡C | Br | 3 | CH₂═CH₂ | Br |
| 3 | NHCOO | I | 3 | NRCOO | I | 3 | C≡C | I | 3 | CH₂═CH₂ | I |
| 3 | NHCOO | F | 3 | NRCOO | F | 3 | C≡C | F | 3 | CH₂═CH₂ | F |
| 3 | NHCOO | CN | 3 | NRCOO | CN | 3 | C≡C | CN | 3 | CH₂═CH₂ | CN |
| 3 | NHCOO | N₃ | 3 | NRCOO | N₃ | 3 | C≡C | N₃ | 3 | CH₂═CH₂ | N₃ |
| 3 | NHCOO | CONH₂ | 3 | NRCOO | CONH₂ | 3 | C≡C | CONH₂ | 3 | CH₂═CH₂ | CONH₂ |
| 3 | NHCOO | CH═CH₂ | 3 | NRCOO | CH═CH₂ | 3 | C≡C | CH═CH₂ | 3 | CH₂═CH₂ | CH═CH₂ |
| 3 | NHCOO | C≡CH | 3 | NRCOO | C≡CH | 3 | C≡C | C≡CH | 3 | CH₂═CH₂ | C≡CH |
| 3 | NHCOO | NH₂ | 3 | NRCOO | NH₂ | 3 | C≡C | NH₂ | 3 | CH₂═CH₂ | NH₂ |
| 3 | NHCOO | NHR | 3 | NRCOO | NHR | 3 | C≡C | NHR | 3 | CH₂═CH₂ | NHR |
| 3 | NHCOO | COH | 3 | NRCOO | COH | 3 | C≡C | COH | 3 | CH₂═CH₂ | COH |
| 3 | NHCOO | COR | 3 | NRCOO | COR | 3 | C≡C | COR | 3 | CH₂═CH₂ | COR |
| 4 | O | OH | 4 | S | OH | 4 | NH | OH | 4 | NR | OH |
| 4 | O | SH | 4 | S | SH | 4 | NH | SH | 4 | NR | SH |
| 4 | O | COOH | 4 | S | COOH | 4 | NH | COOH | 4 | NR | COOH |
| 4 | O | SO₂H | 4 | S | SO₂H | 4 | NH | SO₂H | 4 | NR | SO₂H |
| 4 | O | Cl | 4 | S | Cl | 4 | NH | Cl | 4 | NR | Cl |
| 4 | O | Br | 4 | S | Br | 4 | NH | Br | 4 | NR | Br |
| 4 | O | I | 4 | S | I | 4 | NH | I | 4 | NR | I |
| 4 | O | F | 4 | S | F | 4 | NH | F | 4 | NR | F |
| 4 | O | CN | 4 | S | CN | 4 | NH | CN | 4 | NR | CN |
| 4 | O | N₃ | 4 | S | N₃ | 4 | NH | N₃ | 4 | NR | N₃ |
| 4 | O | CONH₂ | 4 | S | CONH₂ | 4 | NH | CONH₂ | 4 | NR | CONH₂ |
| 4 | O | CH═CH₂ | 4 | S | CH═CH₂ | 4 | NH | CH═CH₂ | 4 | NR | CH═CH₂ |
| 4 | O | C≡CH | 4 | S | C≡CH | 4 | NH | C≡CH | 4 | NR | C≡CH |
| 4 | O | NH₂ | 4 | S | NH₂ | 4 | NH | NH₂ | 4 | NR | NH₂ |
| 4 | O | NHR | 4 | S | NHR | 4 | NH | NHR | 4 | NR | NHR |
| 4 | O | COH | 4 | S | COH | 4 | NH | COH | 4 | NR | COH |
| 4 | O | COR | 4 | S | COR | 4 | NH | COR | 4 | NR | COR |
| 4 | CH₂ | OH | 4 | COR₁R₂ | OH | 4 | CONH | OH | 4 | CONR | OH |
| 4 | CH₂ | SH | 4 | COR₁R₂ | SH | 4 | CONH | SH | 4 | CONR | SH |
| 4 | CH₂ | COOH | 4 | COR₁R₂ | COOH | 4 | CONH | COOH | 4 | CONR | COOH |
| 4 | CH₂ | SO₂H | 4 | COR₁R₂ | SO₂H | 4 | CONH | SO₂H | 4 | CONR | SO₂H |
| 4 | CH₂ | Cl | 4 | COR₁R₂ | Cl | 4 | CONH | Cl | 4 | CONR | Cl |
| 4 | CH₂ | Br | 4 | COR₁R₂ | Br | 4 | CONH | Br | 4 | CONR | Br |
| 4 | CH₂ | I | 4 | COR₁R₂ | I | 4 | CONH | I | 4 | CONR | I |
| 4 | CH₂ | F | 4 | COR₁R₂ | F | 4 | CONH | F | 4 | CONR | F |
| 4 | CH₂ | CN | 4 | COR₁R₂ | CN | 4 | CONH | CN | 4 | CONR | CN |
| 4 | CH₂ | N₃ | 4 | COR₁R₂ | N₃ | 4 | CONH | N₃ | 4 | CONR | N₃ |
| 4 | CH₂ | CONH₂ | 4 | COR₁R₂ | CONH₂ | 4 | CONH | CONH₂ | 4 | CONR | CONH₂ |
| 4 | CH₂ | CH═CH₂ | 4 | COR₁R₂ | CH═CH₂ | 4 | CONH | CH═CH₂ | 4 | CONR | CH═CH₂ |
| 4 | CH₂ | C≡CH | 4 | COR₁R₂ | C≡CH | 4 | CONH | C≡CH | 4 | CONR | C≡CH |
| 4 | CH₂ | NH₂ | 4 | COR₁R₂ | NH₂ | 4 | CONH | NH₂ | 4 | CONR | NH₂ |
| 4 | CH₂ | NHR | 4 | COR₁R₂ | NHR | 4 | CONH | NHR | 4 | CONR | NHR |
| 4 | CH₂ | COH | 4 | COR₁R₂ | COH | 4 | CONH | COH | 4 | CONR | COH |
| 4 | CH₂ | COR | 4 | COR₁R₂ | COR | 4 | CONH | COR | 4 | CONR | COR |
| 4 | SO₂NH | OH | 4 | SO₂NR | OH | 4 | NHCONH | OH | 4 | NRCONR | OH |
| 4 | SO₂NH | SH | 4 | SO₂NR | SH | 4 | NHCONH | SH | 4 | NRCONR | SH |
| 4 | SO₂NH | COOH | 4 | SO₂NR | COOH | 4 | NHCONH | COOH | 4 | NRCONR | COOH |
| 4 | SO₂NH | SO₂H | 4 | SO₂NR | SO₂H | 4 | NHCONH | SO₂H | 4 | NRCONR | SO₂H |
| 4 | SO₂NH | Cl | 4 | SO₂NR | Cl | 4 | NHCONH | Cl | 4 | NRCONR | Cl |
| 4 | SO₂NH | Br | 4 | SO₂NR | Br | 4 | NHCONH | Br | 4 | NRCONR | Br |
| 4 | SO₂NH | I | 4 | SO₂NR | I | 4 | NHCONH | I | 4 | NRCONR | I |
| 4 | SO₂NH | F | 4 | SO₂NR | F | 4 | NHCONH | F | 4 | NRCONR | F |
| 4 | SO₂NH | CN | 4 | SO₂NR | CN | 4 | NHCONH | CN | 4 | NRCONR | CN |
| 4 | SO₂NH | N₃ | 4 | SO₂NR | N₃ | 4 | NHCONH | N₃ | 4 | NRCONR | N₃ |
| 4 | SO₂NH | CONH₂ | 4 | SO₂NR | CONH₂ | 4 | NHCONH | CONH₂ | 4 | NRCONR | CONH₂ |
| 4 | SO₂NH | CH═CH₂ | 4 | SO₂NR | CH═CH₂ | 4 | NHCONH | CH═CH₂ | 4 | NRCONR | CH═CH₂ |
| 4 | SO₂NH | C≡CH | 4 | SO₂NR | C≡CH | 4 | NHCONH | C≡CH | 4 | NRCONR | C≡CH |
| 4 | SO₂NH | NH₂ | 4 | SO₂NR | NH₂ | 4 | NHCONH | NH₂ | 4 | NRCONR | NH₂ |
| 4 | SO₂NH | NHR | 4 | SO₂NR | NHR | 4 | NHCONH | NHR | 4 | NRCONR | NHR |
| 4 | SO₂NH | COH | 4 | SO₂NR | COH | 4 | NHCONH | COH | 4 | NRCONR | COH |
| 4 | SO₂NH | COR | 4 | SO₂NR | COR | 4 | NHCONH | COR | 4 | NRCONR | COR |
| 4 | NHCNHNH | OH | 4 | NRCNHNR | OH | 4 | NHCOO | OH | 4 | NRCOO | OH |
| 4 | NHCNHNH | SH | 4 | NRCNHNR | SH | 4 | NHCOO | SH | 4 | NRCOO | SH |
| 4 | NHCNHNH | COOH | 4 | NRCNHNR | COOH | 4 | NHCOO | COOH | 4 | NRCOO | COOH |
| 4 | NHCNHNH | SO₂H | 4 | NRCNHNR | SO₂H | 4 | NHCOO | SO₂H | 4 | NRCOO | SO₂H |
| 4 | NHCNHNH | Cl | 4 | NRCNHNR | Cl | 4 | NHCOO | Cl | 4 | NRCOO | Cl |
| 4 | NHCNHNH | Br | 4 | NRCNHNR | Br | 4 | NHCOO | Br | 4 | NRCOO | Br |
| 4 | NHCNHNH | I | 4 | NRCNHNR | I | 4 | NHCOO | I | 4 | NRCOO | I |
| 4 | NHCNHNH | F | 4 | NRCNHNR | F | 4 | NHCOO | F | 4 | NRCOO | F |
| 4 | NHCNHNH | CN | 4 | NRCNHNR | CN | 4 | NHCOO | CN | 4 | NRCOO | CN |
| 4 | NHCNHNH | N₃ | 4 | NRCNHNR | N₃ | 4 | NHCOO | N₃ | 4 | NRCOO | N₃ |
| 4 | NHCNHNH | CONH₂ | 4 | NRCNHNR | CONH₂ | 4 | NHCOO | CONH₂ | 4 | NRCOO | CONH₂ |
| 4 | NHCNHNH | CH═CH₂ | 4 | NRCNHNR | CH═CH₂ | 4 | NHCOO | CH═CH₂ | 4 | NRCOO | CH═CH₂ |
| 4 | NHCNHNH | C≡CH | 4 | NRCNHNR | C≡CH | 4 | NHCOO | C≡CH | 4 | NRCOO | C≡CH |
| 4 | NHCNHNH | NH₂ | 4 | NRCNHNR | NH₂ | 4 | NHCOO | NH₂ | 4 | NRCOO | NH₂ |
| 4 | NHCNHNH | NHR | 4 | NRCNHNR | NHR | 4 | NHCOO | NHR | 4 | NRCOO | NHR |
| 4 | NHCNHNH | COH | 4 | NRCNHNR | COH | 4 | NHCOO | COH | 4 | NRCOO | COH |
| 4 | NHCNHNH | COR | 4 | NRCNHNR | COR | 4 | NHCOO | COR | 4 | NRCOO | COR |
| 4 | C≡C | OH | 4 | CH₂═CH₂ | OH | 5 | O | OH | 5 | S | OH |
| 4 | C≡C | SH | 4 | CH₂═CH₂ | SH | 5 | O | SH | 5 | S | SH |
| 4 | C≡C | COOH | 4 | CH₂═CH₂ | COOH | 5 | O | COOH | 5 | S | COOH |
| 4 | C≡C | SO₂H | 4 | CH₂═CH₂ | SO₂H | 5 | O | SO₂H | 5 | S | SO₂H |
| 4 | C≡C | Cl | 4 | CH₂═CH₂ | Cl | 5 | O | Cl | 5 | S | Cl |
| 4 | C≡C | Br | 4 | CH₂═CH₂ | Br | 5 | O | Br | 5 | S | Br |
| 4 | C≡C | I | 4 | CH₂═CH₂ | I | 5 | O | I | 5 | S | I |
| 4 | C≡C | F | 4 | CH₂═CH₂ | F | 5 | O | F | 5 | S | F |
| 4 | C≡C | CN | 4 | CH₂═CH₂ | CN | 5 | O | CN | 5 | S | CN |
| 4 | C≡C | N₃ | 4 | CH₂═CH₂ | N₃ | 5 | O | N₃ | 5 | S | N₃ |
| 4 | C≡C | CONH₂ | 4 | CH₂═CH₂ | CONH₂ | 5 | O | CONH₂ | 5 | S | CONH₂ |
| 4 | C≡C | CH═CH₂ | 4 | CH₂═CH₂ | CH═CH₂ | 5 | O | CH═CH₂ | 5 | S | CH═CH₂ |
| 4 | C≡C | C≡CH | 4 | CH₂═CH₂ | C≡CH | 5 | O | C≡CH | 5 | S | C≡CH |
| 4 | C≡C | NH₂ | 4 | CH₂═CH₂ | NH₂ | 5 | O | NH₂ | 5 | S | NH₂ |
| 4 | C≡C | NHR | 4 | CH₂═CH₂ | NHR | 5 | O | NHR | 5 | S | NHR |
| 4 | C≡C | COH | 4 | CH₂═CH₂ | COH | 5 | O | COH | 5 | S | COH |
| 4 | C≡C | COR | 4 | CH₂═CH₂ | COR | 5 | O | COR | 5 | S | COR |
| 5 | NH | OH | 5 | NR | OH | 5 | CH₂ | OH | 5 | COR₁R₂ | OH |
| 5 | NH | SH | 5 | NR | SH | 5 | CH₂ | SH | 5 | COR₁R₂ | SH |
| 5 | NH | COOH | 5 | NR | COOH | 5 | CH₂ | COOH | 5 | COR₁R₂ | COOH |
| 5 | NH | SO₂H | 5 | NR | SO₂H | 5 | CH₂ | SO₂H | 5 | COR₁R₂ | SO₂H |
| 5 | NH | Cl | 5 | NR | Cl | 5 | CH₂ | Cl | 5 | COR₁R₂ | Cl |
| 5 | NH | Br | 5 | NR | Br | 5 | CH₂ | Br | 5 | COR₁R₂ | Br |
| 5 | NH | I | 5 | NR | I | 5 | CH₂ | I | 5 | COR₁R₂ | I |
| 5 | NH | F | 5 | NR | F | 5 | CH₂ | F | 5 | COR₁R₂ | F |
| 5 | NH | CN | 5 | NR | CN | 5 | CH₂ | CN | 5 | COR₁R₂ | CN |
| 5 | NH | N₃ | 5 | NR | N₃ | 5 | CH₂ | N₃ | 5 | COR₁R₂ | N₃ |
| 5 | NH | CONH₂ | 5 | NR | CONH₂ | 5 | CH₂ | CONH₂ | 5 | COR₁R₂ | CONH₂ |
| 5 | NH | CH═CH₂ | 5 | NR | CH═CH₂ | 5 | CH₂ | CH═CH₂ | 5 | COR₁R₂ | CH═CH₂ |
| 5 | NH | C≡CH | 5 | NR | C≡CH | 5 | CH₂ | C≡CH | 5 | COR₁R₂ | C≡CH |
| 5 | NH | NH₂ | 5 | NR | NH₂ | 5 | CH₂ | NH₂ | 5 | COR₁R₂ | NH₂ |
| 5 | NH | NHR | 5 | NR | NHR | 5 | CH₂ | NHR | 5 | COR₁R₂ | NHR |
| 5 | NH | COH | 5 | NR | COH | 5 | CH₂ | COH | 5 | COR₁R₂ | COH |
| 5 | NH | COR | 5 | NR | COR | 5 | CH₂ | COR | 5 | COR₁R₂ | COR |
| 5 | CONH | OH | 5 | CONR | OH | 5 | SO₂NH | OH | 5 | SO₂NR | OH |
| 5 | CONH | SH | 5 | CONR | SH | 5 | SO₂NH | SH | 5 | SO₂NR | SH |
| 5 | CONH | COOH | 5 | CONR | COOH | 5 | SO₂NH | COOH | 5 | SO₂NR | COOH |
| 5 | CONH | SO₂H | 5 | CONR | SO₂H | 5 | SO₂NH | SO₂H | 5 | SO₂NR | SO₂H |
| 5 | CONH | Cl | 5 | CONR | Cl | 5 | SO₂NH | Cl | 5 | SO₂NR | Cl |
| 5 | CONH | Br | 5 | CONR | Br | 5 | SO₂NH | Br | 5 | SO₂NR | Br |
| 5 | CONH | I | 5 | CONR | I | 5 | SO₂NH | I | 5 | SO₂NR | I |
| 5 | CONH | F | 5 | CONR | F | 5 | SO₂NH | F | 5 | SO₂NR | F |
| 5 | CONH | CN | 5 | CONR | CN | 5 | SO₂NH | CN | 5 | SO₂NR | CN |
| 5 | CONH | N₃ | 5 | CONR | N₃ | 5 | SO₂NH | N₃ | 5 | SO₂NR | N₃ |
| 5 | CONH | CONH₂ | 5 | CONR | CONH₂ | 5 | SO₂NH | CONH₂ | 5 | SO₂NR | CONH₂ |
| 5 | CONH | CH═CH₂ | 5 | CONR | CH═CH₂ | 5 | SO₂NH | CH═CH₂ | 5 | SO₂NR | CH═CH₂ |
| 5 | CONH | C≡CH | 5 | CONR | C≡CH | 5 | SO₂NH | C≡CH | 5 | SO₂NR | C≡CH |
| 5 | CONH | NH₂ | 5 | CONR | NH₂ | 5 | SO₂NH | NH₂ | 5 | SO₂NR | NH₂ |
| 5 | CONH | NHR | 5 | CONR | NHR | 5 | SO₂NH | NHR | 5 | SO₂NR | NHR |
| 5 | CONH | COH | 5 | CONR | COH | 5 | SO₂NH | COH | 5 | SO₂NR | COH |
| 5 | CONH | COR | 5 | CONR | COR | 5 | SO₂NH | COR | 5 | SO₂NR | COR |
| 5 | NHCONH | OH | 5 | NRCONR | OH | 5 | NHCNHNH | OH | 5 | NRCNHNR | OH |
| 5 | NHCONH | SH | 5 | NRCONR | SH | 5 | NHCNHNH | SH | 5 | NRCNHNR | SH |
| 5 | NHCONH | COOH | 5 | NRCONR | COOH | 5 | NHCNHNH | COOH | 5 | NRCNHNR | COOH |
| 5 | NHCONH | SO₂H | 5 | NRCONR | SO₂H | 5 | NHCNHNH | SO₂H | 5 | NRCNHNR | SO₂H |
| 5 | NHCONH | Cl | 5 | NRCONR | Cl | 5 | NHCNHNH | Cl | 5 | NRCNHNR | Cl |
| 5 | NHCONH | Br | 5 | NRCONR | Br | 5 | NHCNHNH | Br | 5 | NRCNHNR | Br |
| 5 | NHCONH | I | 5 | NRCONR | I | 5 | NHCNHNH | I | 5 | NRCNHNR | I |
| 5 | NHCONH | F | 5 | NRCONR | F | 5 | NHCNHNH | F | 5 | NRCNHNR | F |
| 5 | NHCONH | CN | 5 | NRCONR | CN | 5 | NHCNHNH | CN | 5 | NRCNHNR | CN |
| 5 | NHCONH | N₃ | 5 | NRCONR | N₃ | 5 | NHCNHNH | N₃ | 5 | NRCNHNR | N₃ |
| 5 | NHCONH | CONH₂ | 5 | NRCONR | CONH₂ | 5 | NHCNHNH | CONH₂ | 5 | NRCNHNR | CONH₂ |
| 5 | NHCONH | CH═CH₂ | 5 | NRCONR | CH═CH₂ | 5 | NHCNHNH | CH═CH₂ | 5 | NRCNHNR | CH═CH₂ |
| 5 | NHCONH | C≡CH | 5 | NRCONR | C≡CH | 5 | NHCNHNH | C≡CH | 5 | NRCNHNR | C≡CH |
| 5 | NHCONH | NH₂ | 5 | NRCONR | NH₂ | 5 | NHCNHNH | NH₂ | 5 | NRCNHNR | NH₂ |
| 5 | NHCONH | NHR | 5 | NRCONR | NHR | 5 | NHCNHNH | NHR | 5 | NRCNHNR | NHR |
| 5 | NHCONH | COH | 5 | NRCONR | COH | 5 | NHCNHNH | COH | 5 | NRCNHNR | COH |
| 5 | NHCONH | COR | 5 | NRCONR | COR | 5 | NHCNHNH | COR | 5 | NRCNHNR | COR |
| 5 | NRCNHNR | OH | 5 | NHCOO | OH | 5 | NRCOO | OH | 5 | C≡C | OH |
| 5 | NRCNHNR | SH | 5 | NHCOO | SH | 5 | NRCOO | SH | 5 | C≡C | SH |
| 5 | NRCNHNR | COOH | 5 | NHCOO | COOH | 5 | NRCOO | COOH | 5 | C≡C | COOH |
| 5 | NRCNHNR | SO₂H | 5 | NHCOO | SO₂H | 5 | NRCOO | SO₂H | 5 | C≡C | SO₂H |
| 5 | NRCNHNR | Cl | 5 | NHCOO | Cl | 5 | NRCOO | Cl | 5 | C≡C | Cl |
| 5 | NRCNHNR | Br | 5 | NHCOO | Br | 5 | NRCOO | Br | 5 | C≡C | Br |
| 5 | NRCNHNR | I | 5 | NHCOO | I | 5 | NRCOO | I | 5 | C≡C | I |
| 5 | NRCNHNR | F | 5 | NHCOO | F | 5 | NRCOO | F | 5 | C≡C | F |
| 5 | NRCNHNR | CN | 5 | NHCOO | CN | 5 | NRCOO | CN | 5 | C≡C | CN |
| 5 | NRCNHNR | N₃ | 5 | NHCOO | N₃ | 5 | NRCOO | N₃ | 5 | C≡C | N₃ |
| 5 | NRCNHNR | CONH₂ | 5 | NHCOO | CONH₂ | 5 | NRCOO | CONH₂ | 5 | C≡C | CONH₂ |
| 5 | NRCNHNR | CH═CH₂ | 5 | NHCOO | CH═CH₂ | 5 | NRCOO | CH═CH₂ | 5 | C≡C | CH═CH₂ |
| 5 | NRCNHNR | C≡CH | 5 | NHCOO | C≡CH | 5 | NRCOO | C≡CH | 5 | C≡C | C≡CH |
| 5 | NRCNHNR | NH₂ | 5 | NHCOO | NH₂ | 5 | NRCOO | NH₂ | 5 | C≡C | NH₂ |
| 5 | NRCNHNR | NHR | 5 | NHCOO | NHR | 5 | NRCOO | NHR | 5 | C≡C | NHR |
| 5 | NRCNHNR | COH | 5 | NHCOO | COH | 5 | NRCOO | COH | 5 | C≡C | COH |
| 5 | NRCNHNR | COR | 5 | NHCOO | COR | 5 | NRCOO | COR | 5 | C≡C | COR |
| 5 | CH₂═CH₂ | OH | 5 | CH₂═CH₂ | Br | 5 | CH₂═CH₂ | N₃ | 5 | CH₂═CH₂ | NH₂ |
| 5 | CH₂═CH₂ | SH | 5 | CH₂═CH₂ | I | 5 | CH₂═CH₂ | CONH₂ | 5 | CH₂═CH₂ | NHR |
| 5 | CH₂═CH₂ | COOH | 5 | CH₂═CH₂ | F | 5 | CH₂═CH₂ | CH═CH₂ | 5 | CH₂═CH₂ | COH |
| 5 | CH₂═CH₂ | SO₂H | 5 | CH₂═CH₂ | CN | 5 | CH₂═CH₂ | C≡CH | 5 | CH₂═CH₂ | COR |

| | | | |
|---|---|---|---|
| 5 | CH₂═CH₂ | Cl | R, R₁, and R₂ = H, alkyl, alkenyl, alkynyl, aryl, and heterocycle |

TABLE 7

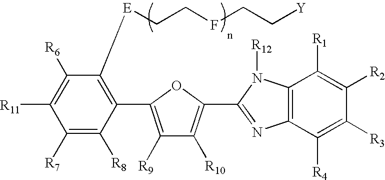

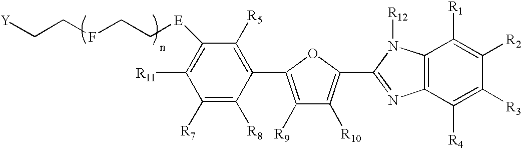

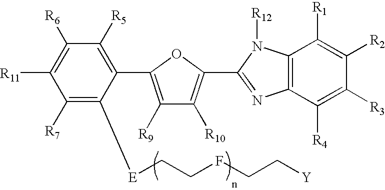

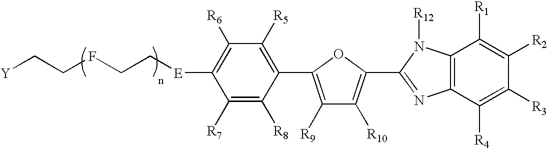

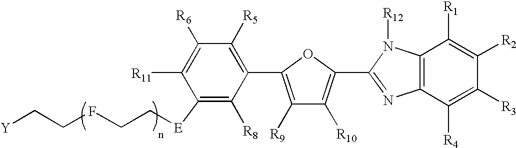

| n | E | F | Y | n | E | F | Y |
|---|---|---|---|---|---|---|---|
| 0 | O | O | OH | 0 | O | S | OH |
| 0 | O | O | NH₂ | 0 | O | S | NH₂ |
| 0 | O | CONR | I | 0 | O | SO₂NR | I |
| 0 | O | NRCONR | COH | 0 | O | NRCNHNR | COH |
| 0 | O | NRCONR | COR | 0 | O | NRCNHNR | COR |
| 0 | O | NRCOO | CH═CH₂ | 0 | O | C≡C | CH═CH₂ |
| 0 | O | CH═CH | NHR | 0 | S | O | NHR |
| 0 | O | CH═CH | CON | 0 | S | O | COH |
| 0 | S | S | NHR | 0 | S | NR | NHR |
| 0 | S | S | COH | 0 | S | NR | COH |
| 0 | S | S | COR | 0 | S | NR | COR |
| 0 | S | CR₁R₂ | COH | 0 | S | CONR | COH |
| 0 | S | CR₁R₂ | COR | 0 | S | CONR | COR |
| 0 | S | SO₂NR | OH | 0 | S | NRCONR | OH |
| 0 | S | SO₂NR | SO₂H | 0 | S | NRCONR | SO₂H |
| 0 | S | NRCNHNR | CONH₂ | 0 | S | NRCOO | CONH₂ |
| 0 | S | NRCNHNR | CH═CH₂ | 0 | S | NRCOO | CH═CH₂ |
| 0 | NR | O | C≡CH | 0 | NR | S | C≡CH |
| 0 | NR | CONR | Cl | 0 | NR | SO₂NR | Cl |
| 0 | NR | CONR | COR | 0 | NR | SO₂NR | COR |
| 0 | NR | NRCONR | OH | 0 | NR | NRCNHNR | OH |
| 0 | NR | NRCONR | SH | 0 | NR | NRCNHNR | SH |
| 0 | NR | NRCONR | CONH₂ | 0 | NR | NRCNHNR | CONH₂ |
| 0 | NR | NRCOO | COR | 0 | NR | | COR |
| 0 | NR | CH═CH | OH | 0 | CR₁R₂ | O | OH |
| 0 | NR | CH═CH | N₃ | 0 | CR₁R₂ | O | N₃ |
| 0 | NR | CH═CH | CONH₂ | 0 | CR₁R₂ | O | CONH₂ |
| 0 | NR | CH═CH | CH═CH₂ | 0 | CR₁R₂ | O | CH═CH₂ |
| 0 | CR₁R₂ | S | COH | 0 | CR₁R₂ | NR | COH |
| 0 | CR₁R₂ | S | COR | 0 | CR₁R₂ | NR | COR |
| 0 | CR₁R₂ | CR₁R₂ | SH | 0 | CR₁R₂ | CONR | SH |
| 0 | CR₁R₂ | CR₁R₂ | COOH | 0 | CR₁R₂ | CONR | COOH |
| 0 | CR₁R₂ | CR₁R₂ | NH₂ | 0 | CR₁R₂ | CONR | NH₂ |
| 0 | CR₁R₂ | SO₂NR | Cl | 0 | CR₁R₂ | NRCONR | Cl |
| 0 | CR₁R₂ | SO₂NR | CN | 0 | CR₁R₂ | NRCONR | CN |
| 0 | CR₁R₂ | SO₂NR | N₃ | 0 | CR₁R₂ | NRCONR | N₃ |
| 0 | CR₁R₂ | NRCNHNR | NHR | 0 | CR₁R₂ | NRCOO | NHR |
| 0 | CR₁R₂ | NRCNHNR | COR | 0 | CR₁R₂ | NRCOO | COR |
| 0 | CR₁R₂ | C≡C | OH | 0 | CR₁R₂ | CH═CH | OH |
| 0 | CR₁R₂ | C≡C | Br | 0 | CR₁R₂ | CH═CH | Br |
| 0 | CONR | O | OH | 0 | CONR | S | OH |
| 0 | CONR | O | SH | 0 | CONR | S | SH |
| 0 | CONR | O | COR | 0 | CONR | S | COR |
| 0 | CONR | NR | OH | 0 | CONR | CR₁R₂ | OH |
| 0 | CONR | NR | COR | 0 | CONR | CR₁R₂ | COR |
| 0 | CONR | CONR | OH | 0 | CONR | SO₂NR | OH |
| 0 | CONR | CONR | SH | 0 | CONR | SO₂NR | SH |
| 0 | CONR | CONR | COOH | 0 | CONR | SO₂NR | COOH |
| 0 | CONR | NRCOO | Br | 0 | CONR | C═C | Br |
| 0 | CONR | NRCOO | CONH₂ | 0 | CONR | C═C | CONH₂ |
| 0 | CONR | CH═CH | CONH₂ | 0 | SO₂NR | O | CONH₂ |
| 0 | CONR | CH═CH | CH═CH₂ | 0 | SO₂NR | O | CH═CH₂ |
| 0 | CONR | CH═CH | NH₂ | 0 | SO₂NR | O | NH₂ |
| 0 | SO₂NR | S | SH | 0 | SO₂NR | NR | SH |
| 0 | SO₂NR | S | COOH | 0 | SO₂NR | NR | COOH |
| 0 | SO₂NR | S | F | 0 | SO₂NR | NR | F |
| 0 | SO₂NR | CR₁R₂ | CONH₂ | 0 | SO₂NR | CONR | CONH₂ |
| 0 | SO₂NR | SO₂NR | F | 0 | SO₂NR | NRCONR | F |
| 0 | SO₂NR | SO₂NR | N₃ | 0 | SO₂NR | NRCONR | N₃ |
| 0 | SO₂NR | SO₂NR | CH═CH₂ | 0 | SO₂NR | NRCONR | CH═CH₂ |
| 0 | SO₂NR | NRCNHNR | SH | 0 | SO₂NR | NRCOO | SH |
| 0 | SO₂NR | NRCNHNR | SO₂H | 0 | SO₂NR | NRCOO | SO₂H |
| 0 | SO₂NR | NRCNHNR | Cl | 0 | SO₂NR | NRCOO | Cl |
| 0 | SO₂NR | C≡C | NHR | 0 | SO₂NR | CH═CH | NHR |
| 0 | SO₂NR | C≡C | COR | 0 | SO₂NR | CH═CH | COR |
| 0 | NRCONR | O | OH | 0 | NRCONR | S | OH |
| 0 | NRCONR | O | SH | 0 | NRCONR | S | SH |
| 0 | NRCONR | O | COOH | 0 | NRCONR | S | COOH |
| 0 | NRCONR | NR | SO₂H | 0 | NRCONR | CR₁R₂ | SO₂H |
| 0 | NRCONR | NR | COH | 0 | NRCONR | CR₁R₂ | COH |
| 0 | NRCONR | NR | COR | 0 | NRCONR | CR₁R₂ | COR |
| 0 | NRCONR | CONR | F | 0 | NRCONR | SO₂NR | F |
| 0 | NRCONR | CONR | CH═CH₂ | 0 | NRCONR | SO₂NR | CH═CH₂ |
| 0 | NRCONR | CONR | C≡CH | 0 | NRCONR | SO₂NR | C≡CH |
| 0 | NRCONR | NRCONR | COR | 0 | NRCONR | NRCNHNR | COR |
| 0 | NRCONR | NRCOO | OH | 0 | NRCONR | C≡C | OH |
| 0 | NRCONR | NRCOO | COH | 0 | NRCONR | C≡C | COH |
| 0 | NRCONR | NRCOO | COR | 0 | NRCONR | | COR |
| 0 | NRCONR | CH═CH | OH | 0 | NRCNHNR | O | OH |
| 0 | NRCONR | CH═CH | SH | 0 | NRCNHNR | O | SH |
| 0 | NRCONR | CH═CH | COOH | 0 | NRCNHNR | O | COOH |
| 0 | NRCNHNR | S | C≡CH | 0 | NRCNHNR | NR | C≡CH |
| 0 | NRCNHNR | S | NH₂ | 0 | NRCNHNR | NR | NH₂ |
| 0 | NRCNHNR | S | NHR | 0 | NRCNHNR | NR | NHR |
| 0 | NRCNHNR | CR₁R₂ | Br | 0 | NRCNHNR | CONR | Br |
| 0 | NRCNHNR | CR₁R₂ | NH₂ | 0 | NRCNHNR | CONR | NH₂ |
| 0 | NRCNHNR | CR₁R₂ | NHR | 0 | NRCNHNR | CONR | NHR |
| 0 | NRCNHNR | SO₂NR | SH | 0 | NRCNHNR | NRCONR | SH |
| 0 | NRCNHNR | SO₂NR | COOH | 0 | NRCNHNR | NRCONR | COOH |
| 0 | NRCNHNR | NRCNHNR | CH | 0 | NRCNHNR | NRCOO | CN |
| 0 | NRCNHNR | NRCNHNR | N₃ | 0 | NRCNHNR | NRCOO | N₃ |
| 0 | NRCNHNR | NRCNHNR | CONH₂ | 0 | NRCNHNR | NRCOO | CONH₂ |
| 0 | NRCNHNR | C≡C | SH | 0 | NRCNHNR | CH═CH | SH |
| 0 | NRCNHNR | C≡C | COOH | 0 | NRCNHNR | CH═CH | COOH |
| 0 | NRCOO | O | CN | 0 | NRCOO | S | CN |
| 0 | NRCOO | O | N₃ | 0 | NRCOO | S | N₃ |
| 0 | NRCOO | O | CONH₂ | 0 | NRCOO | S | CONH₂ |
| 0 | NRCOO | CONR | CN | 0 | NRCOO | SO₂NR | CN |
| 0 | NRCOO | CONR | N₃ | 0 | NRCOO | SO₂NR | N₃ |
| 0 | NRCOO | NRCONR | COH | 0 | NRCOO | NRCNHNR | COH |
| 0 | NRCOO | NRCONR | COR | 0 | NRCOO | NRCNHNR | COR |
| 0 | NRCOO | NRCOO | OH | 0 | NRCOO | C≡C | OH |
| 0 | NRCOO | NRCOO | SH | 0 | NRCOO | C≡C | SH |
| 0 | NRCOO | CH═CH | F | 0 | C≡C | 0 | F |
| 0 | C≡C | S | COOH | 0 | C≡C | NR | COOH |
| 0 | C≡C | S | SO₂H | 0 | C≡C | NR | SO₂H |
| 0 | C≡C | CR₁R₂ | NH₂ | 0 | C≡C | CONR | NH₂ |
| 0 | C≡C | CR₁R₂ | NHR | 0 | C≡C | CONR | NHR |
| 0 | C≡C | CR₁R₂ | COH | 0 | C≡C | CONR | COH |
| 0 | C≡C | SO₂NR | COH | 0 | C≡C | NRCONR | COH |
| 0 | C≡C | SO₂NR | COR | 0 | C≡C | NRCONR | COR |
| 0 | C≡C | NRCNHNR | OH | 0 | C≡C | NRCOO | OH |
| 0 | C≡C | NRCNHNR | SO₂H | 0 | C≡C | NRCOO | SO₂H |
| 0 | C≡C | NRCNHNR | Cl | 0 | C≡C | NRCOO | Cl |
| 0 | C≡C | C≡C | OH | 0 | C≡C | CH═CH | OH |
| 0 | C≡C | C≡C | CN | 0 | C≡C | CH═CH | CN |
| 0 | CH═CH | O | CH═CH₂ | 0 | CH═CH | S | CH═CH₂ |
| 0 | CH═CH | O | C≡CH | 0 | CH═CH | S | C≡CH |
| 0 | CH═CH | O | COR | 0 | CH═CH | S | COR |
| 0 | CH═CH | NR | OH | 0 | CH═CH | CR₁R₂ | OH |
| 0 | CH═CH | NR | SH | 0 | CH═CH | CR₁R₂ | SH |
| 0 | CH═CH | NRCONR | COH | 0 | CH═CH | NRCNHNR | COH |
| 0 | CH═CH | NRCONR | COR | 0 | CH═CH | NRCNHNR | COR |
| 0 | CH═CH | NRCOO | SH | 0 | CH═CH | C≡C | SH |
| 0 | CH═CH | NRCOO | NHR | 0 | CH═CH | C≡C | NHR |
| 0 | CH═CH | NRCOO | COH | 0 | CH═CH | C≡C | COH |
| 0 | CH═CH | CH═CH | OH | 0 | CH═CH | CH═CH | N₃ |
| 0 | CH═CH | CH═CH | SH | 0 | CH═CH | CH═CH | CONH₂ |
| 1 | O | O | C≡CH | 1 | O | S | C≡CH |
| 1 | O | O | NH₂ | 1 | O | S | NH₂ |
| 1 | O | O | NHR | 1 | O | S | NHR |
| 1 | O | NR | NHR | 1 | O | CR₁R₂ | NHR |
| 1 | O | NR | COH | 1 | O | CR₁R₂ | COH |
| 1 | O | CONR | SH | 1 | O | SO₂NR | SH |
| 1 | O | CONR | SO₂H | 1 | O | SO₂NR | SO₂H |
| 1 | O | NRCONR | OH | 1 | O | NRCNHNR | OH |
| 1 | O | NRCONR | SH | 1 | O | NRCNHNR | SH |
| 1 | O | NRCOO | SH | 1 | O | C≡C | SH |
| 1 | O | NRCOO | COOH | 1 | O | C≡C | COOH |
| 1 | O | CH═CH | OH | 1 | S | O | OH |
| 1 | O | CH═CH | COH | 1 | S | O | COH |
| 1 | O | CH═CH | COR | 1 | S | O | COR |
| 1 | S | S | OH | 1 | S | NR | OH |
| 1 | S | S | CH═CH₂ | 1 | S | NR | CH═CH₂ |
| 1 | S | S | NH₂ | 1 | S | NR | NH₂ |
| 1 | S | CR₁R₂ | Cl | 1 | S | CONR | Cl |
| 1 | S | CR₁R₂ | Br | 1 | S | CONR | Br |
| 1 | S | SO₂NR | Br | 1 | S | NRCONR | Br |
| 1 | S | SO₂NR | COH | 1 | S | NRCONR | COH |
| 1 | S | NRCNHNR | COOH | 1 | S | NRCOO | COOH |
| 1 | S | NRCNHNR | F | 1 | S | NRCOO | F |
| 1 | S | C≡C | OH | 1 | S | CH═CH | OH |
| 1 | S | C≡C | SH | 1 | S | CH═CH | SH |
| 1 | S | C≡C | COOH | 1 | S | CH═CH | COOH |
| 1 | S | C≡C | C≡CH | 1 | S | CH═CH | C≡CH |
| 1 | NR | O | SO₂H | 1 | NR | S | SO₂H |
| 1 | NR | O | Cl | 1 | NR | S | Cl |
| 1 | NR | O | CN | 1 | NR | S | CN |
| 1 | NR | NR | CONH₂ | 1 | NR | CR₁R₂ | CONH₂ |
| 1 | NR | NR | CH═CH₂ | 1 | NR | CR₁R₂ | CH═CH₂ |
| 1 | NR | CONR | CONH₂ | 1 | NR | SO₂NR | CONH₂ |
| 1 | NR | CONR | COR | 1 | NR | SO₂NR | COR |
| 1 | NR | NRCONR | NHR | 1 | NR | NRCNHNR | NHR |
| 1 | NR | NRCONR | COH | 1 | NR | NRCNHNR | COH |
| 1 | NR | NRCOO | OH | 1 | NR | C≡C | OH |
| 1 | NR | NRCOO | N₃ | 1 | NR | C≡C | N₃ |
| 1 | NR | NRCOO | CONH₂ | 1 | NR | C≡C | CONH₂ |
| 1 | NR | CH═CH | N₃ | 1 | CR₁R₂ | O | N₃ |
| 1 | NR | CH═CH | CONH₂ | 1 | CR₁R₂ | O | CONH₂ |
| 1 | NR | CH═CH | CH═CH₂ | 1 | CR₁R₂ | O | CH═CH₂ |
| 1 | CR₁R₂ | S | Br | 1 | CR₁R₂ | NR | Br |
| 1 | CR₁R₂ | S | N₃ | 1 | CR₁R₂ | NR | N₃ |
| 1 | CR₁R₂ | S | NHR | 1 | CR₁R₂ | NR | NHR |
| 1 | CR₁R₂ | S | COH | 1 | CR₁R₂ | NR | COH |
| 1 | CR₁R₂ | CR₁R₂ | SO₂H | 1 | CR₁R₂ | COHR | SO₂H |
| 1 | CR₁R₂ | SO₂NR | COOH | 1 | CR₁R₂ | NRCONR | COOH |
| 1 | CR₁R₂ | SO₂NR | SO₂H | 1 | CR₁R₂ | NRCONR | SO₂H |
| 1 | CR₁R₂ | NRCNHNR | CN | 1 | CR₁R₂ | NRCOO | CN |
| 1 | CR₁R₂ | NRCNHNR | COH | 1 | CR₁R₂ | NRCOO | COH |
| 1 | CR₁R₂ | NRCNHNR | COR | 1 | CR₁R₂ | NRCOO | COR |
| 1 | CR₁R₂ | C≡C | SH | 1 | CR₁R₂ | CH═CH | SH |
| 1 | CR₁R₂ | C≡C | COOH | 1 | CR₁R₂ | CH═CH | COOH |
| 1 | CONR | O | OH | 1 | CONR | S | OH |
| 1 | CONR | O | SH | 1 | CONR | S | SH |
| 1 | CONR | O | COOH | 1 | CONR | S | COOH |
| 1 | CONR | NR | CN | 1 | CONR | CR₁R₂ | CN |
| 1 | CONR | NR | N₃ | 1 | CONR | CR₁R₂ | N₃ |
| 1 | CONR | NR | COH | 1 | CONR | CR₁R₂ | COH |
| 1 | CONR | NR | COR | 1 | CONR | CR₁R₂ | COR |
| 1 | CONR | CONR | OH | 1 | CONR | SO₂NR | OH |
| 1 | CONR | CONR | F | 1 | CONR | SO₂NR | F |
| 1 | CONR | CONR | NHR | 1 | CONR | SO₂NR | NHR |
| 1 | CONR | CONR | COR | 1 | CONR | SO₂NR | COR |
| 1 | CONR | NRCONR | OH | 1 | CONR | NRCNHNR | OH |
| 1 | CONR | NRCONR | SO₂H | 1 | CONR | NRCNHNR | SO₂H |
| 1 | CONR | NRCOO | SH | 1 | CONR | C≡C | SH |
| 1 | CONR | NRCOO | COOH | 1 | CONR | C≡C | COOH |
| 1 | CONR | NRCOO | COH | 1 | CONR | C≡C | COH |
| 1 | CONR | CH═CH | Cl | 1 | SO₂HR | O | Cl |
| 1 | CONR | CH═CH | Br | 1 | SO₂NR | O | Br |
| 1 | SO₂NR | S | N₃ | 1 | SO₂NR | NR | N₃ |
| 1 | SO₂NR | S | CONH₂ | 1 | SO₂NR | NR | CONH₂ |
| 1 | SO₂NR | S | COR | 1 | SO₂NR | NR | COR |
| 1 | SO₂NR | CR₁R₂ | SH | 1 | SO₂NR | CONR | SH |
| 1 | SO₂NR | CR₁R₂ | COOH | 1 | SO₂NR | CONR | COOH |
| 1 | SO₂NR | SO₂NR | SO₃H | 1 | SO₂NR | NRCONR | SO₂H |
| 1 | SO₂NR | SO₂NR | Cl | 1 | SO₂NR | NRCONR | Cl |
| 1 | SO₂NR | SO₂NR | Br | 1 | SO₂NR | NRCONR | Br |
| 1 | SO₂NR | SO₂NR | COH | 1 | SO₂NR | NRCONR | COH |
| 1 | SO₂NR | NRCNHNR | OH | 1 | SO₂NR | NRCOO | OH |
| 1 | SO₂NR | NRCNHNR | NH₂ | 1 | SO₂NR | NRCOO | NH₂ |
| 1 | SO₂NR | C≡C | Br | 1 | SO₂NR | CH═CH | Br |
| 1 | SO₂NR | C≡C | COR | 1 | SO₂NR | CH═CH | COR |
| 1 | NRCONR | O | SH | 1 | NRCONR | S | SH |
| 1 | NRCONR | O | NH₂ | 1 | NRCONR | S | NH₂ |
| 1 | NRCONR | NR | Cl | 1 | NRCONR | CR₁R₂ | Cl |
| 1 | NRCONR | NR | I | 1 | NRCONR | CR₁R₂ | I |
| 1 | NRCONR | CONR | F | 1 | NRCONR | SO₂NR | F |
| 1 | NRCONR | CONR | N₃ | 1 | NRCONR | SO₂NR | N₃ |
| 1 | NRCONR | NRCONR | OH | 1 | NRCONR | NRCNHNR | OH |
| 1 | NRCONR | NRCONR | COR | 1 | NRCONR | NRCNHNR | COR |
| 1 | NRCONR | NRCOO | OH | 1 | NRCONR | C≡C | OH |
| 1 | NRCONR | NRCOO | COR | 1 | NRCONR | | COR |
| 1 | NRCONR | CH═CH | OH | 1 | NRCNHNR | O | OH |
| 1 | NRCONR | CH═CH | COOH | 1 | NRCNHNR | O | COOH |
| 1 | NRCNHNR | S | NH₂ | 1 | NRCNHNR | NR | NH₂ |
| 1 | NRCNHNR | S | NHR | 1 | NRCNHNR | NR | NHR |
| 1 | NRCNHNR | S | COH | 1 | NRCNHNR | NR | COH |
| 1 | NRCNHNR | CR₁R₂ | F | 1 | NRCNHNR | CONR | F |
| 1 | NRCNHNR | CR₁R₂ | CN | 1 | NRCNHNR | CONR | CN |
| 1 | NRCNHNR | SO₂NR | CN | 1 | NRCNHNR | NRCONR | CN |
| 1 | NRCNHNR | SO₂NR | NHR | 1 | NRCNHNR | NRCONR | NHR |
| 1 | NRCNHNR | SO₂NR | COH | 1 | NRCNHNR | NRCONR | COH |
| 1 | NRCNHNR | NRCNHNR | Cl | 1 | NRCNHNR | NRCOO | Cl |
| 1 | NRCNHNR | NRCNHNR | Br | 1 | NRCNHNR | NRCOO | Br |
| 1 | NRCNHNR | NRCNHNR | CH═CH₂ | 1 | NRCNHNR | NRCOO | CH═CH₂ |
| 1 | NRCNHNR | C≡C | OH | 1 | NRCNHNR | CH═CH | OH |
| 1 | NRCNHNR | C≡C | SO₂H | 1 | NRCNHNR | CH═CH | SO₂N |
| 1 | NRCNHNR | C≡C | COR | 1 | NRCNHNR | CH═CH | COR |
| 1 | NRCOO | O | F | 1 | NRCOO | S | F |
| 1 | NRCOO | O | N₃ | 1 | NRCOO | S | N₃ |
| 1 | NRCOO | O | CONH₂ | 1 | NRCOO | S | CONH₂ |
| 1 | NRCOO | NR | OH | 1 | NRCOO | CR₁R₂ | OH |
| 1 | NRCOO | NR | SH | 1 | NRCOO | CR₁R₂ | SH |
| 1 | NRCOO | NR | I | 1 | NRCOO | CR₁R₂ | I |
| 1 | NRCOO | CONR | OH | 1 | NRCOO | SO₂NR | OH |
| 1 | NRCOO | CONR | N₃ | 1 | NRCOO | SO₂NR | N₃ |
| 1 | NRCOO | CONR | COR | 1 | NRCOO | SO₂NR | COR |
| 1 | NRCOO | NRCONR | OH | 1 | NRCOO | NRCNHNR | OH |
| 1 | NRCOO | NRCONR | N₃ | 1 | NRCOO | NRCNHNR | N₃ |
| 1 | NRCOO | NRCOO | SH | 1 | NRCOO | C≡C | SH |
| 1 | NRCOO | NRCOO | CH═CH₂ | 1 | NRCOO | C≡C | CH═CH₂ |
| 1 | NRCOO | CH═CH | I | 1 | C≡C | O | I |
| 1 | NRCOO | CH═CH | F | 1 | C≡C | O | F |
| 1 | NRCOO | CH═CH | C≡CH | 1 | C≡C | O | C≡CH |
| 1 | C≡C | S | I | 1 | C≡C | NR | I |
| 1 | C≡C | S | F | 1 | C≡C | NR | F |
| 1 | C≡C | S | CH═CH₂ | 1 | C≡C | NR | CH═CH₂ |
| 1 | C≡C | CR₁R₂ | OH | 1 | C≡C | CONR | OH |
| 1 | C≡C | CR₁R₂ | SH | 1 | C≡C | CONR | SH |
| 1 | C≡C | CR₁R₂ | COOH | 1 | C≡C | CONR | COOH |
| 1 | C≡C | CR₁R₂ | SO₂H | 1 | C≡C | CONR | SO₂H |
| 1 | C≡C | SO₂NR | NHR | 1 | C≡C | NRCONR | NHR |
| 1 | C≡C | NRCNHNR | SH | 1 | C≡C | NRCOO | SH |
| 1 | C≡C | NRCNHNR | SO₂H | 1 | C≡C | NRCOO | SO₂H |
| 1 | C≡C | NRCNHNR | COR | 1 | C≡C | NRCOO | COR |
| 1 | C≡C | C≡C | OH | 1 | C≡C | CH═CH | OH |
| 1 | C≡C | C≡C | COH | 1 | C≡C | CH═CH | COH |
| 1 | C≡C | C≡C | COR | 1 | C≡C | CH═CH | COR |
| 1 | CH═CH | O | OH | 1 | CH═CH | S | OH |
| 1 | CH═CH | O | COOH | 1 | CH═CH | S | COOH |
| 1 | CH═CH | O | COH | 1 | CH═CH | S | COH |
| 1 | CH═CH | NR | SO₂H | 1 | CH═CH | CR₁R₂ | SO₂H |
| 1 | CH═CH | NR | F | 1 | CH═CH | CR₁R₂ | F |
| 1 | CH═CH | NR | COH | 1 | CH═CH | CR₁R₂ | COH |
| 1 | CH═CH | CONR | SH | 1 | CH═CH | SO₂NR | SH |
| 1 | CH═CH | CONR | I | 1 | CH═CH | SO₂NR | I |
| 1 | CH═CH | CONR | F | 1 | CH═CH | SO₂NR | F |
| 1 | CH═CH | NRCONR | CH═CH₂ | 1 | CH═CH | NRCNHNR | CH═CH₂ |
| 1 | CH═CH | NRCONR | C≡CH | 1 | CH═CH | NRCNHNR | C≡CH |
| 1 | CH═CH | NRCONR | NH₂ | 1 | CH═CH | NRCNHNR | NH₂ |
| 1 | CH═CH | NRCOO | COH | 1 | CH═CH | C≡C | COH |
| 1 | CH═CH | NRCOO | COR | 1 | CH═CH | C≡C | COR |
| 1 | CH═CH | CH═CH | OH | 1 | CH═CH | CH═CH | N₃ |
| 1 | CH═CH | CH═CH | Br | 1 | CH═CH | CH═CH | NHR |
| 1 | CH═CH | CH═CH | I | 1 | CH═CH | CH═CH | COH |
| 2 | O | O | F | 2 | O | S | F |
| 2 | O | O | CN | 2 | O | S | CN |
| 2 | O | O | N₃ | 2 | O | S | N₃ |
| 2 | O | NR | Br | 2 | O | CR₂R₂ | Br |
| 2 | O | NR | F | 2 | O | CR₂R₂ | F |
| 2 | O | NR | COR | 2 | O | CR₂R₂ | COR |
| 2 | O | CONR | OH | 2 | O | SO₂NR | OH |
| 2 | O | CONR | SH | 2 | O | SO₂NR | SH |
| 2 | O | CONR | COOH | 2 | O | SO₂NR | COOH |
| 2 | O | NRCONR | N₃ | 2 | O | NRCNHNR | N₃ |
| 2 | O | NRCONR | CONH₂ | 2 | O | NRCNHNR | CONH₂ |
| 2 | O | NRCOO | Cl | 2 | O | C≡C | Cl |
| 2 | O | NRCOO | CH═CH₂ | 2 | O | C≡C | CH═CH₂ |
| 2 | O | CH═CH | SH | 2 | S | O | SH |
| 2 | O | CH═CH | COOH | 2 | S | O | COOH |
| 2 | O | CH═CH | COH | 2 | S | O | COH |
| 2 | S | S | COOH | 2 | S | NR | COOH |
| 2 | S | S | SO₂H | 2 | S | NR | SO₂H |
| 2 | S | S | Cl | 2 | S | NR | Cl |
| 2 | S | S | NHR | 2 | S | NR | NHR |
| 2 | S | CR₂R₂ | CN | 2 | S | CONR | CN |
| 2 | S | CR₂R₂ | C≡CH | 2 | S | CONR | C≡CH |
| 2 | S | CR₂R₂ | NH₂ | 2 | S | CONR | NH₂ |
| 2 | S | SO₂NR | Cl | 2 | S | NRCONR | Cl |
| 2 | S | SO₂NR | Br | 2 | S | NRCONR | Br |
| 2 | S | SO₂NR | N₃ | 2 | S | NRCONR | N₃ |
| 2 | S | NRCNHNR | Br | 2 | S | NRCOO | Br |
| 2 | S | NRCNHNR | I | 2 | S | NRCOO | I |
| 2 | S | NRCNHNR | COR | 2 | S | NRCOO | COR |
| 2 | S | C≡C | OH | 2 | S | CH═CH | OH |
| 2 | S | C≡C | SH | 2 | S | CH═CH | SH |
| 2 | S | C≡C | CH═CH₂ | 2 | S | CH═CH | CH═CH₂ |
| 2 | NR | O | C≡CH | 2 | NR | S | C≡CH |
| 2 | NR | O | NH₂ | 2 | NR | S | NH₂ |
| 2 | NR | O | NHR | 2 | NR | S | NHR |
| 2 | NR | NR | Br | 2 | NR | CR₂R₂ | Br |
| 2 | NR | NR | F | 2 | NR | CR₂R₂ | F |
| 2 | NR | NR | NH₂ | 2 | NR | CR₂R₂ | NH₂ |
| 2 | NR | NR | NHR | 2 | NR | CR₂R₂ | NHR |
| 2 | NR | CONR | CN | 2 | NR | SO₂NR | CN |
| 2 | NR | CONR | COR | 2 | NR | SO₂NR | COR |
| 2 | NR | NRCONR | OH | 2 | NR | NRCNHNR | OH |
| 2 | NR | NRCONR | SH | 2 | NR | NRCNHNR | SH |
| 2 | NR | NRCOO | CH═CH₂ | 2 | NR | C≡C | CH═CH₂ |
| 2 | NR | NRCOO | C≡CH | 2 | NR | C≡C | C≡CH |
| 2 | NR | NRCOO | NH₂ | 2 | NR | C≡C | NH₂ |
| 2 | NR | CH═CH | Br | 2 | CR₂R₂ | O | Br |
| 2 | NR | CH═CH | NH₂ | 2 | CR₂R₂ | OO | NH₂ |
| 2 | NR | CH═CH | COH | 2 | CR₂R₂ | O | COH |
| 2 | NR | CH═CH | COR | 2 | CR₂R₂ | O | COR |
| 2 | CR₂R₂ | S | OH | 2 | CR₂R₂ | NR | OH |
| 2 | CR₂R₂ | S | SH | 2 | CR₂R₂ | NR | SH |
| 2 | CR₂R₂ | S | NH₂ | 2 | CR₂R₂ | NR | NH₂ |
| 2 | CR₂R₂ | CR₂R₂ | CN | 2 | CR₂R₂ | CONR | CN |
| 2 | CR₂R₂ | CR₂R₂ | N₃ | 2 | CR₂R₂ | CONR | N₃ |
| 2 | CR₂R₂ | CR₂R₂ | CONH₂ | 2 | CR₂R₂ | CONR | CONH₂ |
| 2 | CR₂R₂ | CR₂R₂ | CH═CH₂ | 2 | CR₂R₂ | CONR | CH═CH₂ |
| 2 | CR₂R₂ | SO₂NR | OH | 2 | CR₂R₂ | NRCONR | OH |
| 2 | CR₂R₂ | SO₂NR | Br | 2 | CR₂R₂ | NRCONR | Br |
| 2 | CR₂R₂ | SO₂NR | I | 2 | CR₂R₂ | NRCONR | I |
| 2 | CR₂R₂ | SO₂NR | F | 2 | CR₂R₂ | NRCONR | F |
| 2 | CR₂R₂ | NRCNHNR | SH | 2 | CR₂R₂ | NRCOO | SH |
| 2 | CR₂R₂ | NRCNHNR | COOH | 2 | CR₂R₂ | NRCOO | COOH |
| 2 | CR₂R₂ | NRCNHNR | SO₂H | 2 | CR₂R₂ | NRCOO | SO₂H |
| 2 | CR₂R₂ | C≡C | Cl | 2 | CR₂R₂ | CH═CH | Cl |
| 2 | CR₂R₂ | C≡C | NH₂ | 2 | CR₂R₂ | CH═CH | NH₂ |
| 2 | CR₂R₂ | C≡C | COH | 2 | CR₂R₂ | CH═CH | COH |
| 2 | CONR | O | SO₂H | 2 | CONR | S | SO₂H |
| 2 | CONR | O | N₃ | 2 | CONR | S | N₃ |
| 2 | CONR | NR | COOH | 2 | CONR | CR₂R₂ | COOH |
| 2 | CONR | NR | SO₂H | 2 | CONR | CR₂R₂ | SO₂H |
| 2 | CONR | NR | Cl | 2 | CONR | CR₂R₂ | Cl |
| 2 | CONR | CONR | CH═CH₂ | 2 | CONR | SO₂NR | CH═CH₂ |
| 2 | CONR | CONR | C≡CH | 2 | CONR | SO₂NR | C≡CH |
| 2 | CONR | CONR | NH₂ | 2 | CONR | SO₂NR | NH₂ |
| 2 | CONR | NRCONR | NH₂ | 2 | CONH | NRCNHNR | NH₂ |
| 2 | CONR | NRCONR | NHR | 2 | CONR | NRCNHNR | NHR |
| 2 | CONR | NRCOO | CN | 2 | CONR | C≡C | CN |
| 2 | CONR | NRCOO | COR | 2 | CONR | C≡C | COR |
| 2 | CONR | CH═CH | OH | 2 | SO₂NR | O | OH |
| 2 | CONR | CH═CH | Br | 2 | SO₂NR | O | Br |
| 2 | CONR | CH═CH | I | 2 | SO₂NR | O | I |
| 2 | SO₂NR | S | OH | 2 | SO₂NR | NR | OH |
| 2 | SO₂NR | S | SH | 2 | SO₂NR | NR | SH |
| 2 | SO₂NR | S | COH | 2 | SO₂NR | NR | COH |
| 2 | SO₂NR | CR₂R₂ | COOH | 2 | SO₂NR | CONR | COOH |
| 2 | SO₂NR | CR₂R₂ | COR | 2 | SO₂NR | CONR | COR |
| 2 | SO₂NR | SO₂NR | OH | 2 | SO₂NR | NRCONR | OH |
| 2 | SO₂NR | SO₂NR | SH | 2 | SO₂NR | NRCONR | SH |
| 2 | SO₂NR | SO₂NR | COOH | 2 | SO₂NR | NRCONR | COOH |
| 2 | SO₂NR | NRCNHNR | CH═CH₂ | 2 | SO₂NR | NRCOO | CH═CH₂ |
| 2 | SO₂NR | NRCNHNR | COH | 2 | SO₂NR | NRCOO | COH |
| 2 | SO₂NR | NRCNHNR | COR | 2 | SO₂NR | NRCOO | COR |
| 2 | SO₂NR | C≡C | NHR | 2 | SO₂NR | CH═CH | NHR |
| 2 | SO₂NR | C≡C | COH | 2 | SO₂NR | CH═CH | COH |
| 2 | NRCONR | O | COOH | 2 | NRCONR | S | COOH |
| 2 | NRCONR | O | CONH₂ | 2 | NRCONR | S | CONH₂ |
| 2 | NRCONR | O | CH═CH₂ | 2 | NRCONR | S | CH═CH₂ |
| 2 | NRCONR | NR | Cl | 2 | NRCONR | CR₂R₂ | Cl |
| 2 | NRCONR | NR | Br | 2 | NRCONR | CR₂R₂ | Br |
| 2 | NRCONR | CONR | COH | 2 | NRCONR | SO₂NR | COH |
| 2 | NRCONR | CONR | COR | 2 | NRCONR | SO₂NR | COR |
| 2 | NRCONR | NRCONR | SH | 2 | NRCONR | NRCNHNR | SH |
| 2 | NRCONR | NRCONR | CN | 2 | NRCONR | NRCNHNR | CN |
| 2 | NRCONR | NRCOO | F | 2 | NRCONR | C≡C | F |
| 2 | NRCONR | NRCOO | CN | 2 | NRCONR | C≡C | CN |
| 2 | NRCONR | CH═CH | I | 2 | NRCNHNR | O | I |
| 2 | NRCONR | CN═CH | F | 2 | NRCNHNR | O | F |
| 2 | NRCONR | CH═CH | CN | 2 | NRCNHNR | O | CN |
| 2 | NRCNHNR | S | F | 2 | NRCNHNR | NR | F |
| 2 | NRCNHNR | S | COH | 2 | NRCNHNR | NR | COH |
| 2 | NRCNHNR | S | COR | 2 | NRCNHNR | NR | COR |
| 2 | NRCNHNR | CR₂R₂ | COR | 2 | NRCNHNR | CONR | COR |
| 2 | NRCNHNR | SO₂NR | OH | 2 | NRCNHNR | NRCONR | OH |
| 2 | NRCNHNR | SO₂NR | N₃ | 2 | NRCNHNR | NRCONR | N₃ |
| 2 | NRCNHNR | NRCNHNR | CONH₂ | 2 | NRCNHNR | NRCOO | CONH₂ |
| 2 | NRCNHNR | NRCNHNR | COH | 2 | NRCNHNR | NRCOO | COH |
| 2 | NRCNHNR | NRCNHNR | COR | 2 | NRCNHNR | NRCOO | COR |
| 2 | NRCNHNR | C≡C | OH | 2 | NRCNHNR | CH═CH | OH |
| 2 | NRCNHNR | C≡C | SH | 2 | NRCNHNR | CH═CH | SH |
| 2 | NRCNHNR | C≡C | NH₂ | 2 | NRCNHNR | CH═CH | NH₂ |
| 2 | NRCOO | O | I | 2 | NRCOO | S | I |
| 2 | NRCOO | O | C≡CH | 2 | NRCOO | S | C≡CH |
| 2 | NRCOO | O | COR | 2 | NRCOO | S | COR |
| 2 | NRCOO | NR | SH | 2 | NRCOO | CR₂R₂ | SH |
| 2 | NRCOO | NR | COOH | 2 | NRCOO | CR₂R₂ | COOH |
| 2 | NRCOO | CONR | I | 2 | NRCOO | SO₂NR | I |
| 2 | NRCOO | CONR | CN | 2 | NRCOO | SO₂NR | CN |
| 2 | NRCOO | NRCONR | OH | 2 | NRCOO | NRCNHNR | OH |
| 2 | NRCOO | NRCONR | SH | 2 | NRCOO | NRCNHNR | SH |
| 2 | NRCOO | NRCOO | Br | 2 | NRCOO | C≡C | Br |
| 2 | NRCOO | NRCOO | F | 2 | NRCOO | C≡C | F |
| 2 | NRCOO | NRCOO | N₃ | 2 | NRCOO | C≡C | N₃ |
| 2 | NRCOO | CH═CH | CH | 2 | C≡C | O | CN |
| 2 | NRCOO | CH═CH | C≡CH | 2 | C≡C | O | C≡CH |
| 2 | NRCOO | CH═CH | NH₂ | 2 | C≡C | O | NH₂ |
| 2 | C≡C | S | COOH | 2 | C≡C | NR | COOH |
| 2 | C≡C | S | CONH₂ | 2 | C≡C | NR | CONH₂ |
| 2 | C≡C | S | NHR | 2 | C≡C | NR | NHR |
| 2 | C≡C | CR₂R₂ | COOH | 2 | C≡C | CONR | COOH |
| 2 | C≡C | SO₂NR | SH | 2 | C≡C | NRCONR | SH |
| 2 | C≡C | SO₂NR | N₃ | 2 | C≡C | NRCONR | N₃ |
| 2 | C≡C | SO₂NR | CONH₂ | 2 | C≡C | NRCONR | CONH₂ |
| 2 | C≡C | SO₂NR | CH═CH₂ | 2 | C≡C | NRCONR | CH═CH₂ |
| 2 | C≡C | NRCNHNR | I | 2 | C≡C | NRCOO | I |
| 2 | C≡C | NRCNHNR | F | 2 | C≡C | NRCOO | F |
| 2 | C≡C | NRCNHNR | NHR | 2 | C≡C | NRCOO | NHR |
| 2 | C≡C | C≡C | CH═CH₂ | 2 | CH═CH | CH═CH₂ | |
| 2 | C≡C | C≡C | C≡CH | 2 | C≡C | CH═CH | C≡CH |
| 2 | CH═CH | O | CONH₂ | 2 | CH═CH | S | CONH₂ |
| 2 | CH═CH | O | NHR | 2 | CH═CH | S | NHR |
| 2 | CH═CH | O | COR | 2 | CH═CH | S | COR |
| 2 | CH═CH | NR | I | 2 | CH═CH | CR₂R₂ | I |
| 2 | CH═CH | NR | F | 2 | CH═CH | CR₂R₂ | F |
| 2 | CH═CH | NR | CN | 2 | CH═CH | CR₂R₂ | CN |
| 2 | CH═CH | NR | CH═CH₂ | 2 | CH═CH | CR₂R₂ | CH═CH₂ |
| 2 | CH═CH | CONR | C≡CH | 2 | CH═CH | SO₂NR | C≡CH |
| 2 | CH═CH | CONR | NH₂ | 2 | CH═CH | SO₂NR | NH₂ |
| 2 | CH═CH | NRCONR | Cl | 2 | CH═CH | NRCNHNR | Cl |
| 2 | CH═CH | NRCONR | N₃ | 2 | CH═CH | NRCNHNR | N₃ |
| 2 | CH═CH | NRCOO | SH | 2 | CH═CH | C≡C | SH |
| 2 | CH═CH | NRCOO | CONH₂ | 2 | CH═CH | C≡C | CONH₂ |
| 2 | CH═CH | NRCOO | CH═CH₂ | 2 | CH═CH | C≡C | CH═CH₂ |
| 2 | CH═CH | NRCOO | C≡CH | 2 | CH═CH | C≡C | C≡CH |
| 2 | CH═CH | CH═CH | SO₂H | 2 | CH═CH | CH═CH | C≡CH |
| 2 | CH═CH | CH═CH | Cl | 2 | CH═CH | CH═CH | NH₂ |
| 2 | CH═CH | CH═CH | Br | 2 | CH═CH | CH═CH | NHR |
| 3 | O | O | Cl | 3 | O | S | Cl |
| 3 | O | O | I | 3 | O | S | I |
| 3 | O | NR | CONH₂ | 3 | O | CR₃R₂ | CONH₂ |
| 3 | O | NR | CH═CH₂ | 3 | O | CR₃R₂ | CH═CH₂ |
| 3 | O | NR | NH₂ | 3 | O | CR₃R₂ | NH₂ |
| 3 | O | CONR | NH₂ | 3 | O | SO₂NR | NH₂ |
| 3 | O | CONR | NHR | 3 | O | SO₂NR | NHR |
| 3 | O | NRCONR | N₃ | 3 | O | NRCNHNR | N₃ |
| 3 | O | NRCONR | CONH₂ | 3 | O | NRCNHNR | CONH₂ |
| 3 | O | NRCOO | SH | 3 | O | C≡C | SH |
| 3 | O | NRCOO | F | 3 | O | C≡C | F |
| 3 | O | NRCOO | N₃ | 3 | O | C≡C | N₃ |
| 3 | O | NRCOO | C≡CH | 3 | O | C≡C | C≡CH |
| 3 | O | NRCOO | NH₂ | 3 | O | C≡C | NH₂ |
| 3 | O | CH═CH | NH₂ | 3 | S | O | NH₂ |
| 3 | O | CH═CH | COH | 3 | S | O | COH |
| 3 | O | CH═CH | COR | 3 | S | O | COR |
| 3 | S | S | OH | 3 | S | NR | OH |
| 3 | S | S | SH | 3 | S | NR | SH |
| 3 | S | S | NHR | 3 | S | NR | NHR |
| 3 | S | S | COH | 3 | S | NR | COH |
| 3 | S | CR₃R₂ | NH₂ | 3 | S | CONR | NH₂ |
| 3 | S | SO₂HR | SH | 3 | S | NRCONR | SH |
| 3 | S | SO₂NR | COOH | 3 | S | NRCONR | COOH |
| 3 | S | NRCNHNR | I | 3 | S | NRCOO | I |
| 3 | S | NRCNHNR | CONH₂ | 3 | S | NRCOO | CONH₂ |
| 3 | S | NRCNHNR | COR | 3 | S | NRCOO | COR |
| 3 | S | C≡C | OH | 3 | S | CH═CH | OH |
| 3 | S | C≡C | SH | 3 | S | CH═CH | SH |
| 3 | NR | O | CH═CH₂ | 3 | NR | S | CH═CH₂ |
| 3 | NR | O | C≡CH | 3 | NR | S | C≡CH |
| 3 | NR | O | COH | 3 | NR | S | COH |
| 3 | NR | NR | SH | 3 | NR | CR₃R₂ | SH |
| 3 | NR | NH | COOH | 3 | NR | CR₃R₂ | COOH |
| 3 | NR | NR | SO₂H | 3 | NR | CR₃R₂ | SO₂H |
| 3 | NR | CONR | NH₂ | 3 | NR | SO₂NR | NH₂ |
| 3 | NR | CONR | NHR | 3 | NR | SO₂NR | NHR |
| 3 | NR | CONR | COH | 3 | NR | SO₂NR | COH |
| 3 | NR | NRCONR | COOH | 3 | NR | NRCNHNR | COOH |
| 3 | NR | NRCONR | C≡CH | 3 | NR | NRCNHNR | C≡CH |
| 3 | NR | NRCONR | NH₂ | 3 | NR | NRCNHNR | NH₂ |
| 3 | NR | NRCOO | OH | 3 | NR | C≡C | OH |
| 3 | NR | NRCOO | NHR | 3 | NR | C≡C | NHR |
| 3 | NR | CH═CH | COOH | 3 | CR₃R₂ | O | COOH |
| 3 | NR | CH═CH | I | 3 | CR₃R₂ | O | I |
| 3 | CR₃R₂ | S | Br | 3 | CR₃R₂ | NR | Br |
| 3 | CR₃R₂ | CR₃R₂ | CH═CH₂ | 3 | CR₃R₂ | CONR | CH═CH₂ |
| 3 | CR₃R₂ | CR₃R₂ | C≡CH | 3 | CR₃R₂ | CONR | C≡CH |
| 3 | CR₃R₂ | SO₂NR | NH₂ | 3 | CR₃R₂ | NRCONR | NH₂ |
| 3 | CR₃R₂ | SO₂NR | NHR | 3 | CR₃R₂ | NRCONR | NHR |
| 3 | CR₃R₂ | SO₂NR | COH | 3 | CR₃R₂ | NRCONR | COH |
| 3 | CR₃R₂ | NRCNHNR | COOH | 3 | CR₃R₂ | NRCOO | COOH |
| 3 | CR₃R₂ | NRCNHNR | SO₂H | 3 | CR₃R₂ | NRCOO | SO₂H |
| 3 | CR₃R₂ | NRCNHNR | COH | 3 | CR₃R₂ | NRCOO | COH |
| 3 | CR₃R₂ | C≡C | SO₂H | 3 | CR₃R₂ | CH═CH | SO₂H |
| 3 | CR₃R₂ | C≡C | CN | 3 | CR₃R₂ | CH═CH | CN |
| 3 | CONR | O | SO₂H | 3 | CONR | S | SO₂H |
| 3 | CONR | O | Cl | 3 | CONR | S | Cl |
| 3 | CONR | O | Br | 3 | CONR | S | Br |
| 3 | CONR | NR | N₃ | 3 | CONR | CR₃R₂ | N₃ |
| 3 | CONR | NR | CONH₂ | 3 | CONR | CR₃R₂ | CONH₂ |
| 3 | CONR | NR | CH═CH₂ | 3 | CONR | CR₃R₂ | CH═CH₂ |
| 3 | CONR | CONR | C≡CH | 3 | CONR | SO₂NR | C≡CH |
| 3 | CONR | CONR | NH₂ | 3 | CONR | SO₂NR | NH₂ |
| 3 | CONR | NRCONR | I | 3 | CONR | NRCNHNR | I |
| 3 | CONR | NRCONR | N₃ | 3 | CONR | NRCNHNR | N₃ |
| 3 | CONR | NRCOO | COH | 3 | CONR | C≡C | COH |
| 3 | CONR | NRCOO | COR | 3 | CONR | C≡C | COR |
| 3 | CONR | CH═CH | OH | 3 | SO₂NR | O | OH |
| 3 | CONR | CH═CH | SH | 3 | SO₂NR | O | SH |
| 3 | SO₂NR | S | SO₂H | 3 | SO₂NR | NR | SO₂H |
| 3 | SO₂NR | S | COH | 3 | SO₂NR | NR | COH |
| 3 | SO₂NR | S | COR | 3 | SO₂NR | NR | COR |
| 3 | SO₂NR | CR₃R₂ | OH | 3 | SO₂NR | CONR | OH |
| 3 | SO₂NR | CR₃R₂ | SH | 3 | SO₂NR | CONR | SH |
| 3 | SO₂NR | CR₃R₂ | CONH₂ | 3 | SO₂NR | CONR | CONH₂ |
| 3 | SO₂NR | CR₃R₂ | CH═CH₂ | 3 | SO₂NR | CONR | CH═CH₂ |
| 3 | SO₂NR | SO₂NR | SH | 3 | SO₂NR | NRCONR | SH |
| 3 | SO₂NR | SO₂NR | COH | 3 | SO₂NR | NRCONR | COH |
| 3 | SO₂NR | SO₂NR | COR | 3 | SO₂NR | NRCONR | COR |
| 3 | SO₂NR | NRCNHNR | OH | 3 | SO₂NR | NRCOO | OH |
| 3 | SO₂NR | NRCNHNR | SH | 3 | SO₂NR | NRCOO | SH |
| 3 | SO₂NR | C≡C | CH═CH₂ | 3 | SO₂NR | CH═CH | CH═CH₂ |
| 3 | SO₂NR | C≡C | NH₂ | 3 | SO₂NR | CH═CH | NH₂ |
| 3 | SO₂NR | C≡C | NHR | 3 | SO₂NR | CH═CH | NHR |
| 3 | NRCONR | O | Br | 3 | NRCONR | S | Br |
| 3 | NRCONR | O | I | 3 | NRCONR | S | I |
| 3 | NRCONR | NR | F | 3 | NRCONR | CR₃R₂ | F |
| 3 | NRCONR | NR | CN | 3 | NRCONR | CR₃R₂ | CN |
| 3 | NRCONR | CONR | SO₂H | 3 | NRCONR | SO₂NR | SO₂H |
| 3 | NRCONR | CONR | Cl | 3 | NRCONR | SO₂NR | Cl |
| 3 | NRCONR | NRCONR | SH | 3 | NRCONR | NRCNHNR | SH |
| 3 | NRCONR | NRCONR | CONH₂ | 3 | NRCONR | NRCNHNR | CONH₂ |
| 3 | NRCONR | NRCONR | CH═CH₂ | 3 | NRCONR | NRCNHNR | CH═CH₂ |
| 3 | NRCONR | NRCOO | NH₂ | 3 | NRCONR | C≡C | NH₂ |
| 3 | NRCONR | NRCOO | COH | 3 | NRCONR | C≡C | COH |
| 3 | NRCONR | CH═CH | OH | 3 | NRCNHNR | O | OH |
| 3 | NRCONR | CH═CH | CONH₂ | 3 | NRCNHNR | O | CONH₂ |
| 3 | NRCONR | CH═CH | CH═CH₂ | 3 | NRCNHNR | O | CH═CH₂ |
| 3 | NRCNHNR | S | SH | 3 | NRCNHNR | NR | SH |
| 3 | NRCNHNR | S | COOH | 3 | NRCNHNR | NR | COOH |
| 3 | NRCNHNR | S | SO₂H | 3 | NRCNHNR | NR | SO₂H |
| 3 | NRCNHNR | SO₂NR | Br | 3 | NRCNHNR | NRCONR | Br |
| 3 | NRCNHNR | SO₂NR | C≡CH | 3 | NRCNHNR | NRCONR | C≡CH |
| 3 | NRCNHNR | SO₂NR | NH₂ | 3 | NRCNHNR | NRCONR | NH₂ |
| 3 | NRCNHNR | NRCNHNR | COOH | 3 | NRCNHNR | NRCOO | COOH |
| 3 | NRCNHNR | NRCNHNR | SO₂H | 3 | NRCNHNR | NRCOO | SO₂H |
| 3 | NRCNHNR | C≡C | Cl | 3 | NRCNHNR | CH═CH | Cl |
| 3 | NRCNHNR | C≡C | Br | 3 | NRCNHNR | CH═CH | Br |
| a3 | NRCOO | O | SH | 3 | NRCOO | S | SH |
| 3 | NRCOO | O | COOH | 3 | NRCOO | S | COOH |
| 3 | NRCOO | O | SO₂H | 3 | NRCOO | S | SO₂H |
| 3 | NRCOO | NR | F | 3 | NRCOO | CR₃R₂ | F |
| 3 | NRCOO | NR | CN | 3 | NRCOO | CR₃R₂ | CN |
| 3 | NRCOO | NR | COR | 3 | NRCOO | CR₃R₂ | COR |
| 3 | NRCOO | CONR | C≡CH | 3 | NRCOO | SO₂NR | C≡CH |
| 3 | NRCOO | CONR | COH | 3 | NRCOO | SO₂NR | COH |
| 3 | NRCOO | CONR | COR | 3 | NRCOO | SO₂NR | COR |
| 3 | NRCOO | NRCONR | OH | 3 | NRCOO | NRCNHNR | OH |
| 3 | NRCOO | NRCONR | COR | 3 | NRCOO | NRCNHNR | COR |
| 3 | NRCOO | NRCOO | Br | 3 | NRCOO | C≡C | Br |
| 3 | NRCOO | CH═CH | CONH₂ | 3 | C≡C | O | CONH₂ |
| 3 | NRCOO | CH═CH | CH═CH₂ | 3 | C≡C | O | CH═CH₂ |
| 3 | C≡C | S | OH | 3 | C≡C | NR | OH |
| 3 | C≡C | CR₃R₂ | I | 3 | C≡C | CONR | I |
| 3 | C≡C | CR₃R₂ | F | 3 | C≡C | CONR | F |
| 3 | C≡C | CR₃R₂ | NH₂ | 3 | C≡C | CONR | NH₂ |
| 3 | C≡C | SO₂NR | N₃ | 3 | C≡C | NRCONR | N₃ |
| 3 | C≡C | SO₂NR | CONH₂ | 3 | C≡C | NRCONR | CONH₂ |
| 3 | C≡C | SO₂NR | CH═CH₂ | 3 | C≡C | NRCONR | CH═CH₂ |
| 3 | C≡C | NRCNHNR | CH═CH₂ | 3 | C≡C | NRCOO | CH═CH₂ |
| 3 | C≡C | NRCNHNR | C≡CH | 3 | C≡C | NRCOO | C≡CH |
| 3 | C≡C | C≡C | I | 3 | C≡C | CH═CH | I |
| 3 | C≡C | C≡C | C≡CH | 3 | C≡C | CH═CH | C≡CH |
| 3 | C≡C | C≡C | NH₂ | 3 | C≡C | CH═CH | NH₂ |
| 3 | C≡C | C≡C | NHR | 3 | C≡C | CH═CH | NHR |
| 3 | CH═CH | O | COOH | 3 | CH═CH | S | COOH |
| 3 | CH═CH | O | CN | 3 | CH═CH | S | CN |
| 3 | CH═CH | NR | I | 3 | CH═CH | CR₃R₂ | I |
| 3 | CH═CH | NR | F | 3 | CH═CH | CR₃R₂ | F |
| 3 | CH═CH | CONR | CN | 3 | CH═CH | SO₂NR | CN |
| 3 | CH═CH | CONR | N₃ | 3 | CH═CH | SO₂NR | N₃ |
| 3 | CH═CH | CONR | C≡CH | 3 | CH═CH | SO₂NR | C≡CH |
| 3 | CH═CH | NRCONR | NHR | 3 | CH═CH | NRCNHNR | NHR |
| 3 | CH═CH | NRCOO | Br | 3 | CH═CH | C≡C | Br |
| 3 | CH═CH | NRCOO | I | 3 | CH═CH | C≡C | I |
| 3 | CH═CH | CH═CH | Cl | 3 | CH═CH | CH═CH | NH₂ |
| 3 | O | O | OH | 3 | O | S | OH |
| 3 | O | O | SH | 3 | O | S | SH |
| 3 | O | NR | CH═CH₂ | 3 | O | CR₃R₂ | CH═CH₂ |
| 3 | O | NR | C≡CH | 3 | O | CR₃R₂ | C≡CH |
| 3 | O | NR | NH₂ | 3 | O | CR₃R₂ | NH₂ |
| 3 | O | CONR | Br | 3 | O | SO₂NR | Br |
| 3 | O | NRCONR | Br | 3 | O | NRCNHNR | Br |
| 3 | O | NRCONR | CONH₂ | 3 | O | NRCNHNR | CONH₂ |
| 3 | O | NRCOO | COH | 3 | O | C≡C | COH |
| 3 | O | NRCOO | COR | 3 | O | C≡C | COR |
| 3 | O | CH═CH | CONH₂ | 3 | S | O | CONH₂ |
| 3 | O | CH═CH | CH═CH₂ | 3 | S | O | CH═CH₂ |
| 3 | O | CH═CH | C≡CH | 3 | S | O | C≡CH |
| 3 | S | S | CONH₂ | 3 | S | NH | CONH₂ |
| 3 | S | S | CH═CH₂ | 3 | S | NR | CH═CH₂ |
| 3 | S | S | C≡CH | 3 | S | NR | C≡CH |
| 3 | S | S | NH₂ | 3 | S | NR | NH₂ |
| 3 | S | CR₃R₂ | N₃ | 3 | S | CONR | N₃ |
| 3 | S | CR₃R₂ | C≡CH | 3 | S | CONR | C≡CH |
| 3 | S | SO₂NR | Br | 3 | S | NRCONR | Br |
| 3 | S | SO₂NR | NHR | 3 | S | NRCONR | NHR |
| 3 | S | SO₂NR | COH | 3 | S | NRCONR | COH |
| 3 | S | NRCNHNR | N₃ | 3 | S | NRCOO | N₃ |
| 3 | S | NRCNHNR | COR | 3 | S | NRCOO | COR |
| 3 | S | C≡C | OH | 3 | S | CH═CH | OH |
| 3 | S | C≡C | SH | 3 | S | CH═CH | SH |
| 3 | S | C≡C | Br | 3 | S | CH═CH | Br |
| 3 | NR | O | SH | 3 | NR | S | SH |
| 3 | NR | O | COOH | 3 | NR | S | COOH |
| 3 | NR | O | CONH₂ | 3 | NR | S | CONH₂ |
| 3 | NR | O | COR | 3 | NR | S | COR |
| 3 | NR | NR | OH | 3 | NR | CR₃R₂ | OH |
| 3 | NR | NR | I | 3 | NR | CR₃R₂ | I |
| 3 | NR | NR | F | 3 | NR | CR₃R₂ | F |
| 3 | NR | CONR | F | 3 | NR | SO₂NR | F |
| 3 | NR | CONR | CONH₂ | 3 | NR | SO₂NR | CONH₂ |
| 3 | NR | NRCONR | Br | 3 | NR | NRCNHNR | Br |
| | NR | NRCONR | I | 3 | NR | NRCNHNR | I |
| 3 | NR | NRCOO | CN | 3 | NR | C≡C | CN |
| 3 | NR | NRCOO | N₃ | 3 | NR | C≡C | N₃ |
| 3 | NR | NRCOO | CONH₂ | 3 | NR | C≡C | CONH₂ |
| 3 | NR | CH═CH | Cl | 3 | CR₃R₂ | O | Cl |
| 3 | NR | CH═CH | Br | 3 | CR₃R₂ | O | Br |
| 3 | CR₃R₂ | S | COOH | 3 | CR₃R₂ | NR | COOH |
| 3 | CR₃R₂ | S | SO₂H | 3 | CR₃R₂ | NR | SO₂H |
| 3 | CR₃R₂ | S | Cl | 3 | CR₃R₂ | NR | Cl |
| 3 | CR₃R₂ | CR₃R₂ | COOH | 3 | CR₃R₂ | CONR | COOH |
| 3 | CR₃R₂ | CR₃R₂ | I | 3 | CR₃R₂ | CONR | I |
| 3 | CR₃R₂ | CR₃R₂ | CH═CH₂ | 3 | CR₃R₂ | CONR | CH═CH₂ |
| 3 | CR₃R₂ | CR₃R₂ | C≡CH | 3 | CR₃R₂ | CONR | C≡CH |
| 3 | CR₃R₂ | SO₂NR | F | 3 | CR₃R₂ | NRCONR | F |
| 3 | CR₃R₂ | SO₂NR | CH═CH₂ | 3 | CR₃R₂ | NRCONR | CH═CH₂ |
| 3 | CR₃R₂ | SO₂NR | C≡CH | 3 | CR₃R₂ | NRCONR | C≡CH |
| 3 | CR₃R₂ | SO₂NR | NH₂ | 3 | CR₃R₂ | NRCONR | NH₂ |
| 3 | CR₃R₂ | NRCNHNR | OH | 3 | CR₃R₂ | NRCOO | OH |
| 3 | CR₃R₂ | NRCNHNR | SH | 3 | CR₃R₂ | NRCOO | SH |
| 3 | CR₃R₂ | C≡C | C≡CH | 3 | CR₃R₂ | CH═CH | C≡CH |
| 3 | CR₃R₂ | C≡C | NH₂ | 3 | CR₃R₂ | CH═CH | NH₂ |
| 3 | CONR | O | SH | 3 | CONR | S | SH |
| 3 | CONR | O | COOH | 3 | CONR | S | COOH |
| 3 | CONR | O | CONH₂ | 3 | CONR | S | CONH₂ |
| 3 | CONR | NR | I | 3 | CONR | CR₃R₂ | I |
| 3 | CONR | NR | F | 3 | CONR | CR₃R₂ | F |
| 3 | CONR | CONR | OH | 3 | CONR | SO₂NR | OH |
| 3 | CONR | CONR | SH | 3 | CONR | SO₂NR | SH |
| 3 | CONR | CONR | COOH | 3 | CONR | SO₂NR | COOH |
| 3 | CONR | NRCONR | NHR | 3 | CONR | NRCNHNR | NHR |
| 3 | CONR | NRCONR | COH | 3 | CONR | NRCNHNR | COH |
| 3 | CONR | NRCOO | I | 3 | CONR | C≡C | I |
| 3 | CONR | NRCOO | F | 3 | CONR | C≡C | F |
| 3 | CONR | CH═CH | F | 3 | SO₂NR | O | F |
| 3 | CONR | CH═CH | COR | 3 | SO₂NR | O | COR |
| 3 | SO₂NR | S | OH | 3 | SO₂NR | NR | OH |
| 3 | SO₂NR | S | SH | 3 | SO₂NR | NR | SH |
| 3 | SO₂NR | CR₃R₂ | N₃ | 3 | SO₂NR | CONR | N₃ |
| 3 | SO₂NR | CR₃R₂ | CONH₂ | 3 | SO₂NR | CONR | CONH₂ |
| 3 | SO₂NR | SO₂NR | COOH | 3 | SO₂NR | NRCONR | COOH |
| 3 | SO₂NR | SO₂NR | CN | 3 | SO₂NR | NRCONR | CN |
| 3 | SO₂NR | SO₂NR | N₃ | 3 | SO₂NR | NRCONR | N₃ |
| 3 | SO₂NR | SO₂NR | CONH₂ | 3 | SO₂NR | NRCONR | CONH₂ |
| 3 | SO₂NR | NRCNHNR | CN | 3 | SO₂NR | NRCOO | CN |
| 3 | SO₂NR | NRCNHNR | CH═CH₂ | 3 | SO₂NR | NRCOO | CH═CH₂ |
| 3 | SO₂NR | C≡C | SO₂H | 3 | SO₂NR | CH═CH | SO₂H |
| 3 | SO₂NR | C≡C | Cl | 3 | SO₂NR | CH═CH | Cl |
| 3 | SO₂NR | C≡C | Br | 3 | SO₂NR | CH═CH | Br |
| 3 | NRCONR | O | C≡CH | 3 | NRCONR | S | C≡CH |
| 3 | NRCONR | O | NH₂ | 3 | NRCONR | S | NH₂ |
| 3 | NRCONR | NR | Cl | 3 | NRCONR | CR₃R₂ | Cl |
| 3 | NRCONR | NR | Br | 3 | NRCONR | CR₃R₂ | Br |
| 3 | NRCONR | NR | CONH₂ | 3 | NRCONR | CR₃R₂ | CONH₂ |
| 3 | NRCONR | CONR | OH | 3 | NRCONR | SO₂NR | OH |
| 3 | NRCONR | CONR | F | 3 | NRCONR | SO₂NR | F |
| 3 | NRCONR | CONR | CN | 3 | NRCONR | SO₂NR | CN |
| 3 | NRCONR | NRCONR | CONH₂ | 3 | NRCONR | NRCNHNR | CONH₂ |
| 3 | NRCONR | NRCONR | CH═CH₂ | 3 | NRCONR | NRCNHNR | CH═CH₂ |
| 3 | NRCONR | NRCOO | CONH₂ | 3 | NRCONR | C≡C | CONH₂ |
| 3 | NRCONR | NRCOO | COH | 3 | NRCONR | C≡C | COH |
| 3 | NRCONR | CH═CH | SO₂H | 3 | NRCNHNR | O | SO₂H |
| 3 | NRCONR | CH═CH | Cl | 3 | NRCNHNR | O | Cl |
| 3 | NRCONR | CH═CH | F | 3 | NRCNHNR | O | F |
| 3 | NRCNHNR | S | OH | 3 | NRCNHNR | NR | OH |
| 3 | NRCNHNR | S | Br | 3 | NRCNHNR | NR | Br |
| 3 | NRCNHNR | CR₃R₂ | OH | 3 | NRCNHNR | CONR | OH |
| 3 | NRCNHNR | CR₃R₂ | SH | 3 | NRCNHNR | CONR | SH |
| 3 | NRCNHNR | CR₃R₂ | CH═CH₂ | 3 | NRCNHNR | CONR | CH═CH₂ |
| 3 | NRCNHNR | SO₂NR | I | 3 | NRCNHNR | NRCONR | I |
| 3 | NRCNHNR | SO₂NR | NHR | 3 | NRCNHNR | NRCONR | NHR |
| 3 | NRCNHNR | SO₂NR | COH | 3 | NRCNHNR | NRCONR | COH |
| 3 | NRCNHNR | SO₂NR | COR | 3 | NRCNHNR | NRCONR | COR |
| 3 | NRCNHNR | NRCNHNR | N₃ | 3 | NRCNHNR | NRCOO | N₃ |
| 3 | NRCNHNR | NRCNHNR | CONH₂ | 3 | NRCNHNR | NRCOO | CONH₂ |
| 3 | NRCNHNR | NRCNHNR | COR | 3 | NRCNHNR | NRCOO | COR |
| 3 | NRCNHNR | C≡C | OH | 3 | NRCNHNR | CH═CH | OH |
| 3 | NRCNHNR | C≡C | COR | 3 | NRCNHNR | CH═CH | COR |
| 3 | NRCOO | O | OH | 3 | NRCOO | S | OH |
| a3 | NRCOO | O | SH | 3 | NRCOO | S | SH |
| 3 | NRCOO | O | COR | 3 | NRCOO | S | COR |
| 3 | NRCOO | NR | OH | 3 | NRCOO | CR₃R₂ | OH |
| 3 | NRCOO | NR | SH | 3 | NRCOO | CR₃R₂ | SH |
| 3 | NRCOO | NR | COOH | 3 | NRCOO | CR₃R₂ | COOH |
| 3 | NRCOO | CONR | NH₂ | 3 | NRCOO | SO₂NR | NH₂ |
| 3 | NRCOO | CONR | NHR | 3 | NRCOO | SO₂NR | NHR |
| 3 | NRCOO | NRCONR | CH═CH₂ | 3 | NRCOO | NRCNHNR | CH═CH₂ |
| 3 | NRCOO | NRCONR | NHR | 3 | NRCOO | NRCNHNR | NHR |
| 3 | NRCOO | NRCOO | I | 3 | NRCOO | C≡C | I |
| 3 | NRCOO | CH═CH | OH | 3 | C≡C | O | OH |
| 3 | NRCOO | CH═CH | SH | 3 | C≡C | O | SH |
| 3 | NRCOO | CH═CH | COOH | 3 | C≡C | O | COOH |
| 3 | C≡C | S | C≡CH | 3 | C≡C | NR | C≡CH |
| 3 | C≡C | S | NH₂ | 3 | C≡C | NR | NH₂ |
| 3 | C≡C | S | NHR | 3 | C≡C | NR | NHR |
| 3 | C≡C | CR₃R₂ | SO₂H | 3 | C≡C | CONR | SO₂H |
| 3 | C≡C | CR₃R₂ | Cl | 3 | C≡C | CONR | Cl |
| 3 | C≡C | CR₃R₂ | Br | 3 | C≡C | CONR | Br |
| 3 | C≡C | SO₂NR | OH | 3 | C≡C | NRCONR | OH |
| 3 | C≡C | SO₂NR | SH | 3 | C≡C | NRCONR | SH |
| 3 | C≡C | SO₂NR | Br | 3 | C≡C | NRCONR | Br |
| 3 | C≡C | NRCNHNR | CONH₂ | 3 | C≡C | NRCOO | CONH₂ |
| 3 | C≡C | NRCNHNR | NHR | 3 | C≡C | NRCOO | NHR |
| 3 | C≡C | C≡C | C≡CH | 3 | C≡C | CH═CH | C≡CH |
| 3 | C≡C | C≡C | NH₂ | 3 | C≡C | CH═CH | NH₂ |
| 3 | C≡C | C≡C | COR | 3 | C≡C | CH═CH | COR |
| 3 | CH═CH | O | OH | 3 | CH═CH | S | OH |
| 3 | CH═CH | O | SH | 3 | CH═CH | S | SH |
| 3 | CH═CH | O | COOH | 3 | CH═CH | S | COOH |
| 3 | CH═CH | O | SO₂H | 3 | CH═CH | S | SO₂H |
| 3 | CH═CH | O | Cl | 3 | CH═CH | S | Cl |
| 3 | CH═CH | NR | OH | 3 | CH═CH | CR₃R₂ | OH |
| 3 | CH═CH | NR | COOH | 3 | CH═CH | CR₃R₂ | COOH |
| 3 | CH═CH | NR | F | 3 | CH═CH | CR₃R₂ | F |
| 3 | CH═CH | CONR | NH₂ | 3 | CH═CH | SO₂NR | NH₂ |
| 3 | CH═CH | CONR | NHR | 3 | CH═CH | SO₂NR | NHR |
| 3 | CH═CH | CONR | COH | 3 | CH═CH | SO₂NR | COH |
| 3 | CH═CH | CONR | COR | 3 | CH═CH | SO₂NR | COR |
| 3 | CH═CH | NRCONR | OH | 3 | CH═CH | NRCNHNR | OH |
| 3 | CH═CH | NRCOO | CH═CH₂ | 3 | CH═CH | C≡C | CH═CH₂ |
| 3 | CH═CH | NRCOO | NHR | 3 | CH═CH | C≡C | NHR |
| 3 | CH═CH | CH═CH | I | 3 | CH═CH | CH═CH | COH |
| 3 | CH═CH | CH═CH | F | 3 | CH═CH | CH═CH | COR |
| 3 | CH═CH | CH═CH | CN | | | | |
| 3 | O | O | OH | 3 | O | S | OH |
| 3 | O | O | SH | 3 | O | S | SH |
| 3 | O | O | COOH | 3 | O | S | COOH |
| 3 | O | NR | CONH₂ | 3 | O | CR₃R₂ | CONH₂ |
| 3 | O | NR | CH═CH₂ | 3 | O | CR₃R₂ | CH═CH₂ |
| 3 | O | NR | C≡CH | 3 | O | CR₃R₂ | C≡CH |
| 3 | O | CONR | CONH₂ | 3 | O | SO₂NR | CONH₂ |
| 3 | O | CONR | CH═CH₂ | 3 | O | SO₂NR | CH═CH₂ |
| 3 | O | NRCONR | CONH₂ | 3 | O | NRCNHNR | CONH₂ |
| 3 | O | NRCONR | CH═CH₂ | 3 | O | NRCNHNR | CH═CH₂ |
| 3 | O | NRCOO | COOH | 3 | O | C≡C | COOH |
| 3 | O | NRCOO | SO₂H | 3 | O | C≡C | SO₂H |
| 3 | O | NRCOO | Cl | 3 | O | C≡C | Cl |
| 3 | O | CH═CH | SO₂H | 3 | S | O | SO₂H |
| 3 | O | CH═CH | Cl | 3 | S | O | Cl |
| 3 | O | CH═CH | COR | 3 | S | O | COR |
| 3 | S | S | OH | 3 | S | NR | OH |
| 3 | S | S | SH | 3 | S | NR | SH |
| 3 | S | S | COOH | 3 | S | NR | COOH |
| 3 | S | S | SO₂H | 3 | S | NR | SO₂H |
| 3 | S | CR₃R₂ | CONH₂ | 3 | S | CONR | CONH₂ |
| 3 | S | CR₃R₂ | CH═CH₂ | 3 | S | CONR | CH═CH₂ |
| 3 | S | CR₃R₂ | NHR | 3 | S | CONR | NHR |
| 3 | S | SO₂NR | NHR | 3 | S | NRCONR | NHR |
| 3 | S | SO₂NR | COH | 3 | S | NRCONR | COH |
| 3 | S | SO₂NR | COR | 3 | S | NRCONR | COR |
| 3 | S | NRCNHNR | OH | 3 | S | NRCOO | OH |
| 3 | S | NRCNHNR | NH₂ | 3 | S | NRCOO | NH₂ |
| 3 | S | NRCNHNR | NHR | 3 | S | NRCOO | NHR |
| 3 | S | C≡C | I | 3 | S | CH═CH | I |
| 3 | S | C≡C | NH₂ | 3 | S | CH═CH | NH₂ |
| 3 | NR | O | SO₂H | 3 | NR | S | SO₂H |
| 3 | NR | O | F | 3 | NR | S | F |
| 3 | NR | O | CN | 3 | NR | S | CN |
| 3 | NR | O | N₃ | 3 | NR | S | N₃ |
| 3 | NR | O | NH₂ | 3 | NR | S | NH₂ |
| 3 | NR | NR | SH | 3 | NR | CR₃R₂ | SH |
| 3 | NR | NR | COOH | 3 | NR | CR₃R₂ | COOH |
| 3 | NR | CONR | CN | 3 | NR | SO₂NR | CN |
| 3 | NR | CONR | COR | 3 | NR | SO₂NR | COR |
| 3 | NR | NRCONR | OH | 3 | NR | NRCNHNR | OH |
| 3 | NR | NRCONR | NHR | 3 | NR | NRCNHNR | NHR |
| 3 | NR | NRCOO | SO₂H | 3 | NR | C≡C | SO₂H |
| 3 | NR | NRCOO | C≡CH | 3 | NR | C≡C | C≡CH |
| 3 | NR | NRCOO | NH₂ | 3 | NR | C≡C | NH₂ |
| 3 | NR | NRCOO | NHR | 3 | NR | C≡C | NHR |
| 3 | NR | CH═CH | COR | 3 | CR₃R₂ | O | COR |
| 3 | CR₃R₂ | S | OH | 3 | CR₃R₂ | NR | OH |
| 3 | CR₃R₂ | S | SH | 3 | CR₃R₂ | NR | SH |
| 3 | CR₃R₂ | CR₃R₂ | SO₂H | 3 | CR₃R₂ | CONR | SO₂H |
| 3 | CR₃R₂ | CR₃R₂ | Cl | 3 | CR₃R₂ | CONR | Cl |
| 3 | CR₃R₂ | SO₂NR | OH | 3 | CR₃R₂ | NRCONR | OH |
| 3 | CR₃R₂ | SO₂NR | C≡CH | 3 | CR₃R₂ | NRCONR | C≡CH |
| 3 | CR₃R₂ | SO₂NR | NH₂ | 3 | CR₃R₂ | NRCONR | NH₂ |
| 3 | CR₃R₂ | SO₂NR | NHR | 3 | CR₃R₂ | NRCONR | NHR |
| 3 | CR₃R₂ | NRCNHNR | Cl | 3 | CR₃R₂ | NRCOO | Cl |
| 3 | CR₃R₂ | NRCNHNR | COR | 3 | CR₃R₂ | NRCOO | COR |
| 3 | CR₃R₂ | C≡C | Cl | 3 | CR₃R₂ | CH═CH | Cl |
| 3 | CR₃R₂ | C≡C | Br | 3 | CR₃R₂ | CH═CH | Br |
| 3 | CR₃R₂ | C≡C | NHR | 3 | CR₃R₂ | CH═CH | NHR |
| 3 | CONR | O | COR | 3 | CONR | S | COR |
| 3 | CONR | NR | OH | 3 | CONR | CR₃R₂ | OH |
| 3 | CONR | NR | SH | 3 | CONR | CR₃R₂ | SH |
| 3 | CONR | NR | C≡CH | 3 | CONR | CR₃R₂ | C≡CH |
| 3 | CONR | CONR | Br | 3 | CONR | SO₂NR | Br |
| 3 | CONR | CONR | I | 3 | CONR | SO₂NR | I |
| 3 | CONR | CONR | F | 3 | CONR | SO₂NR | F |
| 3 | CONR | NRCONR | OH | 3 | CONR | NRCNHNR | OH |
| 3 | CONR | NRCOO | COOH | 3 | CONR | C≡C | COOH |
| 3 | CONR | NRCOO | SO₂H | 3 | CONR | C≡C | SO₂H |
| 3 | CONR | NRCOO | F | 3 | CONR | C≡C | F |
| 3 | CONR | CH═CH | Cl | 3 | SO₂NR | O | Cl |
| 3 | CONR | CH═CH | NHR | 3 | SO₂NR | O | NHR |
| 3 | SO₂NR | S | OH | 3 | SO₂NR | NR | OH |
| 3 | SO₂NR | S | SH | 3 | SO₂NR | NR | SH |
| 3 | SO₂NR | S | NH₂ | 3 | SO₂NR | NR | NH₂ |
| 3 | SO₂NR | S | NHR | 3 | SO₂NR | NR | NHR |
| 3 | SO₂NR | CR₃R₂ | Cl | 3 | SO₂NR | CONR | Cl |
| 3 | SO₂NR | CR₃R₂ | Br | 3 | SO₂NR | CONR | Br |
| 3 | SO₂NR | SO₂NR | Br | 3 | SO₂NR | NRCONR | Br |
| 3 | SO₂NR | SO₂NR | I | 3 | SO₂NR | NRCONR | I |
| 3 | SO₂NR | NRCNHNR | OH | 3 | SO₂NR | NRCOO | OH |
| 3 | SO₂NR | NRCNHNR | SH | 3 | SO₂NR | NRCOO | SH |
| 3 | SO₂NR | NRCNHNR | COR | 3 | SO₂NR | NRCOO | COR |
| 3 | SO₂NR | C≡C | OH | 3 | SO₂NR | CH═CH | OH |
| 3 | SO₂NR | C≡C | CN | 3 | SO₂NR | CH═CH | CN |
| 3 | NRCONR | O | I | 3 | NRCONR | S | I |
| 3 | NRCONR | O | COH | 3 | NRCONR | S | COH |
| 3 | NRCONR | O | COR | 3 | NRCONR | S | COR |
| 3 | NRCONR | NR | OH | 3 | NRCONR | CR₃R₂ | OH |
| 3 | NRCONR | NR | SH | 3 | NRCONR | CR₃R₂ | SH |
| 3 | NRCONR | CONR | OH | 3 | NRCONR | SO₂NR | OH |
| 3 | NRCONR | CONR | SH | 3 | NRCONR | SO₂NR | SH |
| 3 | NRCONR | CONR | SO₂H | 3 | NRCONR | SO₂NR | SO₂H |
| 3 | NRCONR | NRCONR | I | 3 | NRCONR | NRCNHNR | I |
| 3 | NRCONR | NRCONR | N₃ | 3 | NRCONR | NRCNHNR | N₃ |
| 3 | NRCONR | NRCONR | CONH₂ | 3 | NRCONR | NRCNHNR | CONH₂ |
| 3 | NRCONR | NRCOO | SH | 3 | NRCONR | C≡C | SH |
| 3 | NRCONR | NRCOO | COOH | 3 | NRCONR | C≡C | COOH |
| 3 | NRCONR | CH═CH | CN | 3 | NRCNHNR | O | CN |
| 3 | NRCONR | CH═CH | N₃ | 3 | NRCNHNR | O | N₃ |
| 3 | NRCONR | CH═CH | COR | 3 | NRCNHNR | O | COR |
| 3 | NRCNHNR | S | OH | 3 | NRCNHNR | NR | OH |
| 3 | NRCNHNR | S | COH | 3 | NRCNHNR | NR | COH |
| 3 | NRCNHNR | S | COR | 3 | NRCNHNR | NR | COR |
| 3 | NRCNHNR | CR₃R₂ | Br | 3 | NRCNHNR | CONR | Br |
| 3 | NRCNHNR | CR₃R₂ | N₃ | 3 | NRCNHNR | CONR | N₃ |
| 3 | NRCNHNR | SO₂NR | C≡CH | 3 | NRCNHNR | NRCONR | C≡CH |
| 3 | NRCNHNR | SO₂NR | COH | 3 | NRCNHNR | NRCONR | COH |
| 3 | NRCNHNR | NRCNHNR | NHR | 3 | NRCNHNR | NRCOO | NHR |
| 3 | NRCNHNR | NRCNHNR | COH | 3 | NRCNHNR | NRCOO | COH |
| 3 | NRCNHNR | NRCNHNR | COR | 3 | NRCNHNR | NRCOO | COR |
| 3 | NRCNHNR | C≡C | OH | 3 | NRCNHNR | CH═CH | OH |
| 3 | NRCNHNR | C≡C | Br | 3 | NRCNHNR | CH═CH | Br |
| 3 | NRCNHNR | C≡C | I | 3 | NRCNHNR | CH═CH | I |
| 3 | NRCOO | O | COH | 3 | NRCOO | S | COH |
| 3 | NRCOO | O | COR | 3 | NRCOO | S | COR |
| 3 | NRCOO | NR | CONH₂ | 3 | NRCOO | CR₃R₂ | CONH₂ |
| 3 | NRCOO | NR | CH═CH₂ | 3 | NRCOO | CR₃R₂ | CH═CH₂ |
| 3 | NRCOO | NR | COH | 3 | NRCOO | CR₃R₂ | COH |
| 3 | NRCOO | NR | COR | 3 | NRCOO | CR₃R₂ | COR |
| 3 | NRCOO | CONR | OH | 3 | NRCOO | SO₂NR | OH |
| 3 | NRCOO | CONR | Cl | 3 | NRCOO | SO₂NR | Cl |
| 3 | NRCOO | CONR | CONH₂ | 3 | NRCOO | SO₂NR | CONH₂ |
| 3 | NRCOO | NRCONR | Cl | 3 | NRCOO | NRCNHNR | Cl |
| 3 | NRCOO | NRCONR | N₃ | 3 | NRCOO | NRCNHNR | N₃ |
| 3 | NRCOO | NRCONR | CONH₂ | 3 | NRCOO | NRCNHNR | CONH₂ |
| 3 | NRCOO | NRCONR | CH═CH₂ | 3 | NRCOO | NRCNHNR | CH═CH₂ |
| 3 | NRCOO | NRCOO | Cl | 3 | NRCOO | C≡C | Cl |
| 3 | NRCOO | NRCOO | NH₂ | 3 | NRCOO | C≡C | NH₂ |
| 3 | NRCOO | CH═CH | I | 3 | C≡C | O | I |
| 3 | NRCOO | CH═CH | F | 3 | C≡C | O | F |
| 3 | C≡C | S | CN | 3 | C≡C | NR | CN |
| 3 | C≡C | S | NHR | 3 | C≡C | NR | NHR |
| 3 | C≡C | CR₃R₂ | COOH | 3 | C≡C | CONR | COOH |
| 3 | C≡C | CR₃R₂ | SO₂H | 3 | C≡C | CONR | SO₂H |
| 3 | C≡C | CR₃R₂ | CN | 3 | C≡C | CONR | CN |
| 3 | C≡C | SO₂NR | Cl | 3 | C≡C | NRCONR | Cl |
| 3 | C≡C | SO₂NR | COR | 3 | C≡C | NRCONR | COR |
| 3 | C≡C | NRCNHNR | OH | 3 | C≡C | NRCOO | OH |
| 3 | C≡C | NRCNHNR | F | 3 | C≡C | NRCOO | F |
| 3 | C≡C | NRCNHNR | NH₂ | 3 | C≡C | NRCOO | NH₂ |
| 3 | C≡C | C≡C | I | 3 | C≡C | CH═CH | I |
| 3 | C≡C | C≡C | F | 3 | C≡C | CH═CH | F |
| 3 | C≡C | C≡C | CN | 3 | C≡C | CH═CH | CN |
| 3 | CH═CH | O | F | 3 | CH═CH | S | F |
| 3 | CH═CH | O | CN | 3 | CH═CH | S | CN |
| 3 | CH═CH | NR | CONH₂ | 3 | CH═CH | CR₃R₂ | CONH₂ |
| 3 | CH═CH | NR | CH═CH₂ | 3 | CH═CH | CR₃R₂ | CH═CH₂ |
| 3 | CH═CH | NR | C≡CH | 3 | CH═CH | CR₃R₂ | C≡CH |
| 3 | CH═CH | NR | NH₂ | 3 | CH═CH | CR₃R₂ | NH₂ |
| 3 | CH═CH | CONR | C≡CH | 3 | CH═CH | SO₂NR | C≡CH |
| 3 | CH═CH | CONR | NH₂ | 3 | CH═CH | SO₂NR | NH₂ |
| 3 | CH═CH | NRCONR | I | 3 | CH═CH | NRCNHNR | I |
| 3 | CH═CH | NRCONR | F | 3 | CH═CH | NRCNHNR | F |
| 3 | CH═CH | NRCOO | OH | 3 | CH═CH | C≡C | OH |
| 3 | CH═CH | NRCOO | COOH | 3 | CH═CH | C≡C | COOH |
| 3 | CH═CH | NRCOO | SO₂H | 3 | CH═CH | C≡C | SO₂H |
| 3 | CH═CH | CH═CH | OH | 3 | CH═CH | CH═CH | N₃ |
| 3 | CH═CH | CH═CH | COOH | 3 | CH═CH | CH═CH | CH═CH₂ |
| 3 | CH═CH | CH═CH | CN | | | | |
| 4 | O | O | OH | 4 | O | S | OH |
| 4 | O | O | SH | 4 | O | S | SH |
| 4 | O | O | CONH₂ | 4 | O | S | CONH₂ |
| 4 | O | NR | SH | 4 | O | CR₄R₂ | SH |
| 4 | O | NR | Cl | 4 | O | CR₄R₂ | Cl |
| 4 | O | NR | NHR | 4 | O | CR₄R₂ | NHR |
| 4 | O | CONR | F | 4 | O | SO₂NR | F |
| 4 | O | CONR | CH═CH₂ | 4 | O | SO₂NR | CH═CH₂ |
| 4 | O | CONR | COR | 4 | O | SO₂NR | COR |
| 4 | O | NRCONR | OH | 4 | O | NRCNHNR | OH |
| 4 | O | NRCONR | NHR | 4 | O | NRCNHNR | NHR |
| 4 | O | NRCOO | CN | 4 | O | C≡C | CN |
| 4 | O | NRCOO | NHR | 4 | O | C≡C | NHR |
| 4 | O | CH═CH | Br | 4 | S | O | Br |
| 4 | O | CH═CH | C≡CH | 4 | S | O | C≡CH |
| 4 | O | CH═CH | NH₂ | 4 | S | O | NH₂ |
| 4 | S | S | Br | 4 | S | NR | Br |
| 4 | S | S | N₃ | 4 | S | NR | N₃ |
| 4 | S | S | NH₂ | 4 | S | NR | NH₂ |
| 4 | S | S | NHR | 4 | S | NR | NHR |
| 4 | S | CR₄R₂ | OH | 4 | S | CONR | OH |
| 4 | S | CR₄R₂ | COR | 4 | S | CONR | COR |
| 4 | S | SO₂NR | COOH | 4 | S | NRCONR | COOH |
| 4 | S | SO₂NR | I | 4 | S | NRCONR | I |
| 4 | S | SO₂NR | F | 4 | S | NRCONR | F |
| 4 | S | SO₂NR | COR | 4 | S | NRCONR | COR |
| 4 | S | NRCNHNR | OH | 4 | S | NRCOO | OH |
| 4 | S | NRCNHNR | I | 4 | S | NRCOO | I |
| 4 | S | NRCNHNR | F | 4 | S | NRCOO | F |
| 4 | S | C≡C | SH | 4 | S | CH═CH | SH |
| 4 | NR | O | OH | 4 | NR | S | OH |
| 4 | NR | O | SH | 4 | NR | S | SH |
| 4 | NR | O | NH₂ | 4 | NR | S | NH₂ |
| 4 | NR | NR | SO₂H | 4 | NR | CR₄R₂ | SO₂H |
| 4 | NR | NR | Cl | 4 | NR | CR₄R₂ | Cl |
| 4 | NR | NR | NHR | 4 | NR | CR₄R₂ | NHR |
| 4 | NR | NR | COR | 4 | NR | CR₄R₂ | COR |
| 4 | NR | CONR | OH | 4 | NR | SO₂NR | OH |
| 4 | NR | CONR | NH₂ | 4 | NR | SO₂NR | NH₂ |
| 4 | NR | CONR | NHR | 4 | NR | SO₂NR | NHR |
| 4 | NR | NRCONR | I | 4 | NR | NRCNHNR | I |
| 4 | NR | NRCONR | F | 4 | NR | NRCNHNR | F |
| 4 | NR | NRCOO | OH | 4 | NR | C≡C | OH |
| 4 | NR | NRCOO | CONH₂ | 4 | NR | C≡C | CONH₂ |
| 4 | NR | CH═CH | NH₂ | 4 | CR₄R₂ | OO | NH₂ |
| 4 | NR | CH═CH | NHR | 4 | CR₄R₂ | O | NHR |
| 4 | NH | CH═CH | COR | 4 | CR₄R₂ | O | COR |
| 4 | CR₄R₂ | S | OH | 4 | CR₄R₂ | NR | OH |
| 4 | CR₄R₂ | S | Br | 4 | CR₄R₂ | NR | Br |
| 4 | CR₄R₂ | CR₄R₂ | SO₂H | 4 | CR₄R₂ | CONR | SO₂H |
| 4 | CR₄R₂ | CR₄R₂ | CH═CH₂ | 4 | CR₄R₂ | CONR | CH═CH₂ |
| 4 | CR₄R₂ | CR₄R₂ | C≡CH | 4 | CR₄R₂ | CONR | C≡CH |
| 4 | CR₄R₂ | SO₂NR | F | 4 | CR₄R₂ | NRCONR | F |
| 4 | CR₄R₂ | SO₂NR | CN | 4 | CR₄R₂ | NRCONR | CN |
| 4 | CR₄R₂ | SO₂NR | N₃ | 4 | CR₄R₂ | NRCONR | N₃ |
| 4 | CR₄R₂ | NRCNHNR | CONH₂ | 4 | CR₄R₂ | NRCOO | CONH₂ |
| 4 | CR₄R₂ | NRCNHNR | CH═CH₂ | 4 | CR₄R₂ | NRCOO | CH═CH₂ |
| 4 | CR₄R₂ | NHCNHNR | C≡CH | 4 | CR₄R₂ | NRCOO | C≡CH |
| 4 | CR₄R₂ | C≡C | Cl | 4 | CR₄R₂ | CH═CH | Cl |
| 4 | CR₄R₂ | C≡C | Br | 4 | CR₄R₂ | CH═CH | Br |
| 4 | CR₄R₂ | C≡C | I | 4 | CR₄R₂ | CH═CH | I |
| 4 | CONR | O | COH | 4 | CONR | S | COH |
| 4 | CONR | O | COR | 4 | CONR | S | COR |
| 4 | CONR | NR | OH | 4 | CONR | CR₄R₂ | OH |
| 4 | CONR | NR | Br | 4 | CONR | CR₄R₂ | Br |
| 4 | CONR | NH | N₃ | 4 | CONR | CR₄R₂ | N₃ |
| 4 | CONR | CONR | Br | 4 | CONR | SO₂NR | Br |
| 4 | CONR | CONR | N₃ | 4 | CONR | SO₂NR | N₃ |
| 4 | CONR | CONR | C≡CH | 4 | CONR | SO₂NR | C≡CH |
| 4 | CONR | NRCONR | OH | 4 | CONR | NRCNHNR | OH |
| 4 | CONR | NRCONR | SH | 4 | CONR | NRCNHNR | SH |
| 4 | CONR | NRCONR | COH | 4 | CONR | NRCNHNR | COH |
| 4 | CONR | NRCOO | F | 4 | CONR | C≡C | F |
| 4 | CONR | NRCOO | CN | 4 | CONR | C≡C | CN |
| 4 | CONR | NRCOO | COR | 4 | CONR | C≡C | COR |
| 4 | CONR | CH═CH | OH | 4 | SO₂NR | O | OH |
| 4 | CONR | CH═CH | CN | 4 | SO₂NR | O | CN |
| 4 | CONR | CH═CH | COR | 4 | SO₂NR | O | COR |
| 4 | SO₂NR | S | OH | 4 | SO₂NR | NR | OH |
| 4 | SO₂NR | S | SH | 4 | SO₂NR | NR | SH |
| 4 | SO₂NR | CR₄R₂ | N₃ | 4 | SO₂NR | CONR | N₃ |
| 4 | SO₂NR | CR₄R₂ | NHR | 4 | SO₂NR | CONR | NHR |
| 4 | SO₂NR | CR₄R₂ | COH | 4 | SO₂NR | CONR | COH |
| 4 | SO₂NR | SO₂NR | COOH | 4 | SO₂NR | NRCONR | COOH |
| 4 | SO₂NR | SO₂NR | NHR | 4 | SO₂NR | NRCONR | NHR |
| 4 | SO₂NR | SO₂NR | COH | 4 | SO₂NR | NRCONR | COH |
| 4 | SO₂NR | NRCNHNR | SH | 4 | SO₂NR | NRCOO | SH |
| 4 | SO₂NR | NRCNHNR | COOH | 4 | SO₂NR | NRCOO | COOH |
| 4 | SO₂NR | NRCNHNR | SO₂H | 4 | SO₂NR | NRCOO | SO₂H |
| 4 | SO₂NR | NRCNHNR | Cl | 4 | SO₂NR | NRCOO | Cl |
| 4 | SO₂NR | C≡C | I | 4 | SO₂NR | CH═CH | I |
| 4 | SO₂NR | C≡C | F | 4 | SO₂NR | CH═CH | F |
| 4 | SO₂NR | C≡C | CN | 4 | SO₂NR | CH═CH | CN |
| 4 | NRCONR | O | F | 4 | NRCONR | S | F |
| 4 | NRCONR | O | CN | 4 | NRCONR | S | CN |
| 4 | NRCONR | O | N₃ | 4 | NRCONR | S | N₃ |
| 4 | NRCONR | NR | CONH₂ | 4 | NRCONR | CR₄R₂ | CONH₂ |
| 4 | NRCONR | NR | CH═CH₂ | 4 | NRCONR | CR₄R₂ | CH═CH₂ |
| 4 | NRCONR | NR | C≡CH | 4 | NRCONR | CR₄R₂ | C≡CH |
| 4 | NRCONR | CONR | SH | 4 | NRCONR | SO₂NR | SH |
| 4 | NRCONR | CONR | COOH | 4 | NRCONR | SO₂NR | COOH |
| 4 | NRCONR | NRCONR | CH═CH₂ | 4 | NRCONR | NRCNHNR | CH═CH₂ |
| 4 | NRCONR | NRCOO | SH | 4 | NRCONR | C≡C | SH |
| 4 | NRCONR | NRCOO | COOH | 4 | NRCONR | C≡C | COOH |
| 4 | NRCONR | CH═CH | SO₂H | 4 | NRCNHNR | O | SO₂H |
| 4 | NRCONR | CH═CH | Cl | 4 | NRCNHNR | O | Cl |
| 4 | NRCNHNR | S | Br | 4 | NRCNHNR | NR | Br |
| 4 | NRCNHNR | S | I | 4 | NRCNHNR | NR | I |
| 4 | NRCNHNR | CR₄R₂ | N₃ | 4 | NRCNHNR | CONR | N₃ |
| 4 | NRCNHNR | CR₄R₂ | CONH₂ | 4 | NRCNHNR | CONR | CONH₂ |
| 4 | NRCNHNR | SO₂NR | SO₂H | 4 | NRCNHNR | NRCONR | SO₂H |
| 4 | NRCNHNR | SO₂NR | Cl | 4 | NRCNHNR | NRCONR | Cl |
| 4 | NRCNHNR | SO₂NR | Br | 4 | NRCNHNR | NRCONR | Br |
| 4 | NRCNHNR | NRCNHNR | COR | 4 | NRCNHNR | NRCOO | COR |
| 4 | NRCNHNR | C≡C | Br | 4 | NRCNHNR | CH═CH | Br |
| 4 | NRCOO | O | COH | 4 | NRCOO | S | COH |
| 4 | NRCOO | O | COR | 4 | NRCOO | S | COR |
| 4 | NRCOO | NR | OH | 4 | NRCOO | CR₄R₂ | OH |
| 4 | NRCOO | NR | COH | 4 | NRCOO | CR₄R₂ | COH |
| 4 | NRCOO | NR | COR | 4 | NRCOO | CR₄R₂ | COR |
| 4 | NRCOO | CONR | OH | 4 | NRCOO | SO₂NR | OH |
| 4 | NRCOO | CONR | SH | 4 | NRCOO | SO₂NR | SH |
| 4 | NRCOO | NRCONR | NH₂ | 4 | NRCOO | NRCNHNR | NH₂ |
| 4 | NRCOO | NRCOO | SH | 4 | NRCOO | C≡C | SH |
| 4 | NRCOO | NRCOO | COOH | 4 | NRCOO | C≡C | COOH |
| 4 | NRCOO | CH═CH | COH | 4 | C≡C | O | COH |
| 4 | NRCOO | CH═CH | COR | 4 | C≡C | O | COR |
| 4 | C≡C | S | OH | 4 | C≡C | NR | OH |
| 4 | C≡C | CR₄R₂ | COOH | 4 | C≡C | CONR | COOH |
| 4 | C≡C | CR₄R₂ | SO₂H | 4 | C≡C | CONR | SO₂H |
| 4 | C≡C | SO₂NR | SO₂H | 4 | C≡C | NRCONR | SO₂H |
| 4 | C≡C | SO₂NR | COR | 4 | C≡C | NRCONR | COR |
| 4 | C≡C | NRCNHNR | OH | 4 | C≡C | NRCOO | OH |
| 4 | C≡C | NRCNHNR | SH | 4 | C≡C | NRCOO | SH |
| 4 | C≡C | C≡C | CONH₂ | 4 | C≡C | CH═CH | CONH₂ |
| 4 | C≡C | C≡C | COR | 4 | C≡C | CH═CH | COR |
| 4 | CH═CH | O | OH | 4 | CH═CH | S | OH |
| 4 | CH═CH | O | NH₂ | 4 | CH═CH | S | NH₂ |
| 4 | CH═CH | O | COR | 4 | CH═CH | S | COR |
| 4 | CH═CH | NR | OH | 4 | CH═CH | CR₄R₂ | OH |
| 4 | CH═CH | NR | COH | 4 | CH═CH | CR₄R₂ | COH |
| 4 | CH═CH | CONR | OH | 4 | CH═CH | SO₂NR | OH |
| 4 | CH═CH | CONR | CH═CH₂ | 4 | CH═CH | SO₂NR | CH═CH₂ |
| 4 | CH═CH | CONR | C≡CH | 4 | CH═CH | SO₂NR | C≡CH |
| 4 | CH═CH | CONR | NH₂ | 4 | CH═CH | SO₂NR | NH₂ |
| 4 | CH═CH | NRCONR | C≡CH | 4 | CH═CH | NRCNHNR | C≡CH |
| 4 | CH═CH | NRCONR | NH₂ | 4 | CH═CH | NRCNHNR | NH₂ |
| 4 | CH═CH | NRCOO | I | 4 | CH═CH | C≡C | I |
| 4 | CH═CH | NRCOO | C≡CH | 4 | CH═CH | C≡C | C≡CH |
| 4 | CH═CH | CH═CH | OH | 4 | CH═CH | CH═CH | N₃ |
| 4 | CH═CH | CH═CH | SH | 4 | CH═CH | CH═CH | CONH₂ |
| 4 | CH═CH | CH═CH | Br | 4 | CH═CH | CH═CH | NHR |
| 5 | O | O | CN | 5 | O | S | CN |
| 5 | O | O | N₃ | 5 | O | S | N₃ |
| 5 | O | NR | Br | 5 | O | CR₅R₂ | Br |
| 5 | O | NR | I | 5 | O | CR₅R₂ | I |
| 5 | O | CONR | CONH₂ | 5 | O | SO₂NR | CONH₂ |
| 5 | O | CONR | CH═CH₂ | 5 | O | SO₂NR | CH═CH₂ |
| 5 | O | NRCONR | NHR | 5 | O | NRCNHNR | NHR |
| 5 | O | NRCONR | COH | 5 | O | NRCNHNR | COH |
| 5 | O | NRCOO | OH | 5 | O | C≡C | OH |
| 5 | O | NRCOO | COOH | 5 | O | C≡C | COOH |
| 5 | O | CH═CH | OH | 5 | S | O | OH |
| 5 | O | CH═CH | C≡CH | 5 | S | O | C≡CH |
| 5 | S | S | Cl | 5 | S | NR | Cl |
| 5 | S | S | Br | 5 | S | NR | Br |
| 5 | S | S | I | 5 | S | NR | I |
| 5 | S | S | NH₂ | 5 | S | NR | NH₂ |
| 5 | S | CR₅R₂ | COOH | 5 | S | CONR | COOH |
| 5 | S | CR₅R₂ | NHR | 5 | S | CONR | NHR |
| 5 | S | CR₅R₂ | COH | 5 | S | CONR | COH |
| 5 | S | CR₅R₂ | COR | 5 | S | CONR | COR |
| 5 | S | SO₂NR | Cl | 5 | S | NRCONR | Cl |
| 5 | S | SO₂NR | CN | 5 | S | NRCONR | CN |
| 5 | S | SO₂NR | N₃ | 5 | S | NRCONR | N₃ |
| 5 | S | SO₂NR | COR | 5 | S | NRCONR | COR |
| 5 | S | NRCNHNR | OH | 5 | S | NRCOO | OH |
| 5 | S | NRCNHNR | COR | 5 | S | NRCOO | COR |
| 5 | S | C≡C | OH | 5 | S | CH═CH | OH |
| 5 | S | C≡C | SH | 5 | S | CH═CH | SH |
| 5 | NR | O | SH | 5 | NR | S | SH |
| 5 | NR | O | COOH | 5 | NR | S | COOH |
| 5 | NR | O | SO₂H | 5 | NR | S | SO₂H |
| 5 | NR | NR | OH | 5 | NR | CR₅R₂ | OH |
| 5 | NR | NR | SH | 5 | NR | CR₅R₂ | SH |
| 5 | NR | CONR | OH | 5 | NR | SO₂NR | OH |
| 5 | NR | CONR | COR | 5 | NR | SO₂NR | COR |
| 5 | NR | NRCONR | OH | 5 | NR | NRCNHNR | OH |
| 5 | NR | NRCONR | SH | 5 | NR | NRCNHNR | SH |
| 5 | NR | NRCOO | NH₂ | 5 | NR | C≡C | NH₂ |
| 5 | NR | NRCOO | NHR | 5 | NR | C≡C | NHR |
| 5 | NR | CH═CH | COOH | 5 | CR₅R₂ | O | COOH |
| 5 | NR | CH═CH | SO₂H | 5 | CR₅R₂ | O | SO₂H |
| 5 | CR₅R₂ | S | SO₂H | 5 | CR₅R₂ | NR | SO₂H |
| 5 | CR₅R₂ | S | NH₂ | 5 | CR₅R₂ | NR | NH₂ |
| 5 | CR₅R₂ | S | NHR | 5 | CR₅R₂ | NR | NHR |
| 5 | CR₅R₂ | S | COH | 5 | CR₅R₂ | NR | COH |
| 5 | CR₅R₂ | CR₅R₂ | COOH | 5 | CR₅R₂ | CONR | COOH |
| 5 | CR₅R₂ | CR₅R₂ | F | 5 | CR₅R₂ | CONR | F |
| 5 | CR₅R₂ | SO₂NR | NH₂ | 5 | CR₅R₂ | NRCONR | NH₂ |
| 5 | CR₅R₂ | SO₂NR | NHR | 5 | CR₅R₂ | NRCONR | NHR |
| 5 | CR₅R₂ | SO₂NR | COH | 5 | CR₅R₂ | NRCONR | COH |
| 5 | CR₅R₂ | NRCNHNR | COH | 5 | CR₅R₂ | NRCOO | COH |
| 5 | CR₅R₂ | NRCNHNR | COR | 5 | CR₅R₂ | NRCOO | COR |
| 5 | CR₅R₂ | C≡C | OH | 5 | CR₅R₂ | CH═CH | OH |
| 5 | CR₅R₂ | C≡C | Cl | 5 | CR₃R₂ | CH═CH | Cl |
| 5 | CONR | O | N₃ | 5 | CONR | S | N₃ |
| 5 | CONR | O | COH | 5 | CONR | S | COH |
| 5 | CONR | O | COR | 5 | CONR | S | COR |
| 5 | CONR | NR | OH | 5 | CONR | CR₅R₂ | OH |
| 5 | CONR | NR | NHR | 5 | CONR | CR₅R₂ | NHR |
| 5 | CONR | CONR | COOH | 5 | CONR | SO₂NR | COOH |
| 5 | CONR | CONR | NHR | 5 | CONR | SO₂NR | NHR |
| 5 | CONR | NRCONR | F | 5 | CONR | NRCNHNR | F |
| 5 | CONR | NRCONR | CN | 5 | CONR | NRCNHNR | CN |
| 5 | CONR | NRCOO | OH | 5 | CONR | C≡C | OH |
| 5 | CONR | NRCOO | COH | 5 | CONR | C≡C | COH |
| 5 | CONR | CH═CH | I | 5 | SO₂NR | O | I |
| 5 | CONR | CH═CH | F | 5 | SO₂NR | O | F |
| 5 | CONR | CH═CH | COR | 5 | SO₂NR | O | COR |
| 5 | SO₂NR | S | OH | 5 | SO₂NR | NR | OH |
| 5 | SO₂NR | S | SO₂H | 5 | SO₂NR | NR | SO₂H |
| 5 | SO₂NR | S | Cl | 5 | SO₂NR | NR | Cl |
| 5 | SO₂NR | CR₅R₂ | F | 5 | SO₂NR | CONR | F |
| 5 | SO₂NR | CR₅R₂ | NHR | 5 | SO₂NR | CONR | NHR |
| 5 | SO₂NR | SO₂NR | COOH | 5 | SO₂NR | NRCONR | COOH |
| 5 | SO₂NR | SO₂NR | SO₂H | 5 | SO₂NR | NRCONR | SO₂H |
| 5 | SO₂NR | SO₂NR | Cl | 5 | SO₂NR | NRCONR | Cl |
| 5 | SO₂NR | SO₂NR | Br | 5 | SO₂NR | NRCONR | Br |
| 5 | SO₂NR | NRCNHNR | NH₂ | 5 | SO₂NR | NRCOO | NH₂ |
| 5 | SO₂NR | NRCNHNR | NHR | 5 | SO₂NR | NRCOO | NHR |
| 5 | SO₂NR | C≡C | COOH | 5 | SO₂NR | CH═CH | COOH |
| 5 | SO₂NR | C≡C | COH | 5 | SO₂NR | CH═CH | COH |
| 5 | SO₂NR | C≡C | COR | 5 | SO₂NR | CH═CH | COR |
| 5 | NRCONR | O | OH | 5 | NRCONR | S | OH |
| 5 | NRCONR | O | SH | 5 | NRCONR | S | SH |
| 5 | NRCONR | O | COOH | 5 | NRCONR | S | COOH |
| 5 | NRCONR | O | CONH₂ | 5 | NRCONR | S | CONH₂ |
| 5 | NRCONR | NR | CN | 5 | NRCONR | CR₅R₂ | CN |
| 5 | NRCONR | NR | NHR | 5 | NRCONR | CR₅R₂ | NHR |
| 5 | NRCONR | NR | COH | 5 | NRCONR | CR₅R₂ | COH |
| 5 | NRCONR | CONR | CONH₂ | 5 | NRCONR | SO₂NR | CONH₂ |
| 5 | NRCONR | CONR | COH | 5 | NRCONR | SO₂NR | COH |
| 5 | NRCONR | CONR | COR | 5 | NRCONR | SO₂NR | COR |
| 5 | NRCONR | NRCONR | OH | 5 | NRCONR | NRCNHNR | OH |
| 5 | NRCONR | NRCONR | SH | 5 | NRCONR | NRCNHNR | SH |
| 5 | NRCONR | NRCONR | COOH | 5 | NRCONR | NRCNHNR | COOH |
| 5 | NRCONR | NRCOO | F | 5 | NRCONR | C≡C | F |
| 5 | NRCONR | NRCOO | CN | 5 | NRCONR | C≡C | CN |
| 5 | NRCONR | CH═CH | Cl | 5 | NRCNHNR | O | Cl |
| 5 | NRCONR | CH═CH | Br | 5 | NRCNHNR | O | Br |
| 5 | NRCONR | CH═CH | NH₂ | 5 | NRCNHNR | OO | NH₂ |
| 5 | NRCNHNR | S | CONH₂ | 5 | NRCNHNR | NR | CONH₂ |
| 5 | NRCNHNR | S | CH═CH₂ | 5 | NRCNHNR | NR | CH═CH₂ |
| 5 | NRCNHNR | S | C≡CH | 5 | NRCNHNR | NR | C≡CH |
| 5 | NRCNHNR | S | NH₂ | 5 | NRCNHNR | NR | NH₂ |
| 5 | NRCNHNR | S | NHR | 5 | NRCNHNR | NR | NHR |
| 5 | NRCNHNR | S | COH | 5 | NRCNHNR | NR | COH |
| 5 | NRCNHNR | CR₅R₂ | SO₂H | 5 | NRCNHNR | CONR | SO₂H |
| 5 | NRCNHNR | CR₅R₂ | Cl | 5 | NRCNHNR | CONR | Cl |
| 5 | NRCNHNR | SO₂NR | SO₂H | 5 | NRCNHNR | NRCONR | SO₂H |
| 5 | NRCNHNR | SO₂NR | Cl | 5 | NRCNHNR | NRCONR | Cl |
| 5 | NRCNHNR | SO₂NR | Br | 5 | NRCNHNR | NRCONR | Br |
| 5 | NRCNHNR | SO₂NR | I | 5 | NRCNHNR | NRCONR | I |
| 5 | NRCNHNR | SO₂NR | F | 5 | NRCNHNR | NRCONR | F |
| 5 | NRCNHNR | SO₂NR | CN | 5 | NRCNHNR | NRCONR | CN |
| 5 | NRCNHNR | NRCNHNR | NH₂ | 5 | NRCNHNR | NRCOO | NH₂ |
| 5 | NRCNHNR | NRCNHNR | NHR | 5 | NRCNHNR | NRCOO | NHR |
| 5 | NRCNHNR | NRCNHNR | COH | 5 | NRCNHNR | NRCOO | COH |
| 5 | NRCNHNR | NRCNHNR | COR | 5 | NRCNHNR | NRCOO | COR |
| 5 | NRCNHNR | C≡C | OH | 5 | NRCNHNR | CH═CH | OH |
| 5 | NRCNHNR | C≡C | SH | 5 | NRCNHNR | CH═CH | SH |
| 5 | NRCNHNR | C≡C | I | 5 | NRCNHNR | CH═CH | I |
| 5 | NRCNHNR | C≡C | NHR | 5 | NRCNHNR | CH═CH | NHR |
| 5 | NRCOO | O | COOH | 5 | NRCOO | S | COOH |
| 5 | NRCOO | O | SO₂H | 5 | NRCOO | S | SO₂H |
| 5 | NRCOO | O | NHR | 5 | NRCOO | S | NHR |
| 5 | NRCOO | O | COH | 5 | NRCOO | S | COH |
| 5 | NRCOO | O | COR | 5 | NRCOO | S | COR |
| 5 | NRCOO | NR | OH | 5 | NRCOO | CR₅R₂ | OH |
| 5 | NRCOO | NR | SH | 5 | NRCOO | CR₅R₂ | SH |
| 5 | NRCOO | NR | COOH | 5 | NRCOO | CR₅R₂ | COOH |
| 5 | NRCOO | NR | SO₂H | 5 | NRCOO | CR₅R₂ | SO₂H |
| 5 | NRCOO | CONR | NHR | 5 | NRCOO | SO₂NR | NHR |
| 5 | NRCOO | CONR | COH | 5 | NRCOO | SO₂NR | COH |
| 5 | NRCOO | CONR | COR | 5 | NRCOO | SO₂NR | COR |
| 5 | NRCOO | NRCONR | OH | 5 | NRCOO | NRCNHNR | OH |
| 5 | NRCOO | NRCONR | SN | 5 | NRCOO | NRCNHNR | SH |
| 5 | NRCOO | NRCONR | COOH | 5 | NRCOO | NRCNHNR | COOH |
| 5 | NRCOO | NRCONR | COR | 5 | NRCOO | NRCNHNR | COR |
| 5 | NRCOO | NRCOO | OH | 5 | NRCOO | C≡C | OH |
| 5 | NRCOO | NRCOO | SH | 5 | NRCOO | C≡C | SH |
| 5 | NRCOO | NRCOO | COH | 5 | NRCOO | C≡C | COH |
| 5 | NRCOO | NRCOO | COR | 5 | NRCOO | C≡C | COR |
| 5 | NRCOO | CH═CH | N₃ | 5 | C≡C | O | N₃ |
| 5 | NRCOO | CH═CH | CONH₂ | 5 | C≡C | O | CONH₂ |
| 5 | NRCOO | CH═CH | COH | 5 | C≡C | O | COH |
| 5 | NRCOO | CH═CH | COR | 5 | C≡C | O | COR |
| 5 | C≡C | S | OH | 5 | C≡C | NR | OH |
| 5 | C≡C | S | SH | 5 | C≡C | NR | SH |
| 5 | C≡C | S | COOH | 5 | C≡C | NR | COOH |
| 5 | C≡C | S | NH₂ | 5 | C≡C | NR | NH₂ |
| 5 | C≡C | CR₅R₂ | SH | 5 | C≡C | CONR | SH |
| 5 | C≡C | CR₅R₂ | SO₂H | 5 | C≡C | CONR | SO₂H |
| 5 | C≡C | CR₅R₂ | N₃ | 5 | C≡C | CONR | N₃ |
| 5 | C≡C | CR₅R₂ | COR | 5 | C≡C | CONR | COR |
| 5 | C≡C | SO₂NR | NHR | 5 | C≡C | NRCONR | NHR |
| 5 | C≡C | SO₂NR | COH | 5 | C≡C | NRCONR | COH |
| 5 | C≡C | SO₂NR | COR | 5 | C≡C | NRCONR | COR |
| 5 | C≡C | NRCNHNR | CN | 5 | C≡C | NRCOO | CN |
| 5 | C≡C | NRCNHNR | CH═CH₂ | 5 | C≡C | NRCOO | CH═CH₂ |
| 5 | C≡C | NRCNHNR | C≡CH | 5 | C≡C | NRCOO | C≡CH |
| 5 | C≡C | C≡C | COOH | 5 | C≡C | CH═CH | COOH |
| 5 | CH═CH | O | OH | 5 | CH═CH | S | OH |
| 5 | CH═CH | O | C≡CH | 5 | CH═CH | S | C≡CH |
| 5 | CH═CH | O | NH₂ | 5 | CH═CH | S | NH₂ |
| 5 | CH═CH | O | NHR | 5 | CH═CH | S | NHR |
| 5 | CH═CH | NR | NHR | 5 | CH═CH | CR₅R₂ | NHR |
| 5 | CH═CH | NR | COH | 5 | CH═CH | CR₅R₂ | COH |
| 5 | CH═CH | NR | COR | 5 | CH═CH | CR₅R₂ | COR |
| 5 | CH═CH | CONR | Br | 5 | CH═CH | SO₂NR | Br |
| 5 | CH═CH | CONR | COR | 5 | CH═CH | SO₂NR | COR |
| 5 | CH═CH | NRCONR | Br | 5 | CH═CH | NRCNHNR | Br |
| 5 | CH═CH | NRCOO | OH | 5 | CH═CH | C≡C | OH |
| 5 | CH═CH | CH═CH | COOH | 5 | CH═CH | CH═CH | CH═CH₂ |
| 5 | CH═CH | CH═CH | SO₂H | 5 | CH═CH | CH═CH | C≡CH |

R, R₁, and R₂ = hydrogen, alkyl, alkenyl, alkynyl, aryl, and heterocyclic

TABLE 8

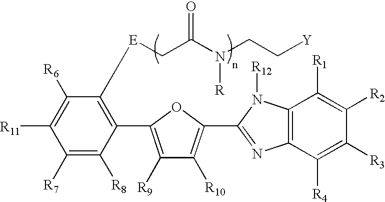

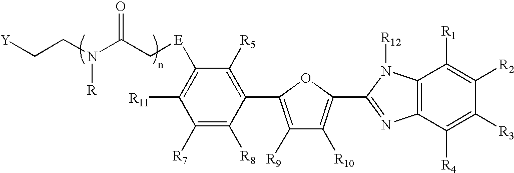

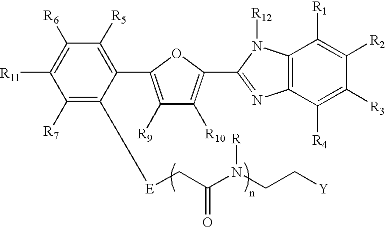

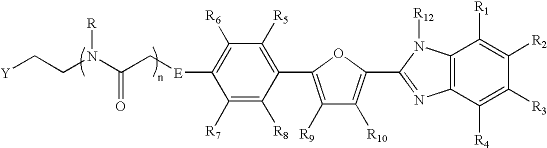

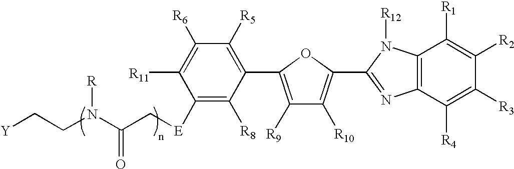

The variables E, Y, and n can have the values provided in Table 6 above. R in the compounds is alky, alkenyl, alkynyl, aromatic, or heterocyclic.

TABLE 9

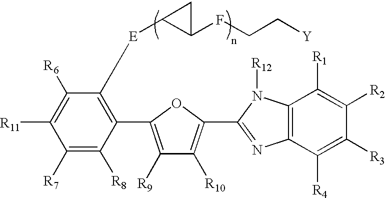

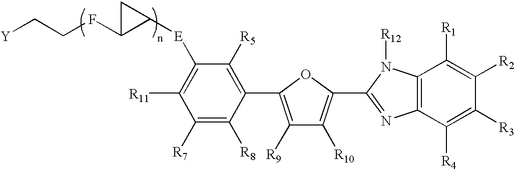

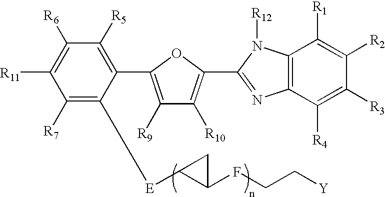

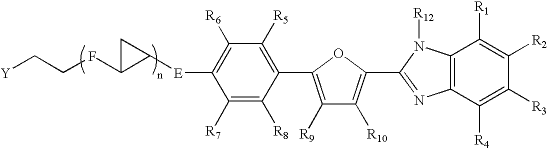

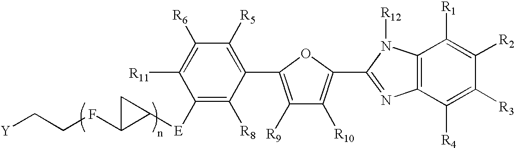

The variables E, F. Y. and n can have the # values provided in Table 7 above.

TABLE 10

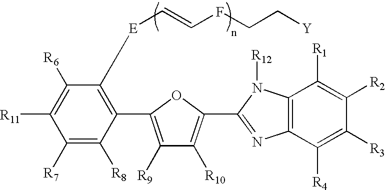

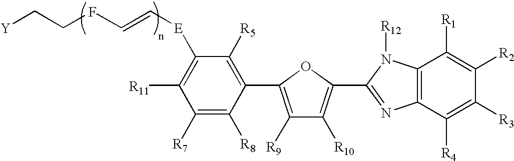

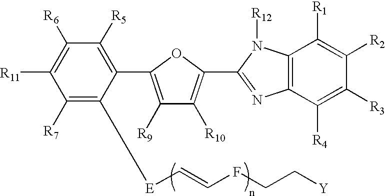

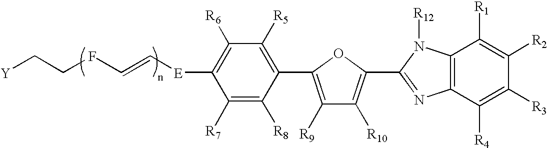

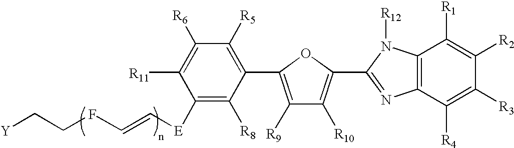

The variables E, F, Y, and n can have the values provided in Table 7 above.

TABLE 11

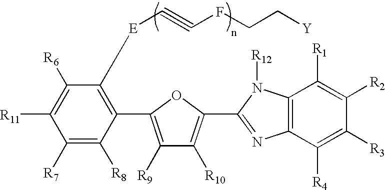

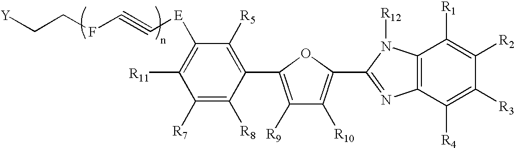

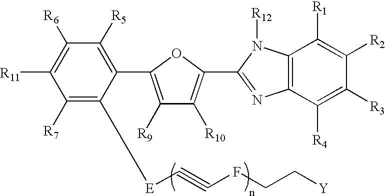

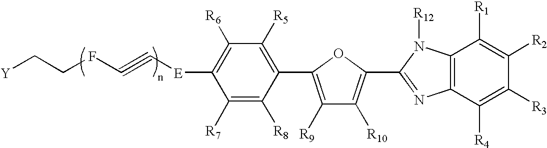

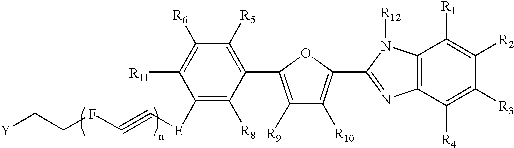

The variables E, F, Y, and n can have the values provided in Table 6 above.

### Example 12

### Preparation of Bi-ligand Libraries of the present invention

This example describes preparation of a bi-ligand library from common ligand mimics of the invention according to the reaction scheme presented in FIG. 6. Compound numbers correspond to the numbers in the figure.

HOBt resin (40 mg, 1.41 mmol/g, Argonaut) was swelled in a mixture of 150 μl dry THF and 50 μl of dry DMF. The resin then was added to a solution of compound 6 (2 eq., 0.226 mmol) dissolved in 153 μl of dry DMF and 10 eq, 0.564 mmol, of DIC. The solution was shaken at room temperature overnight and then washed three times with dry DMF and three times with dry THF.

The resin was added to a solution of the amine (0.4 eq, 0.226 mmol) dissolved in 200 μl dry DMF. The mixture was again shaken at room temperature overnight. The resin was filtered and washed once with 500 μl of dry DMF. The filtrate was collected and vacuum dried to provide compound 13. Amines that have been used for the development of bi-ligand libraries of the invention using this reaction are provided in Table 4.

### Example 13

### Screening of Selected Benzimidazole compounds for Binding to Oxidoreductases

This example describes the screening of two benzimidazole common ligand mimics for binding activity to a variety of dehydrogenases and oxidoreductases.

The benzimidazole compounds 4-[5-(1H-benzoimidazol-2-yl)-furan-2-yl] benzoic acid (compound 6b) and 4-[5-(5-nitro-1H-benzoimidazol-2-yl)-furan-2-yl]-benzoic acid (compound 6d) were produced following the method of Examples 3 and 5. The compounds were screened for binding to the following enzymes: dihydrodipicolinate reductase (DHPR), dihydrofolate reductase (DHFR), aldose reductase (AR), lactate dehydrogenase (LDH), inosine-5′-monophosphate dehydrogenase (IMPDH), alcohol dehydrogenase (ADH), 1-deoxy-D-xylulose-5-phosphate reductase (DOXPR), HMG CoA reductase (HMGCoAR), and glyceraldehyde-3-phosphate dehydrogenase (GAPDH).

### DHPR

For DHPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DHPR was diluted in 10 mM HEPES at a pH of 7.4. DHPS (dihydrodipicolinate synthase) was not diluted and was stored in eppindorf tubes.

| | Stock | Final | Volume needed |
|---|---|---|---|
| ddH₂O | | | 798 μl |
| HEPES (pH 7.8) | 1 M | 0.1 M | 100 μl |
| Pyruvate | 50 mM | 1 mM | 20 μl |
| NADPH | 1 mM | 6 μM | 6 μl |
| L-ASA | 28.8 mM | 40 μM | 13.9 μl |
| DHPS | 1 mg/ml | | 7 μl |
| DHPR | 1:1000 dilution of | | 5 μl |
| | 1 mg/ml stock | | |
| Inhibitor | 15 mM | 100 μM | 6.7 μl |
| | | | (0.67 DMSO) |
| DMSO | 100% | 5% | 43.3 μl |

Total Assay volume = 1000 μl

The L-ASA (L-aspartate semialdehyde) solution was prepared in the following manner. 180 μM stock solution of ASA was prepared. 100 μl of the ASA stock solution was mixed with 150 μl of concentrated NaHCO₃ and 375 μl of H₂O. For use in the assay, 28.8 mM L-ASA was equal to 625 μl of the solution. The L-ASA stock Aft solution was kept at a temperature of −20° C. After dilution, the pH of the 28.8 mM solution was checked and maintained between 1 and 2.

The DHPS reaction was monitored at 340 nm prior to and after addition of the inhibitor to detect background reaction with the inhibitor. The solution for background detection was a 945 μl solution containing 0.1 HEPES (pH 7.8), 1 mM pyruvate, 6 μM NADPH, 40 μM L-ASA, and 7 μl of 1 mg/ml DHPS at 25° C. in the volumes provided above. The sample solution was then mixed and incubated for 10 minutes. Next, 500 nM solutions of the inhibitors and enough DMSO to provide a final DMSO concentration of 5% of the total assay volume were added. The solution was mixed and incubated for an additional 6 minutes.

In DHPR samples, 5 μl of the diluted DHPR enzyme were added. The sample was mixed for 20 seconds and then the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 μM was substituted for inhibitor to yield 70 to 80% inhibition. The substrate was kept at a level at least 10 times the Km. The final concentration of L-ASA was about 1 mM.

### LDH

For LDH analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 780 μl |
| HEPES (pH 7.4) | 1 M | 0.1 M | 100 μl |
| Pyruvate | 50 mM | 2.5 mM | 50 μl |
| NADH | 1 mM | 10 μM | 10 μl |
| LDH | 1:2000 dilution of | | 10 μl |
| | 1 mg/ml stock | | |
| Inhibitor | 15 mM | 100 μM | 6.7 μl (0.67% DMSO) |
| | | | (0.67% DMSO) |
| DMSO | 100% | 5% | 43.3 μl |

Total Assay volume = 1000 μl

The LDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 10 μM NADH, and 2.5 mM of pyruvate. The reaction was then initiated with 10 μl of LDH from Rabbit Muscle (0.5 μg/ml; 1:2000 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.3 μM was substituted for inhibitor to yield 50 to 70% inhibition. The substrate was kept at a level at least 10 times the Km.

### ADH

For ADH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| DdH₂O | | | 787 μl |
| HEPES (pH 8.0) | 1 M | 0.1 M | 100 μl |
| EtOH | 10 M | 130 mM | 13 μl |
| NAD+ | 2 mM | 80 μM | 40 μl |
| ADH | 1:400 dilution of | | 10 μl |
| | 1 mg/ml stock | | |
| Inhibitor | 15 mM | 100 μM | 6.7 μl |
| | | | (0.67% DMSO) |
| DMSO | 100% | 5% | 43.3 μl |

Total Assay volume =1000 μl

The ADH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 990 μl of a solution containing 0.1 M HEPES, pH 8.0, 80 μM NAD+, and 130 mM of ethanol. The reaction was then initiated with 10 μl of ADH from Bakers Yeast (3.3 μg/ml; 1:400 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 15.5 μM was substituted for inhibitor to yield 50 to 60% inhibition. The substrate was kept at a level at least 10 times the Km. The final concentration of pyruvate was about 2.5 mM.

Where only a simple read was desired, as in the case of NAD+ concentration determination, 13 μl (10 M stock) of ethanol was used to drive the reaction, and 10 μl of pure enzyme (1 mg/ml) was used. NAD+was soluble at 2 mM, which allowed the concentration determination step to be skipped. In this situation, the procedure was as follows. All of the ingredients except for the enzyme were mixed together. The solution was mixed well and the absorbance at 340 nm read. The enzyme was added and read again at OD 340 after the absorbance stopped changing, generally 10 to 15 minutes after the enzyme was added.

### DHFR

For DHFR analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below. H₂ folate was dissolved in DMSO to about 10 mM and then diluted with water to a concentration of 0.1 mM.

| | Stock | Final | Volume needed |
|---|---|---|---|
| ddH₂O | | | 616 μl |
| Tris-HC1 (pH 7.0) | 1 M | 0.1 M | 100 μl |
| KCl | 1 mM | 0.15 M | 150 μl |
| H₂ Folate | 0.1 mM | 5 μM | 50 μl |
| NADPH | 2 mM | 52 μM | 26 μl |
| DHFR | 1:85 dilution of | | 8 μl |
| 4 mg/ml stock | | | |
| Inhibitor | 15 mM | 100 μM | 6.7 μl |
| | | | (0.67% DMSO) |
| DMSO | 100% | 5% | 43.3 gl |

Total Assay volume =1000 μl

The DHFR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 992 μl of a solution containing 0.1 M Tris-HCl, pH 7.0, 150 mM KCl, 5 μM H₂ folate, and 52 μM NADH. The oxidation reaction was then initiated with 8 μl of DHFR (0.047 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 always contained the control reaction (no inhibitor), and cuvette #2 always contained the positive control reaction in which Cibacron Blue at 3 μM was substituted for inhibitor to yield 50 to 70% inhibition. The substrate was kept at a level at least 10 times the Km.

### DOXPR

For DOXPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DOXPR was diluted in 10 mM HEPES at a pH of 7.4.

| | Stock | Final | Volume needed |
|---|---|---|---|
| ddH₂O | | | 707 μl |
| HEPES (pH 7.4) | 1 M | 0.1 M | 100 μl |
| DOXP | 10 mM | 1.15 mM | 115 μl |
| NADPH | 1 mM | 8 μM | 8 μl |
| MnC12 | 100 mM | 1 mM | 10 μl |
| DOXPP. | 1:200 dilution of | | 10 μl |
| 2 mg/ml stock | | | |
| Inhibitor | 15 mM | 100 μM | 6.7 μl |
| | | | (0.67% DMSO) |
| DMSO | 100% | 5% | 43.3 μl |

Total Assay volume =1000 μl

The DOXPR reaction was monitored at 340 nm prior to and after addition of the inhibitor to detect background reaction with the inhibitor. Solutions of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 1 mM MnCl₂1.15 mM DOXP, and 8 μM NADPH. The oxidation reaction was then initiated with 10 μl of DOXP reductoisomerase (10 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.32 μM was substituted for inhibitor to yield 70 to 80% inhibition. The substrate was kept at a level at least 10 times the Km.

### GAPDH

For GAPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 739 μl |
| Triethanolamine | 1 M | 25 mM | 125 μl |
| (pH 7.5) | | | |
| GAP | 50 mM | 145 μM | 3 μl |
| NAD+ | 5 mM | 0.211 mM | 42 μl |
| Sodium Arsenate | 200 mM | 5 mM | 25 μl |
| 2-BME | 500 mM | 3 mM | 6 μl |
| GAPDH | 1:200 dilution of | | 10 μl |
| | 1 mg/ml stock | | |
| Inhibitor | 12.5 mM | 100 μM | 8 μM (total 5% |
| | | | DMSO) |
| DMSO | 100% | 5% | 42 μl |

Total Assay volume = 1000 μl

The GAPDH reaction was monitored at 340 nm prior to and after addition of the inhibitor to detect background reaction with the inhibitor. Solutions of 100 μl of the inhibitors incubated for 6 minutes at 25° C. in a 990 μl of a solution containing 125 mM triethanolamine, pH 7.5, 145 μM glyceraldehyde 3-phosphate (GAP), 0.211 mM NAD, 5 mM sodium arsenate, and 3 mM β-metcaptoethanol (2-BME). The reaction was then initiated with 10 gl of E. coli GAPDH (1:200 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in a cuvette was about 5% of the total assay volume. Cuvette #1 contained the control reaction (no inhibitor).

GAP for use in this experiment was deprotected from the diethyl acetal in the following manner. Water was boiled in recrystallizing dish. Dowex (1.5 mg) and GAP (200 mg; SIGMA G-5376) were weighed and placed in a 15 ml conical tube. The Dowex and GAP were resuspended in 2 ml dH₂O, followed by shaking of the tube until the GAP dissolved. The tube was then immersed, while shaking, in the boiling water for 3 minutes. Next, the tube was placed in an ice bath to cool for 5 minutes. As the sample cooled, a resin settled to the bottom of the test tube, allowing removal of the supernatant with a pasteur pipette. The supernatant was filtered through a 0.45 or 0.2 μM cellulose acetate syringe filter.

The filtered supernatant was retained, and another 1 ml of dH₂O was added to the resin tube. The tube was then shaken and centrifuged for 5 minutes at 3,000 rpm. The supernatant was again removed with a pasteur pipette and passed through a 0.45 or 0.2 μM cellulose acetate syringe filter. The two supernatant aliquots were then pooled to provide a total GAP concentration of about 50 mM. The GAP was then divided into 100 μl aliquots and stored at −20° C. until use.

### IMPDH

For IMPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 447 μl |
| Tris-HCl (pH 8.0) | 1 M | 0.1 M | 100 μl |
| KCl | 1 M | 0.25 M | 250 μl |
| NAD+ | 2 mM | 30 μM | 15 μl |
| IMP | 6 mM | 600 μM | 100 μl |
| Glycerol | 10% | 0.3% | 30 μl |
| IMPDH | 0.75 mg/ml, undiluted | | 8 μl |
| Inhibitor | 15 mM | 100 μM | 6.7 μl |
| | | | (0.67% DMSO) |
| DMSO | 100% | 5% | 43.3 μl |

Total Assay volume = 1000 μl

The IMPDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 37° C. in a 992 μl of a solution containing 0.1 M Tris-HCl, pH 8.0, 0.25 M KCl, 0.3% glycerol, 30 μM NAD+, and 600 μM IMP (inosine monophosphate). The reaction was then initiated with 8 μl of IMPDH (0.75 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue was substituted for inhibitor. The substrate was kept at a level at least 10 times the Km.

### HMGCoAR

For HMGCoAR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. The enzyme was diluted in 1 M NaCl. To prepare the dilution buffer, 10 μl of HMGCoAR (1 mg/ml) was mixed with 133 μl of 3 M NaCl solution and 257 μl of 25 mM KH₂PO₄ buffer (pH 7.5; containing 50 mM NaCl, μl mM EDTA (ethylenediaminetetraacetic acid), and 5 mM DTT (dithiothreitol).

| | Stock | Final | Volume needed |
|---|---|---|---|
| ddH₂O | | | 841 μl |
| KH₂PO₄ (pH 7.5) | 1 M | 25 mM | 25 μl |
| HMGCoA | 10 mM | 160 mM | 16 μl |
| NADPH | 1 mM | 13 μM | 13 μl |
| NaCl | 1 M | 50 mM | 50 μl |
| EDTA | 50 mM | 1 mM | 20 μl |
| DTT | 500 mM | 5 mM | 10 μl |
| HMGCoAR | 1:40 dilution of | | 5 μl |
| | 0.65 mg/ml stock | | |
| Inhibitor | 10 mM | 100 μM | 10 μl |
| DMSO | 100% | 2% | 10 μl |

Total Assay volume = 1000 μl

The HMGCoAR reaction was monitored at 340 nm prior to and after addition of the inhibitor to detect background reaction with the inhibitor. Solutions of 100 μM of the inhibitors in DMSO were prepared to provide a final DMSO concentration of 2% of the total assay volume. These solutions were incubated for 6 minutes at 25° C. in a 994 μl of a solution containing 25 mM KH₂PO₄, pH 7.5, 160 μM HMGCoA, 13 μM NADPH, 50 mM NaCl, 1 mM EDTA, and 5 mM DTT. The reaction was then initiated with 5 μl of HMGCoAR enzyme (1:40 dilution of 0.65 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.05 μM was substituted for inhibitor to yield 50 to 70% inhibition. The substrate was kept at a level at least 10 times the Km.

### IPMDH

For IPMDH analysis, the compounds were screened using a kinetic protocol that spectrophotometrically evaluates reduction of NAD.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 407 μl |
| KH₂PO₄ (pH 7.6) | 1 M | 20 mM | 20 μl |
| KCl | 1 M | 0.3 M | 300 μl |
| MNCl₂ | 20 mM | 0.2 mM | 10 μl |
| NAD | 3.3 mM | 109 μM | 33 μl |
| IPM | 2 mM | 340 μM | 170 μl |
| E. coli IPMDH | 1:300 dilution of | 10 μl | |
| | 2.57 mg/ml | stock | |
| Inhibitor | 16 mM | 200 μM | 12.5 μl |
| DMSO | 100% | 5% | 37.5 μl |

Total Assay volume = 1000 μl

The IPMDH reaction was monitored at 340 nm prior to and after addition of the inhibitor to detect background reaction with the inhibitor. Inhibitor was incubated for 5 minutes at 37° C. in a 990 μl of a solution containing 20 mM potassium phosphate, pH 7.6, 0.3 M potassium chloride, 0.2 mM manganese chloride, 109 μM NAD, and 340 μM DL-threo-3-isopropylmalic acid (IPM). The reaction was then initiated with 10 μl of E. coli isopropylmalate dehydrogenase (1:300 dilution of 2.57 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in the cuvette was 5% of the total assay volume. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

### AR

For AR analysis, the compounds were screened using a kinetic protocol that spectrophotometrically measures enzyme activity.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 565.5 μl |
| KH₂PO₄ (pH 7.5) | 1 M | 100 mM | 100 μl |
| Ammonium Sulfate | 1 M | 0.3 M | 300 μl |
| EDTA | 500 mM | 1 mM | 2 μl |
| NADPH | 1 mM | 3.8 μM | 3.8 μl |
| Glyceraldehyde | 100 mM | 171 μM | 1.7 μl |
| DTT | 100 mM | 0.1 mM | 1 μl |
| Human ALDR | 1:5 dilution of | | 10 μl |
| | 0.55 mg/ml stock | | |
| Inhibitor | 12.5 mM | 200 μM | 16 μl |

Total Assay volume = 1000 μl

The AR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 5 minutes at 25° C. in a 990 μl of a solution containing 100 mM potassium phosphate, pH 7.5, 0.3 M ammonium sulfate, 1.0 mM ethylenediaminetetraacetic acid (EDTA), 3.8 μM B-Nicotinamide adenine dinucleotide phosphate (NADPH), 171 μM DL-glyceraldehyde and 0.1 mM DL-dithiothreitol. The reaction was then initiated with 10 μl of Human Aldose Reductase (1:5 dilution of 0.55 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final DMSO concentration in the cuvette was 5%. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

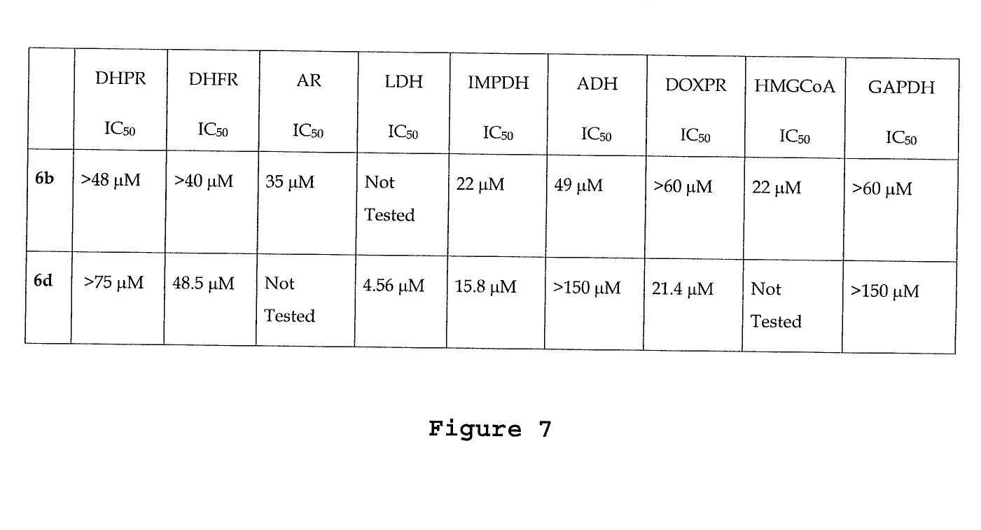

IC₅₀ data for these compounds are presented in FIG. 7. The compound 4-[5-(1H-benzoimidazol-2-yl)-furan-2-yl] benzoic acid demonstrated an IC₅₀ of 35 μM for AR, of 22 μM for IMPDH, of 49 μM for ADH, and of 22 μM for HMGCoAR. The IC50 value for DHPR was greater than 48 μM, and the IC₅₀ value for DHFR was greater than 40 μM. The IC₅₀ value for DOXPR and GAPDH was greater 60 μM.

The compound 4-[5-(5-nitro-1H-benzoimidazol-2-yl)-furan-2-yl]-benzoic acid demonstrated an IC₅₀ of 48.5 μM for DHFR, 4.56 μM for LDH, 15.8 μM for IMPDH, 21.4 μM DOXPR. Additionally, the IC₅₀ value for DHPR was greater than 75 μM, and the IC₅₀ value for ADH and GAPDH was greater than 150 μM.

### Example 14

### Screening of Selected Benzimidazole Compounds for Binding to Dihydrodipicolinate Reductase (DHPR)

This example describes the screening of benzimidazole common ligand mimics for binding activity to a variety of dehydrogenases and oxidoreductases.

The following compounds were produced by the methods of Examples 3, 5, 2, and 4, respectively: 4-[5-(1H-benzoimidazol-2-yl)-furan-2-yl] benzoic acid, 4-[5-(5-nitro-1H-benzoimidazol-2 -yl)-furan-2-yl]-benzoic acid, 4-[5-(5-nitro-1H-benzoimidazol-2-yl)-furan-2-yl]-benzoic acid methyl ester, 4-[5-(4-methyl-1-benzimidazol-2-yl)-furan-2-yl]-benzoic acid, and 4-[5-(5-methyl-1H-benzoimidazol-2-yl)furan-2-yl]-benzoic acid.

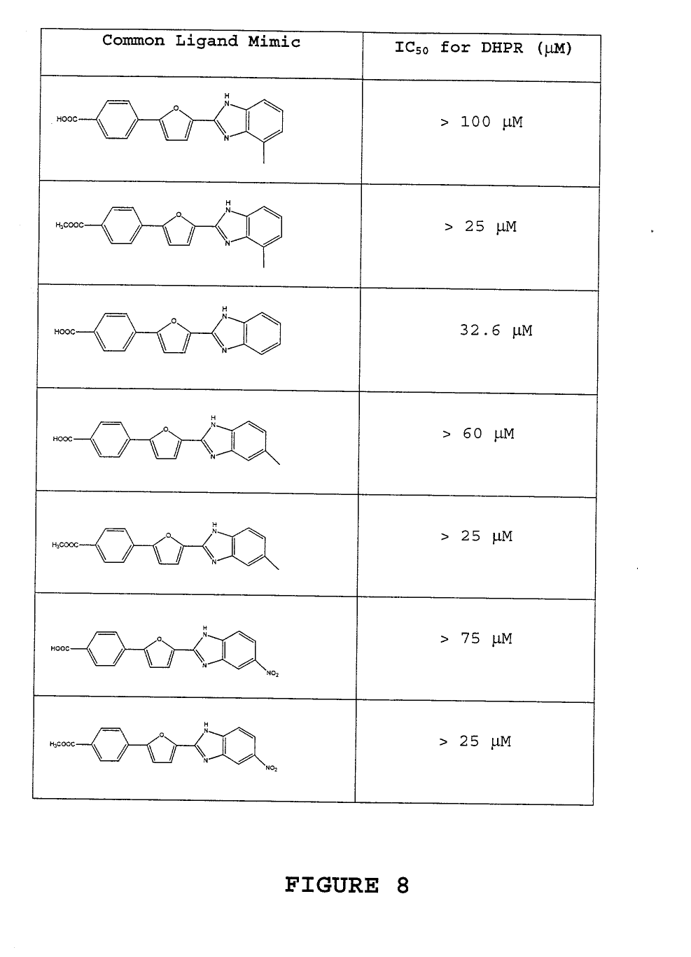

The compounds were screened for binding to DHPR using the assay method described in Example 13. IC₅₀ data for these compounds are presented in FIG. 8. The compound 4-[5-(1H-benzoimidazol-2-yl)-furan-2-yl] benzoic acid demonstrated an IC₅₀ of 32.6 μM. The compound 4-[5-(5-nitro-1H-benzoimidazol-2-yl)-furan-2-yl]-benzoic acid demonstrated and IC₅₀ of greater than 75 μM. The IC₅₀ values for the other compounds tested are as follows: 4-[5-(5-nitro-1H-benzoimidazol-2-yl)-furan-2-yl]-benzoic acid methyl ester (greater than 25 μM); 4-[5-(4-methyl-1H-benzimidazol-2-yl)-furan-2-yl]-benzoic acid (greater than 100 μM); 4-[5-(4-methyl-1H-benzimidazol-2-yl)-furan-2-yl]-benzoic acid (greater than 25 μM); 4-[5-(5-methyl-1H-benzoimidazol-2-yl)furan-2-yl]s-benzoic acid (greater than 60 μM); and 4-[5-(5-methyl-1-benzoimidazol-2-yl)furan-2-yl]-benzoic acid (greater than 25 μM).

### Example 15

### Screening of Biligands for Binding to Dihydrodipicolinate Reductase (DHPR)

This example describes the screening of bi-ligands having benzimidazole common ligand mimics for binding activity to dihydrodipicolinate reductase (DHPR).

Bi-ligands were produced by the methods of Examples 8 and 9. The bi-ligands were screened for binding to DHPR using the assay method described in Example 13. IC₅₀ data for these compounds are presented in FIG. 9. The bi-ligand 21a exhibited IC₅₀ value for dihydrodipicolinate reductase (DHPR) of about 0.758 μM. Bi-ligand 21b exhibited an IC₅₀ value for DHPR of greater than 1.6 μM.

We claim:

1. A compound comprising the formula: 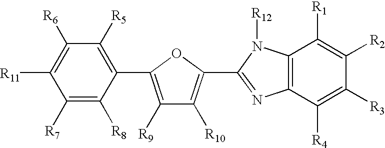
wherein
R₁ to R₁₁ each independently are selected from the group consisting of H, alkyl, alkenyl, alkynyl, aryl, heterocycle, COOH, COOAlkyl, CONR₁₃R₁₄, C(O)R₁₅,OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X;
R₁₂ is H, alkyl, alkenyl, alkynyl, aryl, or heterocycle; and
R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring,
with the proviso that at least one of R₁ to R₁₀ is other than hydrogen.

2. The compound of claim 1, wherein at least one of R₁ to R₁₁ is COOH.

3. The compound of claim 1, wherein at least one of R₁ to R₁₁ is OH.

4. The compound of claim 1, wherein at least one of R₁ to R₁₁ is OAlkyl.

5. The compound of claim 1, wherein at least one of R₁ to R₁₁ is COOAlkyl.

6. The compound of claim 1, wherein at least one of R₁ to R₁₁ is NHCOQR₁₅.

7. The compound of claim 1, wherein two or more of R₁ to R₁₁ are substituted.

8. The compound of claim 1, having the formula 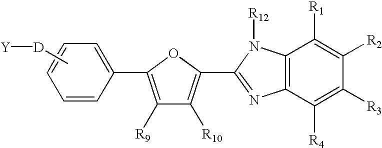
wherein
D is alkylene, alkenylene, alkynylene, aryl, or heterocycle; and
Y is OH, NHR₁₅, SH, COOH, SO₂OH, X, CN, N₃, CONH₂, CONHR₁₅, C≡CH, or CH═CH₂.

9. The compound of claim 1, having the formula 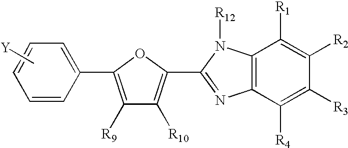
wherein Y is OH, NHR₁₅, SH, COOH, SO₂OH, X, CN, N₃, CONH₂, CONHR₁₅, CH≡CH, or C═CH₂.

10. The compound of claim 1, having the formula 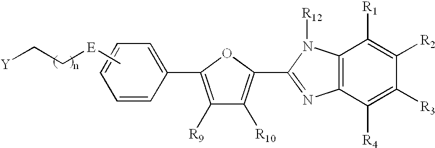
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

11. The compound of claim 1, having the formula 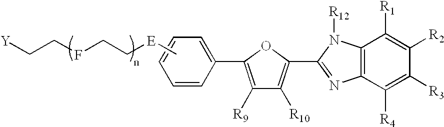
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

12. The compound of claim 1, having the formula 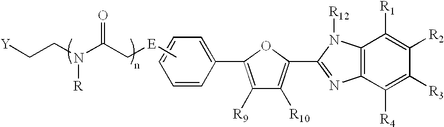
wherein
E is present or absent and when present is O, S, NH, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

13. The compound of claim 1, having the formula 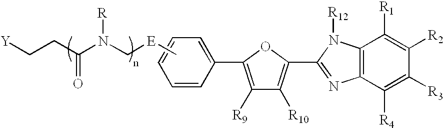
wherein
E is present or absent and when present is O, S, NH, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

14. The compound of claim 1, having the formula 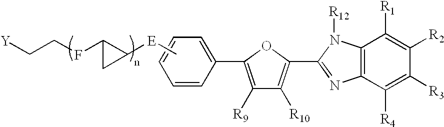
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

15. The compound of claim 1, having the formula 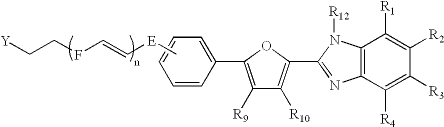
wherein p2 E is present or absent and when present is O, S, NR₁₅, CR₁₄C₁₅, CONR₁₅, SO₂NR₁₅, NR₁₄, CONR₁₅, NR₁₄CNHNR₁₅, NR₁₅COO, C≡C, or CH═CH;
F each independently is selected from the group consisting of CR₁₄C₁₅, CONR₁₅, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

16. The compound of claim 1, having the formula 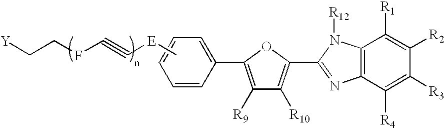
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of CR₁₄C₁₅, CONR₁₅, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

17. The compound of claim 1, having the formula 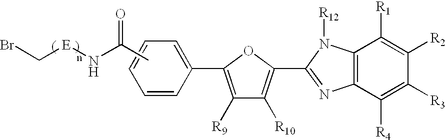
wherein
E is present or absent and when present is O, S, NR₁₅, CR₁₄C₁₅, CONR₁₅, SO₂NR₁₅, NR14CONR₁₅, NR₁₄CNHNR₁₅, NR₁₅COO, C≡C, or CH═CH; and
n is an integer between 0 and 5, inclusive.

18. The compound of claim 1, having the formula 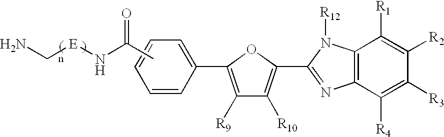
wherein
E is present or absent and when present is CH₂, CH₂CH₂OCH or CH₂CH₂SCH and n is an integer between 1 and 10, inclusive.

19. The compound of claim 18, wherein n is greater than 4 and E is CH₂CH₂OCH or CH₂CH₂SCH.

20. The compound of claim 1, having the formula

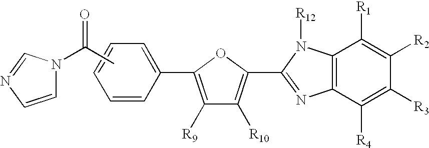

21. A combinatorial library of two or more compounds comprising a common ligand variant of a compound of the formula: 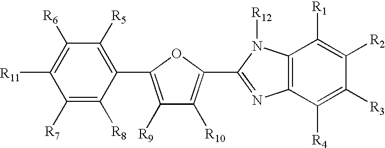
wherein
R₁ to R₁₁ each independently are selected from the group consisting of H, alkyl, alkenyl, alkynyl, aryl, heterocycle, COOH, COOAlkyl, CONR₁₃R₁₄, C(O)R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅,CN, or X;
R₁₂ is H, alkyl, alkenyl, alkynyl, aryl, or heterocycle; and
R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring.

22. The combinatorial library of claim 21, wherein at least one of R₁ to R₁₁ is COOH.

23. The combinatorial library of claim 21, wherein at least one of R₁ to R₁₁ is OH.

24. The combinatorial library of claim 21, wherein at least one of R₁ to R₁₁ is OAlkyl.

25. The combinatorial library of claim 21, wherein at least one of R₁ to R₁₁ is COOAlkyl.

26. The combinatorial library of claim 21, wherein at least one of R₁ to R₁₁ is NHCOR₇.

27. The combinatorial library of claim 21, wherein two or more of R₁ to R₁₁ are substituted.

28. The combinatorial library of claim 21, having the formula 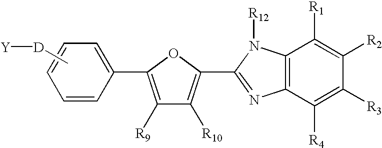
wherein
D is alkylene, alkenylene, alkynylene, aryl, or heterocycle; and
Y is OH, NHR₁₅,SH, COOH, SO₂OH, X, CN, N₃, CONH₂, CONHR₁₅, C≡CH, or CH═CH₂.

29. The combinatorial library of claim 21, having the formula 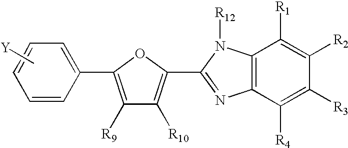
wherein Y is OH, NHR₁₅,SH, COOH, SO₂OH, X, CN, N₃, CONH₂, CONHR₁₅, C≡CH, or CH═CH₂.

30. The combinatorial library of claim 21, having the formula 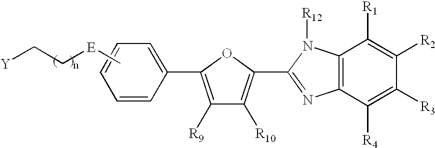
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

31. The combinatorial library of claim 21, having the formula 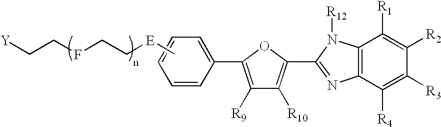
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR14C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

32. The combinatorial library of claim 21, having the formula 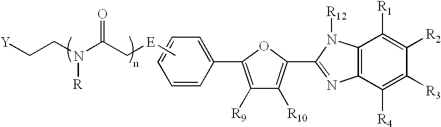
wherein
E is present or absent and,when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

33. The combinatorial library of claim 21, having the formula 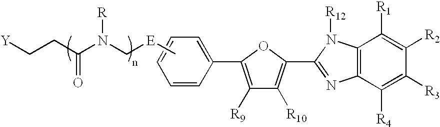
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

34. The combinatorial library of claim 21, having the formula 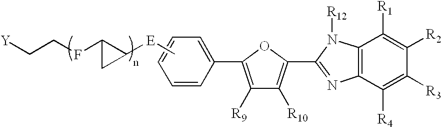
wherein
E is present or absent and when present is O, S, NR₁₅, CR₁₄C₁₅CONR₁₅, SO₂NR₁₅, NR₁₄CONR₁₅, NR₁₄CNHNR₁₅, NR15COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH , X, CN, N₃, CONH₂, CONHR₁₅, C≡CH, or CH═CH₂; and
n is an integer between 0 and 5, inclusive.

35. The combinatorial library of claim 21, having the formula 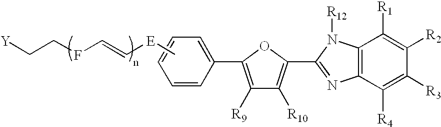
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO C≡C, or CH═CH;
F each independently is selected from the group consisting of CR₁₄C₁₅, CONR₁₅, C≡C, and CH═CH;
Y is OH, NHR₁₅, SH, COOH, SO₂OH , X, CN, N₃, CONH₂, CONHR₁₅, C≡CH, or CH═CH₂; and
n is an integer between 0 and 5, inclusive.

36. The combinatorial library of claim 21, having the formula 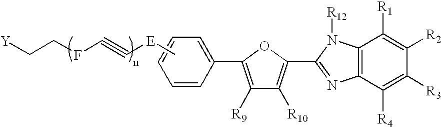
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅,CR₁₄C₁₅, CONR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

37. The combinatorial library of claim 21, having the formula 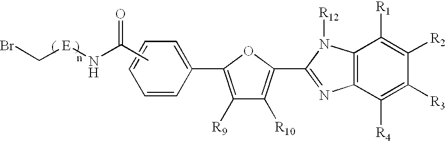
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH; and
n is an integer between 0 and 5, inclusive.

38. The combinatorial library of claim 21, having the formula 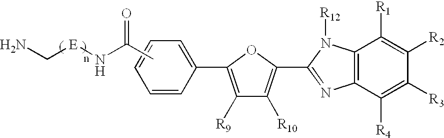
wherein
E is present or absent and when present is CH₂, CH₂CH₂OCH or CH₂CH₂SCH and n is an integer between 1 and 10, inclusive.

39. The combinatorial library of claim 38, wherein n is greater than 4 and E is CH₂CH₂OCH or CH₂CH₂SCH.

40. The combinatorial library of claim 21, having the formula

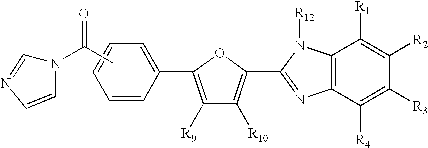

41. A combinatorial library of two or more bi-ligands comprising the reaction product of a specificity ligand and a common ligand mimic having the formula: 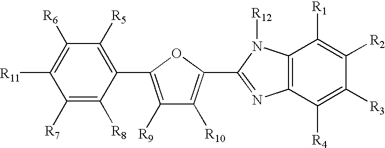
wherein
R₁ to R₁₁ each independently are selected from the group consisting of H, alkyl, alkenyl, alkynyl, aryl, heterocycle, COOH, COOAlkyl, CONR₁₃R₁₄, C (O) R₁₅, OH, OAlkyl, OAc, SH, SR₁₅, SO₃H, S(O)R₁₅, SO₂NR₁₃R₁₄, S(O)₂R₁₅, NH₂, NHR₁₅, NR₁₃R₁₄, NHCOR₁₅, N₃, NO₂, PH₃, PH₂R₁₅, PO₄H₂, H₂PO₃, H₂PO₂, HPO₄R₁₅, PO₂R₁₄R₁₅, CN, or X;
R₁₂ is H, alkyl, alkenyl, alkynyl, aryl, or heterocycle; and
R₁₃, R₁₄, and R₁₅ each independently are hydrogen, alkyl, alkenyl, alkynyl, aryl, or heterocycle, or R₁₃ and R₁₄ together with the nitrogen atom to which they are attached can be joined to form a heterocyclic ring.

42. The combinatorial library of claim 41, wherein at least one of R₁ to R₁₁ is COOH.

43. The combinatorial library of claim 41, wherein at least one of R₁ to R₁₁ is OH.

44. The combinatorial library of claim 41, wherein at least one of R₁ to R₁₁ is OAlkyl.

45. The combinatorial library of claim 41, wherein at least one of R₁ to R₁₁ is COOAlkyl.

46. The combinatorial library of claim 41, wherein at least one of R₁ to R₁₁ is NHCOR₇.

47. The combinatorial library of claim 41, wherein two or more of R₁ to R₁₁ are substituted.

48. The combinatorial library of claim 41, wherein at least one of the compounds is a common ligand variant of a compound having the formula: 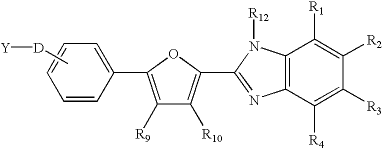
wherein
D is alkylene, alkenylene, alkynylene, aryl, or heterocycle; and
Y is OH, NHR₁₅, SH, COOH, SO₂OH, X, CN, N₃, CONH₂, CONHR₁₅, C≡CH, or CH═CH₂.

49. The combinatorial library of claim 41, wherein at least one of the compounds is a common ligand variant of a compound having the formula: 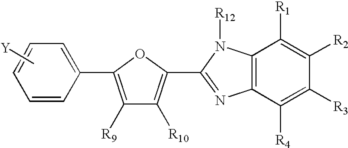
wherein Y is OH, NHR₁₅, SH, COOH, SO₂OH, X, CN, N₃, CONH₂, CONHR₁₅, C≡CH, or CH═CH₂.

50. The combinatorial library of claim 41, wherein at least one of the compounds is a common ligand variant of a compound having the formula: 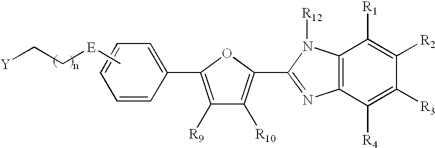
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

51. The combinatorial library of claim 41, wherein at least one of the compounds is a common ligand variant of a compound having the formula: 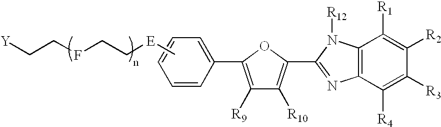
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or C═CH₂; and
n is an integer between 0 and 5, inclusive.

52. The combinatorial library of claim 41, wherein at least one of the compounds is a common ligand variant of a compound having the formula: 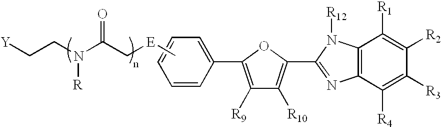
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

53. The combinatorial library of claim 41, wherein at least one of the compounds is a common ligand variant of a compound having the formula: 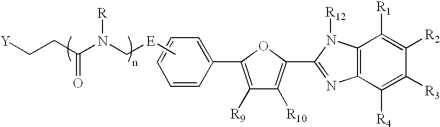
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
Y is OH, NHR₁₅, SH, COOH, SO₂OH, X, CN, N₃, CONH₂, CONHR₁₅, C≡CH, or CH═CH₂;
R is hydrogen, alkyl, alkenyl, alkynyl, aryl, or heterocycle; and
n is an integer between 0 and 5, inclusive.

54. The combinatorial library of claim 41, wherein at least one of the compounds is a common ligand variant of a compound having the formula: 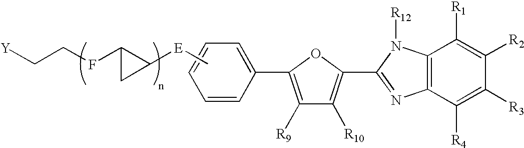
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

55. The combinatorial library of claim 41, wherein at least one of the compounds is a common ligand variant of a compound having the formula: 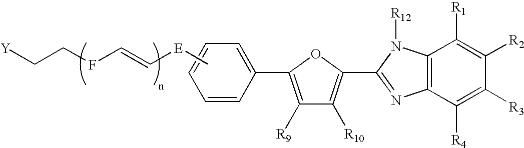
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of CR₁₄C₁₅, CONR₁₅, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

56. The combinatorial library of claim 41, wherein at least one of the compounds is a common ligand variant of a compound having the formula: 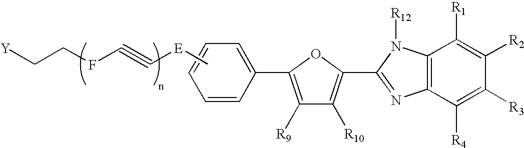
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of CR₁₄C₁₅, CONR₁₅, C≡C, and CH═CH;
Y is OH, NHR₁₅, SH, COOH, SO₂OH, X, CN, N₃, CONH₂, CONHR₁₅, C≡CH, or CH═CH₂; and
n is an integer between 0 and 5, inclusive.

57. The combinatorial library of claim 41, wherein at least one of the compounds is a common ligand variant of a compound having the formula: 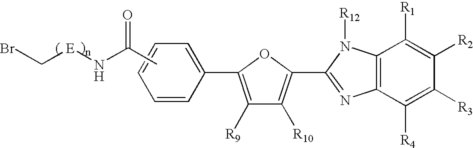
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH; and
n is an integer between 0 and 5, inclusive.

58. The combinatorial library of claim 41, wherein at least one of the compounds is a common ligand variant of a compound having the formula: 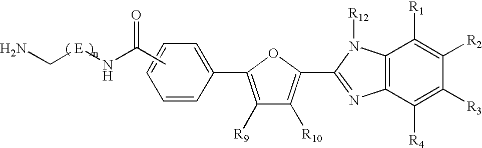
wherein
E is present or absent and when present is O, S, NR₁₅, CR₁₄C₁₅, CONR₁₅, SO₂NR₁₅, NR₁₄CONR₁₅, NR₁₄CNHNR₁₅, NR₁₅COO, C≡C, or CH═CH;
F each independently is selected from the group consisting of O, S, NR₁₅, CR₁₄C₁₅, CONR₁₅, SO₂NR₁₅, NR₁₄CONR₁₅, NR₁₄CNHNR₁₅, NR₁₅COO, C≡C, and CH═CH;
Y is OH, NHR₁₅,SH, COOH, SO₂OH , X, CN, N₃, CONH₂, CONHR₁₅, C≡CH, or CH═CH₂; and
n is an integer between 0 and 5, inclusive.

59. The combinatorial library of claim 58, wherein n is greater than 4 and E is CH₂CH₂OCH or CH₂CH₂SCH.

60. The combinatorial library of claim 41, wherein at least one of the compounds is a common ligand variant of a compound having the formula: [chemical structure]
